(19) United States
(12) Patent Application Publication
(10) Pub. No.: US 2024/0334819 A1
(43) Pub. Date: Oct. 3, 2024

(54) LIGHT EMITTING ELEMENT AND AMINE COMPOUND FOR THE SAME

(71) Applicant: Samsung Display Co., Ltd., Yongin-si (KR)

(72) Inventors: Masatsugu UENO, Yokohama (JP); Ichinori TAKADA, Yokohama (JP)

(73) Assignee: Samsung Display Co., Ltd., Yongin-si (KR)

(21) Appl. No.: 18/620,180

(22) Filed: Mar. 28, 2024

(30) Foreign Application Priority Data
Apr. 3, 2023 (KR) 10-2023-0043532

(51) Int. Cl. H10K 85/60; C09K 11/06; H10K 50/11; H10K 50/15

(52) U.S. Cl. CPC H10K 85/636; C09K 11/06; H10K 50/11; H10K 50/15; H10K 85/6574; H10K 85/6576; C09K 2211/1018

(57) ABSTRACT

Embodiments provide an amine compound and a light emitting element including the amine compound. The light emitting element includes a first electrode, a second electrode disposed on the first electrode, and at least one functional layer disposed between the first electrode and the second electrode and including the amine compound. The amine compound is represented by Formula 1, which is explained in the specification:

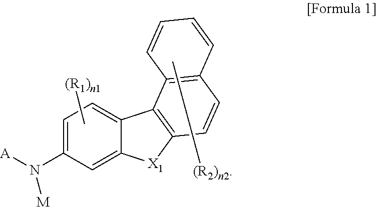

### CROSS-REFERENCE TO RELATED APPLICATION(S)

This application claims priority to and benefits of Korean Patent Application No. 10-2023-0043532 under 35 U.S.C. § 119, filed on Apr. 3, 2023, in the Korean Intellectual Property Office, the entire contents of which are incorporated herein by reference.

### BACKGROUND

### 1. Technical Field

The disclosure relates to a light emitting element and an amine compound for the same.

### 2. Description of the Related Art

Active development continues for an organic electroluminescence display device as an image display device. An organic electroluminescence display device includes a so-called self-luminescent light emitting element in which holes and electrons respectively injected from a first electrode and a second electrode recombine in an emission layer, so that a luminescent material of the emission layer emits light to achieve display.

In the application of a light emitting element to a display device, there is a demand for a light emitting element having high luminous efficiency and a long service life, and continuous development is required on materials for a light emitting element that are capable of stably achieving such characteristics.

Development on materials for a hole transport region having excellent hole transport properties and stability is presently being conducted to contribute to a light emitting element having high efficiency and a long service life.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Embodiments provide a light emitting element exhibiting high efficiency and long service life characteristics and an amine compound included in the light emitting element.

Embodiments provide a light emitting element which may include a first electrode, a second electrode disposed on the first electrode, and at least one functional layer disposed between the first electrode and the second electrode and including an amine compound represented by Formula 1:

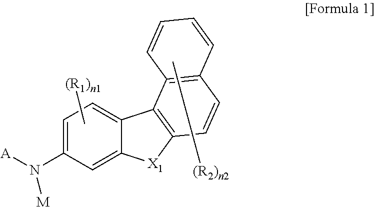

In Formula 1, A may be a group represented by one of Formula 2-1 to Formula 2-3,

M may be a group represented by Formula 3,

X₁ may be O or S,

R₁ and R₂ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,

n1 may be an integer from 0 to 3, and

n2 may be an integer from 0 to 6.

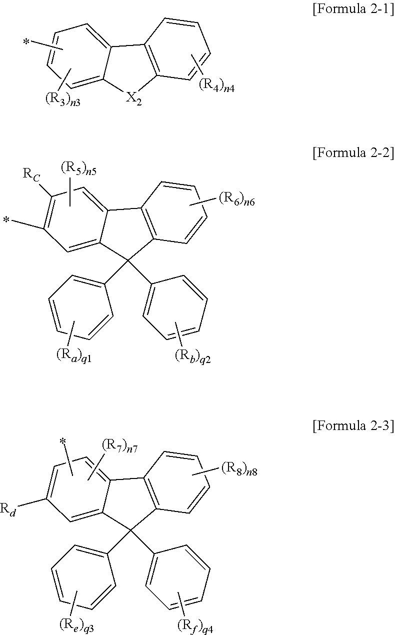

In Formula 2-1 to Formula 2-3,

X₂ may be O or S,

R_{c} may be a hydrogen atom, a deuterium atom, or a substituted or unsubstituted alkyl group having 1 to 20 carbon atoms,

Rₐ, R_{b}, R_{d} to R_{f}, and R₃ to R₈ may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,

n3 may be an integer from 0 to 3,

n4, n6, and n8 may each independently be an integer from 0 to 4,

n5 and n7 may each independently be an integer from 0 to 2,

q1 to q4 each independently be an integer from 0 to 5, and

—* represents a bond to Formula 1.

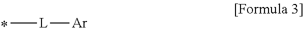

In Formula 3, L may be a direct linkage, a substituted or unsubstituted arylene group having 6 to 30 ring-forming carbon atoms, or a substituted or unsubstituted heteroarylene group having 2 to 30 ring-forming carbon atoms,

Ar may be a substituted or unsubstituted aryl group having 6 to 30 ring-forming carbon atoms, or a substituted or unsubstituted heteroaryl group having 2 to 30 ring-forming carbon atoms and 1 or 2 ring-forming heteroatoms, a group represented by Formula 2-2, or a group represented by Formula 2-3, and

—* may represent a bond to Formula 1,

wherein in the amine compound represented by Formula 1,

a case where the amine compound includes an aromatic fused ring containing sp³ carbon in addition to sp³ carbon contained in a fluorene skeleton represented by Formula 2-2 or Formula 2-3 is excluded,

a case where the amine compound includes a nitrogen-containing heterocycle and a halogen atom is excluded, and

a case where M is a group represented by Formula a-1 or Formula a-2 is excluded.

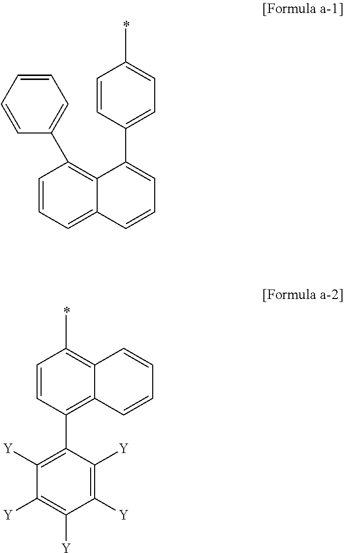

In Formula a-1 and Formula a-2,

Y may be a hydrogen atom or a deuterium atom, and

—* represents a bond to Formula 1.

In Formula 1,

when A is a group represented by Formula 2-2 or Formula 2-3, M does not include a naphthylene moiety,

when A is a group represented by Formula 2-1, and the group represented by Formula 2-1 is linked to N in Formula 1 at carbon 4, n1 is 0,

when A is a group represented by Formula 2-2 or Formula 2-3, n1 is 0,

when A is a group represented by Formula 2-1, X₂ in Formula 2-1 is S, and the group represented by Formula 2-1 is linked to N in Formula 1 at carbon 1, a case where L is a direct linkage or a group represented by Formula a-3 is excluded:

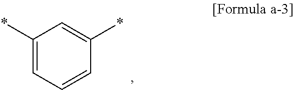 [SMILES: *c1cccc(*)c1]

and

when A is a group represented by Formula 2-1, X₂ in Formula 2-1 is O, and the group represented by Formula 2-1 is linked to N in Formula 1 at carbon 2 or carbon 4, a case where M in Formula 1 is an unsubstituted phenyl group or a phenyl group having a substituent having 0 to 9 carbon atoms is excluded.

In an embodiment, the at least one functional layer may include an emission layer, a hole transport region disposed between the first electrode and the emission layer, and an electron transport region disposed between the emission layer and the second electrode; and the hole transport region may include the amine compound.

In an embodiment, the hole transport region may include a hole injection layer disposed on the first electrode, and the hole transport layer disposed on the hole injection layer; and the hole transport layer may include the amine compound.

In an embodiment, a functional layer that is included in the hole transport region may be adjacent to the emission layer and may include the amine compound.

In an embodiment, the amine compound may be a monoamine compound.

In an embodiment, the amine compound may be represented by one of Formula 1-1-1 to Formula 1-1-3:

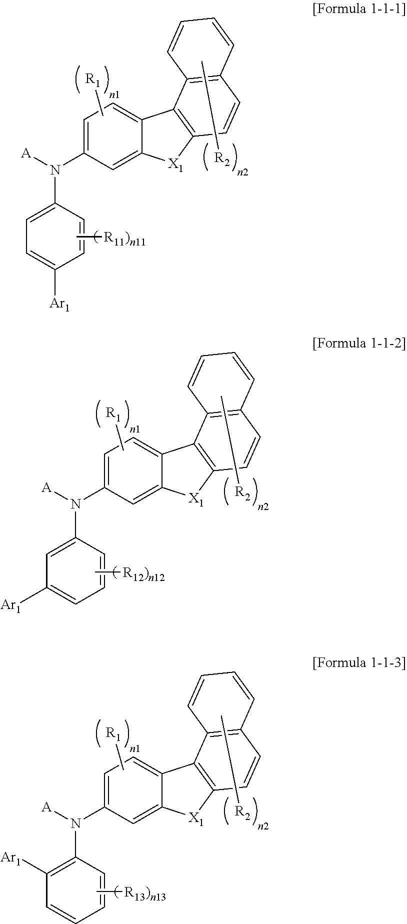

In Formula 1-1-1 to Formula 1-1-3,

R₁₁ to R₁₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,

n₁₁ to n13 may each independently be an integer from 0 to 4, and

Ar₁ may be a group represented by one of Formula A-1 to Formula A-4:

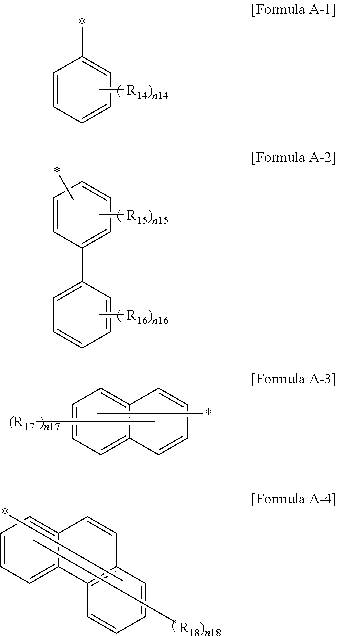

In Formula A-1 to Formula A-4, R₁₄ to R₁₈ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; n14 and n16 may each independently be an integer from 0 to 5; n15 may be an integer from 0 to 4; n17 may be an integer from 0 to 7; and n18 may be an integer from 0 to 9.

In Formula 1-1-1 to Formula 1-1-3, when A is a group represented by Formula 2-2 or Formula 2-3, Ar₁ may be a group represented by Formula A-1, Formula A-2, or Formula A-4, and

A, X₁, R₁, R₂, n1, and n2 are the same as defined in Formula 1.

In an embodiment, the amine compound may be represented by one of Formula 1-2-1 to Formula 1-2-7:

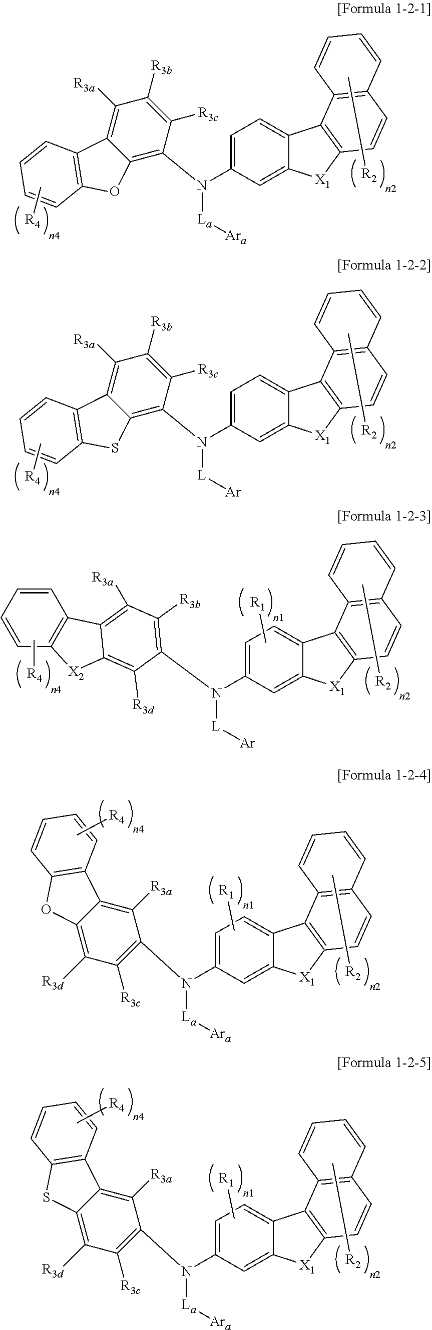

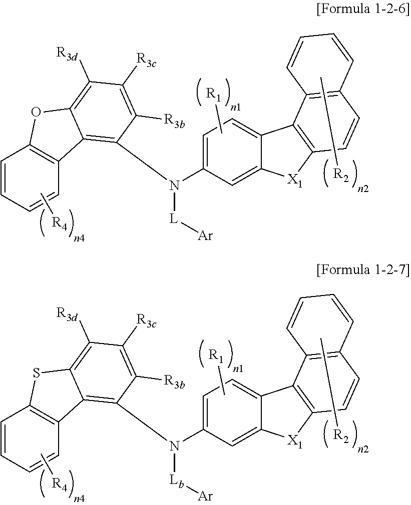

In Formula 1-2-1 to Formula 1-2-7, R₃ₐ to R_{3d} may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; Lₐ may be a substituted or unsubstituted arylene group having 6 to 30 ring-forming carbon atoms or a substituted or unsubstituted heteroarylene group having 2 to 30 ring-forming carbon atoms; and Arₐ may be a substituted or unsubstituted aryl group having 6 to 30 ring-forming carbon atoms, or a substituted or unsubstituted heteroaryl group having 2 to 30 ring-forming carbon atoms, except that Arₐ may not be a substituted or unsubstituted phenyl group.

In Formula 1-2-7, L_{b} may be a substituted or unsubstituted arylene group having 6 to 30 ring-forming carbon atoms or a substituted or unsubstituted heteroarylene group having 2 to 30 ring-forming carbon atoms; and a case where L_{b} is a group represented by Formula a-3 is excluded.

In Formula 1-2-1 to Formula 1-2-7, L, Ar, X₁, R₁, R₂, n1, n2, X₂, R₄, and n4 are the same as defined in Formula 1 and Formula 2.

In an embodiment, the amine compound may be represented by one of Formula 1-3-1 to Formula 1-3-4:

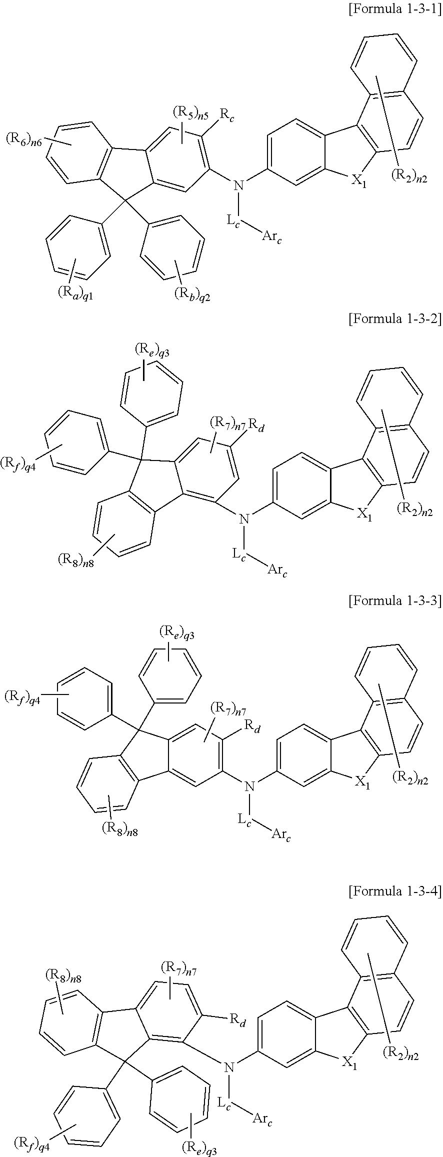

In Formula 1-3-1 to Formula 1-3-4, L_{c} may be a direct linkage, a substituted or unsubstituted arylene group having 6 to 30 ring-forming carbon atoms, or a substituted or unsubstituted heteroarylene group having 2 to 30 ring-forming carbon atoms, except that L_{c} may not be a substituted or unsubstituted naphthalene group; Ar_{c} may be a substituted or unsubstituted aryl group having 6 to 30 ring-forming carbon atoms or a substituted or unsubstituted heteroaryl group having 2 to 30 ring-forming carbon atoms, except that Ar_{c} may not be a substituted or unsubstituted naphthalene group.

In Formula 1-3-1 to Formula 1-3-4, X₁, R₂, n2, R₅ to R₈, n5 to n8, Rₐ to R_{f}, and q1 to q4 are the same as defined in Formula 1, Formula 2-2, and Formula 2-3.

In an embodiment, the amine compound may be represented by Formula 1-4-1 or Formula 1-4-2:

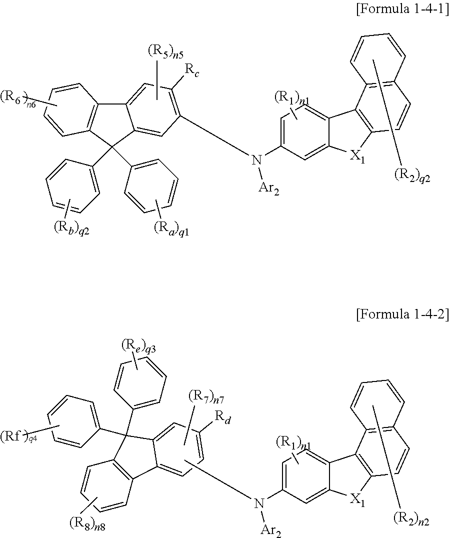

In Formula 1-4-1 and Formula 1-4-2, Ar₂ may be a group represented by one of Formula B-1 to Formula B-7:

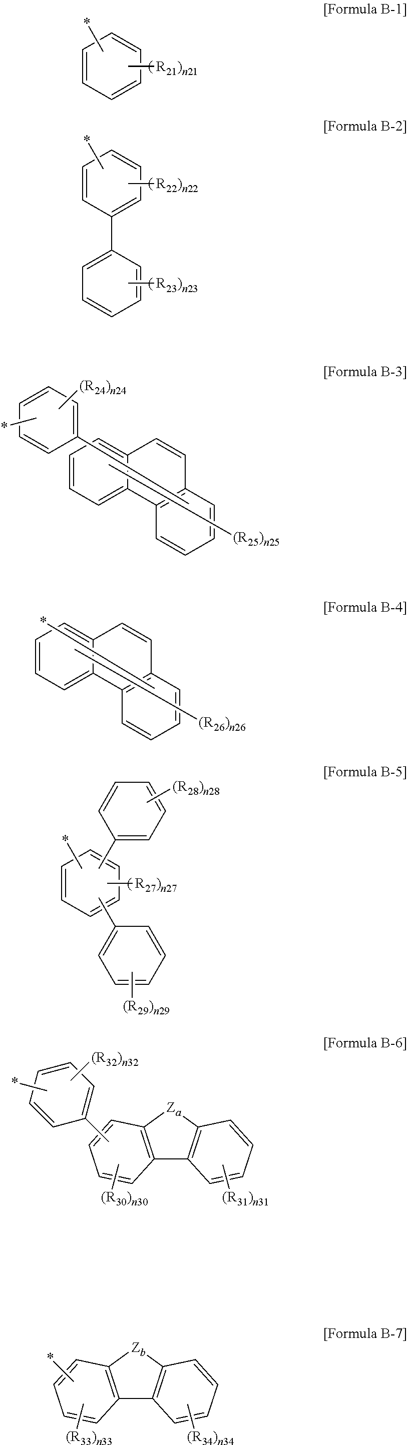

In Formula B-1 to Formula B-7, Zₐ and Z_{b}, may each independently be O or S; R₂₁ to R₃₄ may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring; n21, n23, n28, and n29 may each independently be an integer from 0 to 5; n22, n24, n31, n32, and n34 may each independently be an integer from 0 to 4; n25 and n26 may each independently be an integer from 0 to 9; and n27, n30, and n33 may each independently an integer from 0 to 3.

In Formula 1-4-1 and Formula 1-4-2, X₁, R₁, R₂, n1, n2, R₅ to R₈, n5 to n8, Rₐ to R_{f}, and q1 to q4 are the same as defined in Formula 1, Formula 2-2, and Formula 2-3.

In an embodiment, the amine compound may be represented by Formula 1-5-1 or Formula 1-5-2:

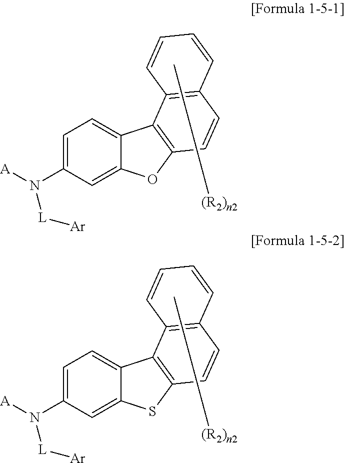

In Formula 1-5-1 and Formula 1-5-2, A, L, Ar, R₂, and n2 are the same as defined in Formula 1.

In an embodiment, the amine compound may be represented by one of Formula 1-6-1 to Formula 1-6-4:

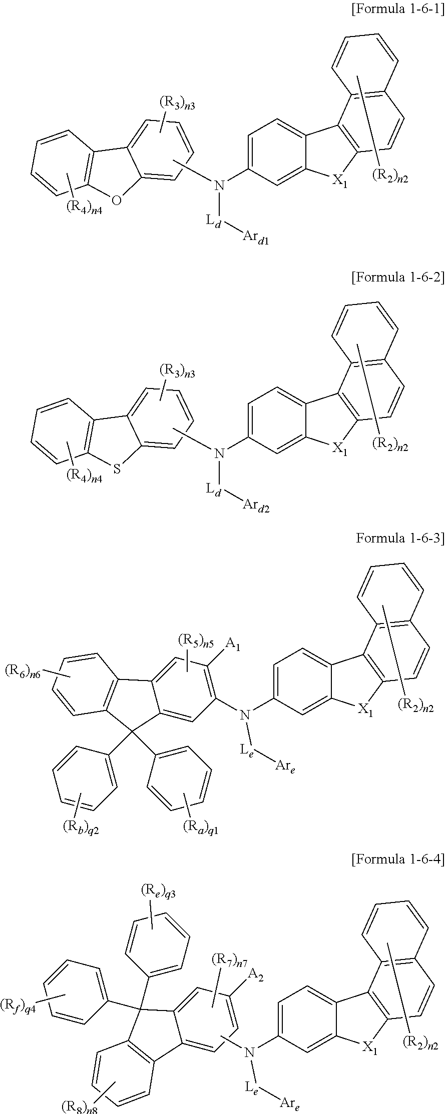

In Formula 1-6-1 to Formula 1-6-4, A₁ and A₂ may each independently be a hydrogen atom or a deuterium atom; L_{d} may be a group represented by one of Formula L-1 to Formula L-5; Ar_{d1} may be a group represented by Formula 2-2, a group represented by Formula 2-3, a substituted or unsubstituted naphthyl group, a substituted or unsubstituted anthracene group, a substituted or unsubstituted phenanthrene group, a substituted or unsubstituted dibenzofuran group, or a substituted or unsubstituted dibenzothiophene group; Ar_{d2} may be a group represented by Formula 2-2, a group represented by Formula 2-3, a substituted or unsubstituted phenyl group, a substituted or unsubstituted biphenyl group, a substituted or unsubstituted naphthyl group, a substituted or unsubstituted phenanthrene group, a substituted or unsubstituted anthracene group, a substituted or unsubstituted dibenzofuran group, or a substituted or unsubstituted dibenzothiophene group; Lₑ may be a direct linkage, or a group represented by one of Formula L-1 to Formula L-5; and Arₑ may be a group represented by Formula 2-2, a group represented by Formula 2-3 or a group represented by one of Formula C-1 to Formula C-5:

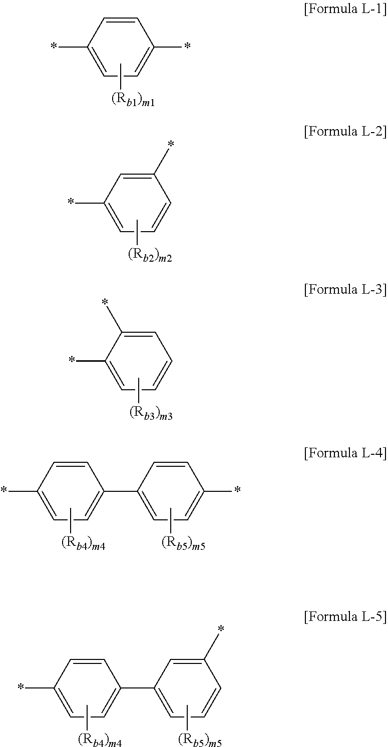

In Formula L-1 to Formula L-5, R_{b1} to R_{b7} may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; and m1 to m7 may each independently be an integer from 0 to 4.

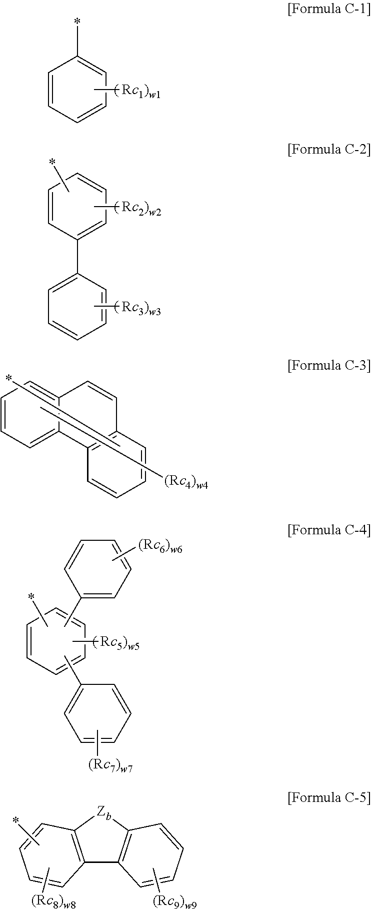

In Formula C-1 to Formula C-5, Z_{c} may be O or S; R_{c1} to R_{c9} may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring; w1, w3, w6, and w7 may each independently be an integer from 0 to 5; w2 and w9 may each independently be an integer from 0 to 4; w4 may be an integer from 0 to 9; and w5 and w8 may each independently be an integer from 0 to 3.

In Formula 1-6-1 to Formula 1-6-4, X₁, R₂ to R₈, n2 to n8, Rₐ, R_{b}, Rₑ, R_{f}, and q1 to q4 are the same as defined in Formula 1, Formula 2-2, and Formula 2-3.

In an embodiment, in Formula 1, M may be a group selected from Substituent Group B; and Ar may be a group selected from Substituent Group C:

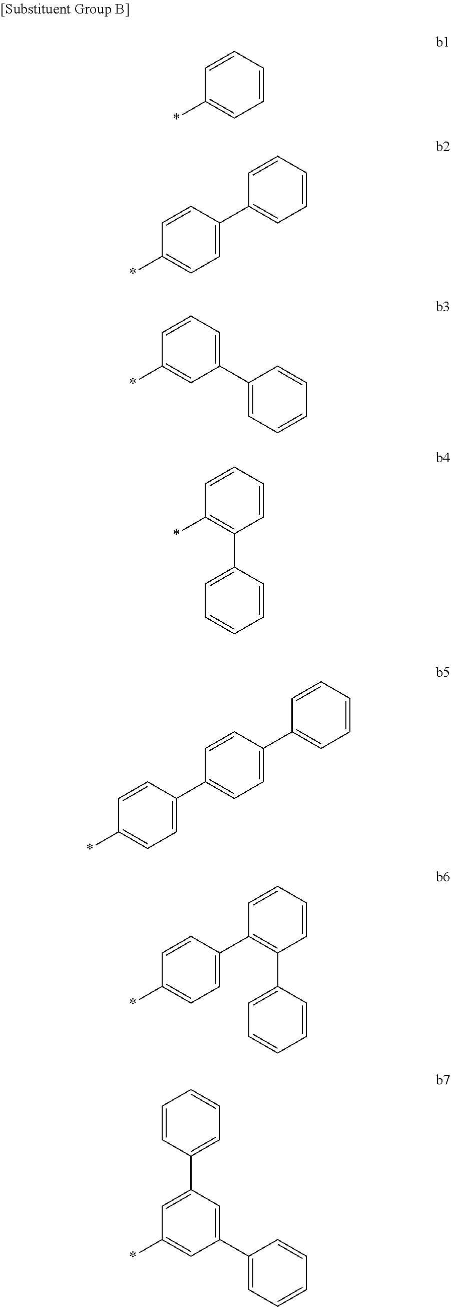

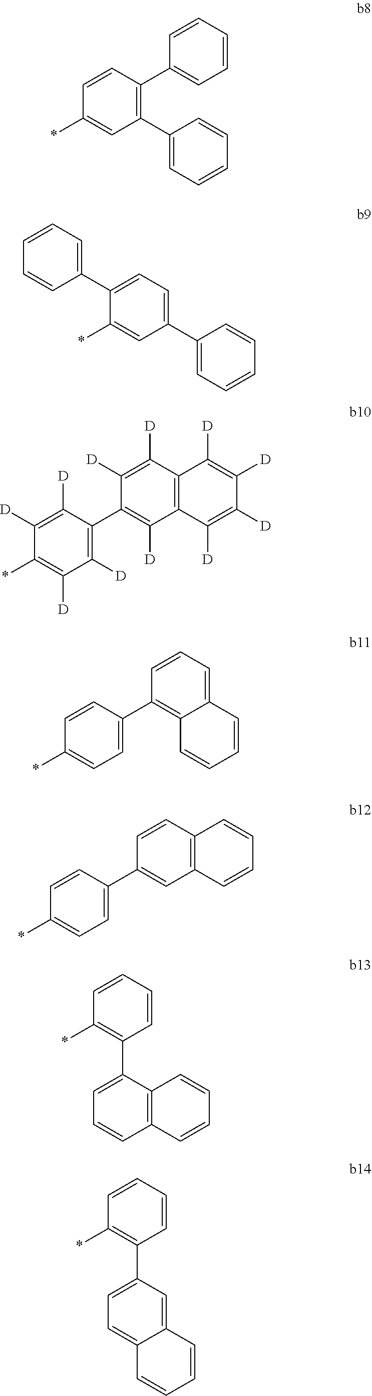

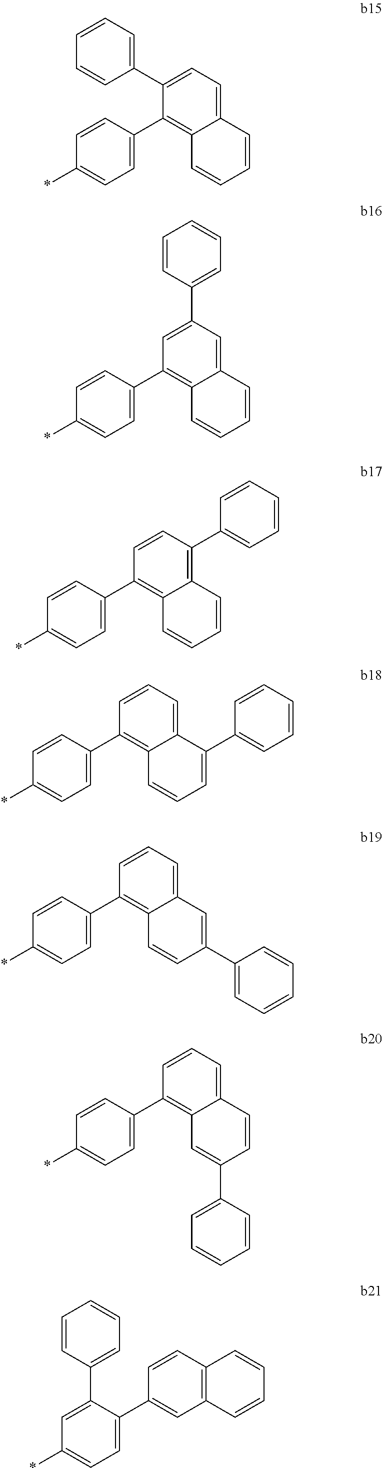

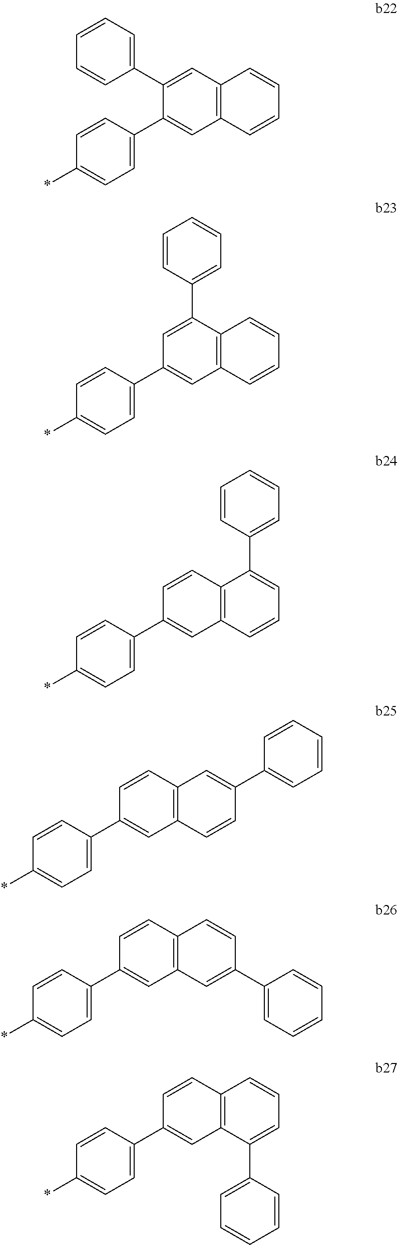 [SMILES: *c1ccc(-c2cc3ccccc3cc2-c2ccccc2)cc1 ; *c1ccc(-c2cc(-c3ccccc3)c3ccccc3c2)cc1 ; *c1ccc(-c2ccc3c(-c4ccccc4)cccc3c2)cc1 ; *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1]

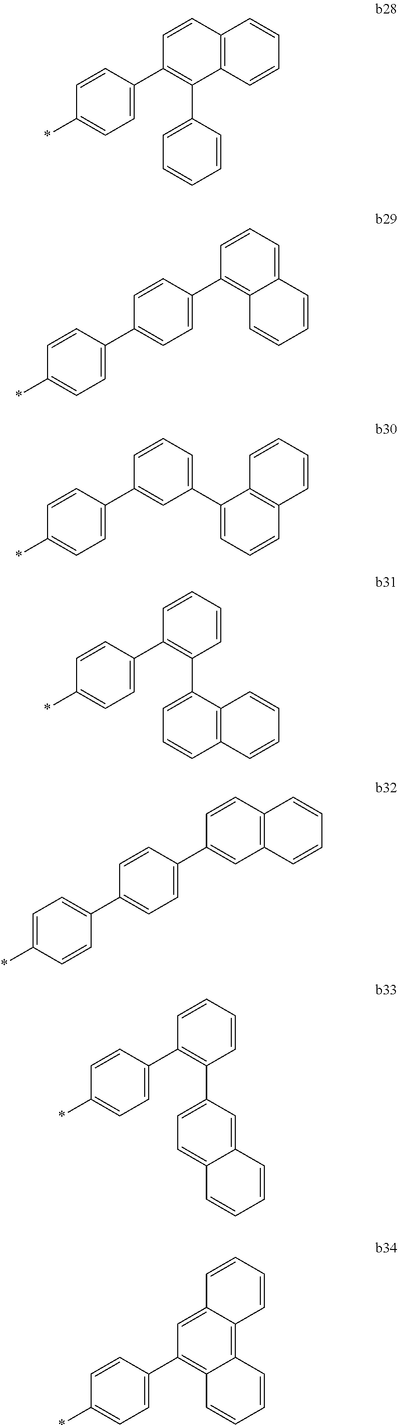

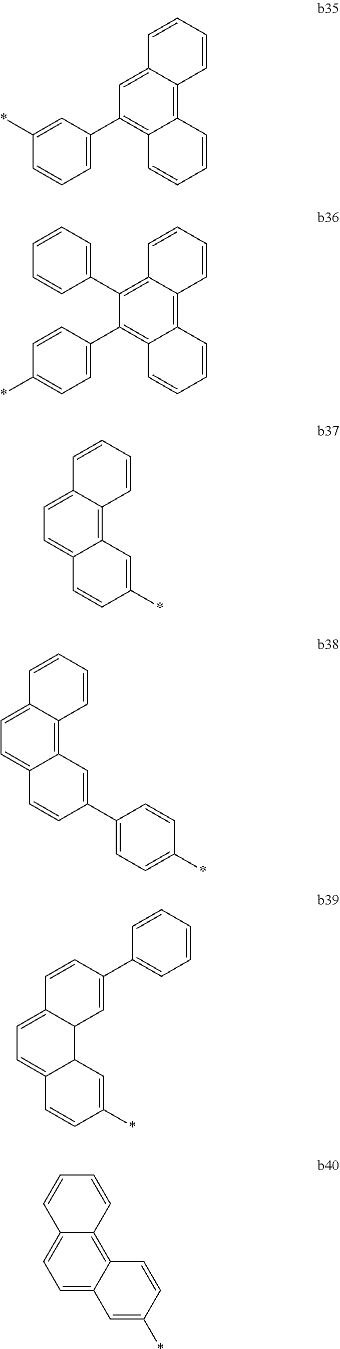

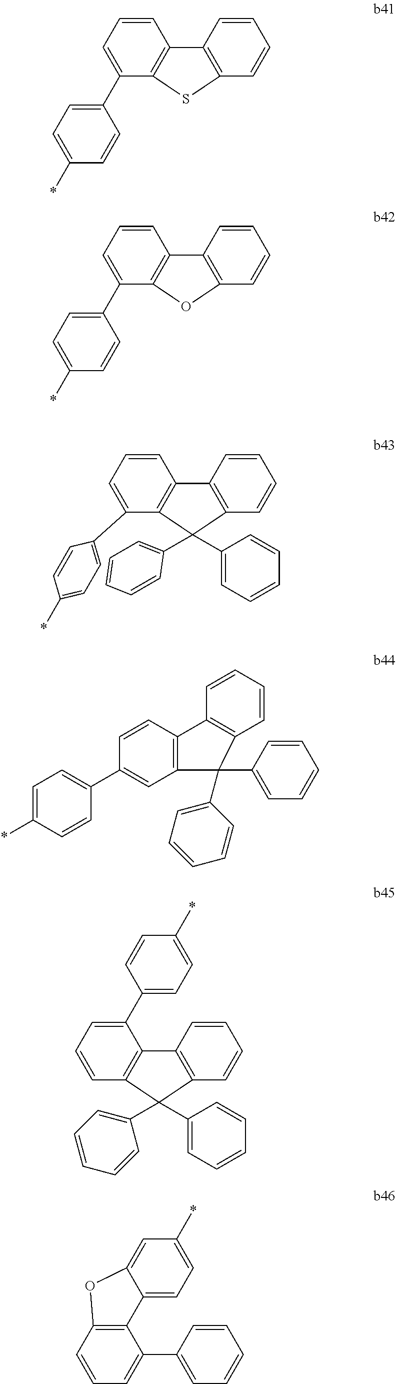

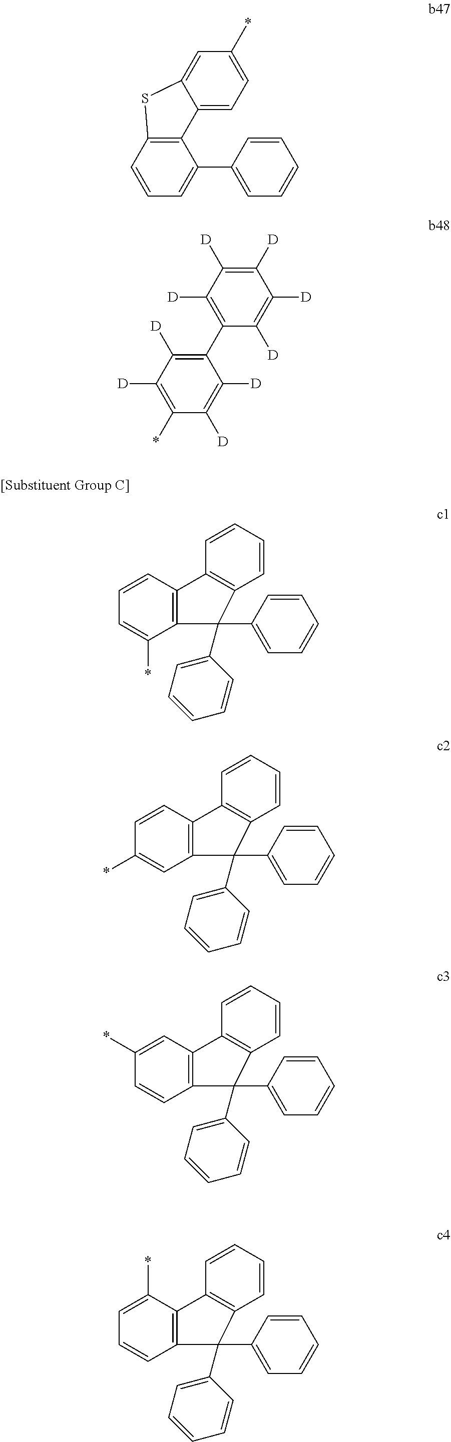

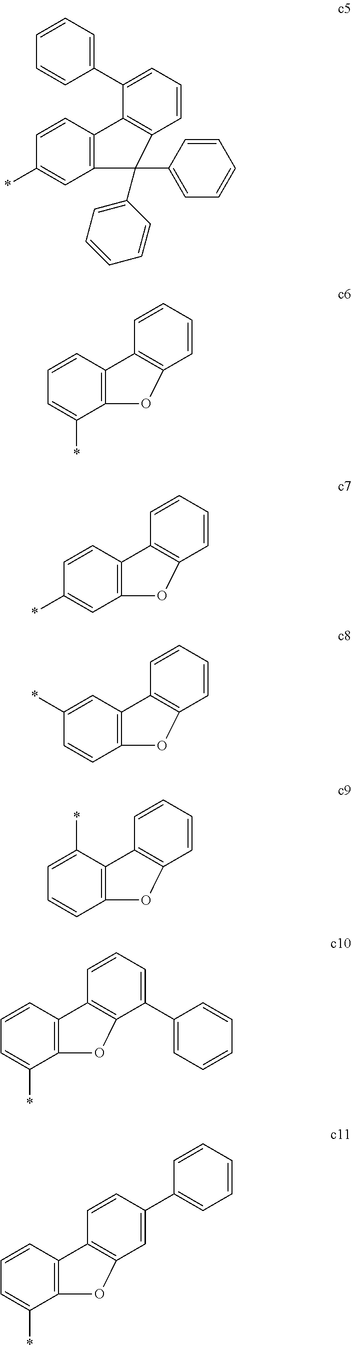

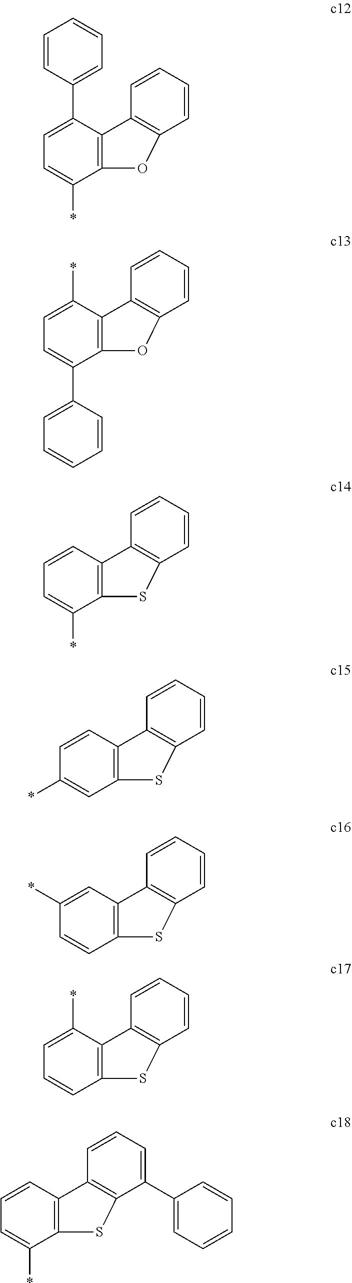

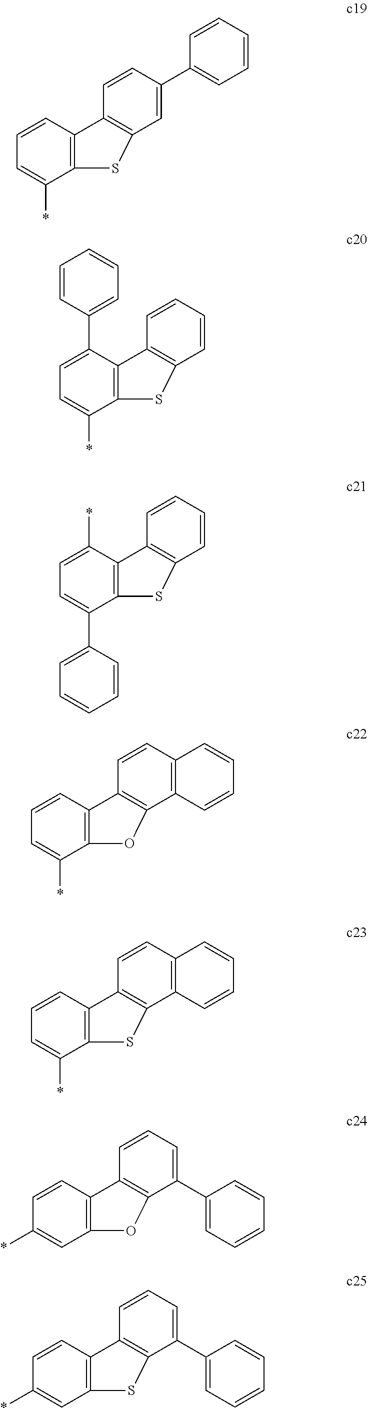

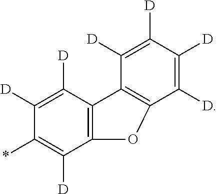

In an embodiment, the amine compound may be represented by Formula 1-7; and the amine compound may meet one of the combinations in Compound Combination Table 1:

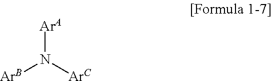

In Formula 1-7, Ar^{A} may be a group selected from Substituent Group A; Ar^{B} may be a group selected from Substituent Group B; and Ar^{C} may be a group selected from Substituent Group C.

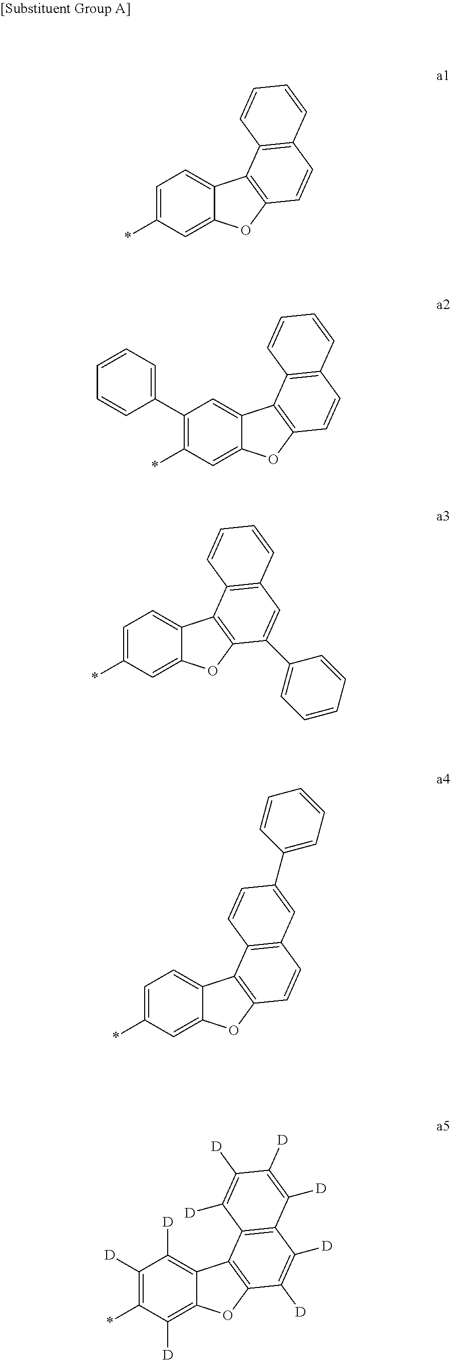 [SMILES: *c1ccc2c(c1)oc1ccc3ccccc3c12 ; *c1cc2oc3ccc4ccccc4c3c2cc1-c1ccccc1 ; *c1ccc2c(c1)oc1c(-c3ccccc3)cc3ccccc3c12 ; *c1ccc2c(c1)oc1ccc3cc(-c4ccccc4)ccc3c12 ; *c1c([2H])c([2H])c2c(oc3c([2H])c([2H])c4c([2H])c([2H])c([2H])c([2H])c4c32)c1[2H]]

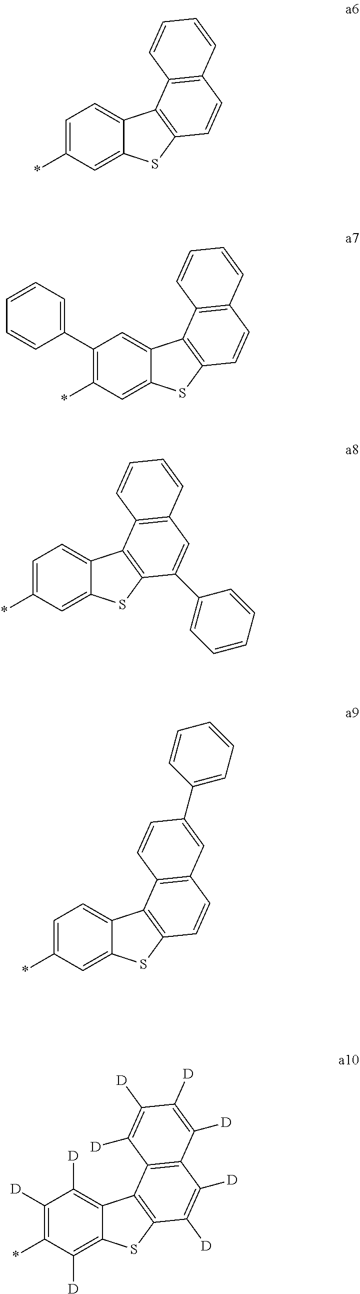 [SMILES: *c1ccc2c(c1)sc1ccc3ccccc3c12 ; *c1cc2sc3ccc4ccccc4c3c2cc1-c1ccccc1 ; *c1ccc2c(c1)sc1c(-c3ccccc3)cc3ccccc3c12 ; *c1ccc2c(c1)sc1ccc3cc(-c4ccccc4)ccc3c12 ; *c1c([2H])c([2H])c2c(sc3c([2H])c([2H])c4c([2H])c([2H])c([2H])c([2H])c4c32)c1[2H]]

Compound Combination Table 1 is explained below.

Embodiments provide an amine compound which may be represented by Formula 1, which is explained herein.

In an embodiment, the amine compound represented by Formula 1 may be represented by one of Formula 1-1-1 to Formula 1-1-3, which are explained herein.

In an embodiment, the amine compound represented by Formula 1 may be represented by one of Formula 1-2-1 to Formula 1-2-7, which are explained herein.

In an embodiment, the amine compound represented by Formula 1 may be represented by one of Formula 1-3-1 to Formula 1-3-4, which are explained herein.

In an embodiment, the amine compound represented by Formula 1 may be represented by Formula 1-4-1 or Formula 1-4-2, which are explained herein.

In an embodiment, the amine compound represented by Formula 1 may be represented by one of Formula 1-6-1 to Formula 1-6-4, which are explained herein.

In an embodiment, the amine compound represented by Formula 1 may be represented by Formula 1-7; and the amine compound may meet one of the combinations in Compound Combination Table 1. Formula 1-7 is explained herein, and Compound Combination Table 1 is explained below.

It is to be understood that the embodiments above are described in a generic and explanatory sense only and not for the purpose of limitation, and the disclosure is not limited to the embodiments described above.

### BRIEF DESCRIPTION OF THE FIGURES

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a schematic plan view of a display device according to an embodiment;

FIG. 2 is a schematic cross-sectional view of the display device according to the embodiment;

FIG. 3 is a schematic cross-sectional view of a light emitting element according to an embodiment;

FIG. 4 is a schematic cross-sectional view of a light emitting element according to an embodiment;

FIG. 5 is a schematic cross-sectional view of a light emitting element according to an embodiment;

FIG. 6 is a schematic cross-sectional view of a light emitting element according to an embodiment;

FIG. 7 is a schematic cross-sectional view of a display device according to an embodiment;

FIG. 8 is a schematic cross-sectional view of a display device according to an embodiment;

FIG. 9 is a schematic cross-sectional view of a display device according to an embodiment;

FIG. 10 is a schematic cross-sectional view of a display device according to an embodiment; and

FIG. 11 is a schematic diagram of a vehicle in which display devices are disposed according to embodiments.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and/or like reference characters refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may describe a group that is substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the term “bonded to an adjacent group to form a ring” may be interpreted as a group that is bonded to an adjacent group to form a substituted or unsubstituted hydrocarbon ring or a substituted or unsubstituted heterocycle. The hydrocarbon ring may be an aliphatic hydrocarbon ring or an aromatic hydrocarbon ring. The heterocycle may be an aliphatic heterocycle or an aromatic heterocycle. The hydrocarbon ring and the heterocycle may each independently be monocyclic or polycyclic. A ring that is formed by adjacent groups being bonded to each other may itself be connected to another ring to form a spiro structure.

In the specification, the term “adjacent group” may be interpreted as a substituent that is substituted for an atom which is directly linked to an atom substituted with a corresponding substituent, as another substituent that is substituted for an atom which is substituted with a corresponding substituent, or as a substituent that is sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, or an iodine atom.

In the specification, an alkyl group may be linear or branched. The number of carbon atoms in an alkyl group may be 1 to 50, 1 to 30, 1 to 20, 1 to 10, or 1 to 6. Examples of an alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a i-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a cycloalkyl group may be a cyclic alkyl group. The number of carbon atoms in a cycloalkyl group may be 3 to 50, 3 to 30, 3 to 20, or 3 to 10. Examples of a cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments are not limited thereto.

In the specification, an alkenyl group may be a hydrocarbon group that includes at least one carbon-carbon double bond in the middle or at a terminus of an alkyl group having 2 or more carbon atoms. An alkenyl group may be linear or branched. The number of carbon atoms in an alkenyl group is not particularly limited, but may be 2 to 30, 2 to 20, or 2 to 10. Examples of an alkenyl group may include a vinyl group, a 1-butenyl group, a 1-pentenyl group, a 1,3-butadienyl aryl group, a styrenyl group, a styryl vinyl group, etc., but embodiments are not limited thereto.

In the specification, an alkynyl group may be a hydrocarbon group including at least one carbon-carbon triple bond in the middle or at a terminus of an alkyl group having 2 or more carbon atoms. An alkynyl group may be linear or branched. The number of carbon atoms in an alkynyl group is not particularly limited, but may be 2 to 30, 2 to 20, or 2 to 10. Examples of an alkynyl group may include an ethynyl group, a propynyl group, etc., but embodiments are not limited thereto.

In the specification, a hydrocarbon ring group may be any functional group or substituent derived from an aliphatic hydrocarbon ring. For example, a hydrocarbon ring group may be a saturated hydrocarbon ring group having 5 to 20 ring-forming carbon atoms.

In the specification, an aryl group may be any functional group or substituent derived from an aromatic hydrocarbon ring. An aryl group may be monocyclic or polycyclic. The number of ring-forming carbon atoms in an aryl group may be 6 to 30, 6 to 20, or 6 to 15. Examples of an aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments are not limited thereto.

In the specification, a fluorenyl group may be substituted, and two substituents may be bonded to each other to form a spiro structure. Examples of a substituted fluorenyl group may include the groups shown below. However, embodiments are not limited thereto.

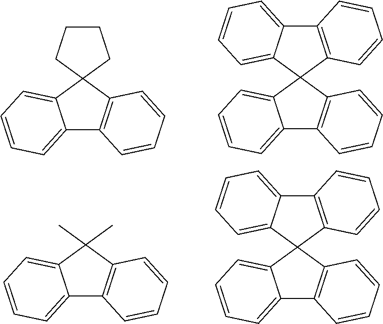

In the specification, a heterocyclic group may be any functional group or substituent derived from a ring that includes at least one of B, O, N, P, Si, S, and Se as a heteroatom. A heterocyclic group may be aliphatic or aromatic. An aromatic heterocyclic group may be a heteroaryl group. An aliphatic heterocycle and an aromatic heterocycle may each independently be monocyclic or polycyclic.

In the specification, a heterocyclic group may include at least one of B, O, N, P, Si, S, and Se as a heteroatom. If a heterocyclic group includes two or more heteroatoms, the two or more heteroatoms may be the same as or different from each other. A heterocyclic group may be monocyclic or polycyclic. A heterocyclic group may be a heteroaryl group. The number of ring-forming carbon atoms in a heterocyclic group may be 2 to 30, 2 to 20, or 2 to 10.

In the specification, an aliphatic heterocyclic group may include at least one of B, O, N, P, Si, S, and Se as a heteroatom. The number of ring-forming carbon atoms in an aliphatic heterocyclic group may be 2 to 30, 2 to 20, or 2 to 10. Examples of an aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

In the specification, a heteroaryl group may include at least one of B, O, N, P, Si, S, and Se as a heteroatom. If a heteroaryl group includes two or more heteroatoms, the two or more heteroatoms may be the same as or different from each other. A heteroaryl group may be monocyclic or polycyclic. The number of ring-forming carbon atoms in a heteroaryl group may be 2 to 30, 2 to 20, or 2 to 10. Examples of a heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments are not limited thereto.

In the specification, the above description of an aryl group may be applied to an arylene group, except that an arylene group is a divalent group. In the specification, the above description of a heteroaryl group may be applied to a heteroarylene group, except that a heteroarylene group is a divalent group.

In the specification, a silyl group may be an alkylsilyl group or an arylsilyl group. Examples of a silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments are not limited thereto.

In the specification, the number of carbon atoms in a carbonyl group is not particularly limited, but may be 1 to 40, 1 to 30, or 1 to 20. For example, a carbonyl group may have one of the following structures, but embodiments are not limited thereto.

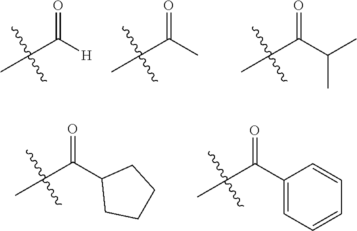

In the specification, the number of carbon atoms in a sulfinyl group or a sulfonyl group is not particularly limited, but may be 1 to 30. A sulfinyl group may be an alkyl sulfinyl group or an aryl sulfinyl group. A sulfonyl group may be an alkyl sulfonyl group or an aryl sulfonyl group.

In the specification, a thio group may be an alkylthio group or an arylthio group. A thio group may be a sulfur atom that is bonded to an alkyl group or an aryl group as defined above. Examples of a thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments are not limited thereto.

In the specification, an oxy group may be an oxygen atom that is bonded to an alkyl group or an aryl group as defined above. An oxy group may be an alkoxy group or an aryl oxy group. An alkoxy group may be linear, branched, or cyclic. The number of carbon atoms in an alkoxy group is not particularly limited, but may be, for example, 1 to 20 or 1 to 10. Examples of an oxy group may include a methoxy group, an ethoxy group, an n-propoxy group, an isopropoxy group, a butoxy group, a pentyloxy group, a hexyloxy group, an octyloxy group, a nonyloxy group, a decyloxy group, a benzyloxy group, etc., but embodiments are not limited thereto.

In the specification, a boron group may be a boron atom that is bonded to an alkyl group or an aryl group as defined above. A boron group may be an alkyl boron group or an aryl boron group. Examples of a boron group may include a dimethylboron group, a trimethylboron group, a t-butyldimethylboron group, a diphenylboron group, a phenylboron group, etc., but embodiments are not limited thereto.

In the specification, the number of carbon atoms in an amine group is not particularly limited, but may be 1 to 30. An amine group may be an alkyl amine group or an aryl amine group. Examples of an amine group may include a methylamine group, a dimethylamine group, a phenylamine group, a diphenylamine group, a naphthylamine group, a 9-methyl-anthracenylamine group, etc., but embodiments are not limited thereto.

In the specification, an alkyl group within an alkylthio group, an alkylsulfoxy group, an alkylaryl group, an alkylamino group, an alkyl boron group, an alkyl silyl group, or an alkyl amine group may be the same as an example of an alkyl group as described above.

In the specification, an aryl group within an aryloxy group, an arylthio group, an arylsulfoxy group, an arylamino group, an arylboron group, an arylsilyl group, or an arylamine group may be the same as an example of an aryl group as described above.

In the specification, a direct linkage may be a single bond.

In the specification, the symbols

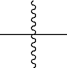

and —* each represent a bond to a neighboring atom in a corresponding formula or moiety.

Hereinafter, embodiments will be described with reference to the accompanying drawings.

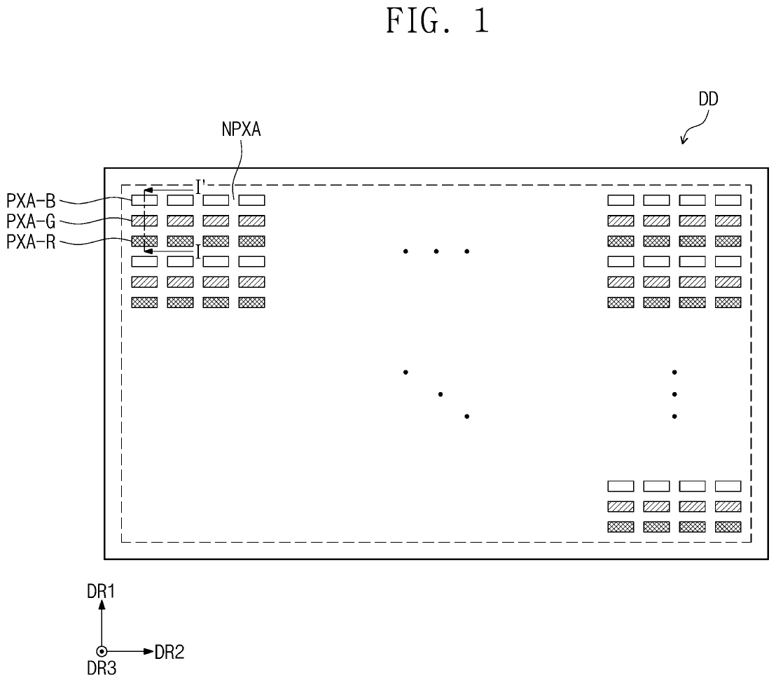

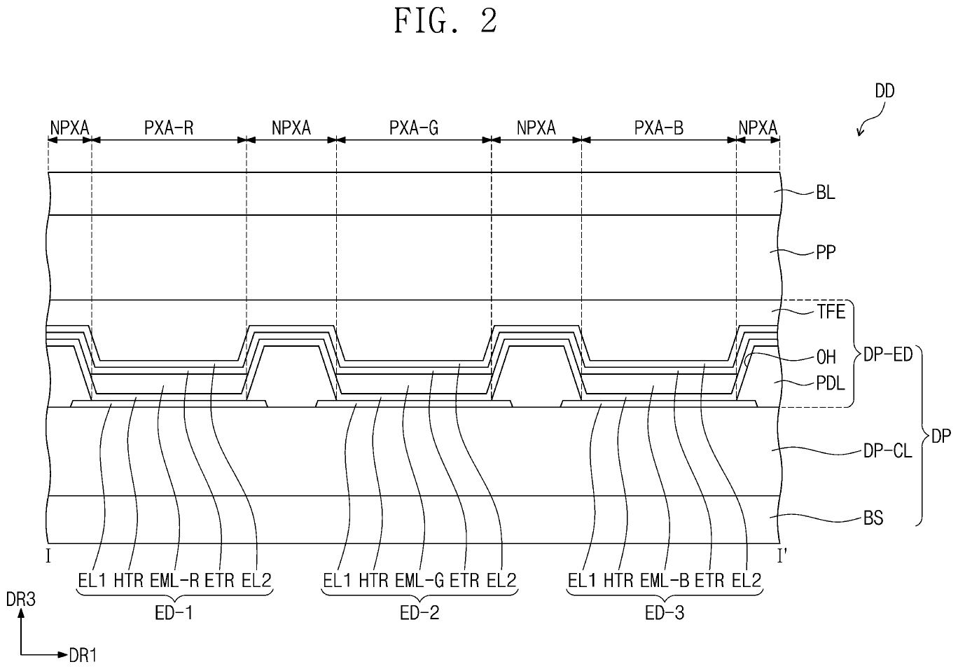

FIG. 1 is a schematic plan view of an embodiment of a display device DD. FIG. 2 is a schematic cross-sectional view of the display device DD according to an embodiment. FIG. 2 is a schematic cross-sectional view illustrating a part taken along line I-I′ of FIG. 1.

The display device DD may include a display panel DP and an optical layer PP disposed on the display panel DP. The display panel DP includes light emitting elements ED-1, ED-2, and ED-3. The display device DD may include multiples of each of the light emitting elements ED-1, ED-2, and ED-3. The optical layer PP may be disposed on the display panel DP and may control light that is reflected at the display panel DP from an external light. The optical layer PP may include, for example, a polarization layer or a color filter layer. Although not shown in the drawings, in an embodiment, the optical layer PP may be omitted from the display device DD.

A base substrate BL may be disposed on the optical layer PP. The base substrate BL may provide a base surface on which the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display device DD according to an embodiment may further include a filling layer (not shown). The filling layer (not shown) may be disposed between a display device layer DP-ED and the base substrate BL. The filling layer (not shown) may be an organic material layer. The filling layer (not shown) may include at least one of an acrylic-based resin, a silicone-based resin, and an epoxy-based resin.

The display panel DP may include a base layer BS, a circuit layer DP-CL provided on the base layer BS, and the display device layer DP-ED. The display device layer DP-ED may include a pixel defining film PDL, light emitting elements ED-1, ED-2, and ED-3 disposed between portions of the pixel defining film PDL, and an encapsulation layer TFE disposed on the light emitting elements ED-1, ED-2, and ED-3.

The base layer BS may provide a base surface on which the display device layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). The transistors (not shown) may each include a control electrode, an input electrode, and an output electrode. For example, the circuit layer DP-CL may include a switching transistor and a driving transistor for driving the light emitting elements ED-1, ED-2, and ED-3 of the display device layer DP-ED.

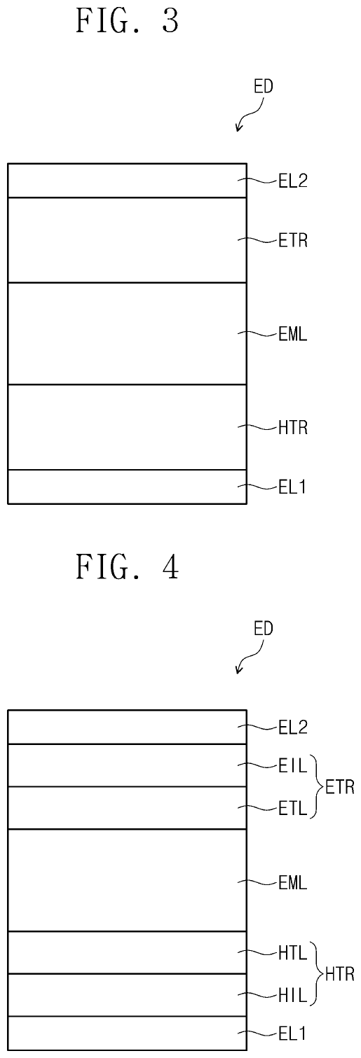

The light emitting elements ED-1, ED-2, and ED-3 may each have a structure of a light emitting element ED of an embodiment according to any of FIGS. 3 to 6, which will be described later. The light emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are disposed in openings OH defined in the pixel defining film PDL, and a hole transport region HTR, an electron transport region ETR, and a second electrode EL2 are each provided as a common layer for the light emitting elements ED-1, ED-2, and ED-3. However, embodiments are not limited thereto, Although not shown in FIG. 2, in an embodiment, the hole transport region HTR and the electron transport region ETR may each be provided by being patterned in the openings OH defined in the pixel defining film PDL. For example, in an embodiment, the hole transport region HTR, the emission layers EML-R, EML-G, and EML-B, and the electron transport region ETR of the light emitting elements ED-1, ED-2, and ED-3 may each be provided by being patterned by an inkjet printing method.

The encapsulation layer TFE may cover the light emitting elements ED-1, ED-2, and ED-3. The encapsulation layer TFE may seal the display device layer DP-ED. The encapsulation layer TFE may be a thin film encapsulation layer. The encapsulation layer TFE may be formed of a single layer or multiple layers. The encapsulation layer TFE may include at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display device layer DP-ED from moisture and/or oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments are not limited thereto.

The encapsulation layer TFE may be disposed on the second electrode EL2 and may be disposed to fill the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region that emits light respectively generated by the light emitting devices ED-1, ED-2, and ED-3. The light emitting regions PXA-R, PXA-G, and PXA-B may be spaced apart from each other in a plan view.

The light emitting regions PXA-R, PXA-G, and PXA-B may each be a region separated by the pixel defining film PDL. The non-light emitting regions NPXA may be areas between the adjacent light emitting regions PXA-R, PXA-G, and PXA-B, and which may correspond to the pixel defining film PDL. In an embodiment, the light emitting regions PXA-R, PXA-G, and PXA-B may each correspond to a pixel. The pixel defining film PDL may separate the light emitting elements ED-1, ED-2, and ED-3. The emission layers EML-R, EML-G, and EML-B of the light emitting elements ED-1, ED-2, and ED-3 may be disposed in openings OH defined in the pixel defining film PDL and separated from each other.

The light emitting regions PXA-R, PXA-G, and PXA-B may be arranged into groups according to the color of light generated from the light emitting elements ED-1, ED-2, and ED-3. In the display device DD according to an embodiment illustrated in FIGS. 1 and 2, three light emitting regions PXA-R, PXA-G, and PXA-B, which respectively emit red light, green light, and blue light, are illustrated as an example. For example, the display device DD may include a red light emitting region PXA-R, a green light emitting region PXA-G, and a blue light emitting region PXA-B, which are distinct from each other.

In the display device DD according to an embodiment, the light emitting devices ED-1, ED-2, and ED-3 may emit light having wavelengths that are different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may respectively correspond to the first light emitting element ED-1, the second light emitting element ED-2, and the third light emitting element ED-3.

However, embodiments are not limited thereto, and the first to third light emitting elements ED-1, ED-2, and ED-3 may emit light in a same wavelength range, or at least one light emitting element may emit a light in a wavelength range that is different from the remainder. For example, the first to third light emitting elements ED-1, ED-2, and ED-3 may each emit blue light.

The light emitting regions PXA-R, PXA-G, and PXA-B in the display device DD according to an embodiment may be arranged in a stripe configuration. Referring to FIG. 1, the red light emitting regions PXA-R, the green light emitting regions PXA-G, and the blue light emitting regions PXA-B may be respectively arranged along a second directional axis DR2. In another embodiment, the red light emitting region PXA-R, the green light emitting region PXA-G, and the blue light emitting region PXA-B may be alternately arranged in this repeating order along a first directional axis DR1.

FIGS. 1 and 2 illustrate that the light emitting regions PXA-R, PXA-G, and PXA-B all have a similar area, but embodiments are not limited thereto. In an embodiment, the light emitting regions PXA-R, PXA-G, and PXA-B may be different in size or shape from each other, according to a wavelength range of emitted light. For example, the areas of the light emitting regions PXA-R, PXA-G, and PXA-B may be areas in a plan view that are defined by the first directional axis DR1 and the second directional axis DR2.

An arrangement of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various combinations, according to the display quality characteristics that are required for the display device DD. For example, the light emitting regions PXA-R, PXA-G, and PXA-B may be arranged in a pentile configuration (such as PENTILE™) or in a diamond configuration (such as Diamond Pixel™).

The areas of the light emitting regions PXA-R, PXA-G, and PXA-B may be different in size from each other. For example, in an embodiment, an area of a green light emitting region PXA-G may be smaller than an area of a blue light emitting region PXA-B, but embodiments are not limited thereto.

Hereinafter, FIG. 3 to FIG. 6 are each a schematic cross-sectional view of a light emitting element according to an embodiment. The light emitting element ED according to an embodiment may include a first electrode EL1, a second electrode EL2 oppositely disposed to the first electrode EL1, and at least one functional layer disposed between the first electrode EL1 and the second electrode EL2. The light emitting element ED may include an amine compound according to an embodiment, which will be explained later, in the at least one functional layer.

The light emitting element ED may include a hole transport region HTR, an emission layer EML, an electron transport region ETR, or the like, as the at least one functional layer. Referring to FIG. 3, the light emitting element ED according to an embodiment may include a first electrode EL1, a hole transport region HTR, an emission layer EML, an electron transport region ETR, and a second electrode EL2.

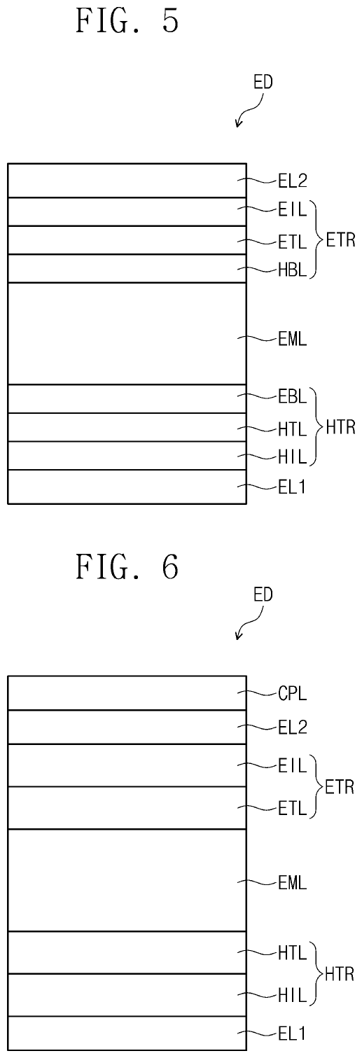

In comparison to FIG. 3, FIG. 4 is a schematic cross-sectional view of a light emitting element ED according to an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 is a schematic cross-sectional view of a light emitting element ED according to an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 is a schematic cross-sectional view of a light emitting element ED according to an embodiment that includes a capping layer CPL disposed on a second electrode EL2.

The light emitting element ED may include an amine compound according to an embodiment, which will be explained later, in a hole transport region HTR. The light emitting element ED of an embodiment may include an amine compound according to an embodiment in at least one of a hole injection layer HIL, a hole transport layer HTL, and an electron blocking layer EBL of the hole transport region HTR. For example, in the light emitting element ED, the hole transport layer HTL may include the amine compound according to an embodiment.

The first electrode EL1 has conductivity. The first electrode EL1 may be formed of a metal material, a metal alloy, or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. The first electrode EL1 may include Ag, Mg, Cu, Al, Pt, Pd, Au, Ni, Nd, Ir, Cr, Li, Ca, LiF, Mo, Ti, W, In, Sn, Zn, an oxide thereof, a compound thereof, or a mixture thereof.

If the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In another embodiment, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments are not limited thereto. In an embodiment, the first electrode EL1 may include the above-described metal materials, combinations of at least two metal materials of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

The hole transport region HTR may be provided on the first electrode EL1. The hole transport region HTR may be a layer consisting of a single material, a layer including different materials, or a structure including multiple layers including different materials.

The hole transport region HTR may include at least one of a hole injection layer HIL, a hole transport layer HTL, and an electron blocking layer EBL. Although not shown in the drawings, in an embodiment, the hole transport region HTR may include multiple hole transport layers that are provided as a stacked structure.

In an embodiment, the hole transport region HTR may have a single layer structure of a hole injection layer HIL or a hole transport layer HTL, or may have a structure of a single layer formed of a hole injection material and a hole transport material. In an embodiment, the hole transport region HTR may have a structure of a single layer including different materials, or may have a structure in which a hole injection layer HIL/hole transport layer HTL, a hole injection layer HIL/hole transport layer HTL/buffer layer (not shown), a hole injection layer HIL/buffer layer (not shown), or a hole transport layer HTL/buffer layer (not shown), are stacked in its respective stated order from the first electrode EL1, but embodiments are not limited thereto.

A thickness of the hole transport region HTR may be, for example, in a range of about 50 Å to about 15,000 Å. The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The light emitting element ED may include the amine compound according to an embodiment in the hole transport region HTR. In the light emitting element ED, the hole transport region HTR may include a hole injection layer HIL or a hole transport layer HTL, and the hole transport layer HTL may include the amine compound according to an embodiment. In an embodiment, a functional layer that is included in the hole transport region HTR may be adjacent (for example, directly adjacent) to the emission layer EML and may include the amine compound according to an embodiment.

The amine compound according to an embodiment may include an amine group and a first substituent, a second substituent, and a third substituent which are linked to the amine group. For example, the amine compound may include an amine group in the form of a core nitrogen atom, and a structure in which the first to third substituents may be bonded to the core nitrogen atom.

The first substituent may include a benzonaphthofuran moiety or a benzonaphthothiophene moiety. In the amine compound according to an embodiment, the benzonaphthofuran moiety or the benzonaphthothiophene moiety may be bonded to the nitrogen atom, and the oxygen atom of the benzonaphthofuran moiety or the sulfur atom of the benzonaphthothiophene moiety may be bonded at a meta position to the nitrogen atom. The first substituent may be directly bonded to the core nitrogen atom. The second substituent may include a dibenzofuran moiety, a dibenzothiophene moiety, or a 9,9-diphenylfluorene moiety. In an embodiment, the numbers of carbon atoms of the second substituent may be assigned as represented by Formula S1. The second substituent may be directly bonded to the core nitrogen atom. The third substituent may be an aryl group or a heteroaryl group bonded to the core nitrogen atom via an arylene linker or a heteroarylene linker, or may be directly bonded to the core nitrogen atom without a linker.

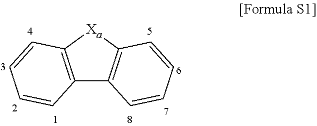

With respect to the carbon numbering of the second substituent, in the case where the first substituent is disposed such that Xₐ may be disposed on the top of the first substituent as in Formula S1, the numbers may be assigned in a clockwise direction from the carbon atom, which is at the meta-position with Xₐ and disposed at the bottom, from among the carbon atoms constituting the left benzene ring, and the carbon number at the condensation position may be excluded. For convenience of description, substituents linked to benzene rings at either side in Formula S1 are omitted. Although not shown in Formula S1, the second substituent may have at least one substituent in addition to hydrogen atoms.

In Formula S1, Xₐ may be O, S, or CRR′. In Formula S1, R and R′ may each be an unsubstituted phenyl group. In Formula S1, when Xₐ is O, the second substituent may include a dibenzofuran moiety. In Formula S1, when Xₐ is S, the second substituent may include a dibenzothiophene moiety. In Formula S1, when Xₐ is CRR′, the second substituent may include a 9,9-diphenylfluorene moiety.

In an embodiment, the amine compound may be a monoamine compound that includes a single amine group. The amine compound according to an embodiment may be a monoamine compound having a single amine group which does not form a ring in the molecular structure thereof.

In an embodiment, the amine compound may be represented by Formula 1:

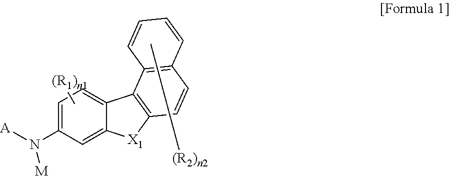

In Formula 1, A may be a group represented by one of Formula 2-1 to Formula 2-3.

In Formula 1, M may be a group represented by Formula 3.

In Formula 1, X₁ may be O or S.

In Formula 1, R₁ and R₂ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁ and R₂ may each independently be a hydrogen atom or a deuterium atom.

In Formula 1, n1 may be an integer from 0 to 3. If n1 is 0, the amine compound may not be substituted with R₁. A case where n1 is 3 and R₁ groups are all hydrogen atoms may be the same as a case where n1 is 0. If n1 is 2 or greater, multiple R₁ groups may all be the same, or at least one thereof may be different from the remainder.

In Formula 1, n2 may be an integer from 0 to 6. If n2 is 0, the amine compound may not be substituted with R₂. A case where n2 is 6 and R₂ groups are all hydrogen atoms may be the same as a case where n2 is 0. If n2 is 2 or more, multiple R₂ groups may all be the same, or at least one thereof may be different from the remainder.

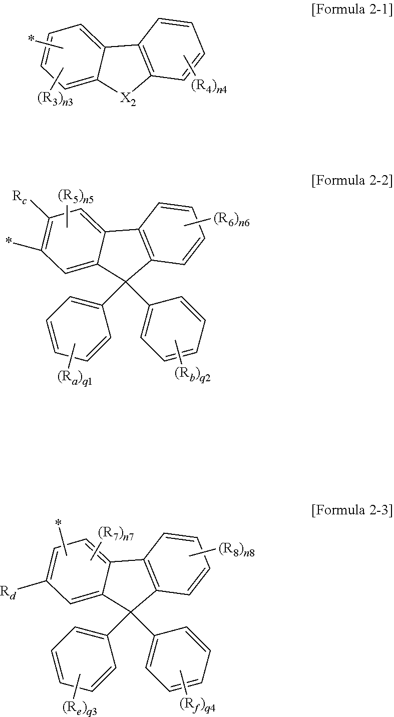

In Formula 2-1, X₂ may be O or S.

In Formula 2-2, R_{c} may be a hydrogen atom, a deuterium atom, or a substituted or unsubstituted alkyl group having 1 to 20 carbon atoms. For example, R_{c} may be a hydrogen atom or a deuterium atom.

In Formula 2-1 to Formula 2-3, Rₐ, R_{b}, R_{d} to R_{f}, and R₃ to R₈ may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. For example, Rₐ, R_{b}, R_{d} to R_{f}, and R₃ to R₈ may each independently be a hydrogen atom or a deuterium atom. In an embodiment, in Formula 2-1, multiple R₄ groups may be provided, and the R₄ groups may be bonded to each other to form a benzonaphthofuran ring or a benzonaphthothiophene ring such as

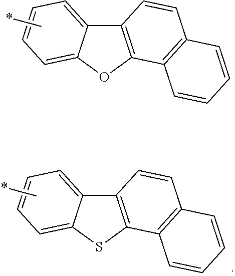

In Formula 2-1, n3 may be an integer from 0 to 3. If n3 is 0, the amine compound may not be substituted with R₃. A case where n3 is 3 and R₃ groups are all hydrogen atoms may be the same as a case where n3 is 0. If n3 is 2 or more, multiple R₃ groups may all be the same, or at least one thereof may be different from the remainder.

In Formula 2-1 to Formula 2-3, n4, n6, and n8 may each independently be an integer from 0 to 4. If n4, n6, and n8 are each 0, the amine compound may not be substituted with R₄, R₆, and R₈. A case where n4, n6, and n8 are each 4 and R₄ groups, R₆ groups, and R₈ groups are all hydrogen atoms may be the same as a case where n4, n6, and n8 are each 0. If n4, n6, and n8 are each 2 or greater, multiple groups of each of R₄, R₆, and R₈ may be the same or at least one thereof may be different from the remainder.

In Formula 2-2 and Formula 2-3, n5 and n7 may each independently be an integer from 0 to 2. If n5 and n7 are each 0, the amine compound may not be substituted with R₅ and R₇. A case where n5 and n7 are each 2 and R₅ groups and R₇ groups are all hydrogen atoms may be the same as a case where n5 and n7 are each 0. If n5 and n7 are each 2, two R₅ groups and two R₇ groups may be the same or at least one thereof may be different from the remainder.

In Formula 2-2 and Formula 2-3, q1 to q4 may each independently be an integer from 0 to 5. If q1 to q4 are each 0, the amine compound may not be substituted with Rₐ, R_{b}, Rₑ, and R_{f}. A case where q1 to q4 are each 5 and Rₐ groups, R_{b} groups, Rₑ groups, and R_{f} groups are all hydrogen atoms may be the same as a case where q1 to q4 are each 0. If q1 to q4 are each 2 or greater, multiple groups of each of Rₐ, R_{b}, Rₑ and R_{f} may be the same or at least one thereof may be different from the remainder.

In Formula 2-1 to Formula 2-3, —* represents a bond to Formula 1.

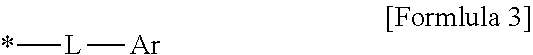

In Formula 3, L may be a direct linkage, a substituted or unsubstituted arylene group having 6 to 30 ring-forming carbon atoms, or a substituted or unsubstituted heteroarylene group having 2 to 30 ring-forming carbon atoms. For example, L may be a direct linkage or a substituted or unsubstituted a phenylene group.

In Formula 3, Ar may be a substituted or unsubstituted aryl group having 6 to 30 ring-forming carbon atoms, or a substituted or unsubstituted heteroaryl group having 2 to 30 ring-forming carbon atoms and having 1 or 2 ring-forming heteroatoms, a group represented by Formula 2-2, or a group represented by Formula 2-3. For example, Ar may be a substituted or unsubstituted naphthyl group, a substituted or unsubstituted phenyl group, or a substituted or unsubstituted phenanthrene group, a group represented by Formula 2-2, or a group represented by Formula 2-3. In an embodiment, when Ar is a heteroaryl group, the heteroaryl group may include at most 2 heteroatoms as ring-forming atoms. When Ar is a heteroaryl group, the heteroaryl group may include one or two heteroatoms as ring-forming atoms. In the amine compound according to an embodiment, when Ar includes a heteroatom, the heteroatom may be an oxygen atom or a sulfur atom. In the amine compound according to an embodiment, when Ar includes a heteroatom, Ar may not include a nitrogen atom as the heteroatom.

In Formula 3, —* represents a bond to Formula 1.

In the amine compound represented by Formula 1, a case is excluded where Ar includes an aromatic fused ring containing sp³ carbon in addition to sp³ carbon contained in a fluorene skeleton represented by Formula 2-2 or Formula 2-3. In the amine compound represented by Formula 1, a case is excluded where Ar includes an aromatic fused ring in which an aliphatic hydrocarbon ring in addition to the substituents represented by Formula 2-2 and Formula 2-3 is fused. In the amine compound represented by Formula 1, Ar may not include a fused ring having a structure in which a cycloalkyl ring in the molecular structure is fused to an aromatic hydrocarbon ring in addition to the fluorene groups represented by Formula 2-2 and Formula 2-3. For example, in the amine compound represented by Formula 1, Ar may not include a fused ring having a structure in which a cycloalkyl ring is fused in an aryl ring as in S1 below. In an embodiment, in the amine compound represented by Formula 1, a case is excluded where each of R₁ and R₂ includes an aromatic fused ring containing sp³ carbon. For example, a case where each of R₁ and R₂ includes a substituted or unsubstituted fluorene group may be excluded. The fused ring having a structure in which a cycloalkyl ring is fused to an aryl ring may contribute to the deterioration in service life of the light emitting element because the fused ring is thermally and chemically unstable due to the cycloalkyl skeleton containing sp³ carbon. According to embodiments, the amine compound represented by Formula 1 excludes a case of including an aromatic fused ring containing sp³ carbon in addition to sp³ carbon contained in the fluorene skeletons represented by Formula 2-2 and Formula 2-3 so that the thermal and chemical stability may be improved and thus improved element service life characteristics may be exhibited.

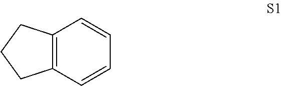

In the amine compound represented by Formula 1, a case is excluded where the amine compound includes a nitrogen-containing heterocycle and a halogen atom.

In an embodiment, a case is excluded where the amine compound represented by Formula 1 includes a nitrogen-containing heterocycle. For example, a case where the amine compound represented by Formula 1 includes a substituted or unsubstituted carbazole group may be excluded. The nitrogen-containing heterocycle contains a nitrogen atom and thus has a great effect on charge transport properties of the molecule, so that the charge transport properties may deteriorate and thus the luminous efficiency may deteriorate. According to embodiments, in the amine compound, a case of including the nitrogen-containing heterocycle is excluded, and thus the charge transport properties are improved, thereby improving the luminous efficiency.

In an embodiment, a case is excluded where the amine compound represented by Formula 1 includes a halogen atom. The amine compound represented by Formula 1 may not include a halogen atom such as F, Br, Cl, or I in the molecular structure thereof. When the amine compound includes a halogen atom in the molecular structure thereof, the amine compound may have high reaction activity due to the inclusion of a halogen atom, and thus the stability of the compound deteriorates so that when the compound is applied to a light emitting element, the element service life may deteriorate. According to embodiments, a case is excluded where the halogen atom is included in the amine compound, and thus the stability of the compound is improved so that an effect of improving the service life of the light emitting element may be exhibited.

In Formula 1, a case is excluded where M is a group represented by Formula a-1 or Formula a-2. The substituent represented by Formula a-1 has a linking structure of “phenyl-naphthalene-phenyl”, and when this substituent is bonded to the nitrogen atom (N) in Formula 1, the twist in the linking structure may deteriorate the stability of the compound. The substituent represented by Formula a-2 has a structure in which a naphthylene skeleton is directly linked to the nitrogen atom (N) in Formula 1, thereby causing excessive interactions between the naphthylene moiety and the amine group, so that the stability of the compound may deteriorate.

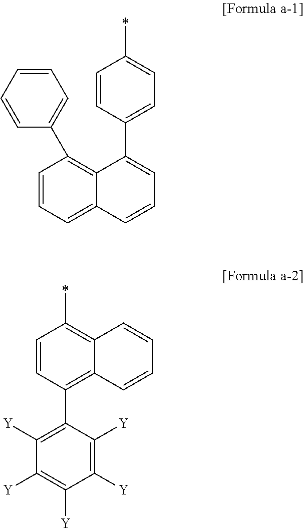

In Formula a-2, Y may be a hydrogen atom or a deuterium atom.

In Formula a-1 and Formula a-2, —*represents a bond to Formula 1.

In the amine compound represented by Formula 1, when A is a group represented by Formula 2-2 or Formula 2-3, M in Formula 1 above may not include a naphthylene moiety. For example, in Formula 1, when the fluorene moiety represented by Formula 2-2 or Formula 2-3 is linked to the nitrogen atom (N) in Formula 1, in Formula 3 represented by M, L may not be a substituted or unsubstituted naphthylene group, and Ar may not be a substituted or unsubstituted naphthyl group. When the amine compound represented by Formula 1 includes both a bulky 9,9-diphenylfluorene moiety and a naphthyl group, the stability of the compound may deteriorate. According to embodiments, when A in Formula 1 is a group represented by Formula 2-2 or Formula 2-3, a case where M includes a naphthylene moiety is excluded, and thus the stability of the molecule may be improved, which may contribute to improving an element service life.

In the amine compound represented by Formula 1, when A is a group represented by Formula 2-1, X₂ in Formula 2-1 is S, and the group represented by Formula 2-1 is linked to N in Formula 1 at carbon 1 of Formula 2-1, a case where L is a direct linkage or a group represented by Formula a-3 is excluded. In an embodiment, when A is a group represented by Formula 2-1 and Formula 2-1 is a group represented by Formula 2-1a, L may not be a direct linkage or a m-phenylene group represented by Formula a-3. When the dibenzothiophene group is linked to the amine group at carbon 1, in the case where L is a direct linkage or a m-phenylene group represented by Formula a-3, a large twist is generated around the nitrogen, and thus the stability of the molecule may not be effectively maintained.

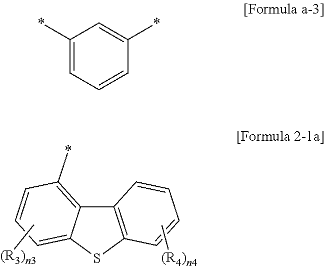

In Formula 2-1a, R₃, R₄, n3, and n4 are the same as defined in Formula 2-1.

In the amine compound represented by Formula 1, when A is a group represented by Formula 2-1, X₂ in Formula 2-1 is O, and the group represented by Formula 2-1 is linked to the nitrogen atom (N) in Formula 1 at carbon 2 or carbon 4 of Formula 2-1, a case where M in Formula 1 is a group represented by Formula 2-1b or Formula 2-1c is excluded.

In an embodiment, when A is a group represented by Formula 2-1 and Formula 2-1 is a group represented by Formula 2-1b or Formula 2-1c, a case where M in Formula 1 is an unsubstituted phenyl group or a phenyl group having a substituent having 0 to 9 carbon atoms is excluded.

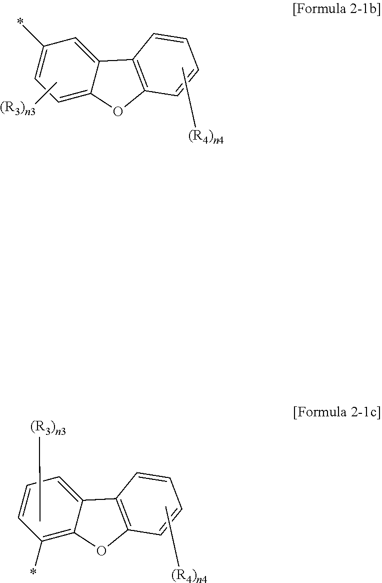

In Formula 2-1b and Formula 2-1c, R₃, R₄, n3, and n4 are the same as defined in Formula 2-1.

In the amine compound represented by Formula 1, when A is a group represented by Formula 2-1 and the group represented by Formula 2-1 is linked to N in Formula 1 at carbon 4, n1 is 0.

In the amine compound represented by Formula 1, when A is a group represented by Formula 2-2 or Formula 2-3, n1 is 0.

In an embodiment, the amine compound represented by Formula 1 may be represented b one of Formula 1-1-1 to Formula 1-1-3:

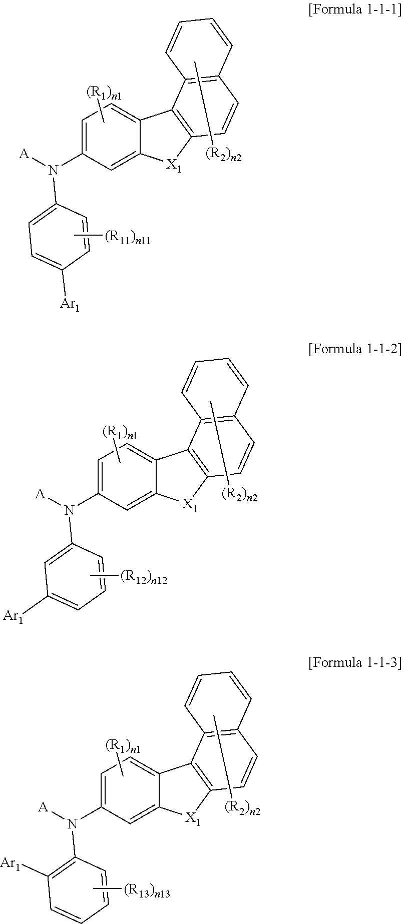

Formula 1-1-1 to Formula 1-1-3 each represent a case where L in Formula 1 is further defined as a substituted or unsubstituted phenylene group, and the position at which L is linked to the center nitrogen atom is further defined.

In Formula 1-1-1 to Formula 1-1-3, R₁₁ to R₁₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁₁ to R₁₃ may be a hydrogen atom or a deuterium atom.

In Formula 1-1-1 to Formula 1-1-3, n1 to n13 may each independently be an integer from 0 to 4. If n1 to n13 are each 0, the amine compound may not be substituted with R₁₁ to R₁₃. A case where n1 to n13 are each 4 and R₁₁ groups, R₁₂ groups, and R₁₃ groups are all hydrogen atoms may be the same as a case where n1 to n13 are each 0. If n11 to n13 are each 2 or greater, multiple groups of each of R₁₁ to R₁₃ may be the same or at least one thereof may be different from the remainder.

In Formula 1-1-1 to Formula 1-1-3, Ar₁ may be a group represented by one of Formula A-1 to Formula A-4:

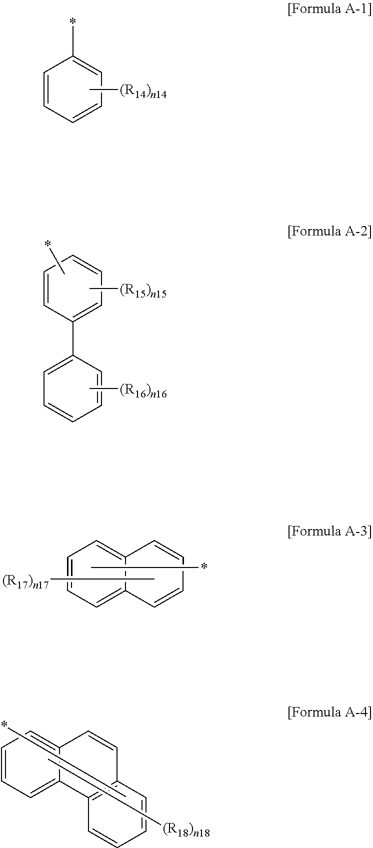

In Formula A-1 to Formula A-4, R₁₄ to R₁₈ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁₄ to R₁₈ may each independently be a hydrogen atom or a deuterium atom.

In Formula A-1 and Formula A-2, n14 and n16 may each independently be an integer from 0 to 5. If n14 and n16 are each 0, the amine compound may not be substituted with R₁₄ and R₁₆. A case where n14 and n16 are each 5 and R₁₄ group and R₁₆ groups are all hydrogen atoms may be the same as a case where n14 and n16 are each 0. When n14 and n16 are each 2 or greater, multiple R₁₄ groups and multiple R₁₆ groups may be the same or at least one thereof may be different from the remainder.

In Formula A-2, n15 may be an integer from 0 to 4. If n15 is 0, the amine compound may not be substituted with R₁₅. A case where n15 is 4 and R₁₅ groups are all hydrogen atoms may be the same as a case where n15 is 0. If n15 is 2 or more, multiple R₁₅ groups may all be the same, or at least one thereof may be different from the remainder.

In Formula A-3, n17 may be an integer from 0 to 7. If n17 is 0, the amine compound may not be substituted with R₁₇. A case where n17 is 7 and R₁₇ groups are all hydrogen atoms may be the same as a case where n17 is 0. If n17 is 2 or greater, multiple R₁₇ groups may all be the same, or at least one thereof may be different from the remainder.

In Formula A-4, n18 may be an integer from 0 to 9. If n18 is 0, the amine compound may not be substituted with R₁₈. A case where n18 is 9 and R₁₈ groups are all hydrogen atoms may be the same as a case where n18 is 0. If n18 is 2 or more, multiple R₁₈ groups may all be the same, or at least one thereof may be different from the remainder.

In Formula 1-1-1 to Formula 1-1-3, when A is a group represented by Formula 2-2 or Formula 2-3, Ar₁ may be a group represented by Formula A-1, Formula A-2, or Formula A-4.

In Formula 1-1-1 to Formula 1-1-3, A, X₁, R₁, R₂, n1, and n2 are the same as defined in Formula 1.

In an embodiment, the compound represented by Formula 1 may be represented by one of Formula 1-1-4 to Formula 1-1-6:

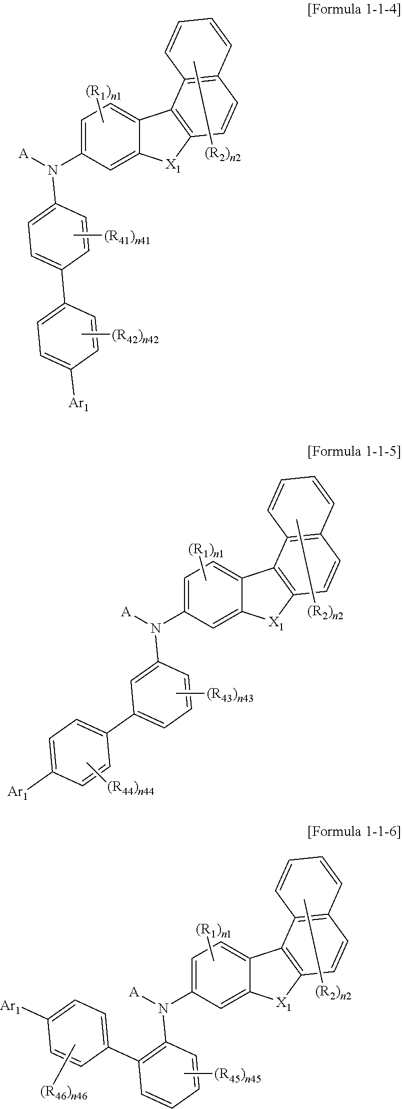

Formula 1-1-4 to Formula 1-1-6 each represent a case where L in Formula 1 is further defined as a substituted or unsubstituted biphenylene group, and the position at which L is linked to the center nitrogen atom is further defined.

In Formula 1-1-4 to Formula 1-1-6, R₄₁ to R₄₆ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₄₁ to R₄₆ may each independently be a hydrogen atom or a deuterium atom.

In Formula 1-1-4 to Formula 1-1-6, n41 to n46 may each independently be an integer from 0 to 4. If n41 to n46 are each 0, the amine compound may not be substituted with R₄₁ to R₄₆. A case where n41 to n46 are each 4 and groups of each of R₄₁ to R₄₆ are all hydrogen atoms may be the same as a case where n41 to n46 are each 0. If n41 to n46 are each 2 or greater, multiple groups of each of R₄₁ to R₄₆ may be the same or at least one thereof may be different from the remainder.

In an embodiment, the amine compound represented by Formula 1 may be represented by one of Formula 1-2-1 to Formula 1-2-7

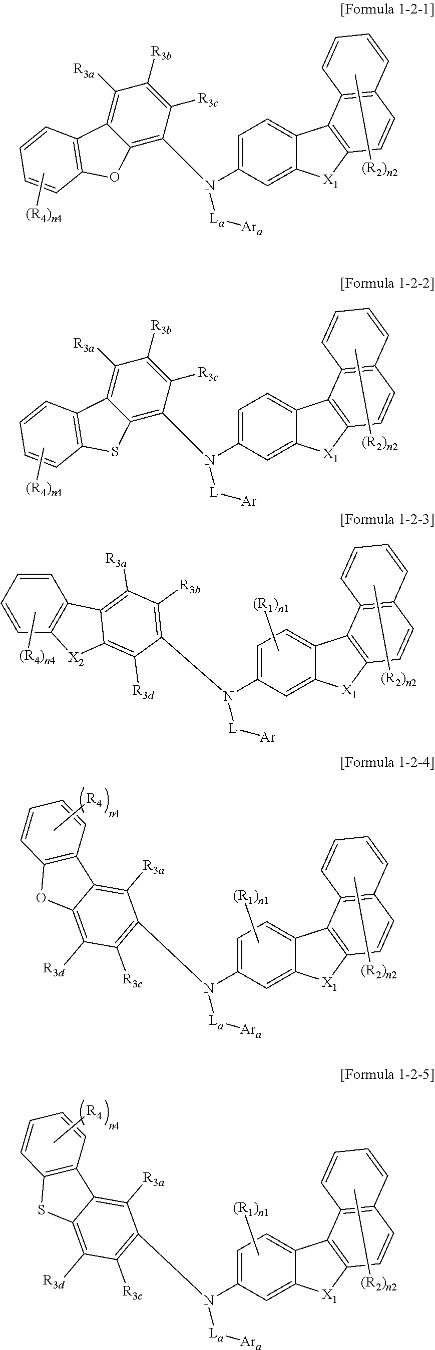

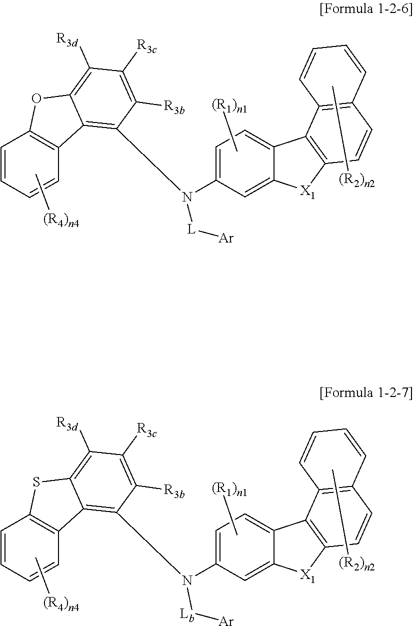

In Formula 1-2-1 and Formula 1-2-7, R₃ₐ to R_{3d} may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₃ₐ to R_{3d} may each independently be a hydrogen atom or a deuterium atom.

In Formula 1-2-1 and Formula 1-2-4, Lₐ may be a substituted or unsubstituted arylene group having 6 to 30 ring-forming carbon atoms or a substituted or unsubstituted heteroarylene group having 2 to 30 ring-forming carbon atoms. For example, Lₐ may be a substituted or unsubstituted phenylene group.

In Formula 1-2-1 and Formula 1-2-4, Arₐ may be a substituted or unsubstituted aryl group having 6 to 30 ring-forming carbon atoms or a substituted or unsubstituted heteroaryl group having 2 to 30 ring-forming carbon atoms, except that Arₐ may not be a substituted or unsubstituted phenyl group. For example, Arₐ may be a substituted or unsubstituted naphthyl group, or a substituted or unsubstituted phenanthrene group.

In Formula 1-2-7, L_{b} may be a substituted or unsubstituted arylene group having 6 to 30 ring-forming carbon atoms or a substituted or unsubstituted heteroarylene group having 2 to 30 ring-forming carbon atoms. For example, L_{b} may be a substituted or unsubstituted phenylene group. In Formula 1-2-7, a case where L_{b} is a group represented by Formula a-3 may be excluded. For example, a case where L_{b} is a m-phenylene group represented by Formula a-3 may be excluded.

In Formula 1-2-1 to Formula 1-2-7, L, Ar, X₁, R₁, R₂, n1, n2, X₂, R₄, and n4 are the same as defined in Formula 1 and Formula 2.

In an embodiment, the amine compound represented by Formula 1 may be represented by one of Formula 1-3-1 to Formula 1-3-4:

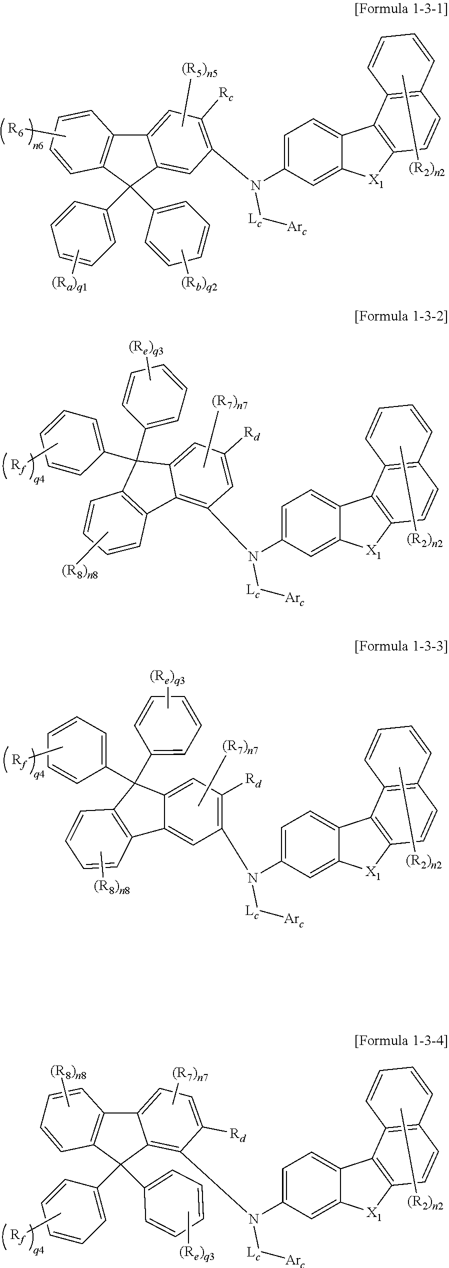

In Formula 1-3-1 to Formula 1-3-4, L_{c} may be a direct linkage, a substituted or unsubstituted arylene group having 6 to 30 ring-forming carbon atoms, or a substituted or unsubstituted heteroarylene group having 2 to 30 ring-forming carbon atoms, except that L_{c} may not be a substituted or unsubstituted naphthalene group. For example, L_{c} may be a substituted or unsubstituted phenylene group.

In Formula 1-3-1 to Formula 1-3-4, Ar_{c} may be a substituted or unsubstituted aryl group having 6 to 30 ring-forming carbon atoms or a substituted or unsubstituted heteroaryl group having 2 to 30 ring-forming carbon atoms, except that Ar_{c} may not be a substituted or unsubstituted naphthyl group. For example, Ar_{c} may be a substituted or unsubstituted phenyl group.

In Formula 1-3-1 to Formula 1-3-4, X₁, R₂, n2, R₅ to R₈, n5 to n8, Rₐ to R_{f}, and q1 to q4 are the same as defined in Formula 1, Formula 2-2, and Formula 2-3.

In an embodiment, the amine compound represented by Formula 1 may be represented by Formula 1-4-1 or Formula 1-4-2:

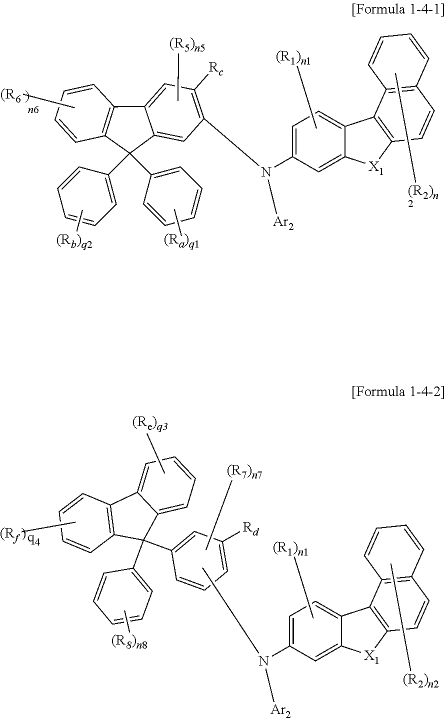

In Formula 1-4-1 and Formula 1-4-2, Ar₂ may be a group represented by one of Formula B-1 to Formula B-7:

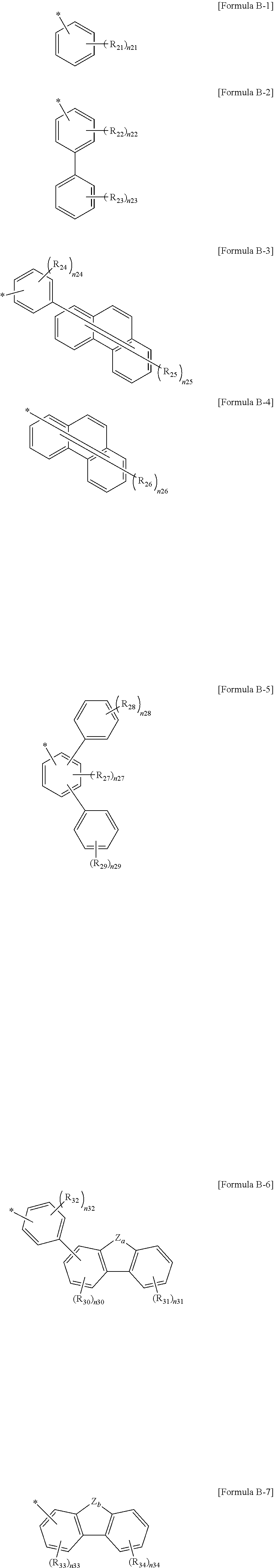

In Formula B-7, Zₐ and Z_{b} may each independently be O or S.

In Formula B-1 to Formula B-7, R₂₁ to R₃₄ may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. For example, R₂₁ to R₃₄ may each independently be a hydrogen atom, a deuterium atom, or a substituted or unsubstituted phenyl group.

In Formula B-1, Formula B-2, and Formula B-5, n21, n23, n28, and n29 may each independently be an integer from 0 to 5. If n21, n23, n28, and n29 are each 0, the amine compound may not be substituted with R₂₁, R₂₃, R₂₈, and R₂₉. A case where n21, n23, n28, and n29 are each 5 and R₂₁ groups, R₂₃ groups, R₂₈ groups, and R₂₉ groups are all hydrogen atoms may be the same as a case where n21, n23, n28, and n29 are each 0. If n21, n23, n28, and n29 are each 2 or greater, multiple groups of each of R₂₁, R₂₃, R₂₈, and R₂₉ may be the same or at least one thereof may be different from the remainder.

In Formula B-2, Formula B-3, Formula B-6, and Formula B-7, n22, n24, n31, n32, and n34 may each independently be an integer from 0 to 4. If n22, n24, n31, n32, and n34 are each 0, the amine compound may not be substituted with R₂₂, R₂₄, R₃₁, R₃₂, and R₃₄. A case where n22, n24, n31, n32, and n34 are each 4 and R₂₂ groups, R₂₄ groups, R₃₁ groups, R₃₂ groups, and R₃₄ groups are all hydrogen atoms may be the same as a case where n22, n24, n31, n32, and n34 are each 0. If n22, n24, n31, n32, and n34 are each 2 or greater, multiple groups of each of R₂₂, R₂₄, R₃₁, R₃₂, and R₃₄ may be the same or at least one thereof may be different from the remainder.

In Formula B-3 and Formula B-4, n25 and n26 may each independently be an integer from 0 to 9. If n25 and n26 are each 0, the amine compound may not be substituted with R₃₂ and R₂₆. A case where n25 and n26 are each 9 and R₂ groups and R₂₆ groups are all hydrogen atoms may be the same as a case where n25 and n26 are each 0. If n25 and n26 are each 2 or greater, multiple R₂₅ groups and multiple R₂₆ groups may be the same or at least one thereof may be different from the remainder.

In Formula B-5 and Formula B-6, n27, n30, and n33 may each independently be an integer from 0 to 3. If n27, n30, and n33 are each 0, the amine compound may not be substituted with each of R₂₇, R₃₀, and R₃₃. A case where n27, n30, and n33 are each 3 and R₂₇ groups, R₃₀ groups, and R₃₃ groups are all hydrogen atoms may be the same as a case where n27, n30, and n33 are each 0. If n27, n30, and n33 are each 2 or greater, multiple R₂₅ groups and multiple R₂₆ groups may be the same or at least one thereof may be different from the remainder.

In Formula 1-4-1 and Formula 1-4-2, X₁, R₁, R₂, n1, n2, R₅ to R₈, n5 to n8, Rₐ to R_{f}, and q1 to q4 are the same as defined in Formula 1, Formula 2-2, and Formula 2-3.

In an embodiment, the amine compound represented by Formula 1 may be represented by Formula 1-5-1 or Formula 1-5-2:

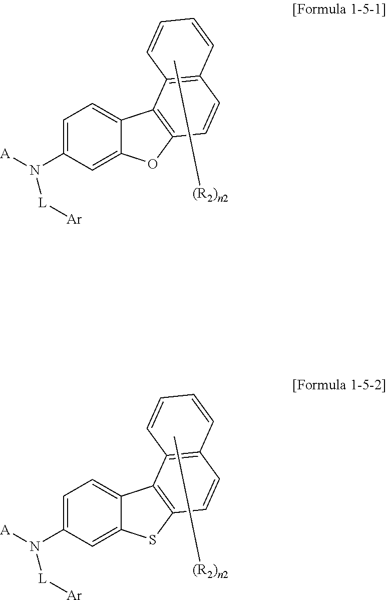

In Formula 1-5-1 and Formula 1-5-2, A, L, Ar, R₂, and n2 are the same as defined in Formula 1.

In an embodiment, the amine compound represented by Formula 1 may be represented by one of Formula 1-6-1 to Formula 1-6-4:

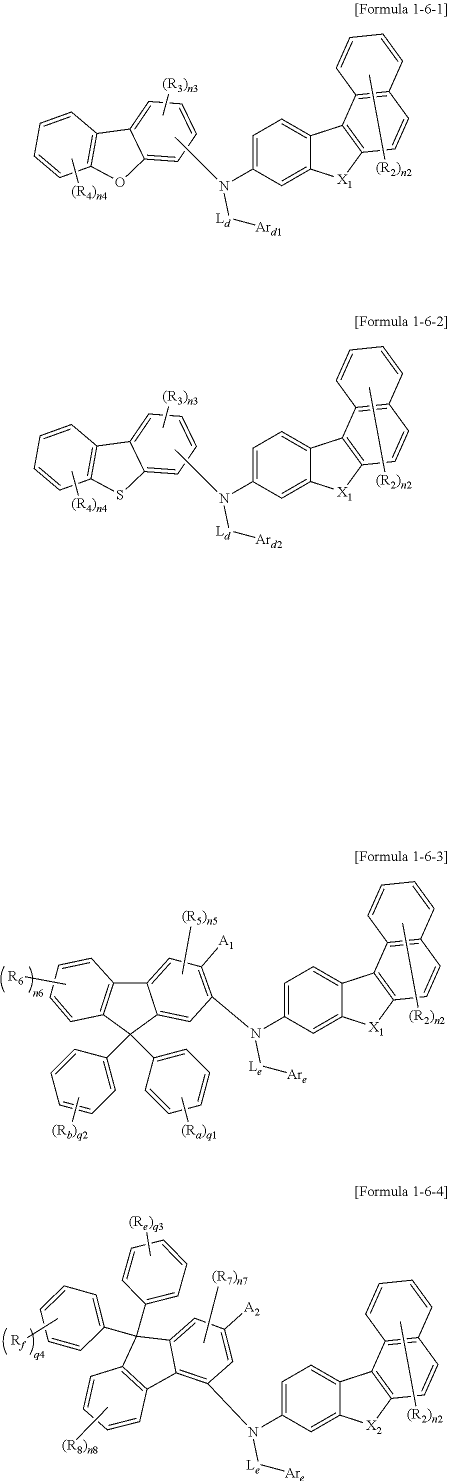

In Formula 1-6-3 and Formula 1-6-4, A₁ and A₂ may each independently be a hydrogen atom, or a deuterium atom.

In Formula 1-6-1 and Formula 1-6-2, L_{d} may be a group represented by one of Formula L-1 to Formula L-5.

In Formula 1-6-1, Ar_{d1} may be a group represented by Formula 2-2, a group represented by Formula 2-3, a substituted or unsubstituted naphthyl group, a substituted or unsubstituted anthracene group, a substituted or unsubstituted phenanthrene group, a substituted or unsubstituted dibenzofuran group, or a substituted or unsubstituted dibenzothiophene group.

In Formula 1-6-2, Ar_{d2} may be a group represented by Formula 2-2, a group represented by Formula 2-3, a substituted or unsubstituted phenyl group, a substituted or unsubstituted biphenyl group, a substituted or unsubstituted naphthyl group, a substituted or unsubstituted phenanthrene group, a substituted or unsubstituted anthracene group, a substituted or unsubstituted dibenzofuran group, or a substituted or unsubstituted dibenzothiophene group.

In Formula 1-6-3 and Formula 1-6-4, Lₑ may be a direct linkage, or a group represented by one of Formula L-1 to Formula L-5.

In Formula 1-6-3 and Formula 1-6-4, Arₑ may be a group represented by Formula 2-2, a group represented by Formula 2-3, or a group represented by one of Formula C-1 to Formula C-5:

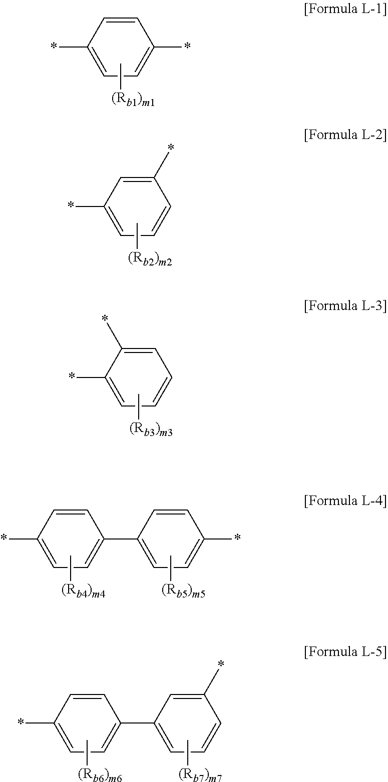

In Formula L-1 to Formula L-5, R_{b1} to R_{b7} may each independently be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R_{b1} to R_{b7} may each independently be a hydrogen atom or a deuterium atom.

In Formula L-1 to Formula L-5, m1 to m7 may each independently be an integer from 0 to 4. If m1 to m7 are each 0, the amine compound may not be substituted with R_{b1} to R_{b7}. A case where m1 to m7 are each 4 and groups of each of R_{b1} to R_{b7}'s are all hydrogen atoms may be the same as a case where m1 to m7 are each 0. If m1 to m7 are each 2 or more, multiple groups of each of R_{b1} to R_{b7} may be the same or at least one thereof may be different from the remainder.

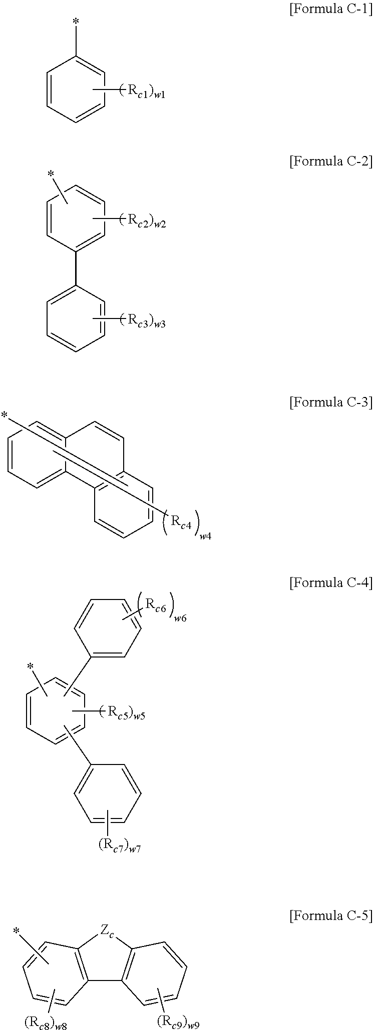

In Formula C-5, Z_{c} may be O or S.

In Formula C-1 to Formula C-5, R_{c1} to R_{c9} may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. For example, R_{c1} to R_{c9} may each independently be a hydrogen atom, a deuterium atom, or a substituted or unsubstituted phenyl group.

In Formula C-1, Formula C-2, and Formula C-4, w1, w3, w6, and w7 may each independently be an integer from 0 to 5. If w1, w3, w6, and w7 are each 0, the amine compound may not be substituted with R_{c1}, R_{c3}, R_{c6}, and R_{c7}. A case where w1, w3, w6, and w7 are each 5 and R_{c1} groups, R_{c3} groups, Re6 groups, and Re groups are all hydrogen atoms may be the same as a case where w1, w3, w6, and w7 are each 0. If w1, w3, w6, and w7 are each 2 or more, multiple groups of each of R_{c1}, R_{c3}, R_{c6}, and R_{c7} may be the same or at least one thereof may be different from the remainder.

In Formula C-2 and Formula C-5, w2 and w9 may each independently be an integer from 0 to 4. If w2 and w9 are each 0, the amine compound may not be substituted with R_{c2} and R_{c9}. A case where w2 and w9 are each 4 and R_{c2} groups and R_{c9} groups are all hydrogen atoms may be the same as a case where w2 and w9 are each 0. If w2 and w9 are each 2 or more, multiple R_{c2} groups and multiple R_{c9} groups may be the same or at least one thereof may be different from the remainder.

In Formula C-3, w4 may be an integer from 0 to 9. If w4 is 0, the amine compound may not be substituted with R_{c4}. A case where w4 is 9 and R_{c4} groups are all hydrogen atoms may be the same as a case where w4 is 0. If w4 is 2 or more, multiple R_{c4} groups may be the same or at least one thereof may be different from the remainder.

In Formula C-4 and Formula C-5, w5 and w8 may each independently be an integer from 0 to 3. If w5 and w8 are each 0, the amine compound may not be substituted with R_{c5} and R_{c8}. A case where w5 and w8 are each 3 and R_{c5} groups and R_{c8} groups are all hydrogen atoms may be the same as a case where w5 and w8 are each 0. If w5 and w8 are each 2 or more, multiple R_{c5} groups and multiple R_{c8} groups may be the same or at least one thereof may be different from the remainder.

In Formula 1-6-1 to Formula 1-6-4, X₁, R₂ to R₈, n2 to n8, Rₐ, R_{b}, Rₑ, R_{f}, and q1 to q4 are the same as defined in Formula 1, Formula 2-2, and Formula 2-3.

In an embodiment, in Formula 1, M may be a group represented by one of Formula D-1 to D-5:

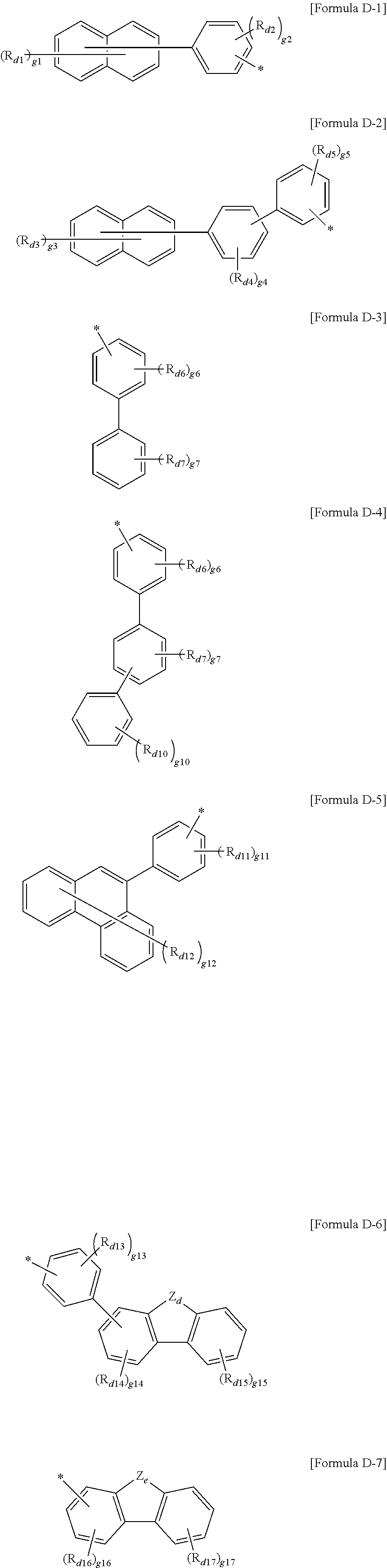

In Formula D-6 and Formula D-7, Z_{d} and Zₑ may each independently be C(R_{d18})(R_{d19}), O, or S.

In Formula D-1 to Formula D-7, R_{d1} to R_{d19} may each independently be a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. For example, R_{d1} to R_{d17} may each independently be a hydrogen atom or a deuterium atom.

In Formula D-1 to Formula D-6, g2, g4, g5, g6, g8, g9, g1, g13, g15, and g17 may each independently be an integer from 0 to 4. If g2, g4, g5, g6, g8, g9, g1, g13, g15, and g17 are each 0, the amine compound may not be substituted with R_{d2}, R_{d4}, R_{d5}, R_{d6}, R_{d8}, R_{d9}, R_{d11}, R_{d13}, R_{d15}, and R_{d17}. A case where g2, g4, g5, g6, g8, g9, g11, g13, g15, and g17 are each 4, and R_{d2} groups, R_{d4} groups, R_{d5} groups, R_{d6} groups, R_{d8} groups, R_{d9} groups, R_{d11} groups, R_{d13} groups, R_{d15} is groups, and R_{d17} groups are all hydrogen atoms may be the same as a case where g2, g4, g5, g6, g8, g9, g11, g13, g15, and g17 are each 0. If g2, g4, g5, g6, g8, g9, g11, g13, g15, and g17 are each 2 or greater, multiple groups of each of R_{d2}, R_{d4}, R_{d5}, R_{d6}, R_{d8}, R_{d9}, R_{d11}, R_{d13}, R_{d15}, and R_{d17} may be the same or at least one thereof may be different from the remainder.

In Formula D-1 and Formula D-2, g1 and g3 may each independently be an integer from 0 to 7. If g1 and g3 are each 0, the amine compound may not be substituted with R_{d1} and R_{d3}. A case where g1 and g3 are each 7 and R_{d1} groups and R_{d3} groups are all hydrogen atoms may be the same as a case where g1 and g3 are each 0. If g1 and g3 are each 2 or more, multiple R_{d1} groups and multiple R_{d3} groups may be the same or at least one thereof may be different from the remainder.

In Formula D-3 and Formula D-4, g7 and g10 may each independently be an integer from 0 to 5. If g7 and g10 are each 0, the amine compound may not be substituted with R_{d7} and R_{d10}. A case where g7 and g10 are each 5 and R_{d7} groups and R_{d10} groups are all hydrogen atoms may be the same as a case where g7 and g10 are each 0. If g7 and g10 are each 2 or more, multiple R_{d}y groups and multiple R_{d10} groups may be the same or at least one thereof may be different from the remainder.

In Formula D-5, g12 may be an integer from 0 to 9. If g12 is 0, the amine compound may not be substituted with R_{d12}. A case where g12 is 9 and R_{d12} groups are all hydrogen atoms may be the same as a case where g12 is 0. If g12 is 2 or more, multiple R_{d12} groups may be the same or at least one thereof may be different from the remainder.

In Formula D-6 and Formula D-7, g14 and g16 may each independently be an integer from 0 to 3. If g14 and g16 are each 0, the amine compound may not be substituted with R_{d14} and R_{d16}. A case where g14 and g16 are each 3 and R_{d14} groups and R_{d16} groups are all hydrogen atoms may be the same as a case where g14 and g16 are each 0. If g14 and g16 are each 2 or more, multiple R_{d14} groups and multiple R_{d16} groups may be the same or at least one thereof may be different from the remainder.

In Formula D-1 to Formula D-7, —* represents a bond to Formula 1.

In an embodiment, in Formula 1, M may be a group selected from Substituent Group B, and Ar may be a group selected from Substituent Group C:

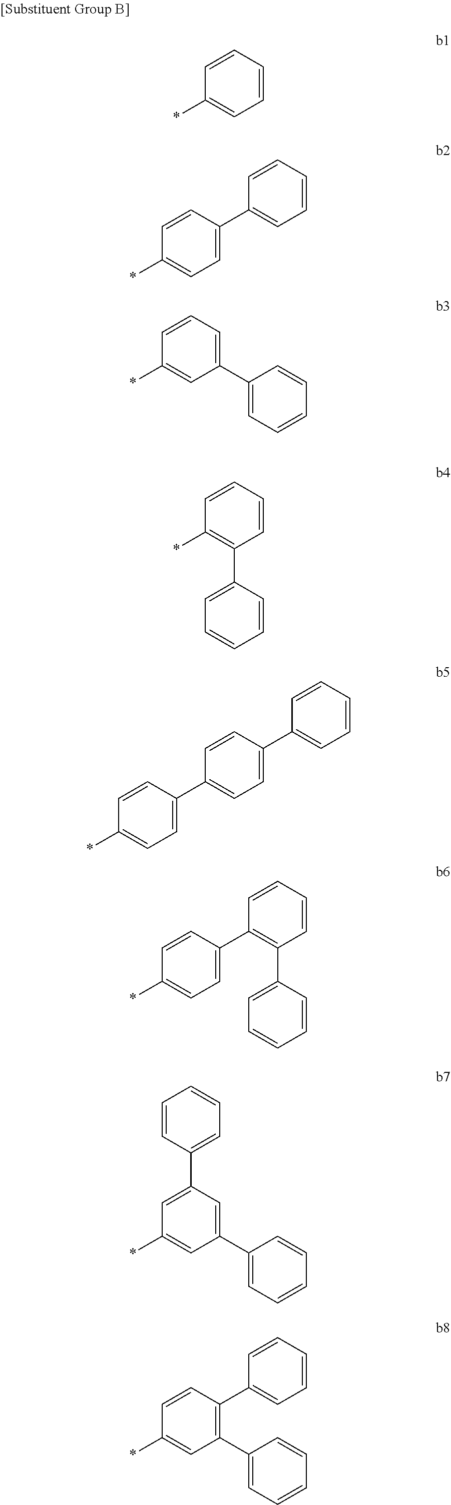

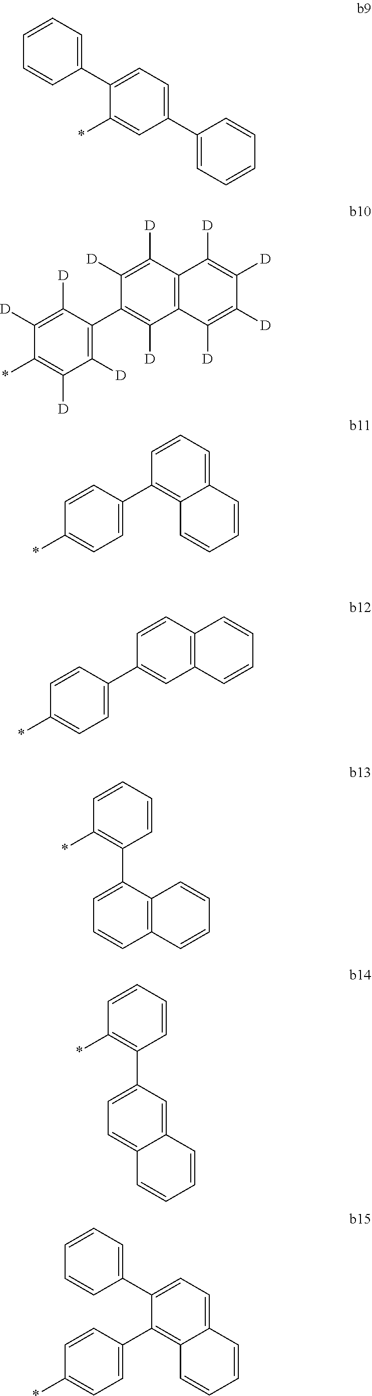

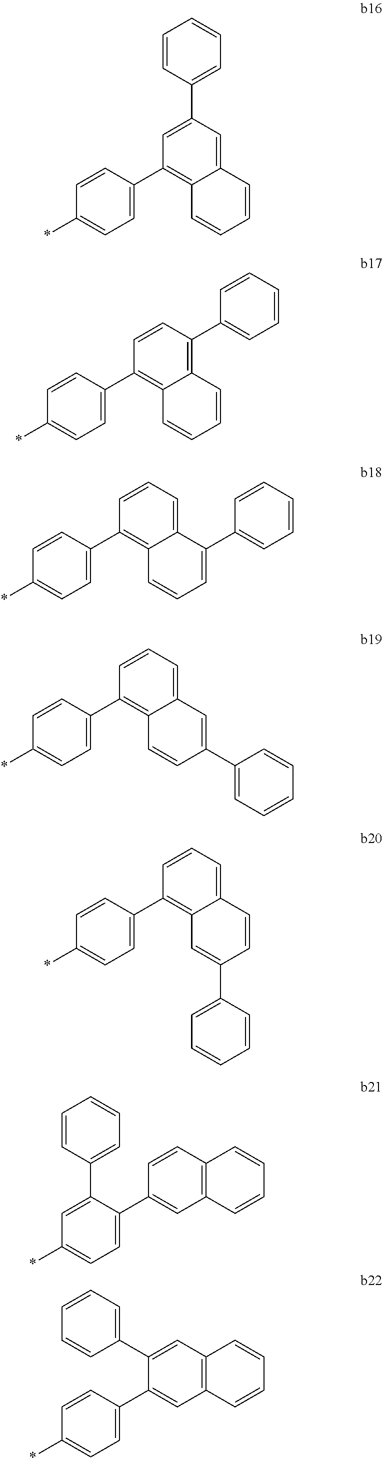

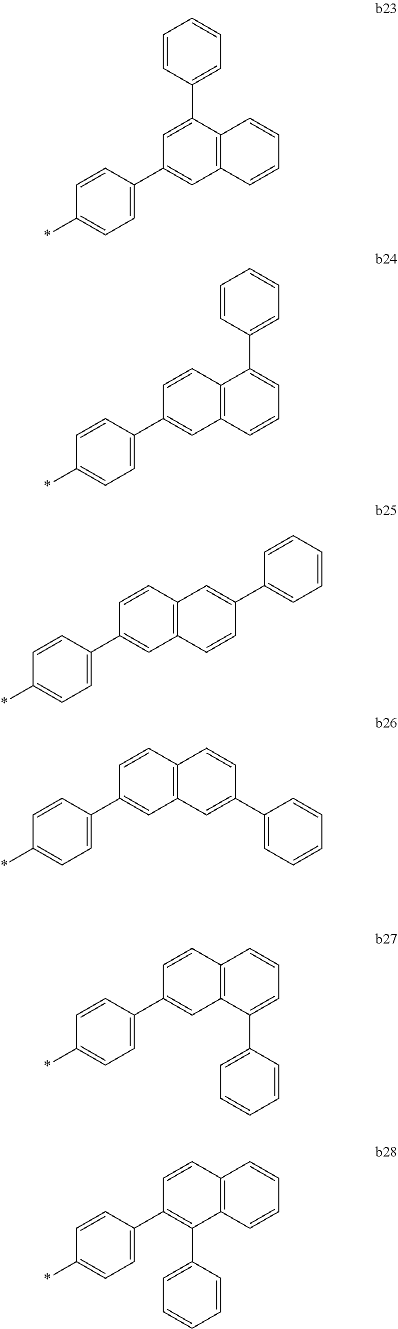

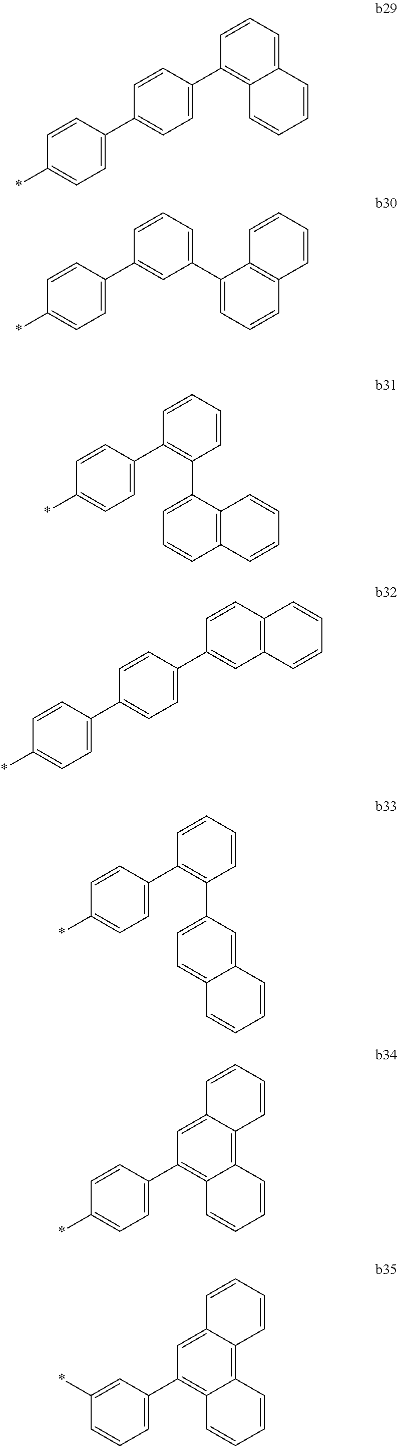

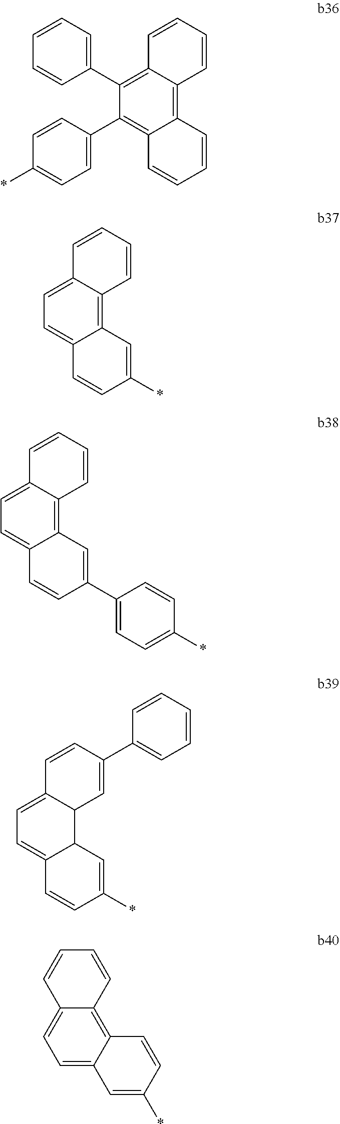

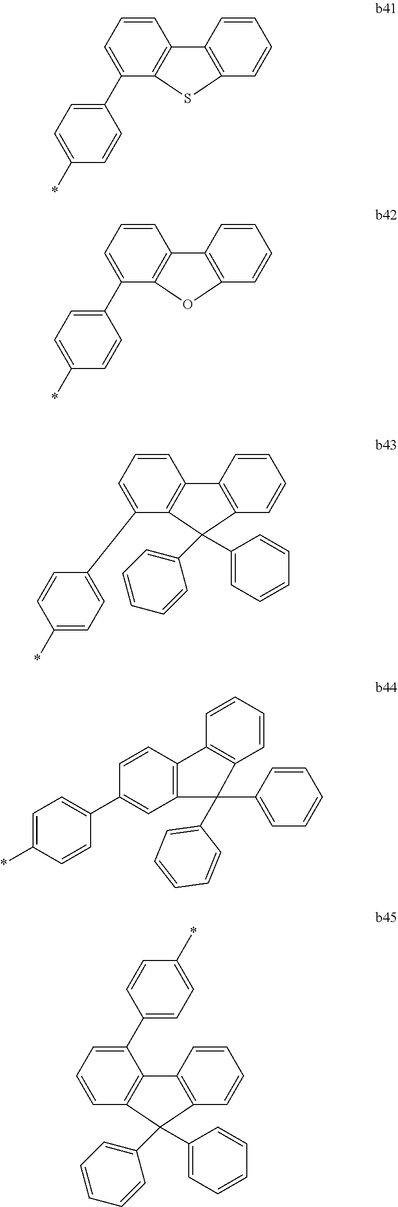 [SMILES: *c1ccc(-c2cccc3c2sc2ccccc23)cc1 ; *c1ccc(-c2cccc3c2oc2ccccc23)cc1 ; *c1ccc(-c2cccc3c2C(c2ccccc2)(c2ccccc2)c2ccccc2-3)cc1 ; *c1ccc(-c2ccc3c(c2)C(c2ccccc2)(c2ccccc2)c2ccccc2-3)cc1 ; *c1ccc(-c2cccc3c2-c2ccccc2C3(c2ccccc2)c2ccccc2)cc1]

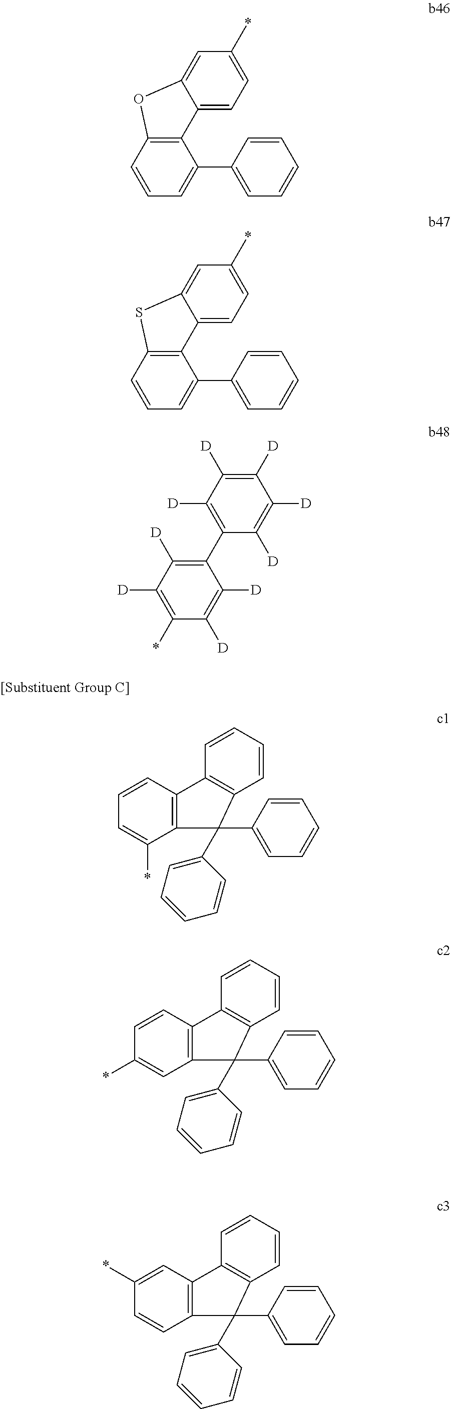

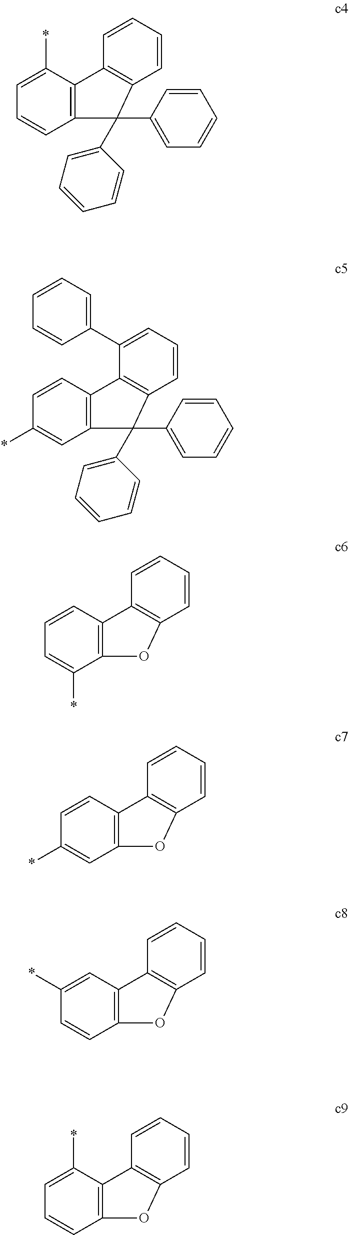 [SMILES: *c1cccc2c1-c1ccccc1C2(c1ccccc1)c1ccccc1 ; *c1ccc2c(c1)C(c1ccccc1)(c1ccccc1)c1cccc(-c3ccccc3)c1-2 ; *c1cccc2c1oc1ccccc12 ; *c1ccc2c(c1)oc1ccccc12 ; *c1ccc2oc3ccccc3c2c1 ; *c1cccc2oc3ccccc3c12]

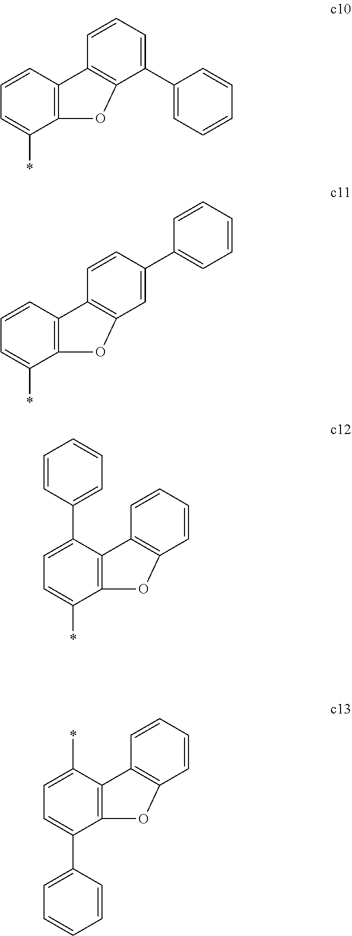 [SMILES: *c1cccc2c1oc1c(-c3ccccc3)cccc12 ; *c1cccc2c1oc1cc(-c3ccccc3)ccc12 ; *c1ccc(-c2ccccc2)c2c1oc1ccccc12 ; *c1ccc(-c2ccccc2)c2oc3ccccc3c12]

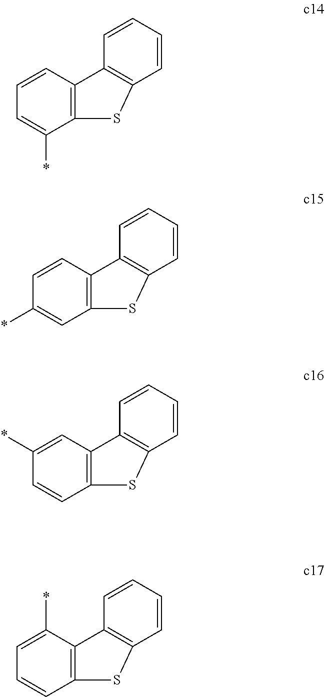 [SMILES: *c1cccc2c1sc1ccccc12 ; *c1ccc2c(c1)sc1ccccc12 ; *c1ccc2sc3ccccc3c2c1 ; *c1cccc2sc3ccccc3c12]

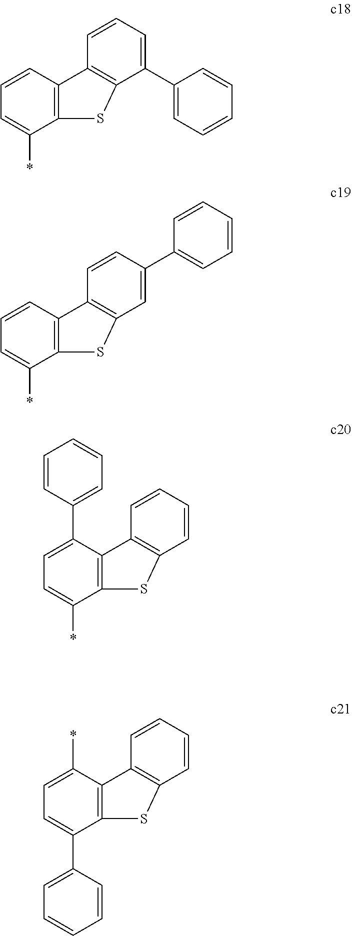 [SMILES: *c1cccc2c1sc1c(-c3ccccc3)cccc12 ; *c1cccc2c1sc1cc(-c3ccccc3)ccc12 ; *c1ccc(-c2ccccc2)c2c1sc1ccccc12 ; *c1ccc(-c2ccccc2)c2sc3ccccc3c12]

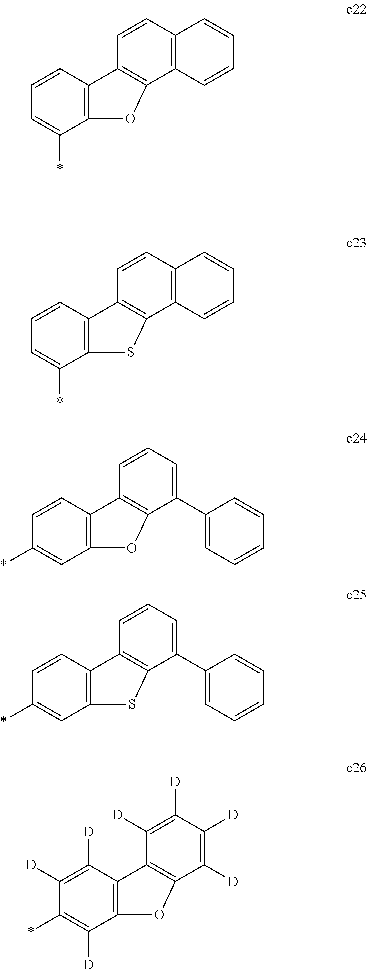 [SMILES: *c1cccc2c1oc1c3ccccc3ccc21 ; *c1cccc2c1sc1c3ccccc3ccc21 ; *c1ccc2c(c1)oc1c(-c3ccccc3)cccc12 ; *c1ccc2c(c1)sc1c(-c3ccccc3)cccc12 ; *c1c([2H])c([2H])c2c(oc3c([2H])c([2H])c([2H])c([2H])c32)c1[2H]]

In an embodiment, the amine compound represented by Formula 1 according to an embodiment may include at least one deuterium atom as a substituent. At least one of A, R₁, and R₂ in Formula 1, and L and Ar in Formula 3 may include a deuterium atom, or a substituent containing a deuterium atom. For example, the amine compound according to an embodiment may be a compound in which at least one hydrogen atom is optionally substituted with a deuterium atom.

In an embodiment, the amine compound represented by Formula 1 may be represented by Formula 1-7, and the amine compound may meet one of the combinations in Compound Combination Table 1.

The hole transport region HTR of the light emitting element ED according to an embodiment may include at least one amine compound that meets a combination in Compound Combination Table 1. For example, the hole transport layer HTL of the light emitting element ED may include at least one amine compound that meets a combination in Compound Combination Table 1.

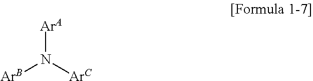

In Formula 1-7, Ar^{A} may be a group selected from Substituent Group A, Ar^{B} may be a group selected from Substituent Group B, and Ar^{C} may be a group selected from Substituent Group C.

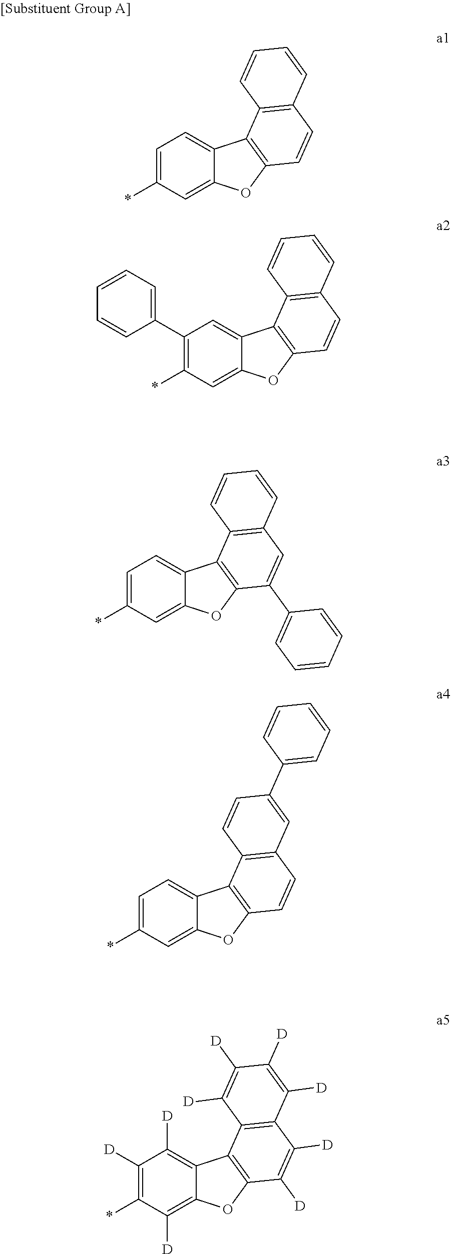

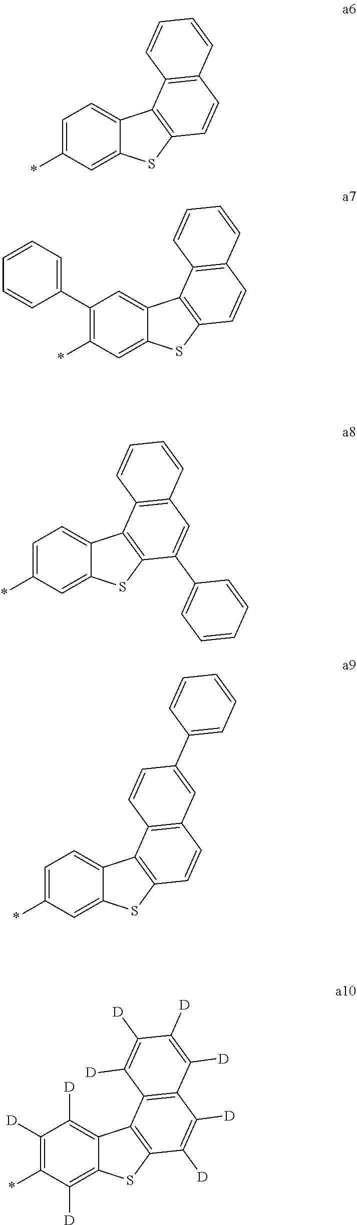

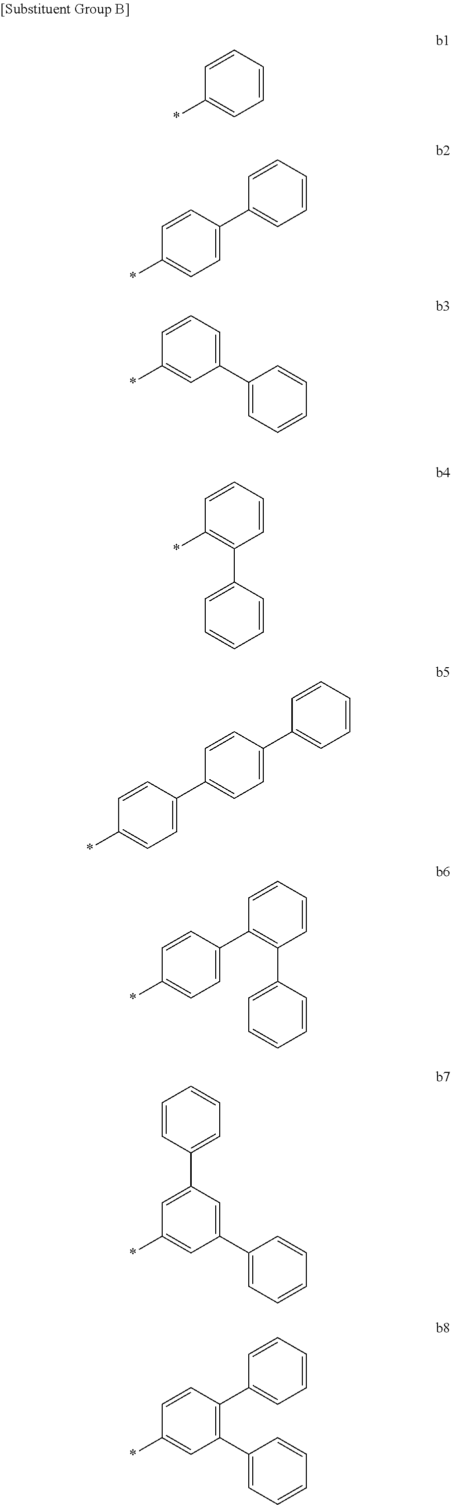

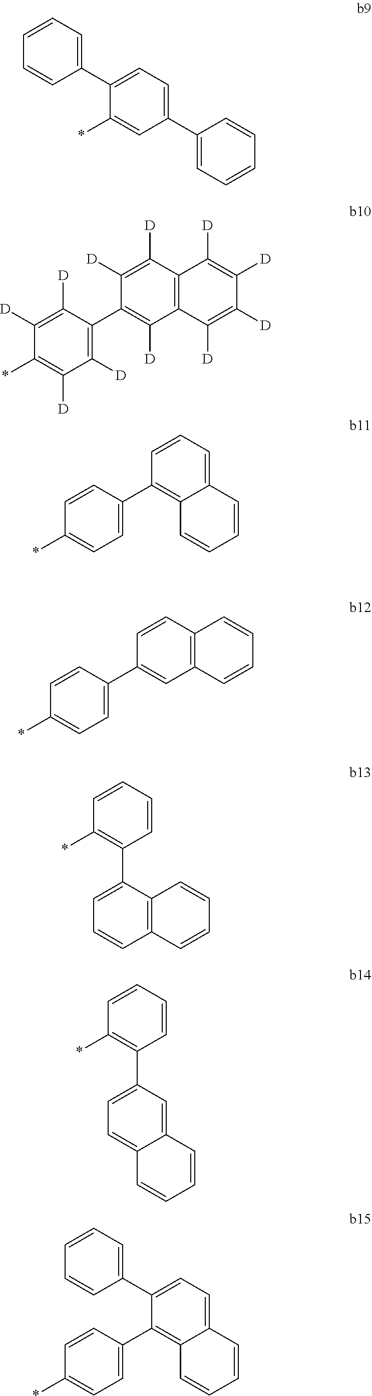

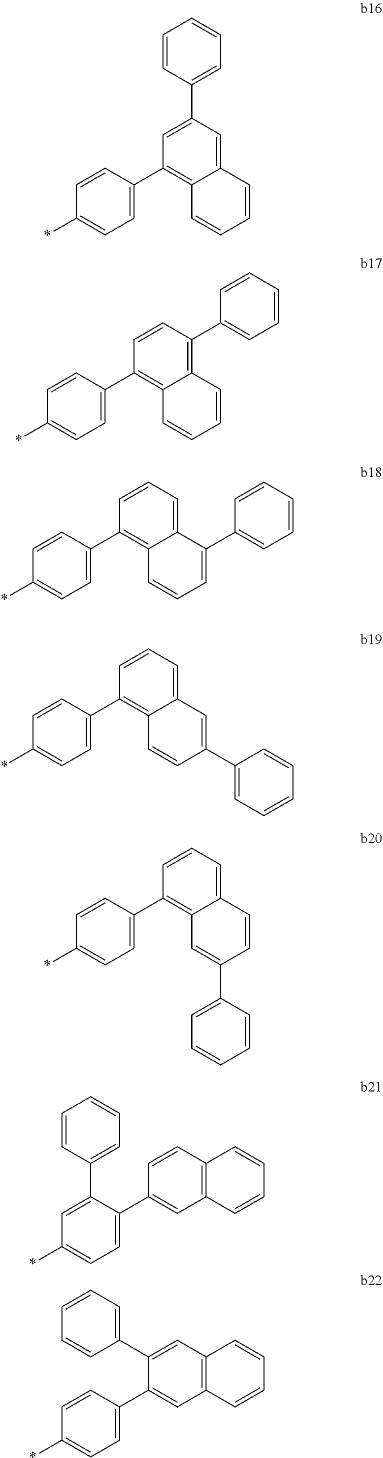

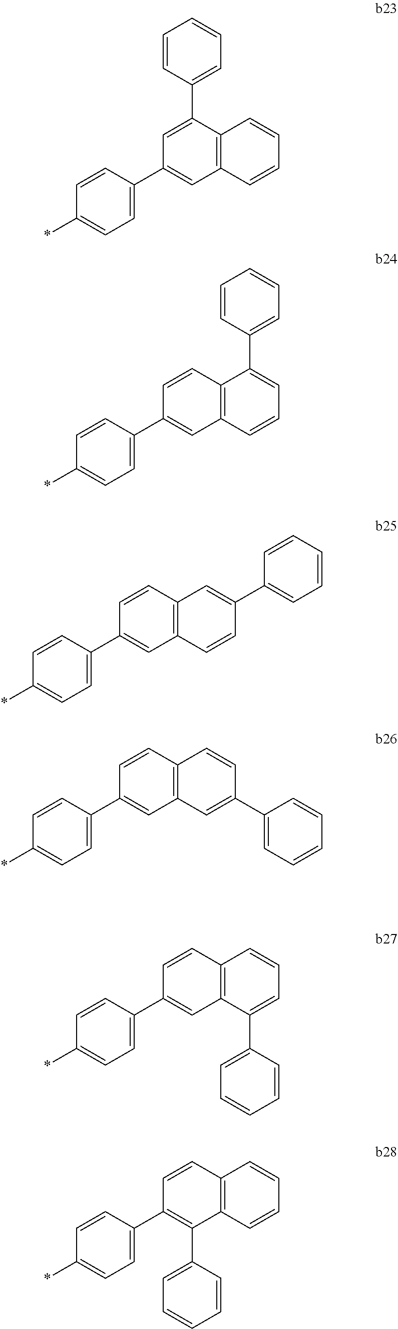

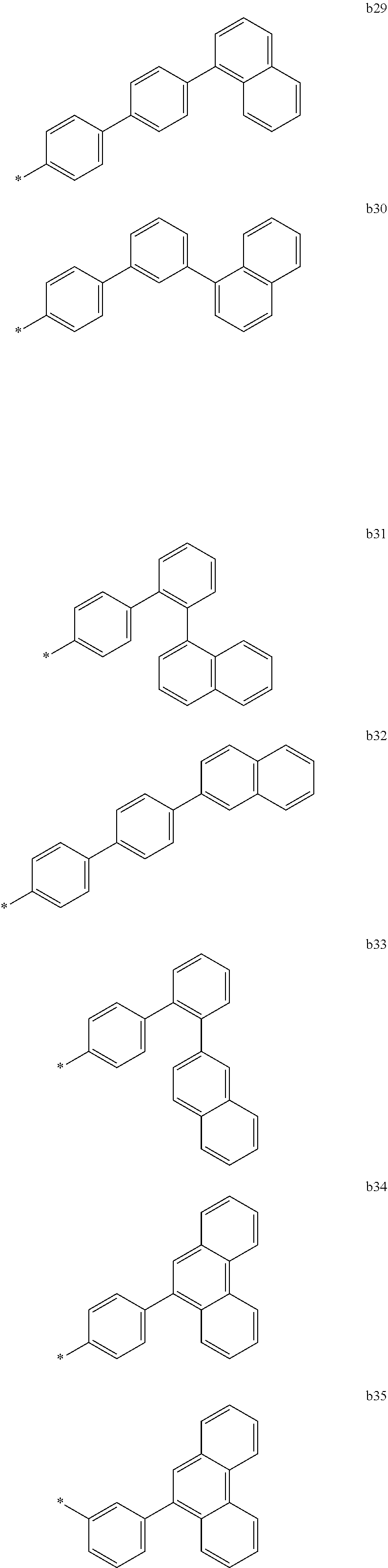

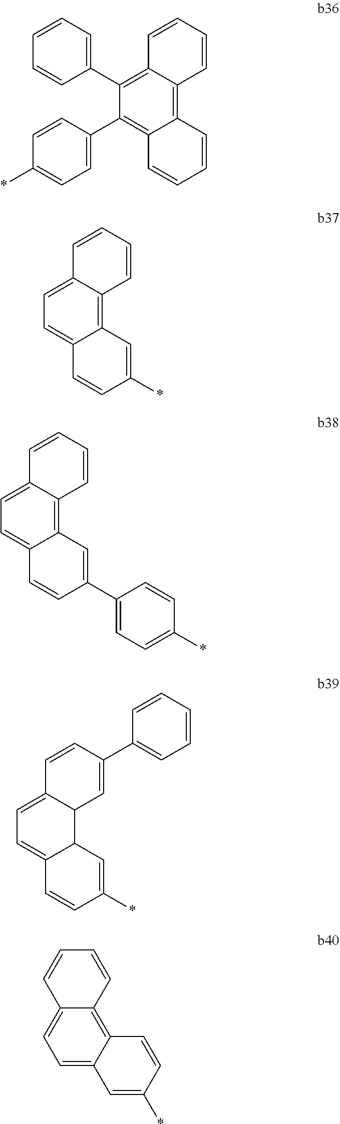

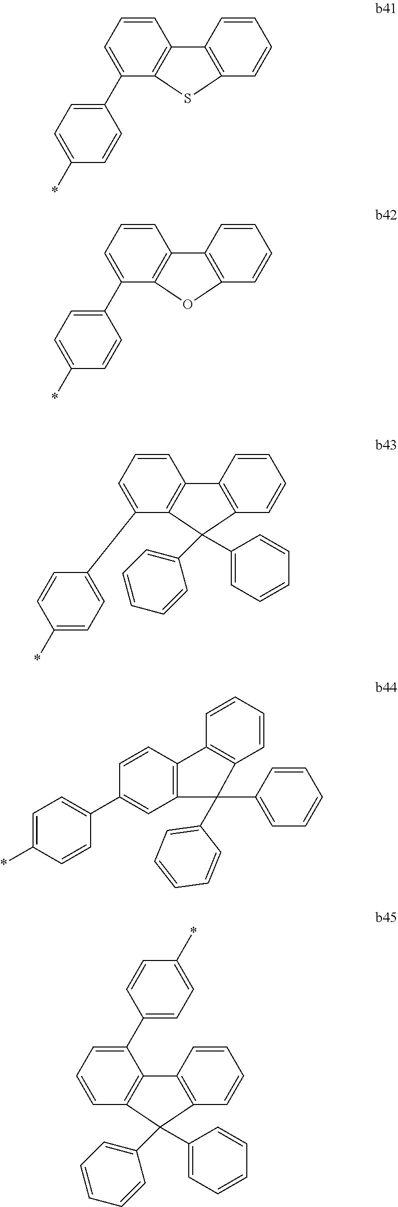 [SMILES: *c1ccc(-c2cccc3c2sc2ccccc23)cc1 ; *c1ccc(-c2cccc3c2oc2ccccc23)cc1 ; *c1ccc(-c2cccc3c2C(c2ccccc2)(c2ccccc2)c2ccccc2-3)cc1 ; *c1ccc(-c2ccc3c(c2)C(c2ccccc2)(c2ccccc2)c2ccccc2-3)cc1 ; *c1ccc(-c2cccc3c2-c2ccccc2C3(c2ccccc2)c2ccccc2)cc1]

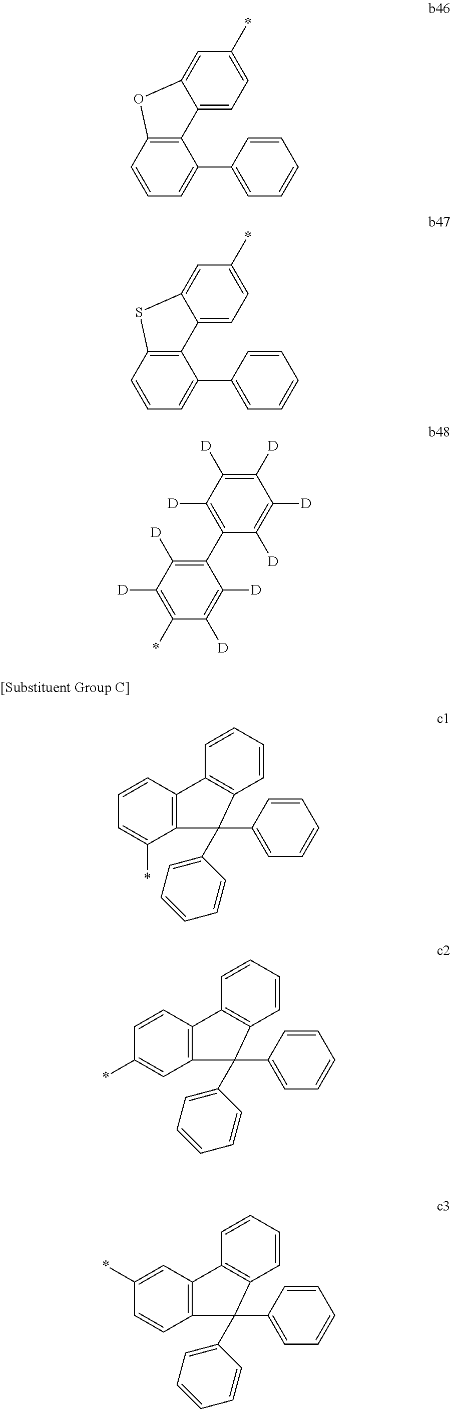

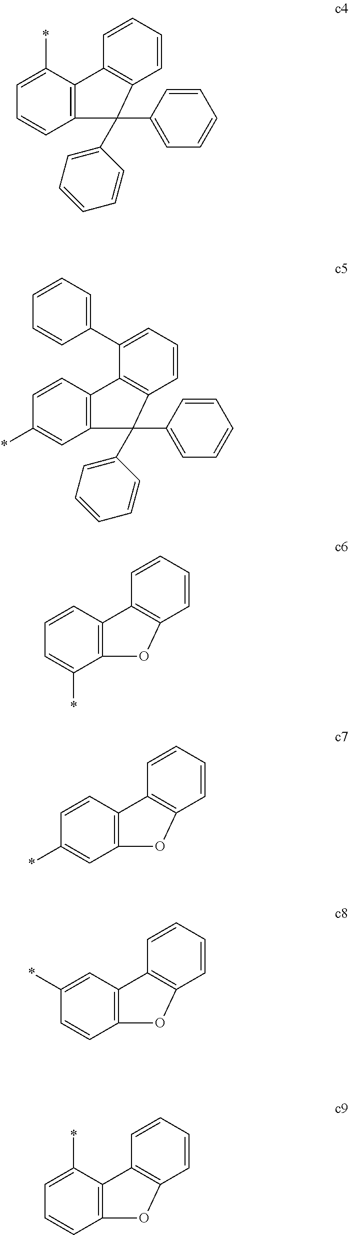 [SMILES: *c1cccc2c1-c1ccccc1C2(c1ccccc1)c1ccccc1 ; *c1ccc2c(c1)C(c1ccccc1)(c1ccccc1)c1cccc(-c3ccccc3)c1-2 ; *c1cccc2c1oc1ccccc12 ; *c1ccc2c(c1)oc1ccccc12 ; *c1ccc2oc3ccccc3c2c1 ; *c1cccc2oc3ccccc3c12]

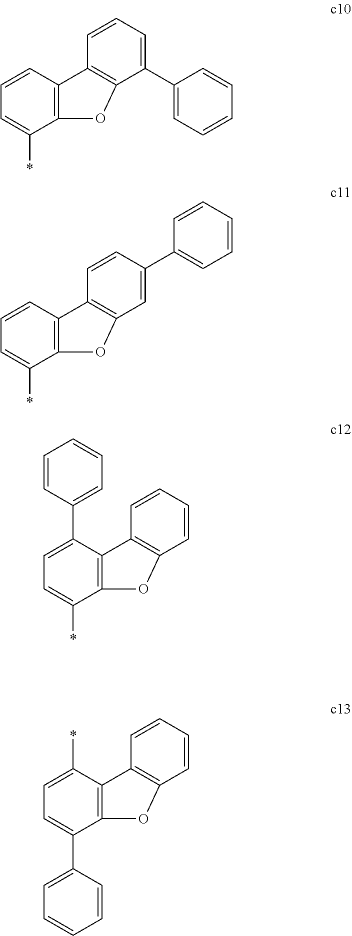 [SMILES: *c1cccc2c1oc1c(-c3ccccc3)cccc12 ; *c1cccc2c1oc1cc(-c3ccccc3)ccc12 ; *c1ccc(-c2ccccc2)c2c1oc1ccccc12 ; *c1ccc(-c2ccccc2)c2oc3ccccc3c12]

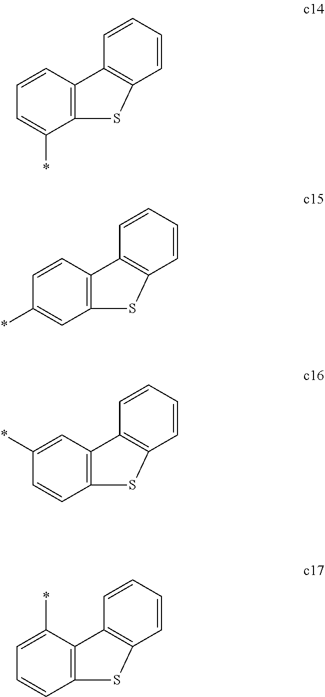 [SMILES: *c1cccc2c1sc1ccccc12 ; *c1ccc2c(c1)sc1ccccc12 ; *c1ccc2sc3ccccc3c2c1 ; *c1cccc2sc3ccccc3c12]

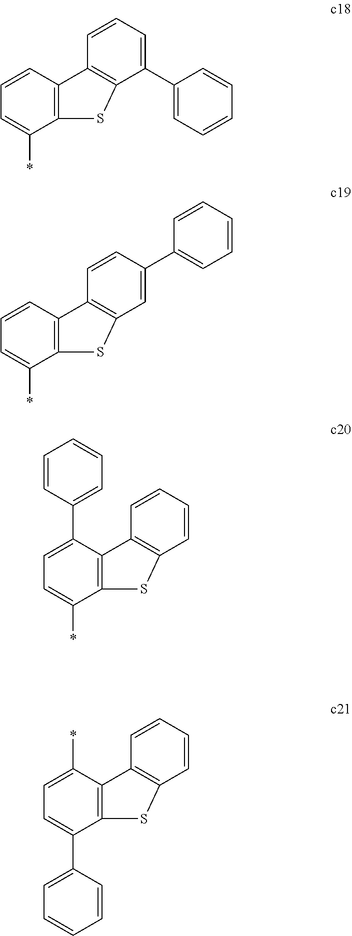 [SMILES: *c1cccc2c1sc1c(-c3ccccc3)cccc12 ; *c1cccc2c1sc1cc(-c3ccccc3)ccc12 ; *c1ccc(-c2ccccc2)c2c1sc1ccccc12 ; *c1ccc(-c2ccccc2)c2sc3ccccc3c12]

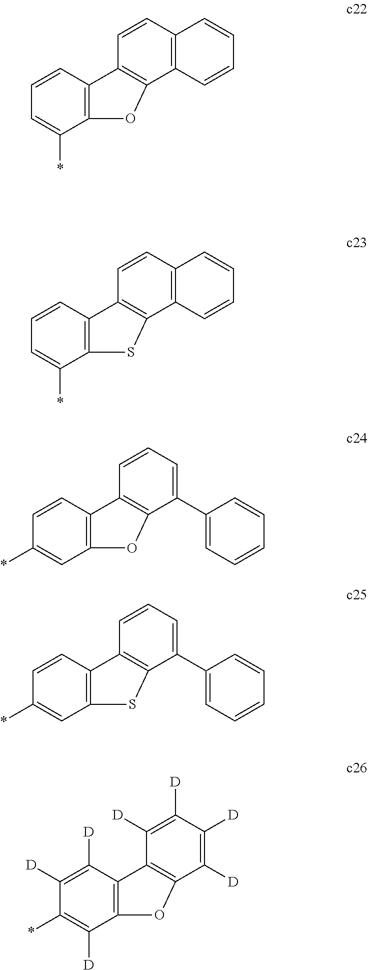 [SMILES: *c1cccc2c1oc1c3ccccc3ccc21 ; *c1cccc2c1sc1c3ccccc3ccc21 ; *c1ccc2c(c1)oc1c(-c3ccccc3)cccc12 ; *c1ccc2c(c1)sc1c(-c3ccccc3)cccc12 ; *c1c([2H])c([2H])c2c(oc3c([2H])c([2H])c([2H])c([2H])c32)c1[2H]]

[Compound Combination Table 1]

| No. | Ar^{A} | Ar^{B} | Ar^{C} |
|---|---|---|---|
| 1 | a1 | b11 | c7 |
| 2 | a2 | b11 | c7 |
| 3 | a3 | b11 | c7 |
| 4 | a4 | b11 | c7 |
| 5 | a5 | b11 | c7 |
| 6 | a6 | b11 | c7 |
| 7 | a7 | b11 | c7 |
| 8 | a8 | b11 | c7 |
| 9 | a9 | b11 | c7 |
| 10 | a10 | b11 | c7 |
| 11 | a1 | b1 | c1 |
| 12 | a1 | b1 | c2 |
| 13 | a1 | b1 | c3 |
| 14 | a1 | b1 | c4 |
| 15 | a1 | b1 | c5 |
| 16 | a1 | b1 | c7 |
| 17 | a1 | b1 | c9 |
| 18 | a1 | b1 | c13 |
| 19 | a1 | b1 | c14 |
| 20 | a1 | b1 | c15 |
| 21 | a1 | b1 | c16 |
| 22 | a1 | b1 | c17 |
| 23 | a1 | b1 | c18 |
| 24 | a1 | b1 | c19 |
| 25 | a1 | b1 | c20 |
| 26 | a1 | b1 | c21 |
| 27 | a1 | b1 | c24 |
| 28 | a1 | b2 | c1 |
| 29 | a1 | b2 | c2 |
| 30 | a1 | b2 | c3 |
| 31 | a1 | b2 | c4 |
| 32 | a1 | b2 | c5 |
| 33 | a1 | b2 | c7 |
| 34 | a1 | b2 | c9 |
| 35 | a1 | b2 | c13 |
| 36 | a1 | b2 | c14 |
| 37 | a1 | b2 | c15 |
| 38 | a1 | b2 | c16 |
| 39 | a1 | b2 | c17 |
| 40 | a1 | b2 | c18 |
| 41 | a1 | b2 | c19 |
| 42 | a1 | b2 | c20 |
| 43 | a1 | b2 | c21 |
| 44 | a1 | b2 | c24 |
| 45 | a1 | b2 | c25 |
| 46 | a1 | b2 | c26 |
| 47 | a1 | b3 | c1 |
| 48 | a1 | b3 | c2 |
| 49 | a1 | b3 | c3 |
| 50 | a1 | b3 | c4 |
| 51 | a1 | b3 | c5 |
| 52 | a1 | b3 | c7 |
| 53 | a1 | b3 | c9 |
| 54 | a1 | b3 | c13 |
| 55 | a1 | b3 | c14 |
| 56 | a1 | b3 | c15 |
| 57 | a1 | b3 | c16 |
| 58 | a1 | b3 | c18 |
| 59 | a1 | b3 | c19 |
| 60 | a1 | b3 | c20 |
| 61 | a1 | b3 | c21 |
| 62 | a1 | b3 | c24 |
| 63 | a1 | b11 | c25 |
| 64 | a1 | b11 | c26 |
| 65 | a1 | b11 | c6 |
| 66 | a1 | b11 | c9 |
| 67 | a1 | b11 | c10 |
| 68 | a1 | b11 | c11 |
| 69 | a1 | b11 | c12 |
| 70 | a1 | b11 | c13 |
| 71 | a1 | b11 | c14 |
| 72 | a1 | b11 | c15 |
| 73 | a1 | b11 | c16 |
| 74 | a1 | b11 | c17 |
| 75 | a1 | b11 | c18 |
| 76 | a1 | b11 | c19 |
| 77 | a1 | b11 | c20 |
| 78 | a1 | b11 | c21 |
| 79 | a1 | b11 | c22 |
| 80 | a1 | b11 | c23 |
| 81 | a1 | b11 | c24 |
| 82 | a1 | b12 | c6 |
| 83 | a1 | b12 | c7 |
| 84 | a1 | b12 | c9 |
| 85 | a1 | b12 | c10 |
| 86 | a1 | b12 | c11 |
| 87 | a1 | b12 | c12 |
| 88 | a1 | b12 | c13 |
| 89 | a1 | b12 | c14 |
| 90 | a1 | b12 | c15 |
| 91 | a1 | b12 | c16 |
| 92 | a1 | b12 | c17 |
| 93 | a1 | b12 | c18 |
| 94 | a1 | b12 | c19 |
| 95 | a1 | b12 | c20 |
| 96 | a1 | b12 | c21 |
| 97 | a1 | b12 | c22 |
| 98 | a1 | b12 | c23 |
| 99 | a1 | b12 | c24 |
| 100 | a1 | b12 | c25 |
| 101 | a1 | b12 | c26 |
| 102 | a1 | b34 | c6 |
| 103 | a1 | b34 | c7 |
| 104 | a1 | b34 | c8 |
| 105 | a1 | b34 | c9 |
| 106 | a1 | b34 | c10 |
| 107 | a1 | b34 | c11 |
| 108 | a1 | b34 | c12 |
| 109 | a1 | b34 | c13 |
| 110 | a1 | b34 | c14 |
| 111 | a1 | b34 | c15 |
| 112 | a1 | b34 | c16 |
| 113 | a1 | b34 | c17 |
| 114 | a1 | b34 | c18 |
| 115 | a1 | b34 | c19 |
| 116 | a1 | b34 | c20 |
| 117 | a1 | b34 | c21 |
| 118 | a1 | b34 | c22 |
| 119 | a1 | b34 | c23 |
| 120 | a1 | b34 | c24 |
| 121 | a1 | b34 | c25 |
| 122 | a1 | b34 | c26 |
| 123 | a1 | b42 | c1 |
| 124 | a1 | b42 | c2 |
| 125 | a1 | b42 | c3 |
| 126 | a1 | b42 | c4 |
| 127 | a1 | b42 | c5 |
| 128 | a1 | b42 | c6 |
| 129 | a1 | b42 | c7 |
| 130 | a1 | b42 | c8 |
| 131 | a1 | b42 | c9 |
| 132 | a1 | b42 | c10 |
| 133 | a1 | b42 | c11 |
| 134 | a1 | b42 | c12 |
| 135 | a1 | b42 | c13 |
| 136 | a1 | b42 | c14 |
| 137 | a1 | b42 | c15 |
| 138 | a1 | b42 | c16 |
| 139 | a1 | b42 | c18 |
| 140 | a1 | b42 | c19 |
| 141 | a1 | b42 | c20 |
| 142 | a1 | b42 | c21 |
| 143 | a1 | b42 | c22 |
| 144 | a1 | b42 | c23 |
| 145 | a1 | b42 | c24 |
| 146 | a1 | b42 | c25 |
| 147 | a1 | b42 | c26 |
| 148 | a1 | c2 | b4 |
| 149 | a1 | c2 | b5 |
| 150 | a1 | c2 | b6 |
| 151 | a1 | c2 | b7 |
| 152 | a1 | c2 | b8 |
| 153 | a1 | c2 | b9 |
| 154 | a1 | c2 | b41 |
| 155 | a1 | c2 | b43 |
| 156 | a1 | c2 | b44 |
| 157 | a1 | c2 | b45 |
| 158 | a1 | c2 | b46 |
| 159 | a1 | c2 | b47 |
| 160 | a1 | c2 | c1 |
| 161 | a1 | c2 | c2 |
| 162 | a1 | c2 | c3 |
| 163 | a1 | c2 | c4 |
| 164 | a1 | c2 | c5 |
| 165 | a1 | c2 | c6 |
| 166 | a1 | c2 | c7 |
| 167 | a1 | c2 | c8 |
| 168 | a1 | c2 | c9 |
| 169 | a1 | c2 | c10 |
| 170 | a1 | c2 | c11 |
| 171 | a1 | c2 | c12 |
| 172 | a1 | c2 | c13 |
| 173 | a1 | c2 | c14 |
| 174 | a1 | c2 | c15 |
| 175 | a1 | c2 | c16 |
| 176 | a1 | c2 | c17 |
| 177 | a1 | c2 | c18 |
| 178 | a1 | c2 | c19 |
| 179 | a1 | c2 | c20 |
| 180 | a1 | c2 | c21 |
| 181 | a1 | c2 | c24 |
| 182 | a1 | c2 | c25 |
| 183 | a1 | c2 | c26 |
| 184 | a1 | c7 | b4 |
| 185 | a1 | c7 | b5 |
| 186 | a1 | c7 | b6 |
| 187 | a1 | c7 | b7 |
| 188 | a1 | c7 | b8 |
| 189 | a1 | c7 | b9 |
| 190 | a1 | c7 | b10 |
| 191 | a1 | c7 | b13 |
| 192 | a1 | c7 | b14 |
| 193 | a1 | c7 | b15 |
| 194 | a1 | c7 | b16 |
| 195 | a1 | c7 | b17 |
| 196 | a1 | c7 | b18 |
| 197 | a1 | c7 | b19 |
| 198 | a1 | c7 | b20 |
| 199 | a1 | c7 | b21 |
| 200 | a1 | c7 | b22 |
| 201 | a1 | c7 | b23 |
| 202 | a1 | c7 | b24 |
| 203 | a1 | c7 | b25 |
| 204 | a1 | c7 | b26 |
| 205 | a1 | c7 | b27 |
| 206 | a1 | c7 | b28 |
| 207 | a1 | c7 | b29 |
| 208 | a1 | c7 | b30 |
| 209 | a1 | c7 | b31 |
| 210 | a1 | c7 | b32 |
| 211 | a1 | c7 | b33 |
| 212 | a1 | c7 | b35 |
| 213 | a1 | c7 | b36 |
| 214 | a1 | c7 | b37 |
| 215 | a1 | c7 | b38 |
| 216 | a1 | c7 | b39 |
| 217 | a1 | c7 | b40 |
| 218 | a1 | c7 | b41 |
| 219 | a1 | c7 | b43 |
| 220 | a1 | c7 | b44 |
| 221 | a1 | c7 | b45 |
| 222 | a1 | c7 | b46 |
| 223 | a1 | c7 | b47 |
| 224 | a1 | c7 | c1 |
| 225 | a1 | c7 | c3 |
| 226 | a1 | c7 | c4 |
| 227 | a1 | c7 | c5 |
| 228 | a1 | c7 | c6 |
| 229 | a1 | c7 | c7 |
| 230 | a1 | c7 | c8 |
| 231 | a1 | c7 | c9 |
| 232 | a1 | c7 | c10 |
| 233 | a1 | c7 | c11 |
| 234 | a1 | c7 | c12 |
| 235 | a1 | c7 | c13 |
| 236 | a1 | c7 | c14 |
| 237 | a1 | c7 | c15 |
| 238 | a1 | c7 | c16 |
| 239 | a1 | c7 | c17 |
| 240 | a1 | c7 | c18 |
| 241 | a1 | c7 | c19 |
| 242 | a1 | c7 | c20 |
| 243 | a1 | c7 | c21 |
| 244 | a1 | c7 | c22 |
| 245 | a1 | c7 | c23 |
| 246 | a1 | c7 | c24 |
| 247 | a1 | c7 | c25 |
| 248 | a1 | c7 | c26 |
| 249 | a1 | c14 | b4 |
| 250 | a1 | c14 | b5 |
| 251 | a1 | c14 | b6 |
| 252 | a1 | c14 | b7 |
| 253 | a1 | c14 | b8 |
| 254 | a1 | c14 | b9 |
| 255 | a1 | c14 | b10 |
| 256 | a1 | c14 | b13 |
| 257 | a1 | c14 | b14 |
| 258 | a1 | c14 | b15 |
| 259 | a1 | c14 | b16 |
| 260 | a1 | c14 | b17 |
| 261 | a1 | c14 | b18 |
| 262 | a1 | c14 | b19 |
| 263 | a1 | c14 | b20 |
| 264 | a1 | c14 | b21 |
| 265 | a1 | c14 | b22 |
| 266 | a1 | c14 | b23 |
| 267 | a1 | c14 | b24 |
| 268 | a1 | c14 | b25 |
| 269 | a1 | c14 | b26 |
| 270 | a1 | c14 | b27 |
| 271 | a1 | c14 | b28 |
| 272 | a1 | c14 | b29 |
| 273 | a1 | c14 | b30 |
| 274 | a1 | c14 | b31 |
| 275 | a1 | c14 | b32 |
| 276 | a1 | c14 | b33 |
| 277 | a1 | c14 | b35 |
| 278 | a1 | c14 | b36 |
| 279 | a1 | c14 | b37 |
| 280 | a1 | c14 | b38 |
| 281 | a1 | c14 | b39 |
| 282 | a1 | c14 | b40 |
| 283 | a1 | c14 | b41 |
| 284 | a1 | c14 | b43 |
| 285 | a1 | c14 | b44 |
| 286 | a1 | c14 | b45 |
| 287 | a1 | c14 | b46 |
| 288 | a1 | c14 | b47 |
| 289 | a1 | c14 | c1 |
| 290 | a1 | c14 | c3 |
| 291 | a1 | c14 | c4 |
| 292 | a1 | c14 | c5 |
| 293 | a1 | c14 | c6 |
| 294 | a1 | c14 | c8 |
| 295 | a1 | c14 | c9 |
| 296 | a1 | c14 | c10 |
| 297 | a1 | c14 | c11 |
| 298 | a1 | c14 | c12 |
| 299 | a1 | c14 | c13 |
| 300 | a1 | c14 | c14 |
| 301 | a1 | c14 | c15 |
| 302 | a1 | c14 | c16 |
| 303 | a1 | c14 | c18 |
| 304 | a1 | c14 | c19 |
| 305 | a1 | c14 | c20 |
| 306 | a1 | c14 | c21 |
| 307 | a1 | c14 | c22 |
| 308 | a1 | c14 | c23 |
| 309 | a1 | c14 | c24 |
| 310 | a1 | c14 | c25 |
| 311 | a1 | c14 | c26 |
| 312 | a1 | c15 | b4 |
| 313 | a1 | c15 | b5 |
| 314 | a1 | c15 | b6 |
| 315 | a1 | c15 | b7 |
| 316 | a1 | c15 | b8 |
| 317 | a1 | c15 | b9 |
| 318 | a1 | c15 | b10 |
| 319 | a1 | c15 | b13 |
| 320 | a1 | c15 | b14 |
| 321 | a1 | c15 | b15 |
| 322 | a1 | c15 | b16 |
| 323 | a1 | c15 | b17 |
| 324 | a1 | c15 | b18 |
| 325 | a1 | c15 | b19 |
| 326 | a1 | c15 | b20 |
| 327 | a1 | c15 | b21 |
| 328 | a1 | c15 | b22 |
| 329 | a1 | c15 | b23 |
| 330 | a1 | c15 | b24 |
| 331 | a1 | c15 | b25 |
| 332 | a1 | c15 | b26 |
| 333 | a1 | c15 | b27 |
| 334 | a1 | c15 | b28 |
| 335 | a1 | c15 | b29 |
| 336 | a1 | c15 | b30 |
| 337 | a1 | c15 | b31 |
| 338 | a1 | c15 | b32 |
| 339 | a1 | c15 | b33 |
| 340 | a1 | c15 | b35 |
| 341 | a1 | c15 | b36 |
| 342 | a1 | c15 | b37 |
| 343 | a1 | c15 | b38 |
| 344 | a1 | c15 | b39 |
| 345 | a1 | c15 | b40 |
| 346 | a1 | c15 | b41 |
| 347 | a1 | c15 | b43 |
| 348 | a1 | c15 | b44 |
| 349 | a1 | c15 | b45 |
| 350 | a1 | c15 | b46 |
| 351 | a1 | c15 | b47 |
| 352 | a1 | c15 | c1 |
| 353 | a1 | c15 | c3 |
| 354 | a1 | c15 | c4 |
| 355 | a1 | c15 | c5 |
| 356 | a1 | c15 | c6 |
| 357 | a1 | c15 | c8 |
| 358 | a1 | c15 | c9 |
| 359 | a1 | c15 | c10 |
| 360 | a1 | c15 | c11 |
| 361 | a1 | c15 | c12 |
| 362 | a1 | c15 | c13 |
| 363 | a1 | c15 | c15 |
| 364 | a1 | c15 | c16 |
| 365 | a1 | c15 | c17 |
| 366 | a1 | c15 | c18 |
| 367 | a1 | c15 | c19 |
| 368 | a1 | c15 | c20 |
| 369 | a1 | c15 | c21 |
| 370 | a1 | c15 | c22 |
| 371 | a1 | c15 | c23 |
| 372 | a1 | c15 | c24 |
| 373 | a1 | c15 | c25 |
| 374 | a1 | c15 | c26 |
| 375 | a1 | c24 | b4 |
| 376 | a1 | c24 | b5 |
| 377 | a1 | c24 | b6 |
| 378 | a1 | c24 | b7 |
| 379 | a1 | c24 | b8 |
| 380 | a1 | c24 | b9 |
| 381 | a1 | c24 | b10 |
| 382 | a1 | c24 | b13 |
| 383 | a1 | c24 | b14 |
| 384 | a1 | c24 | b15 |
| 385 | a1 | c24 | b16 |
| 386 | a1 | c24 | b17 |
| 387 | a1 | c24 | b18 |
| 388 | a1 | c24 | b19 |
| 389 | a1 | c24 | b20 |
| 390 | a1 | c24 | b21 |
| 391 | a1 | c24 | b22 |
| 392 | a1 | c24 | b23 |
| 393 | a1 | c24 | b24 |
| 394 | a1 | c24 | b25 |
| 395 | a1 | c24 | b26 |
| 396 | a1 | c24 | b27 |
| 397 | a1 | c24 | b28 |
| 398 | a1 | c24 | b29 |
| 399 | a1 | c24 | b30 |
| 400 | a1 | c24 | b31 |
| 401 | a1 | c24 | b32 |
| 402 | a1 | c24 | b33 |
| 403 | a1 | c24 | b35 |
| 404 | a1 | c24 | b36 |
| 405 | a1 | c24 | b37 |
| 406 | a1 | c24 | b38 |
| 407 | a1 | c24 | b39 |
| 408 | a1 | c24 | b40 |
| 409 | a1 | c24 | b41 |
| 410 | a1 | c24 | b43 |
| 411 | a1 | c24 | b44 |
| 412 | a1 | c24 | b45 |
| 413 | a1 | c24 | b46 |
| 414 | a1 | c24 | b47 |
| 415 | a1 | c24 | c1 |
| 416 | a1 | c24 | c3 |
| 417 | a1 | c24 | c4 |
| 418 | a1 | c24 | c5 |
| 419 | a1 | c24 | c6 |
| 420 | a1 | c24 | c8 |
| 421 | a1 | c24 | c9 |
| 422 | a1 | c24 | c10 |
| 423 | a1 | c24 | c11 |
| 424 | a1 | c24 | c12 |
| 425 | a1 | c24 | c13 |
| 426 | a1 | c24 | c16 |
| 427 | a1 | c24 | c17 |
| 428 | a1 | c24 | c18 |
| 429 | a1 | c24 | c19 |
| 430 | a1 | c24 | c20 |
| 431 | a1 | c24 | c21 |
| 432 | a1 | c24 | c22 |
| 433 | a1 | c24 | c23 |
| 434 | a1 | c24 | c24 |
| 435 | a1 | c24 | c25 |
| 436 | a1 | c24 | c26 |
| 437 | a6 | b1 | c1 |
| 438 | a6 | b1 | c2 |
| 439 | a6 | b1 | c3 |
| 440 | a6 | b1 | c4 |
| 441 | a6 | b1 | c5 |
| 442 | a6 | b1 | c7 |
| 443 | a6 | b1 | c9 |
| 444 | a6 | b1 | c13 |
| 445 | a6 | b1 | c14 |
| 446 | a6 | b1 | c15 |
| 447 | a6 | b1 | c16 |
| 448 | a6 | b1 | c17 |
| 449 | a6 | b1 | c18 |
| 450 | a6 | b1 | c19 |
| 451 | a6 | b1 | c20 |
| 452 | a6 | b1 | c21 |
| 453 | a6 | b1 | c24 |
| 454 | a6 | b2 | c1 |
| 455 | a6 | b2 | c2 |
| 456 | a6 | b2 | c3 |
| 457 | a6 | b2 | c4 |
| 458 | a6 | b2 | c5 |
| 459 | a6 | b2 | c7 |
| 460 | a6 | b2 | c9 |
| 461 | a6 | b2 | c13 |
| 462 | a6 | b2 | c14 |
| 463 | a6 | b2 | c15 |
| 464 | a6 | b2 | c16 |
| 465 | a6 | b2 | c17 |
| 466 | a6 | b2 | c18 |
| 467 | a6 | b2 | c19 |
| 468 | a6 | b2 | c20 |
| 469 | a6 | b2 | c21 |
| 470 | a6 | b2 | c24 |
| 471 | a6 | b2 | c25 |
| 472 | a6 | b2 | c26 |
| 473 | a6 | b3 | c1 |
| 474 | a6 | b3 | c2 |
| 475 | a6 | b3 | c3 |
| 476 | a6 | b3 | c4 |
| 477 | a6 | b3 | c5 |
| 478 | a6 | b3 | c7 |
| 479 | a6 | b3 | c9 |
| 480 | a6 | b3 | c13 |
| 481 | a6 | b3 | c16 |
| 482 | a6 | b3 | c15 |
| 483 | a6 | b3 | c16 |
| 484 | a6 | b3 | c18 |
| 485 | a6 | b3 | c19 |
| 486 | a6 | b3 | c20 |
| 487 | a6 | b3 | c21 |
| 488 | a6 | b3 | c24 |
| 489 | a6 | b11 | c25 |
| 490 | a6 | b11 | c26 |
| 491 | a6 | b11 | c6 |
| 492 | a6 | b11 | c9 |
| 493 | a6 | b11 | c10 |
| 494 | a6 | b11 | c11 |
| 495 | a6 | b11 | c12 |
| 496 | a6 | b11 | c13 |
| 497 | a6 | b11 | c14 |
| 498 | a6 | b11 | c15 |
| 499 | a6 | b11 | c16 |
| 500 | a6 | b11 | c17 |
| 501 | a6 | b11 | c18 |
| 502 | a6 | b11 | c19 |
| 503 | a6 | b11 | c20 |
| 504 | a6 | b11 | c21 |
| 505 | a6 | b11 | c22 |
| 506 | a6 | b11 | c23 |
| 507 | a6 | b11 | c24 |
| 508 | a6 | b12 | c6 |
| 509 | a6 | b12 | c7 |
| 510 | a6 | b12 | c9 |
| 511 | a6 | b12 | c10 |
| 512 | a6 | b12 | c11 |
| 513 | a6 | b12 | c12 |
| 514 | a6 | b12 | c13 |
| 515 | a6 | b12 | c14 |
| 516 | a6 | b12 | c15 |
| 517 | a6 | b12 | c16 |
| 518 | a6 | b12 | c17 |
| 519 | a6 | b12 | c18 |
| 520 | a6 | b12 | c19 |
| 521 | a6 | b12 | c20 |
| 522 | a6 | b12 | c21 |
| 523 | a6 | b12 | c22 |
| 524 | a6 | b12 | c23 |
| 525 | a6 | b12 | c24 |
| 526 | a6 | b12 | c25 |
| 527 | a6 | b12 | c26 |
| 528 | a6 | b34 | c6 |
| 529 | a6 | b34 | c7 |
| 530 | a6 | b34 | c8 |
| 531 | a6 | b34 | c9 |
| 532 | a6 | b34 | c10 |
| 533 | a6 | b34 | c11 |
| 534 | a6 | b34 | c12 |
| 535 | a6 | b34 | c13 |
| 536 | a6 | b34 | c14 |
| 537 | a6 | b34 | c15 |
| 538 | a6 | b34 | c16 |
| 539 | a6 | b34 | c17 |
| 540 | a6 | b34 | c18 |
| 541 | a6 | b34 | c19 |
| 542 | a6 | b34 | c20 |
| 543 | a6 | b34 | c21 |
| 544 | a6 | b34 | c22 |
| 545 | a6 | b34 | c23 |
| 546 | a6 | b34 | c24 |
| 547 | a6 | b34 | c25 |
| 548 | a6 | b34 | c26 |
| 549 | a6 | b42 | c1 |
| 550 | a6 | b42 | c2 |
| 551 | a6 | b42 | c3 |
| 552 | a6 | b42 | c4 |
| 553 | a6 | b42 | c5 |
| 554 | a6 | b42 | c6 |
| 555 | a6 | b42 | c7 |
| 556 | a6 | b42 | c8 |
| 557 | a6 | b42 | c9 |
| 558 | a6 | b42 | c10 |
| 559 | a6 | b42 | c11 |
| 560 | a6 | b42 | c12 |
| 561 | a6 | b42 | c13 |
| 562 | a6 | b42 | c14 |
| 563 | a6 | b42 | c15 |
| 564 | a6 | b42 | c16 |
| 565 | a6 | b42 | c18 |
| 566 | a6 | b42 | c19 |
| 567 | a6 | b42 | c20 |
| 568 | a6 | b42 | c21 |
| 569 | a6 | b42 | c22 |
| 570 | a6 | b42 | c23 |
| 571 | a6 | b42 | c24 |
| 572 | a6 | b42 | c25 |
| 573 | a6 | b42 | c26 |
| 574 | a6 | c2 | b4 |
| 575 | a6 | c2 | b5 |
| 576 | a6 | c2 | b6 |
| 577 | a6 | c2 | b7 |
| 578 | a6 | c2 | b8 |
| 579 | a6 | c2 | b9 |
| 580 | a6 | c2 | b41 |
| 581 | a6 | c2 | b43 |
| 582 | a6 | c2 | b44 |
| 583 | a6 | c2 | b45 |
| 584 | a6 | c2 | b46 |
| 585 | a6 | c2 | b47 |
| 586 | a6 | c2 | c1 |
| 587 | a6 | c2 | c2 |
| 588 | a6 | c2 | c3 |
| 589 | a6 | c2 | c4 |
| 590 | a6 | c2 | c5 |
| 591 | a6 | c2 | c6 |
| 592 | a6 | c2 | c7 |
| 593 | a6 | c2 | c8 |
| 594 | a6 | c2 | c9 |
| 595 | a6 | c2 | c10 |
| 596 | a6 | c2 | c11 |
| 597 | a6 | c2 | c12 |
| 598 | a6 | c2 | c13 |
| 599 | a6 | c2 | c14 |
| 600 | a6 | c2 | c15 |
| 601 | a6 | c2 | c16 |
| 602 | a6 | c2 | c17 |
| 603 | a6 | c2 | c18 |
| 604 | a6 | c2 | c19 |
| 605 | a6 | c2 | c20 |
| 606 | a6 | c2 | c21 |
| 607 | a6 | c2 | c24 |
| 608 | a6 | c2 | c25 |
| 609 | a6 | c2 | c26 |
| 610 | a6 | c2 | b4 |
| 611 | a6 | c7 | b5 |
| 612 | a6 | c7 | b6 |
| 613 | a6 | c7 | b7 |
| 614 | a6 | c7 | b8 |
| 615 | a6 | c7 | b9 |
| 616 | a6 | c7 | b10 |
| 617 | a6 | c7 | b13 |
| 618 | a6 | c7 | b14 |
| 619 | a6 | c7 | b15 |
| 620 | a6 | c7 | b16 |
| 621 | a6 | c7 | b17 |
| 622 | a6 | c7 | b18 |
| 623 | a6 | c7 | b19 |
| 624 | a6 | c7 | b20 |
| 625 | a6 | c7 | b21 |
| 626 | a6 | c7 | b22 |
| 627 | a6 | c7 | b23 |
| 628 | a6 | c7 | b24 |
| 629 | a6 | c7 | b25 |
| 630 | a6 | c7 | b26 |
| 631 | a6 | c7 | b27 |
| 632 | a6 | c7 | b28 |
| 633 | a6 | c7 | b29 |
| 634 | a6 | c7 | b30 |
| 635 | a6 | c7 | b31 |
| 636 | a6 | c7 | b32 |
| 637 | a6 | c7 | b33 |
| 638 | a6 | c7 | b35 |
| 639 | a6 | c7 | b36 |
| 640 | a6 | c7 | b37 |
| 641 | a6 | c7 | b38 |
| 642 | a6 | c7 | b39 |
| 643 | a6 | c7 | b40 |
| 644 | a6 | c7 | b41 |
| 645 | a6 | c7 | b43 |
| 646 | a6 | c7 | b44 |
| 647 | a6 | c7 | b45 |
| 648 | a6 | c7 | b46 |
| 649 | a6 | c7 | b47 |
| 650 | a6 | c7 | c1 |
| 651 | a6 | c7 | c3 |
| 652 | a6 | c7 | c4 |
| 653 | a6 | c7 | c5 |
| 654 | a6 | c7 | c6 |
| 655 | a6 | c7 | c7 |
| 656 | a6 | c7 | c8 |
| 657 | a6 | c7 | c9 |
| 658 | a6 | c7 | c10 |
| 659 | a6 | c7 | c11 |
| 660 | a6 | c7 | c12 |
| 661 | a6 | c7 | c13 |
| 662 | a6 | c7 | c14 |
| 663 | a6 | c7 | c15 |
| 664 | a6 | c7 | c16 |
| 665 | a6 | c7 | c17 |
| 666 | a6 | c7 | c18 |
| 667 | a6 | c7 | c19 |
| 668 | a6 | c7 | c20 |
| 669 | a6 | c7 | c21 |
| 670 | a6 | c7 | c22 |
| 671 | a6 | c7 | c23 |
| 672 | a6 | c7 | c24 |
| 673 | a6 | c7 | c25 |
| 674 | a6 | c7 | c26 |
| 675 | a6 | c14 | b4 |
| 676 | a6 | c14 | b5 |
| 677 | a6 | c14 | b6 |
| 678 | a6 | c14 | b7 |
| 679 | a6 | c14 | b8 |
| 680 | a6 | c14 | b9 |
| 681 | a6 | c14 | b10 |
| 682 | a6 | c14 | b13 |
| 683 | a6 | c14 | b14 |
| 684 | a6 | c14 | b15 |
| 685 | a6 | c14 | b16 |
| 686 | a6 | c14 | b17 |
| 687 | a6 | c14 | b18 |
| 688 | a6 | c14 | b19 |
| 689 | a6 | c14 | b20 |
| 690 | a6 | c14 | b21 |
| 691 | a6 | c14 | b22 |
| 692 | a6 | c14 | b23 |
| 693 | a6 | c14 | b24 |
| 694 | a6 | c14 | b25 |
| 695 | a6 | c14 | b26 |
| 696 | a6 | c14 | b27 |
| 697 | a6 | c14 | b28 |
| 698 | a6 | c14 | b29 |
| 699 | a6 | c14 | b30 |
| 700 | a6 | c14 | b31 |
| 701 | a6 | c14 | b32 |
| 702 | a6 | c14 | b33 |
| 703 | a6 | c14 | b35 |
| 704 | a6 | c14 | b36 |
| 705 | a6 | c14 | b37 |
| 706 | a6 | c14 | b38 |
| 707 | a6 | c14 | b39 |
| 708 | a6 | c14 | b40 |
| 709 | a6 | c14 | b41 |
| 710 | a6 | c14 | b43 |
| 711 | a6 | c14 | b44 |
| 712 | a6 | c14 | b45 |
| 713 | a6 | c14 | b46 |
| 714 | a6 | c14 | b47 |
| 715 | a6 | c14 | c1 |
| 716 | a6 | c14 | c3 |
| 717 | a6 | c14 | c4 |
| 718 | a6 | c14 | c5 |
| 719 | a6 | c14 | c6 |
| 720 | a6 | c14 | c8 |
| 721 | a6 | c14 | c9 |
| 722 | a6 | c14 | c10 |
| 723 | a6 | c14 | c11 |
| 724 | a6 | c14 | c12 |
| 725 | a6 | c14 | c13 |
| 726 | a6 | c14 | c14 |
| 727 | a6 | c14 | c15 |
| 728 | a6 | c14 | c16 |
| 729 | a6 | c14 | c18 |
| 730 | a6 | c14 | c19 |
| 731 | a6 | c14 | c20 |
| 732 | a6 | c14 | c21 |
| 733 | a6 | c14 | c22 |
| 734 | a6 | c14 | c23 |
| 735 | a6 | c14 | c24 |
| 736 | a6 | c14 | c25 |
| 737 | a6 | c14 | c26 |
| 738 | a6 | c15 | b4 |
| 739 | a6 | c15 | b5 |
| 740 | a6 | c15 | b6 |
| 741 | a6 | c15 | b7 |
| 742 | a6 | c15 | b8 |
| 743 | a6 | c15 | b9 |
| 744 | a6 | c15 | b10 |
| 745 | a6 | c15 | b13 |
| 746 | a6 | c15 | b14 |
| 747 | a6 | c15 | b15 |
| 748 | a6 | c15 | b16 |
| 749 | a6 | c15 | b17 |
| 750 | a6 | c15 | b18 |
| 751 | a6 | c15 | b19 |
| 752 | a6 | c15 | b20 |
| 753 | a6 | c15 | b21 |
| 754 | a6 | c15 | b22 |
| 755 | a6 | c15 | b23 |
| 756 | a6 | c15 | b24 |
| 757 | a6 | c15 | b25 |
| 758 | a6 | c15 | b26 |
| 759 | a6 | c15 | b27 |
| 760 | a6 | c15 | b28 |
| 761 | a6 | c15 | b29 |
| 762 | a6 | c15 | b30 |
| 763 | a6 | c15 | b31 |
| 764 | a6 | c15 | b32 |
| 765 | a6 | c15 | b33 |
| 766 | a6 | c15 | b35 |
| 767 | a6 | c15 | b36 |
| 768 | a6 | c15 | b37 |
| 769 | a6 | c15 | b38 |
| 770 | a6 | c15 | b39 |
| 771 | a6 | c15 | b40 |
| 772 | a6 | c15 | b41 |
| 773 | a6 | c15 | b43 |
| 774 | a6 | c15 | b44 |
| 775 | a6 | c15 | b45 |
| 776 | a6 | c15 | b46 |
| 777 | a6 | c15 | b47 |
| 778 | a6 | c15 | c1 |
| 779 | a6 | c15 | c3 |
| 780 | a6 | c15 | c4 |
| 781 | a6 | c15 | c5 |
| 782 | a6 | c15 | c6 |
| 783 | a6 | c15 | c8 |
| 784 | a6 | c15 | c9 |
| 785 | a6 | c15 | c10 |
| 786 | a6 | c15 | c11 |
| 787 | a6 | c15 | c12 |
| 788 | a6 | c15 | c13 |
| 789 | a6 | c15 | c15 |
| 790 | a6 | c15 | c16 |
| 791 | a6 | c15 | c17 |
| 792 | a6 | c15 | c18 |
| 793 | a6 | c15 | c19 |
| 794 | a6 | c15 | c20 |
| 795 | a6 | c15 | c21 |
| 796 | a6 | c15 | c22 |
| 797 | a6 | c15 | c23 |
| 798 | a6 | c15 | c24 |
| 799 | a6 | c15 | c25 |
| 800 | a6 | c15 | c26 |
| 801 | a6 | c24 | b4 |
| 802 | a6 | c24 | b5 |
| 803 | a6 | c24 | b6 |
| 804 | a6 | c24 | b7 |
| 805 | a6 | c24 | b8 |
| 806 | a6 | c24 | b9 |
| 807 | a6 | c24 | b10 |
| 808 | a6 | c24 | b13 |
| 809 | a6 | c24 | b14 |
| 810 | a6 | c24 | b15 |
| 811 | a6 | c24 | b16 |
| 812 | a6 | c24 | b17 |
| 813 | a6 | c24 | b18 |
| 814 | a6 | c24 | b19 |
| 815 | a6 | c24 | b20 |
| 816 | a6 | c24 | b21 |
| 817 | a6 | c24 | b22 |
| 818 | a6 | c24 | b23 |
| 819 | a6 | c24 | b24 |
| 820 | a6 | c24 | b25 |
| 821 | a6 | c24 | b26 |
| 822 | a6 | c24 | b27 |
| 823 | a6 | c24 | b28 |
| 824 | a6 | c24 | b29 |
| 825 | a6 | c24 | b30 |
| 826 | a6 | c24 | b31 |
| 827 | a6 | c24 | b32 |
| 828 | a6 | c24 | b33 |
| 829 | a6 | c24 | b35 |
| 830 | a6 | c24 | b36 |
| 831 | a6 | c24 | b37 |
| 832 | a6 | c24 | b38 |
| 833 | a6 | c24 | b39 |
| 834 | a6 | c24 | b40 |
| 835 | a6 | c24 | b41 |
| 836 | a6 | c24 | b43 |
| 837 | a6 | c24 | b44 |
| 838 | a6 | c24 | b45 |
| 839 | a6 | c24 | b46 |
| 840 | a6 | c24 | b47 |
| 841 | a6 | c24 | c1 |
| 842 | a6 | c24 | c3 |
| 843 | a6 | c24 | c4 |
| 844 | a6 | c24 | c5 |
| 845 | a6 | c24 | c6 |
| 846 | a6 | c24 | c8 |
| 847 | a6 | c24 | c9 |
| 848 | a6 | c24 | c10 |
| 849 | a6 | c24 | c11 |
| 850 | a6 | c24 | c12 |
| 851 | a6 | c24 | c13 |
| 852 | a6 | c24 | c16 |
| 853 | a6 | c24 | c17 |
| 854 | a6 | c24 | c18 |
| 855 | a6 | c24 | c19 |
| 856 | a6 | c24 | c20 |
| 857 | a6 | c24 | c21 |
| 858 | a6 | c24 | c22 |
| 859 | a6 | c24 | c23 |
| 860 | a6 | c24 | c24 |
| 861 | a6 | c24 | c25 |
| 862 | a6 | c24 | c26 |
| 863 | a5 | b10 | c26 |
| 864 | a1 | b10 | c7 |
| 865 | a1 | b48 | c7 |
| 866 | a1 | b48 | c14 |
| 867 | a1 | b10 | c14 |
| 868 | a1 | b48 | c14 |

The amine compound according to an embodiment may include the first substituent, the second substituent, and the third substituent linked to the core nitrogen atom, thereby achieving high efficiency and a long service life of the light emitting element.

The amine compound according to an embodiment may include an amine group, and the first to third substituents may each be bonded to the amine group of the amine compound. The first substituent may include a benzonaphthofuran moiety or a benzonaphthothiophene moiety. The first substituent may have a feature in that an oxygen atom of the benzonaphthofuran moiety or a sulfur atom of the benzonaphthothiophene moiety is at a meta position to the nitrogen atom of the amine. The second substituent may include a dibenzofuran moiety, a dibenzothiophene moiety, or a 9,9-diphenylfluorene moiety. The second substituent may be directly bonded to the core nitrogen atom. The third substituent may be linked to the core nitrogen atom via an arylene linker or a heteroarylene linker, or may be directly linked to the core nitrogen atom without a linker. The amine compound according to an embodiment may have excellent electrical stability and high charge transport ability due to the introduction of such a substituent and the specification of the substitution position. Accordingly, the service life of the amine compound according to an embodiment may be improved. The light emitting device according to an embodiment including the amine compound of an embodiment may have an improvement in luminous efficiency and service life.

In the light emitting element ED according to an embodiment, the hole transport region HTR may further include a compound represented by Formula H-1.

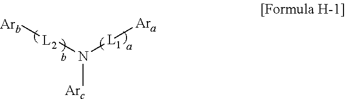

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H-1, a and b may each independently be an integer from 0 to 10. If a or b is 2 or more, multiple L₁ groups or multiple L₂ groups may each independently be a substituted or unsubstituted arylene group of 6 to 30 ring-forming carbon atoms or a substituted or unsubstituted heteroarylene group of 2 to 30 ring-forming carbon atoms.

In Formula H-1, Arₐ and Ar_{b} may each independently be a substituted or unsubstituted aryl group of 6 to 30 ring-forming carbon atoms or a substituted or unsubstituted heteroaryl group of 2 to 30 ring-forming carbon atoms. In Formula H-1, Ar_{c} may be a substituted or unsubstituted aryl group of 6 to 30 ring-forming carbon atoms.

In an embodiment, a compound represented by Formula H-1 may be a monoamine compound. In another embodiment, a compound represented by Formula H-1 may be a diamine compound in which at least one of Arₐ to Ar_{c} includes an amine group as a substituent. In still another embodiment, a compound represented by Formula H-1 may be a carbazole-based compound in which at least one of Arₐ and Ar_{b} includes a substituted or unsubstituted carbazole group, or may be a fluorene-based compound in which at least one of Arₐ and Ar_{b} includes a substituted or unsubstituted fluorene group.

The compound represented by Formula H-1 may be any compound selected from Compound Group H. However, the compounds listed in Compound Group H are only examples, and the compound represented by Formula H-1 is not limited to Compound Group H.

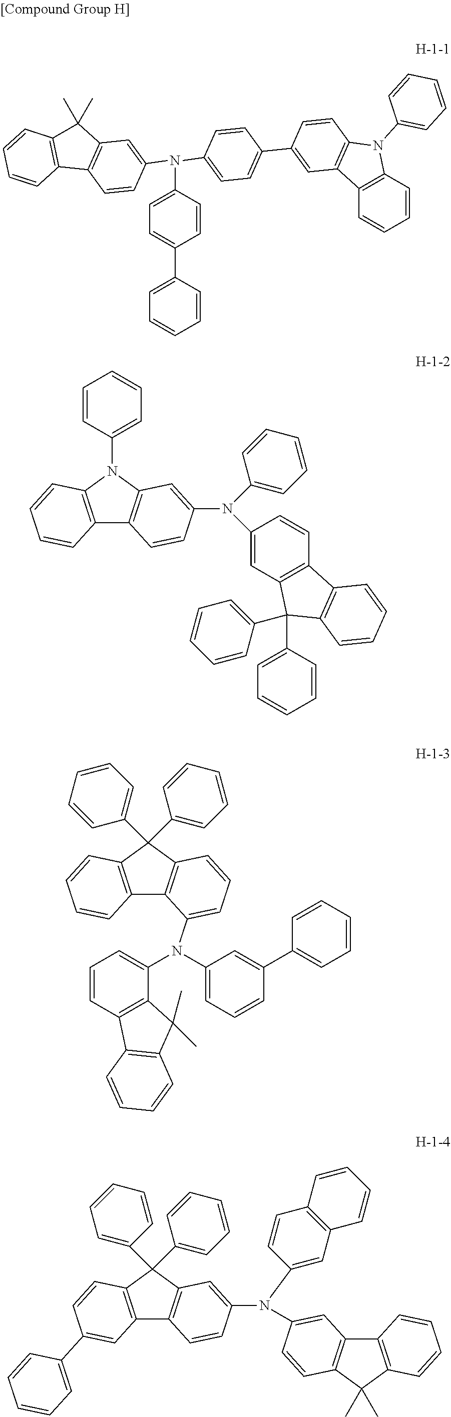 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

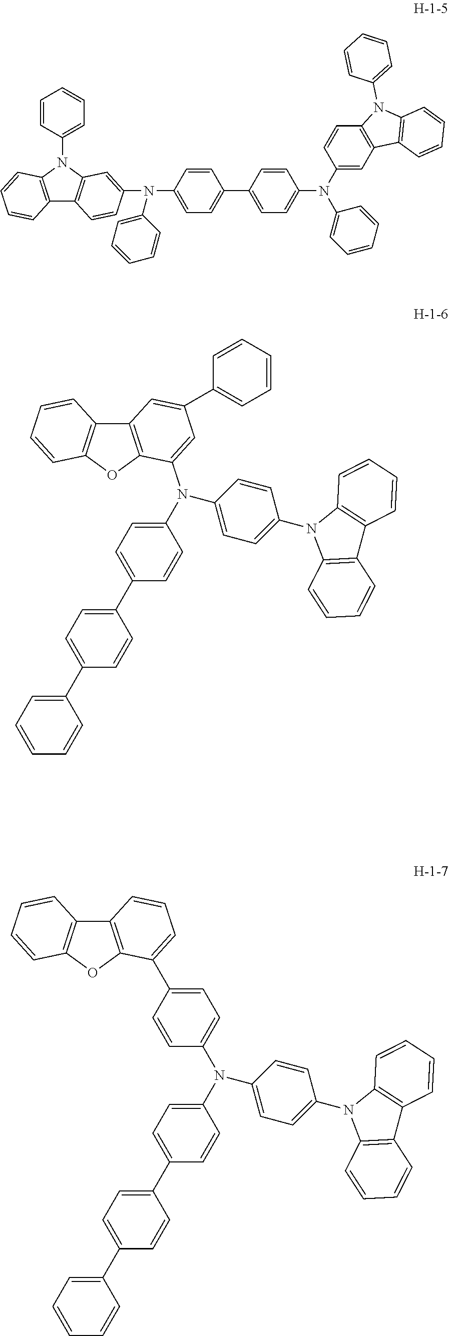 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

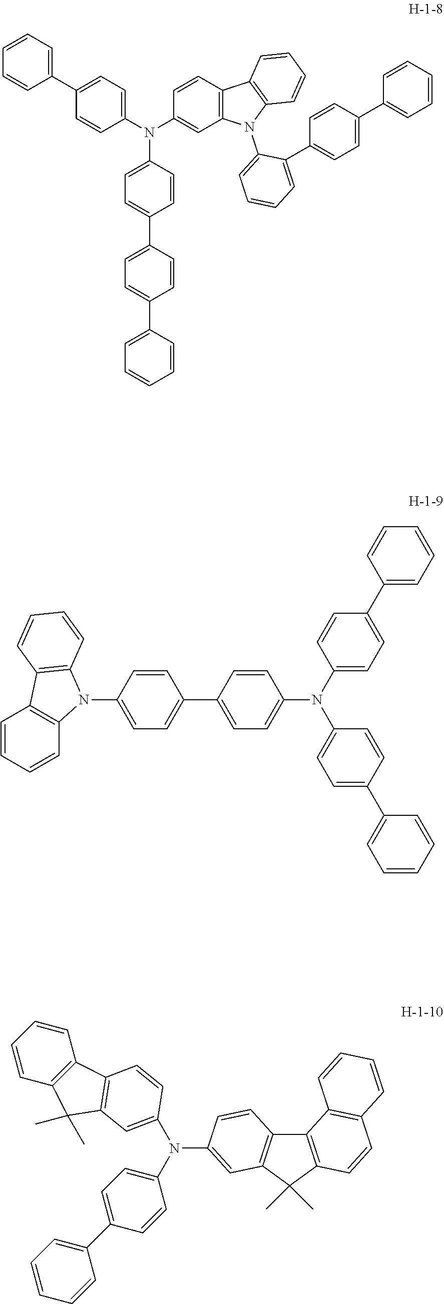 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

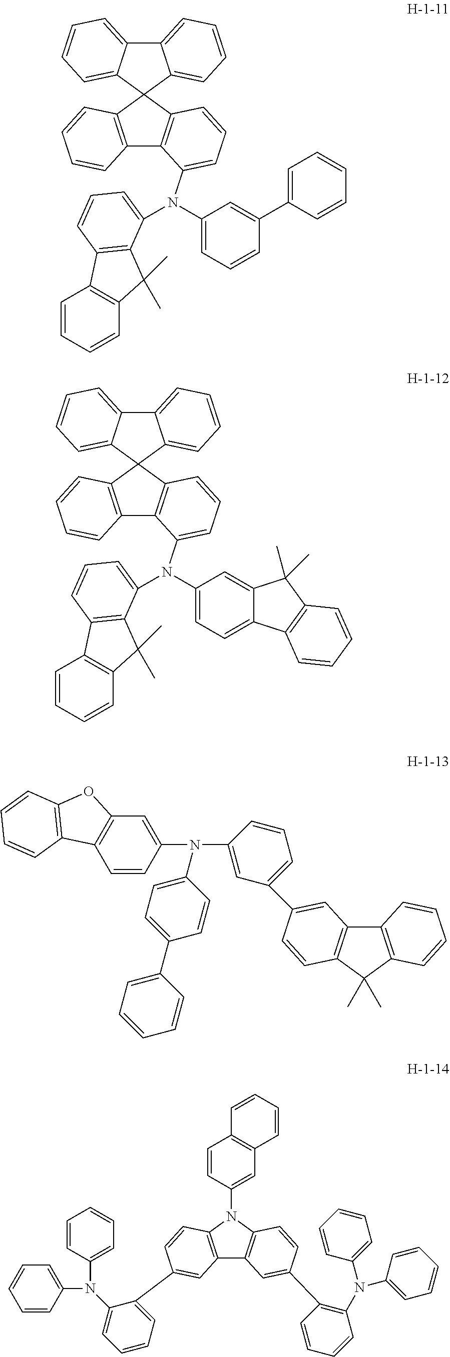

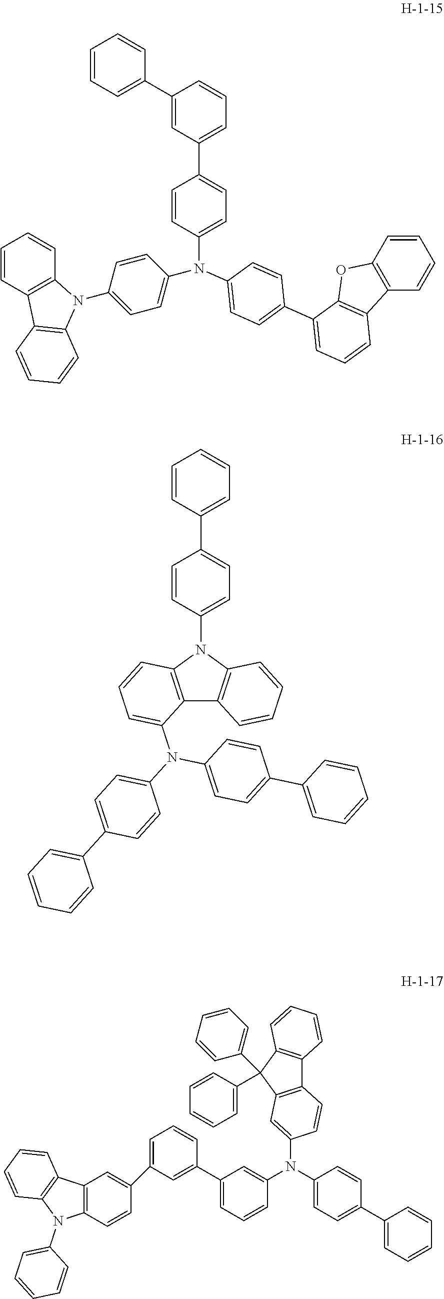 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

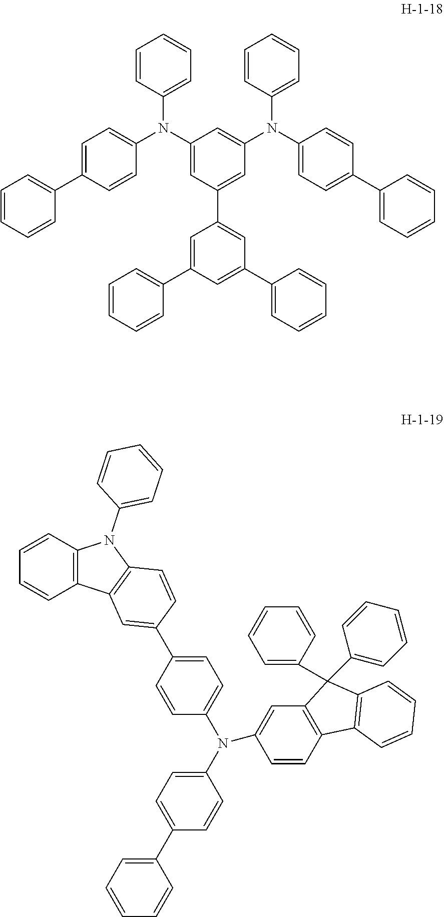 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In embodiments, the hole transport region HTR may further include a hole transport material of the related art.

For example, the hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one of a hole injection layer HIL, a hole transport layer HTL, and an electron blocking layer EBL.

A thickness of the hole transport region HTR may be in a range of about 100 Å to about 10,000 Å. For example, the thickness of the hole transport region HTR may be in a range of about 100 Å to about 5,000 Å. If the hole transport region HTR includes a hole injection layer HIL, a thickness of the hole injection region HIL may be in a range of about 30 Å to about 1,000 Å. If the hole transport region HTR includes a hole transport layer HTL, a thickness of the hole transport layer HTL may be in a range of about 30 Å to about 1,000 Å. If the hole transport region HTR includes an electron blocking layer EBL, a thickness of the electron blocking layer EBL may be in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase of a driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a metal halide compound, a quinone derivative, a metal oxide, and a cyano group-containing compounds, but embodiments are not limited thereto.

For example, the p-dopant may include a metal halide compound such as CuI and RbI; a quinone derivative such as tetracyanoquinodimethane (TCNQ) and 2,3,5,6-tetrafluoro-7,7′,8,8-tetracyanoquinodimethane (F4-TCNQ); a metal oxide such as tungsten oxide and molybdenum oxide; a cyano group-containing compound such as dipyrazino[2,3-f: 2′,3′-h]quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

As described above, the hole transport region HTR may further include a buffer layer (not shown), in addition to a hole injection layer HIL, a hole transport layer HTL, and an electron blocking layer EBL. The buffer layer (not shown) may compensate for a resonance distance according to a wavelength of light emitted from an emission layer EML and may increase emission efficiency. Materials which may be included in the buffer layer (not shown) may include materials which may be included in the hole transport region HTR.

The emission layer EML may be provided on the hole transport region HTR. The emission layer EML may have a thickness in a range of about 100 Å to about 1,000 Å. For example, the emission layer EML may have a thickness in a range of about 100 Å to about 300 Å. The emission layer EML may be a layer consisting of a single material, a layer including different materials, or a structure including multiple layers including different materials.

In the light emitting element ED according to an embodiment, the emission layer EML may emit blue light. The light emitting element ED may include the amine compound according to an embodiment in a hole transport region HTR and may show high efficiency and long-life characteristics in a blue emission region. However, embodiments are not limited thereto.

In the light emitting element ED according to an embodiment, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In the light emitting elements ED according to embodiments, as shown in each of FIG. 3 to FIG. 6, the emission layer EML may include a host and a dopant.

In an embodiment, the emission layer EML may include a compound represented by Formula E-1. The compound represented by Formula E-1 may be used as a fluorescence host material.

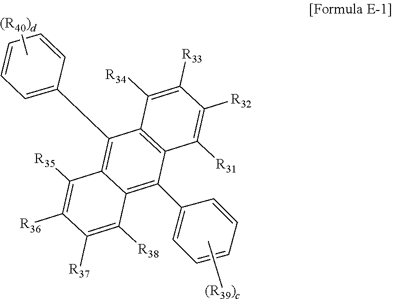

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. For example, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

In an embodiment, the compound represented by Formula E-1 may be any compound selected from Compound E1 to Compound E19.

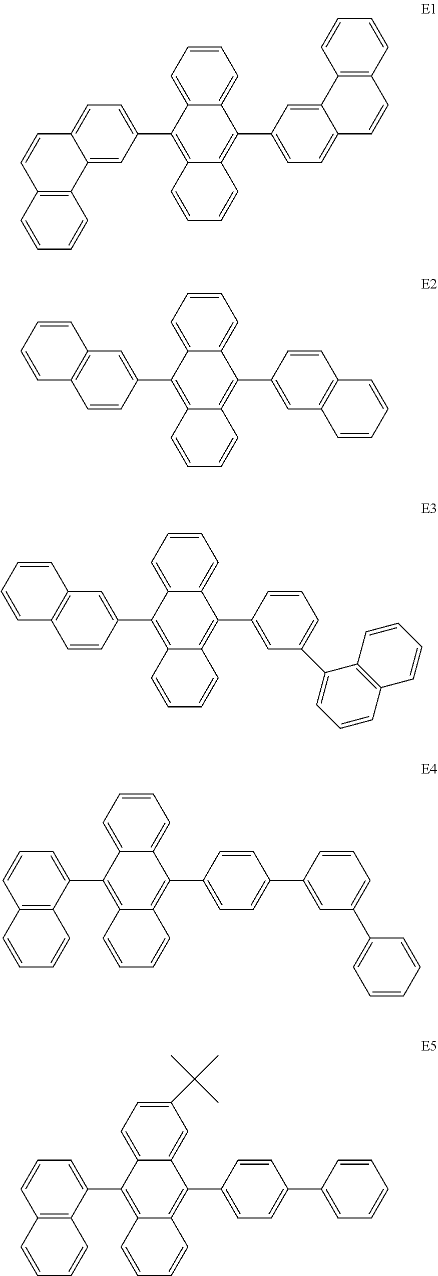

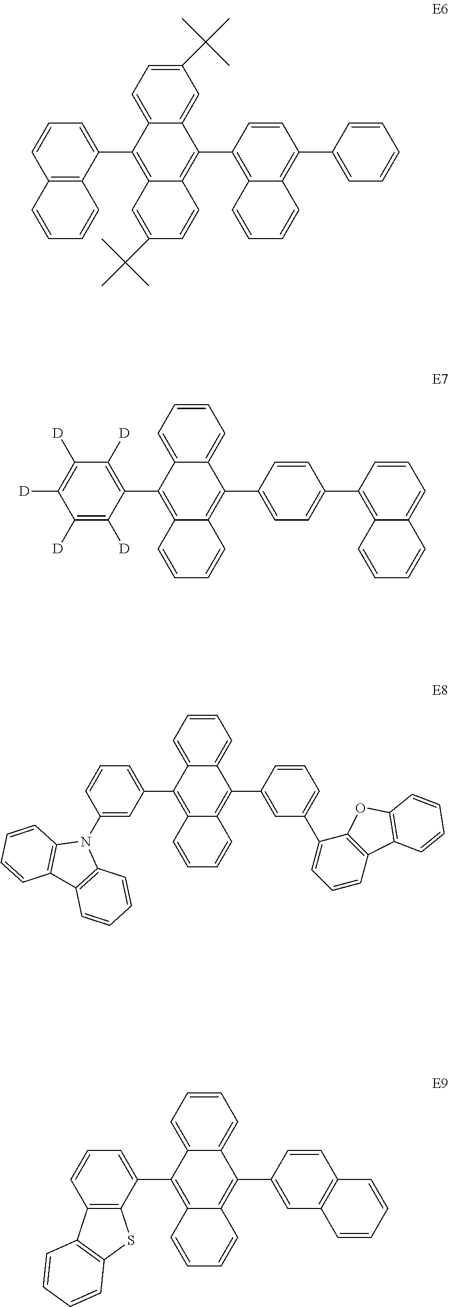

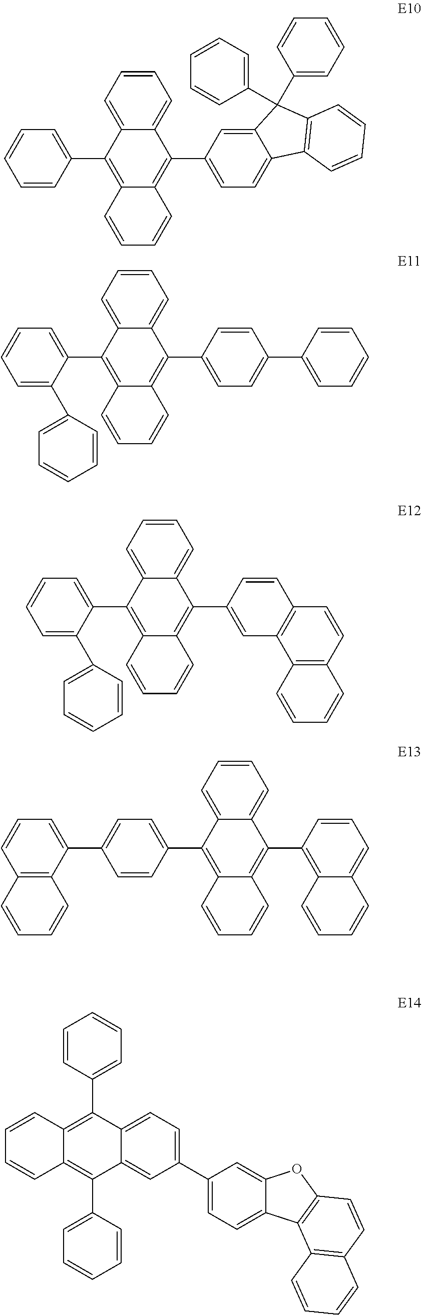

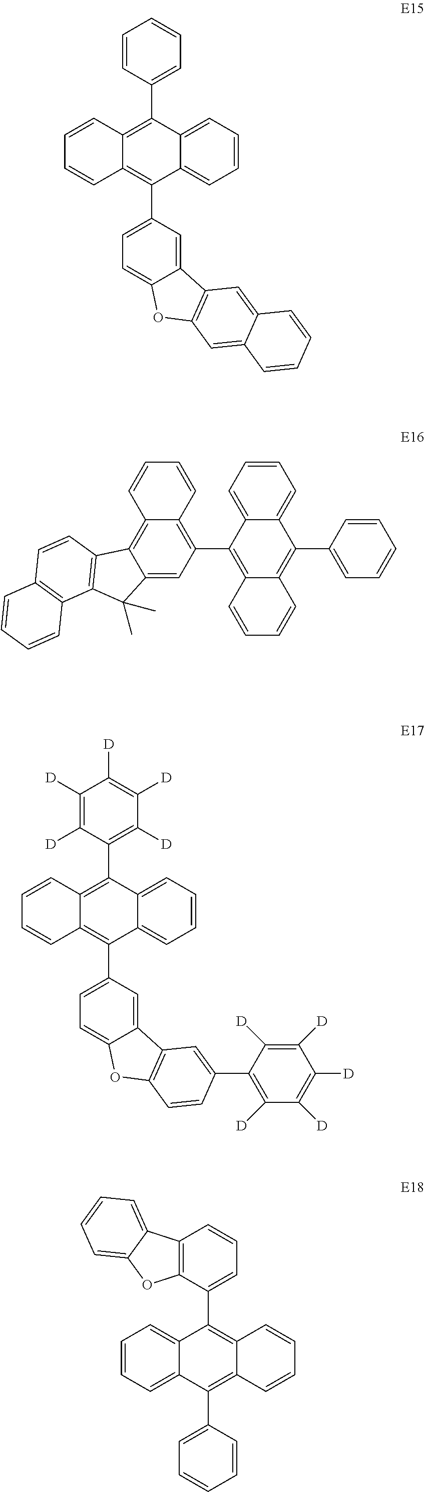

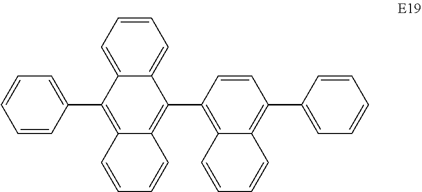 [SMILES: c1ccc(-c2ccc(-c3c4ccccc4c(-c4ccccc4)c4ccccc34)c3ccccc23)cc1]

In an embodiment, the emission layer EML may include a compound represented by Formula E-2a or Formula E-2b. The compound represented by Formula E-2a or Formula E-2b may be used as a phosphorescence host material.

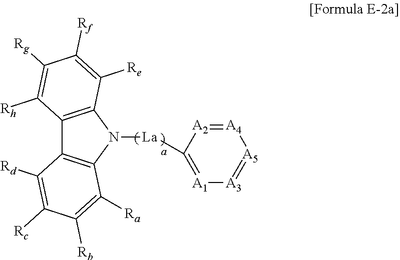

In Formula E-2a, a may be an integer from 0 to 10; and Lₐ may be a direct linkage, a substituted or unsubstituted arylene group of 6 to 30 ring-forming carbon atoms, or a substituted or unsubstituted heteroarylene group of 2 to 30 ring-forming carbon atoms. If a is 2 or more, multiple Lₐ groups may each independently be a substituted or unsubstituted arylene group of 6 to 30 ring-forming carbon atoms or a substituted or unsubstituted heteroarylene group of 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. For example, Rₐ to Rᵢ may be bonded to an adjacent group to form a hydrocarbon ring or a heterocycle including N, O, S, etc. as a ring-forming atom.

In Formula E-2a, two or three of A₁ to A₅ may each be N, and the remainder of A₁ to A₅ may each independently be C(Rᵢ).

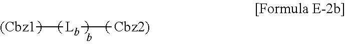

In Formula E-2b, Cbz1 and Cbz2 may each independently be an unsubstituted carbazole group, or a carbazole group substituted with an aryl group of 6 to 30 ring-forming carbon atoms. In Formula E-2b, L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b may be an integer from 0 to 10. If b is 2 or more, multiple L_{b} groups may each independently be a substituted or unsubstituted arylene group of 6 to 30 ring-forming carbon atoms or a substituted or unsubstituted heteroarylene group of 2 to 30 ring-forming carbon atoms.

The compound represented by Formula E-2a or Formula E-2b may be any compound selected from Compound Group E-2. However, the compounds listed in Compound Group E-2 are only examples, and the compound represented by Formula E-2a or Formula E-2b is not limited to Compound Group E-2.

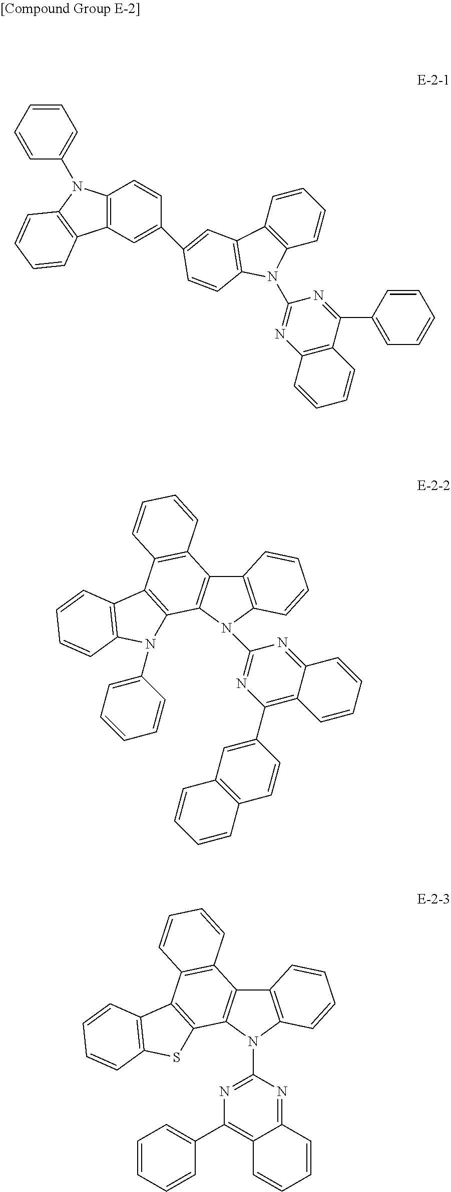 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

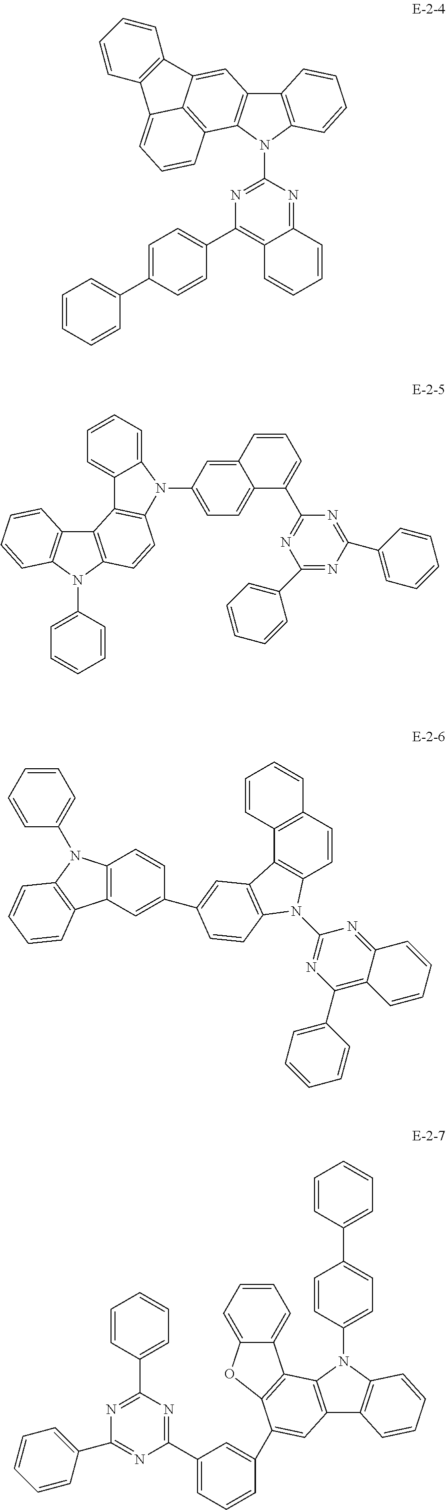 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

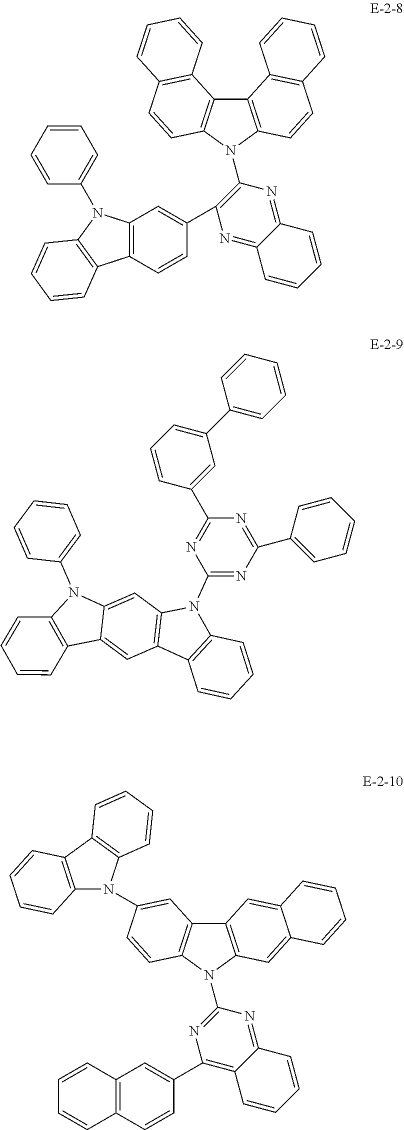 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

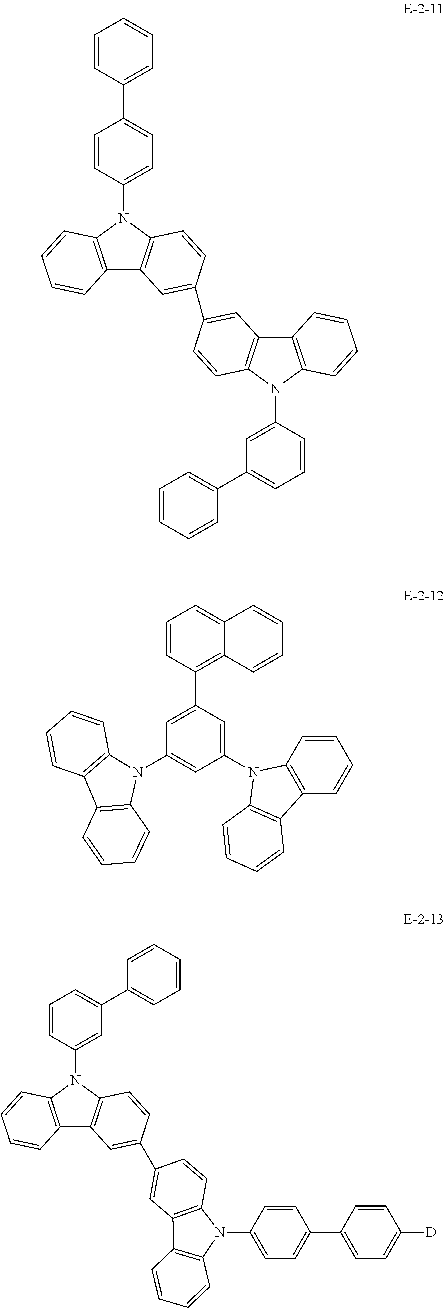 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

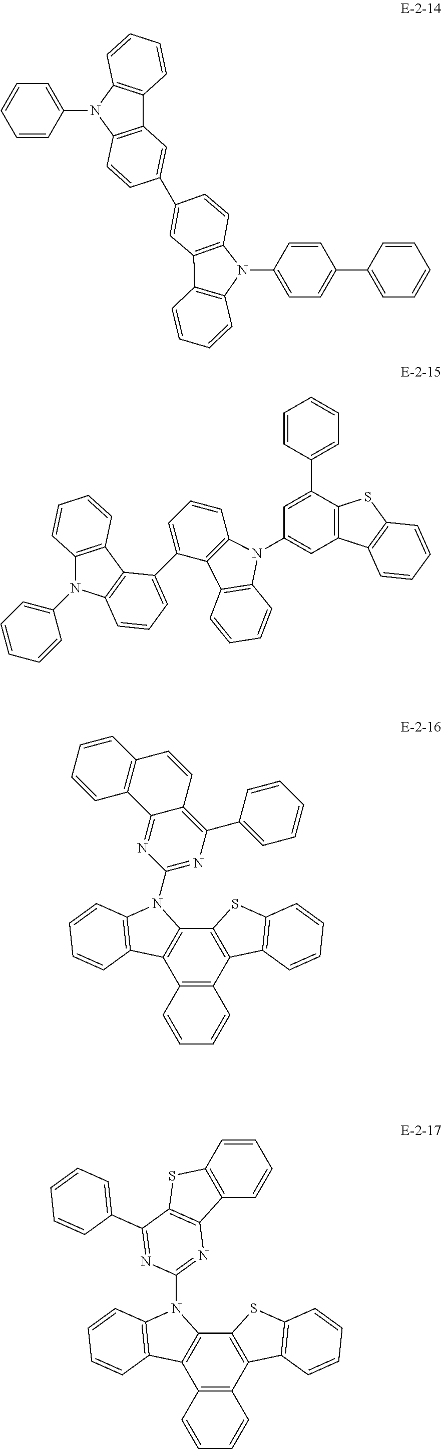 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

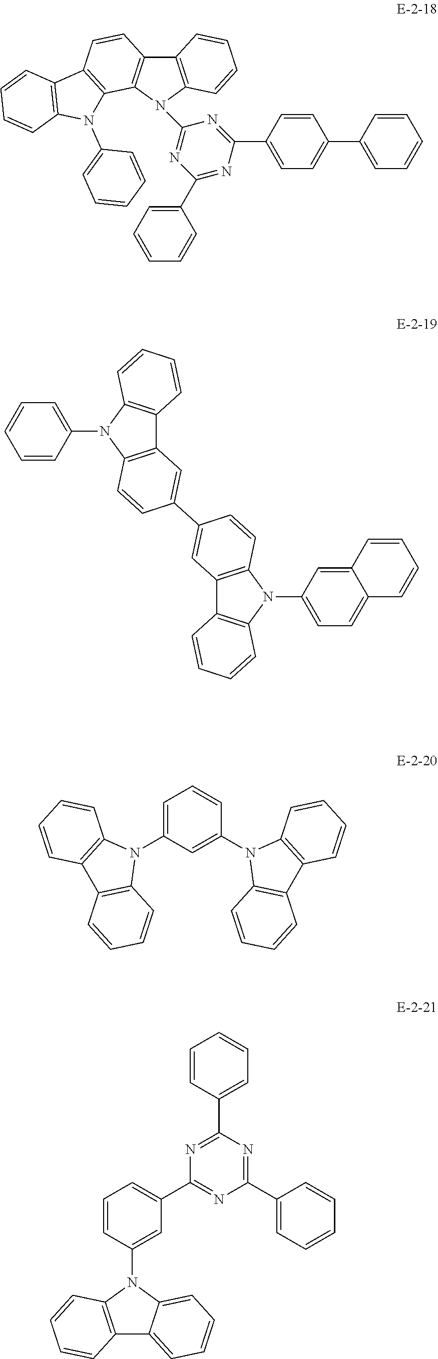 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

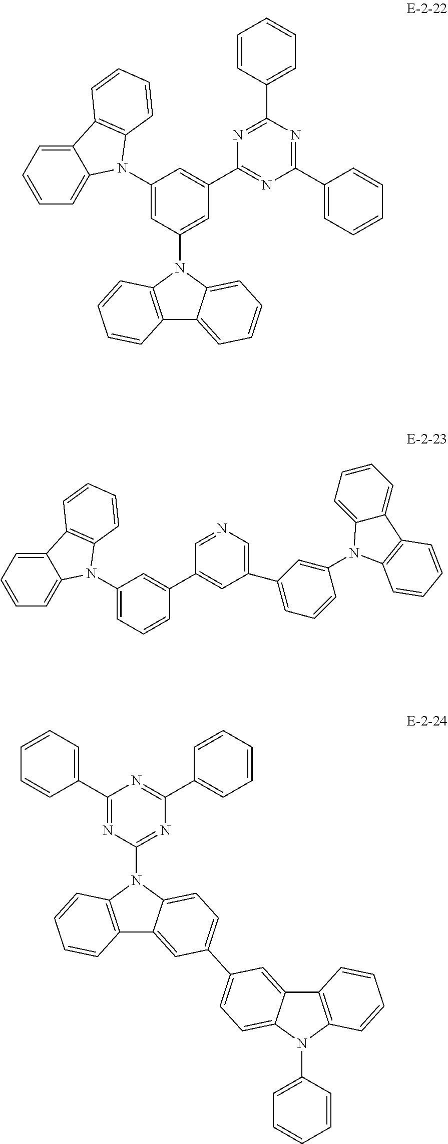 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl]ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments are not limited thereto. For example, tris(8-hydroxyquinolinato)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

In an embodiment, the emission layer EML may include a compound represented by Formula M-a or Formula M-b. The compound represented by Formula M-a or Formula M-b may be used as a phosphorescence dopant material. In an embodiment, the compound represented by Formula M-a or Formula M-b may be used as an auxiliary dopant material.

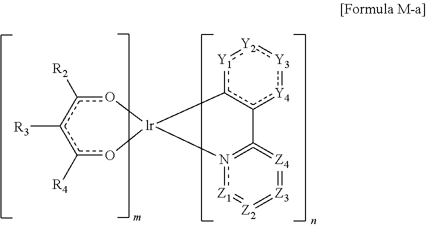

In Formula M-a, Y₁ to Y₄ and Z₁ to Z₄ may each independently be C(R₁) or N; and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. In Formula M-a, m may be 0 or 1, and n may be 2 or 3. In Formula M-a, if m is 0, n may be 3, and if m is 1, n may be 2.

The compound represented by Formula M-a may be any compound selected from Compounds M-a1 to M-a25. However, Compounds M-a1 to M-a25 are only examples, and the compound represented by Formula M-a is not limited to Compounds M-a1 to M-a25.

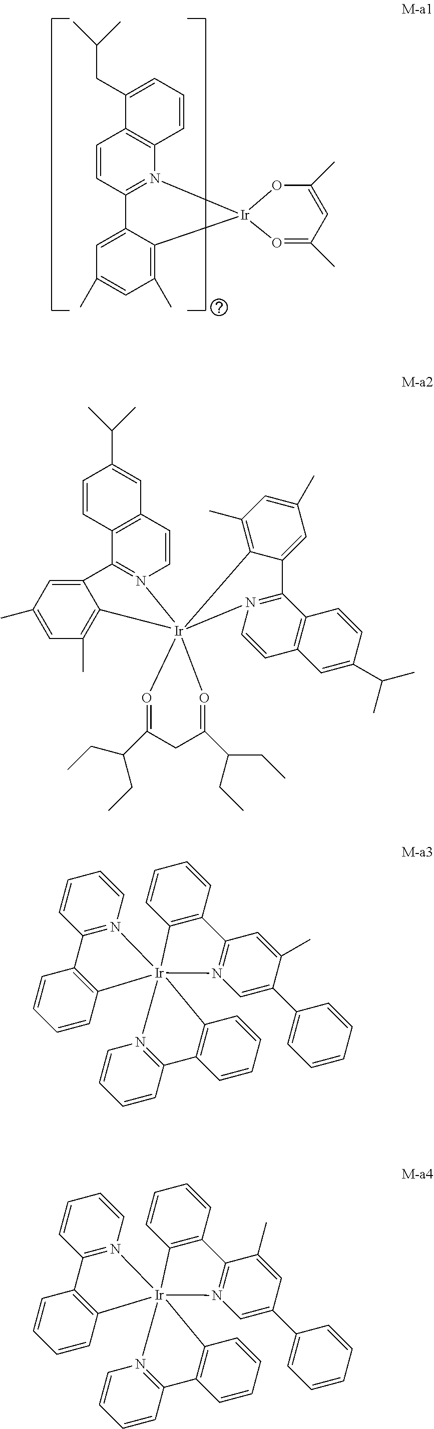

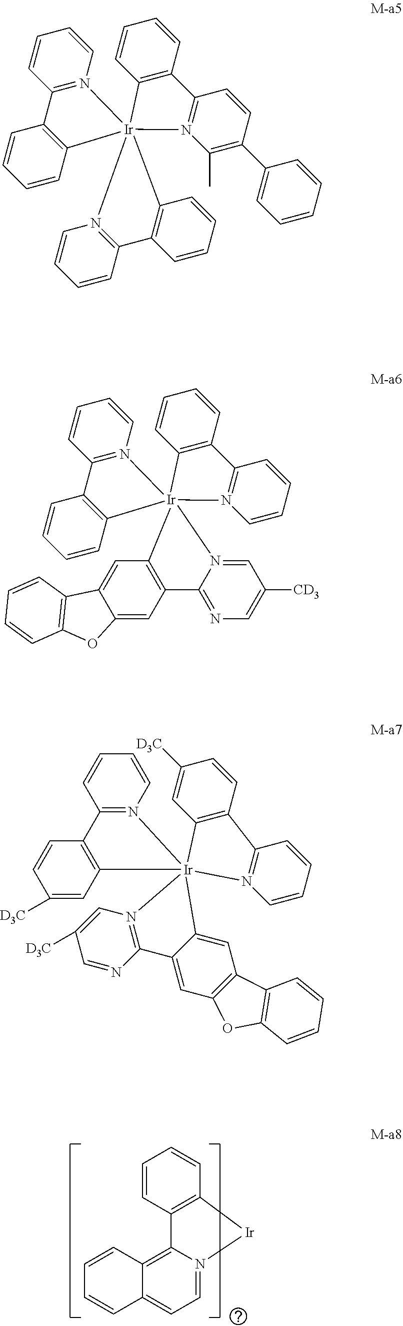

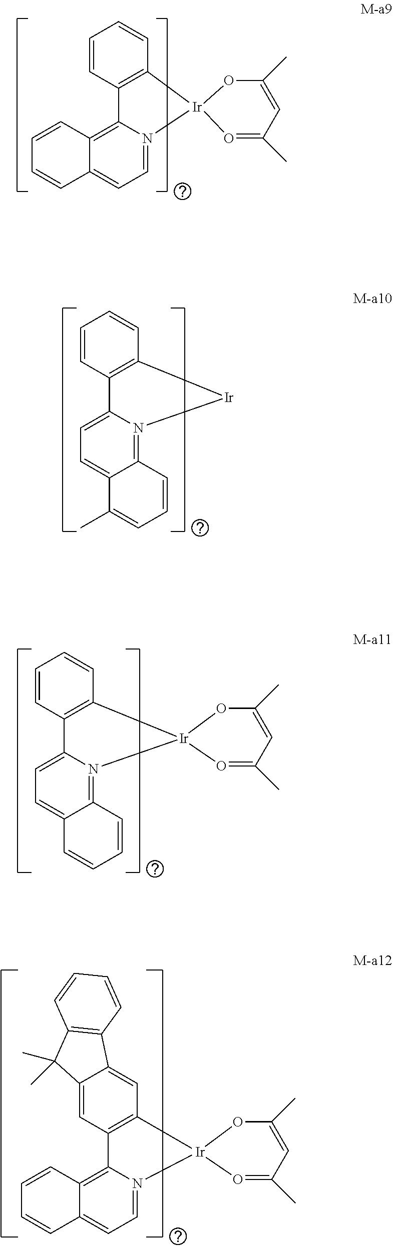

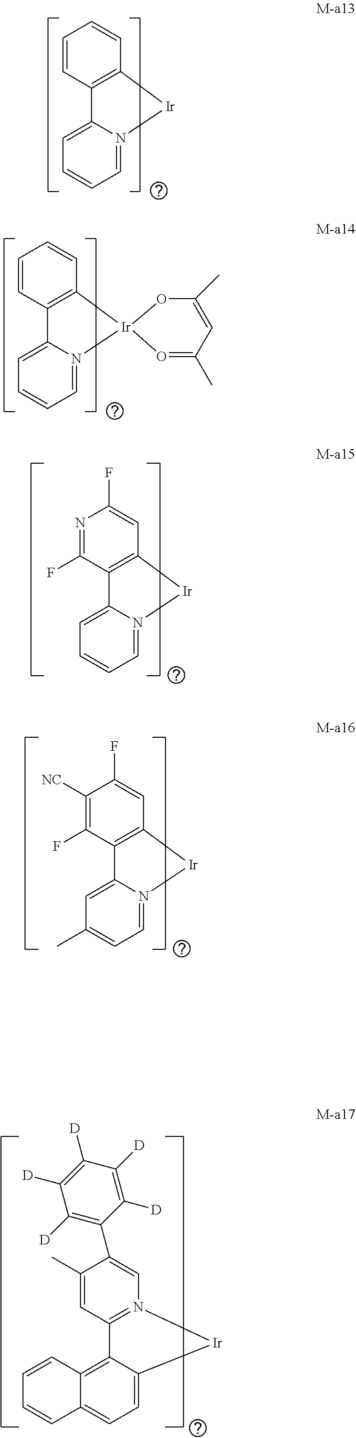

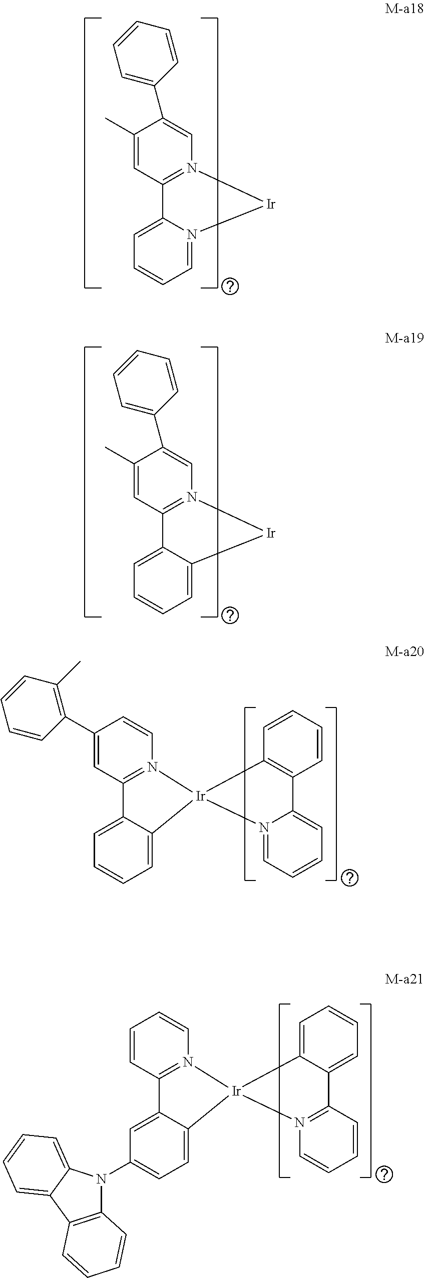

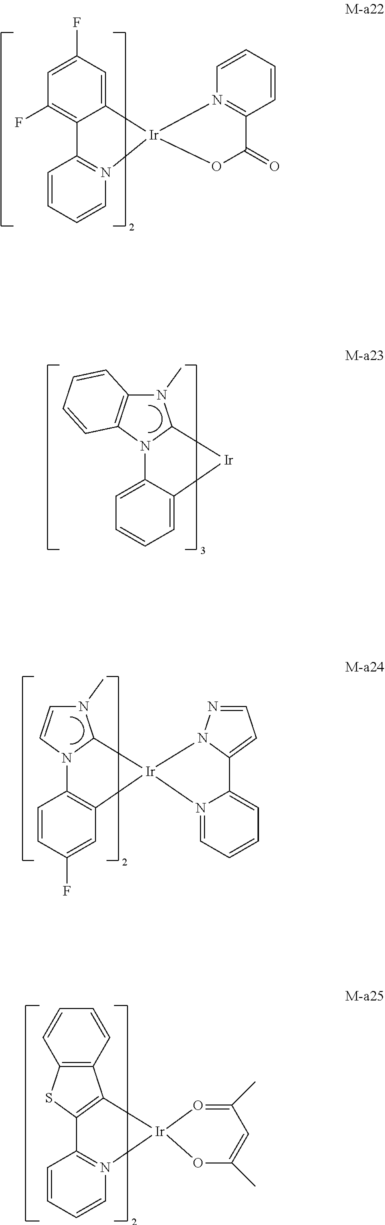

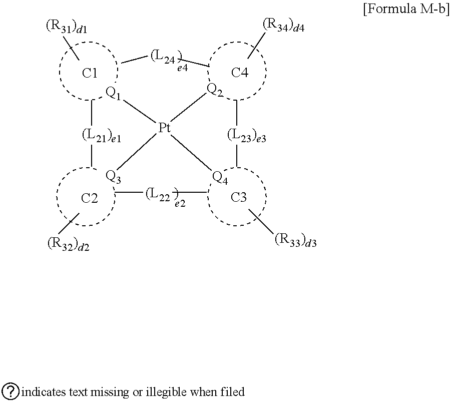

In Formula M-b, Q₁ to Q₄ may each independently be C or N; and C1 to C4 may each independently be a substituted or unsubstituted hydrocarbon ring of 5 to 30 ring-forming carbon atoms, or a substituted or unsubstituted heterocycle of 2 to 30 ring-forming carbon atoms. In Formula M-b, L₂₁ to L₂₄ may each independently be a direct linkage,

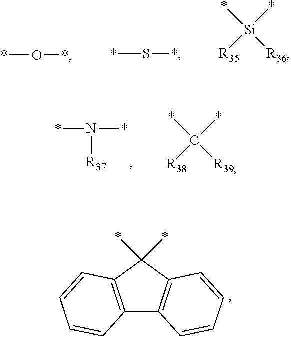

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms; and e1 to e4 may each independently be 0 or 1. In Formula M-b, R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring; and d1 to d4 may each independently be an integer from 0 to 4.

The compound represented by Formula M-b may be used as a blue phosphorescence dopant or a green phosphorescence dopant. In an embodiment, the compound represented by Formula M-b may be an auxiliary dopant and may be further included in the emission layer EML.

The compound represented by Formula M-b may be any compound selected from Compound M-b-1 to Compound M-b-11. However, Compound M-b-1 to Compound M-b-11 are only examples, and the compound represented by Formula M-b is not limited to Compound M-b-1 to Compound M-b-11.

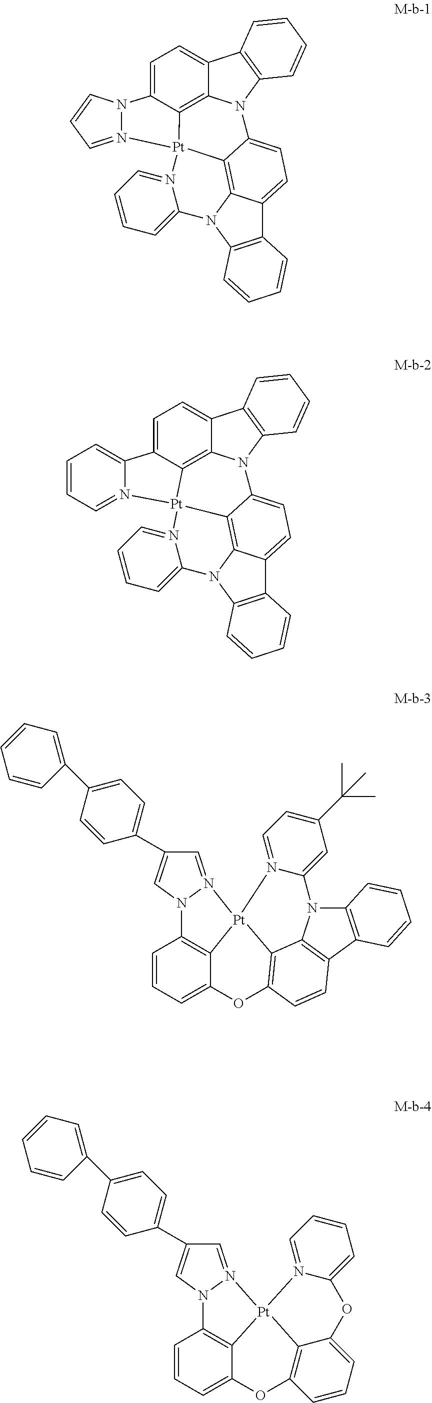

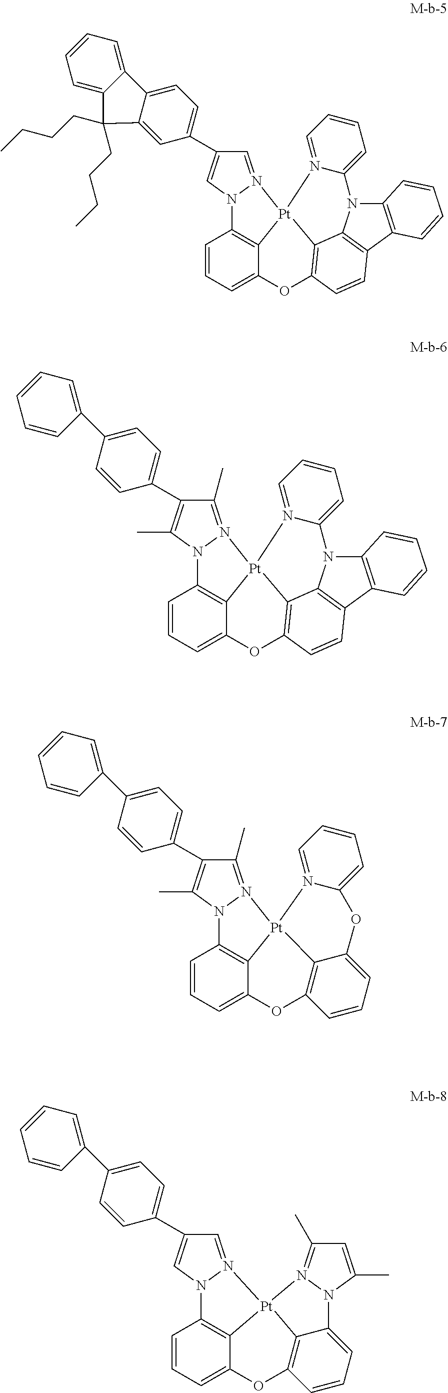

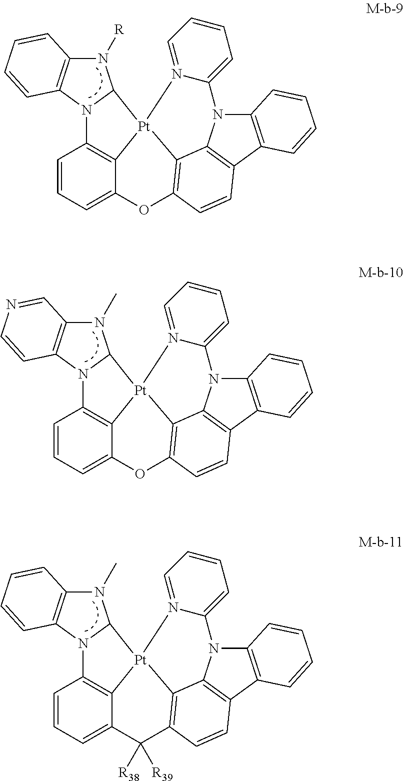

In Compound M-b-1 to Compound M-b-11, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In an embodiment, the emission layer EML may include a compound represented by one of Formula F-a to Formula F-c. The compound represented by one of Formula F-a to Formula F-c may be used as a fluorescence dopant material.

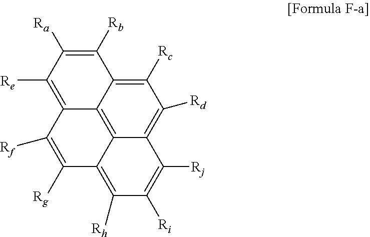

In Formula F-a, two of Rₐ to Rⱼ may each independently be substituted with a group represented by *—NAr₁Ar₂. The remainder of Rₐ to Rⱼ which are not substituted with the group represented by *—NAr₁Ar₂ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In the group represented by *—NAr₁Ar₂, Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, at least one of Ar₁ and Ar₂ may each independently be a heteroaryl group including O or S as a ring-forming atom.

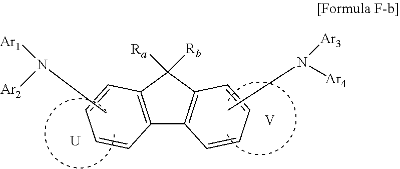

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring.

In Formula F-b, Ar₁ to Ar₄ may each independently be a substituted or unsubstituted aryl group of 6 to 30 ring-forming carbon atoms or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms or a substituted or unsubstituted heterocycle of 2 to 30 ring-forming carbon atoms.

In Formula F-b, the number of rings represented by U and V may each independently be 0 or 1. If the number of U or V is 1, a fused ring may be present at the portion indicated by U or V, and if the number of U or V is 0, a fused ring may not be present at the portion indicated by U or V. If the number of U is 0 and the number of V is 1, or if the number of U is 1 and the number of V is 0, a fused ring having the fluorene core of Formula F-b may be a cyclic compound with four rings. If the number of U and V are each 0, a fused ring having the fluorene core of Formula F-b may be a cyclic compound with three rings. If the number of U and V are each 1, a fused ring having the fluorene core of Formula F-b may be a cyclic compound with five rings.

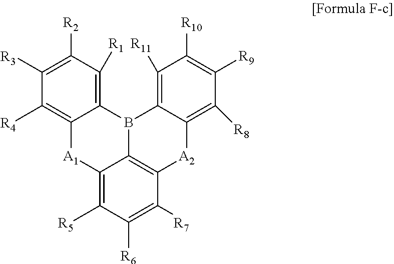

In Formula F-c, A₁ and A₂ may each independently be O, S, Se, or N(Rₘ); and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring.

In Formula F-c, A₁ and A₂ may each independently be bonded to a substituent of an adjacent ring to form a fused ring. For example, if A₁ and A₂ are each independently N(Rₘ), A₁ may be combined with R₄ or R₅ to form a ring. For example, A₂ may be combined with R₇ or R₈ to form a ring.

In an embodiment, the emission layer EML may include as a dopant material of the related art, a styryl derivative (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene or a derivative thereof (for example, 2,5,8,11-tetra-t-butylperylene (TBP)), pyrene or a derivative thereof (for example, 1,1-dipyrene, 1,4-dipyrenylbenzene, and 1,4-bis(N,N-diphenylamino)pyrene), etc.

In an embodiment, if multiple emission layers EML are included, at least one emission layer EML may include a phosphorescent dopant material of the related art. For example, a phosphorescent dopant may include a metal complex including iridium (Ir), platinum (Pt), osmium (Os), gold (Au), titanium (Ti), zirconium (Zr), hafnium (H), europium (Eu), terbium (Tb), or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescence dopant. However, embodiments are not limited thereto.

In an embodiment, the emission layer EML may include a hole transport host and an electron transport host. The emission layer EML may include an auxiliary dopant and a light emitting dopant. The auxiliary dopant may include a phosphorescent dopant material or a thermally activated delayed fluorescent dopant. For example, in an embodiment, the emission layer EML may include a hole transport host, an electron transport host, an auxiliary dopant, and a light emitting dopant.

An exciplex may be formed by the hole transport host and the electron transport host in the emission layer EML. A triplet energy of the exciplex formed by the hole transport host and the electron transport host may correspond to T1, which is a gap between a lowest unoccupied molecular orbital (LUMO) energy level of the electron transport host and a highest occupied molecular orbital (HOMO) energy level of the hole transport host.

In an embodiment, the triplet energy (T1) of the exciplex formed by the hole transport host and the electron transport host may be in a range about 2.4 eV to about 3.0 eV. The triplet energy of the exciplex may be a value that is smaller than an energy gap of each host material. Accordingly, the exciplex may have a triplet energy equal to or less than about 3.0 eV, which is an energy gap between the hole transport host and the electron transport host.

In an embodiment, the emission layer may include a quantum dot.

In embodiments, the quantum dot may be a crystal of a semiconductor compound. The quantum dot may emit light in various emission wavelengths according to a size of the crystal. The quantum dot may emit light in various emission wavelengths by adjusting an elemental ratio of a quantum dot compound.

A diameter of the quantum dot may be, for example, in a range of about 1 nm to about 10 nm.

The quantum dot may be synthesized by a chemical bath deposition, a metal organic chemical vapor deposition, a molecular beam epitaxy or a similar process therewith.

A chemical bath deposition is a method of mixing an organic solvent and a precursor material and growing a quantum dot particle crystal. While growing the crystal, the organic solvent may serve as a dispersant that is coordinated on a surface of the quantum dot crystal and may control the growth of the crystal. Accordingly, a chemical bath deposition process may be more advantageous when compared to a vapor deposition method such as metal organic chemical vapor deposition (MOCVD) and molecular beam epitaxy (MBE), and the growth of a quantum dot particle may be controlled through a low-cost process.

In an embodiment, the quantum dot may be a Group II-VI compound, a Group III-VI compound, a Group I-III-VI compound, a Group III-V compound, a Group III-II-V compound, a Group IV-VI compound, a Group IV element, a Group IV compound, or any combination thereof.

Examples of a Group II-VI compound may include: a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof; or any combination thereof.

In an embodiment, a Group II-VI compound may further include a Group I metal and/or a Group IV element. Examples of a Group I-II-VI compound may include CuSnS or CuZnS. Examples of a Group l-IV-VI compound may include ZnSnS and the like. Examples of a Group I-II-IV-VI compound may include: quaternary compounds selected from the group consisting of Cu₂ZnSnS₂, Cu₂ZnSnS₄, Cu₂ZnSnSe₄, Ag₂ZnSnS₂, or any mixture thereof.

Examples of a Group III-VI compound may include: a binary compound such as In₂S₃, and In₂Se₃; a ternary compound such as InGaS₃, and InGaSe₃; or any combination thereof.

Examples of a Group I-III-VI compound may include: a ternary compound selected from the group consisting of AgInS, AgInS₂, CuInS, CuInS₂, AgGaS₂, CuGaS₂, CuGaO₂, AgGaO₂, AgAlO₂, and mixtures thereof; a quaternary compound such as AgInGaS₂, and CuInGaS₂; or any combination thereof.

Examples of a Group III-V compound may include: a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof; or any combination thereof. In an embodiment, a Group III-V compound may further include a Group II metal. Examples of a Group III-II-V compound may include InZnP, etc.

Examples of a Group IV-VI compound may include: a binary compound selected from the group consisting of SnS, SnSe, SnTe, PbS, PbSe, PbTe, and any mixture thereof; a ternary compound selected from the group consisting of SnSeS, SnSeTe, SnSTe, PbSeS, PbSeTe, PbSTe, SnPbS, SnPbSe, SnPbTe, and any mixture thereof; a quaternary compound selected from the group consisting of SnPbSSe, SnPbSeTe, SnPbSTe, and any mixture thereof; or any combination thereof.

Examples of a Group II-IV-V compound may include a ternary compound selected from the group consisting of ZnSnP, ZnSnP₂, ZnSnAs₂, ZnGeP₂, ZnGeAs₂, CdSnP₂, CdGeP₂, and any mixture thereof.

Examples of a Group IV element may include Si, Ge, and any mixture thereof. Examples of a Group IV compound may include a binary compound selected from the group consisting of SiC, SiGe, and any mixture thereof.

Each element included in a multi-element compound such as a binary compound, a ternary compound, and a quaternary compound may be present in a particle at a uniform concentration or at a non-uniform concentration. For example, a formula may indicate the elements included in a compound, but an elemental ratio in the compound may vary. For example, AgInGaS₂ may indicate AgInₓGa₁₋ₓS₂ (where x may be a real number between 0 and 1).

A binary compound, a ternary compound or a quaternary compound may be present in a particle at a uniform concentration or may be present in a particle at a partially different concentration distribution state. In an embodiment, a quantum dot may have a core/shell structure in which one quantum dot surrounds another quantum dot. An interface between the core and the shell may have a concentration gradient in which the concentration of a material that is present in the shell decreases toward the core.

In embodiments, the quantum dot may have the above-described core-shell structure including a core that includes a nanocrystal and a shell surrounding the core. The shell of a quantum dot may serve as a protection layer for preventing the chemical deformation of the core to maintain semiconductor properties and/or may serve as a charging layer for imparting the quantum dot with electrophoretic properties. The shell may be a single layer or a multilayer. Examples of a shell of a quantum dot may include a metal oxide, a non-metal oxide, a semiconductor compound, or any combination thereof.

Examples of a metal oxide or a non-metal oxide may include: a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO; a ternary compound such as MgAl₂O₄, CoFe₂O₄, NiFe₂O₄, or CoMn₂O₄; or any combination thereof, but embodiments are not limited thereto.

Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width at half maximum (FWHM) of an emission wavelength spectrum equal to or less than about 45 nm. For example, the quantum dot may have a FWHM of an emission wavelength spectrum equal to or less than about 40 nm. For example, the quantum dot may have a FWHM of an emission wavelength spectrum equal to or less than about 30 nm. Within any of the above ranges, color purity or color reproducibility may be improved. Light that is emitted through a quantum dot may be emitted in all directions, so that light viewing angle properties may be improved.

The shape of a quantum dot may be any shape that is used in the related art. For example, a quantum dot may have a spherical shape, a pyramidal shape, a multi-arm shape, or a cubic shape, or a quantum dot may be in the form of a nanoparticle, a nanotube, a nanowire, a nanofiber, a nanoplate particle, etc.

As a size of the quantum dot or an elemental ration of the quantum dot compound is adjusted, the energy band gap may be accordingly controlled to obtain light of various wavelengths from the quantum dot emission layer. Therefore, by using quantum dots as described above (for example, using quantum dots of different sizes or having different elemental ratios in the quantum dot compound), a light emitting element that emits light of various wavelengths may be achieved. For example, the size of the quantum dots or the elemental ratio of a quantum dot compound may be adjusted to emit red light, green light, and/or blue light. For example, quantum dots may be configured to emit white light by combining light of various colors.

In the light emitting element ED according to an embodiment as shown in each of FIG. 3 to FIG. 6, the electron transport region ETR may be provided on the emission layer EML. The electron transport region ETR may include at least one of an electron blocking layer HBL, an electron transport layer ETL, and an electron injection layer EIL. However, embodiments are not limited thereto.

The electron transport region ETR may be a layer consisting of a single material, a layer including different materials, or a structure including multiple layers including different materials.

For example, the electron transport region ETR may have a single layer structure of an electron injection layer EIL or an electron transport layer ETL, or may have a single layer structure formed of an electron injection material and an electron transport material. In other embodiments, the electron transport region ETR may have a single layer structure formed of different materials, or may have a structure in which an electron transport layer ETL/electron injection layer EIL, or a hole blocking layer HBL/electron transport layer ETL/electron injection layer EL, are stacked in its respective stated order from the emission layer EML, but embodiments are not limited thereto. A thickness of the electron transport region ETR may be, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

In the light emitting element ED according to an embodiment, the electron transport region ETR may include a compound represented by Formula ET-2.

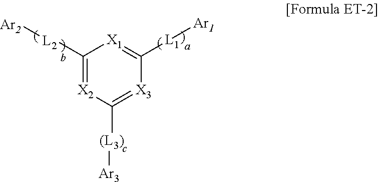

In Formula ET-2, at least one of X₁ to X₃ may each be N; and the remainder of X₁ to X₃ may each independently be C(Rₐ). In Formula ET-2, Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula ET-2, Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, a to c may each independently be an integer from 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. If a to c are each 2 or more, multiple groups of each of L₁ to L₃ may each independently be a substituted or unsubstituted arylene group of 6 to 30 ring-forming carbon atoms or a substituted or unsubstituted heteroarylene group of 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), or a mixture thereof, without limitation.

In an embodiment, the electron transport region ETR may include at least one compound selected from Compounds ET1 to ET36.

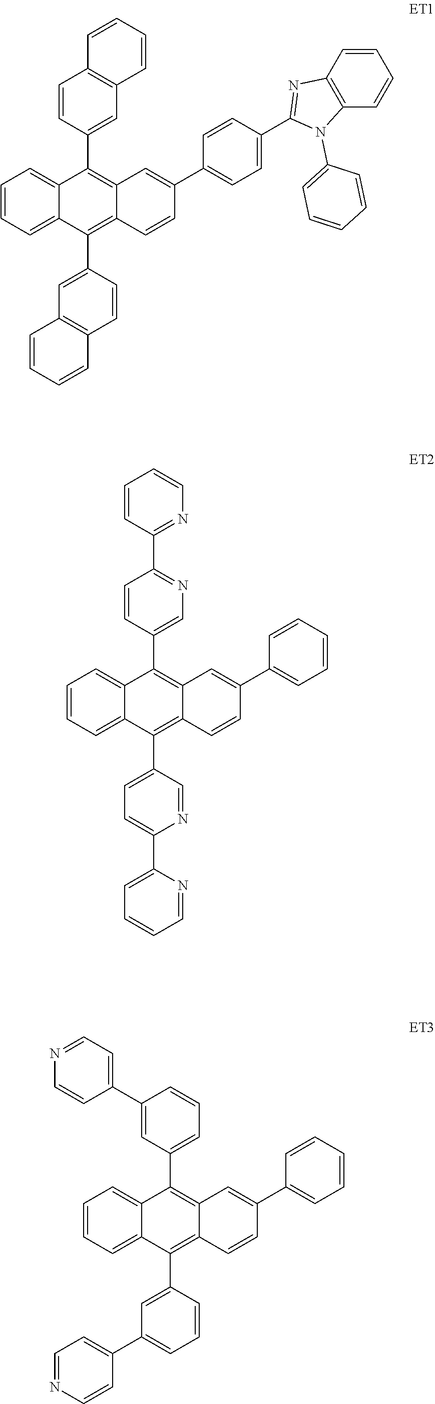 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

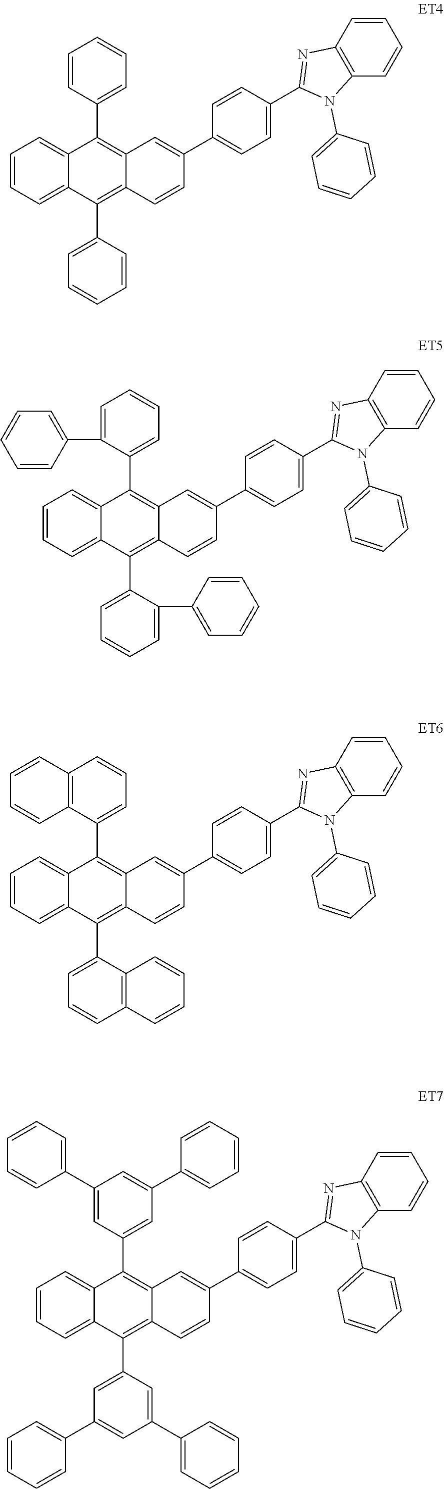 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

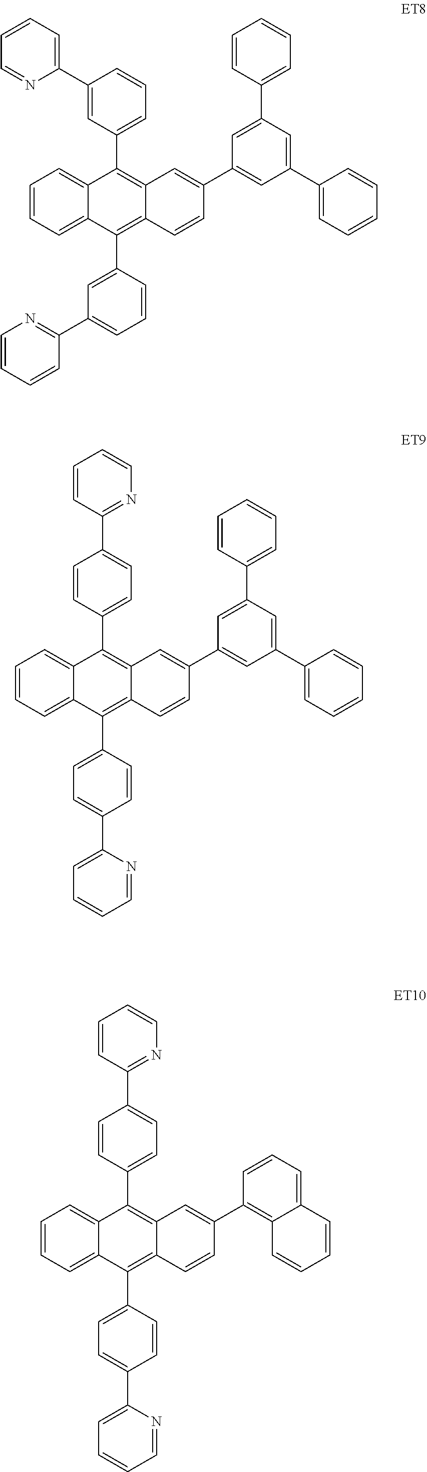 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

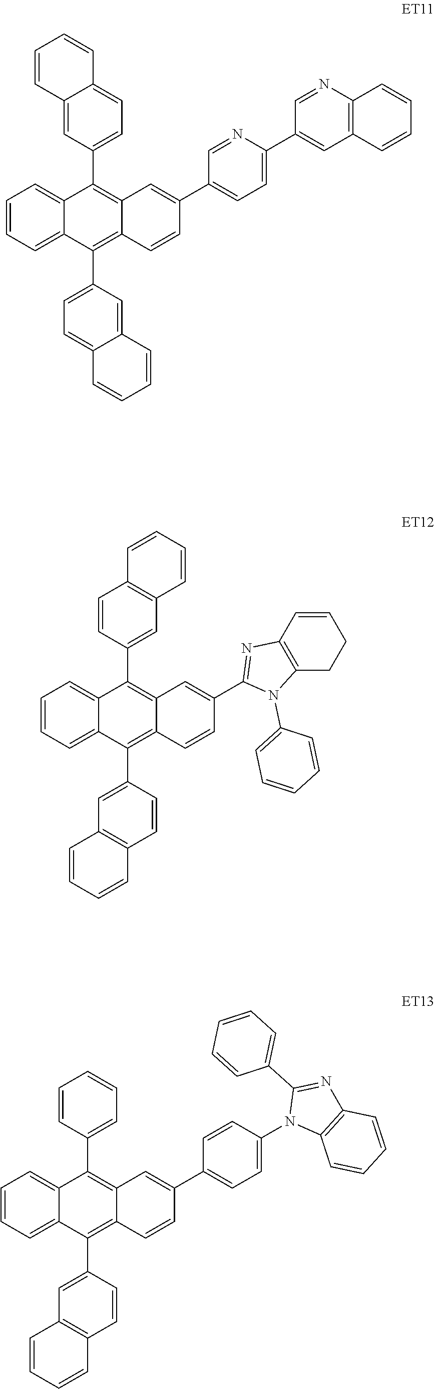 [SMILES: c1ccc2cc(-c3c4ccccc4c(-c4ccc5ccccc5c4)c4cc(-c5ccc(-c6cnc7ccccc7c6)nc5)ccc34)ccc2c1 ; C1=Cc2nc(-c3ccc4c(-c5ccc6ccccc6c5)c5ccccc5c(-c5ccc6ccccc6c5)c4c3)n(-c3ccccc3)c2CC1 ; c1ccc(-c2c3ccccc3c(-c3ccc4ccccc4c3)c3ccc(-c4ccc(-n5c(-c6ccccc6)nc6ccccc65)cc4)cc23)cc1]

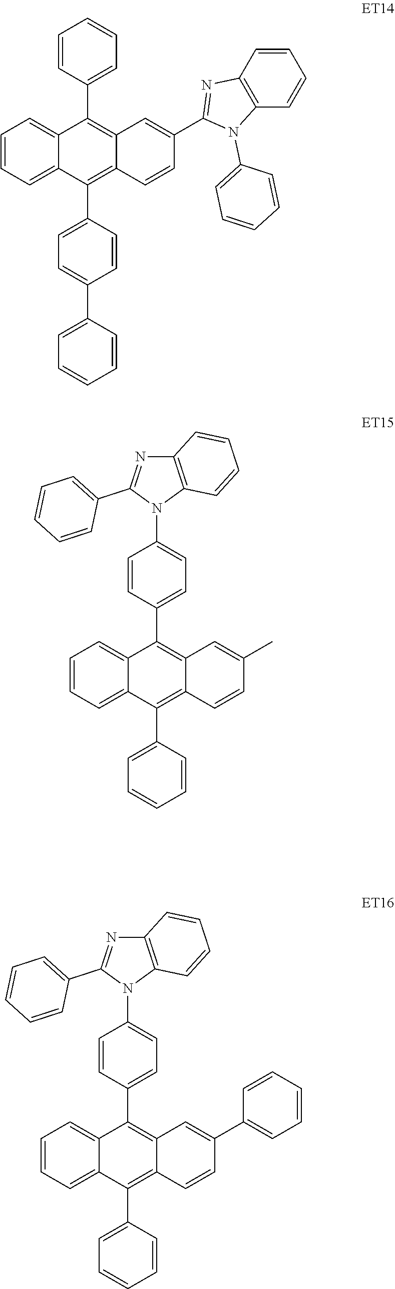 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

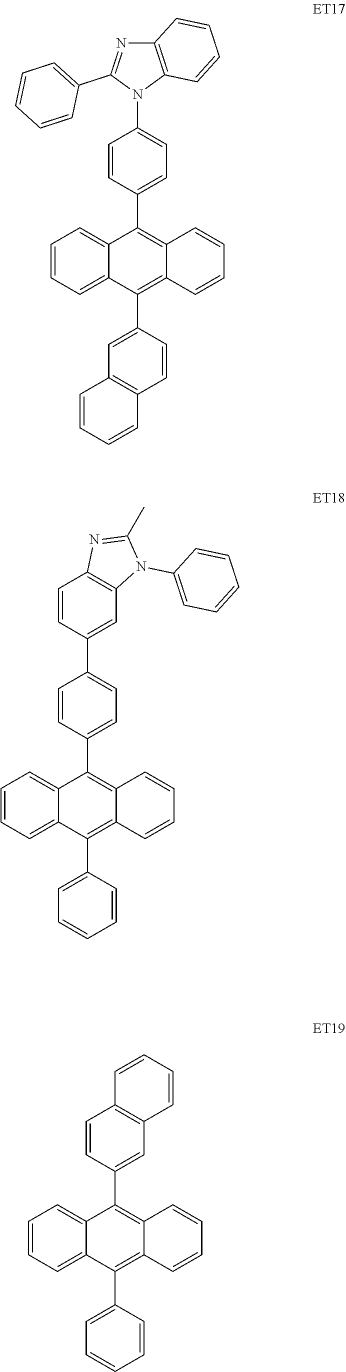

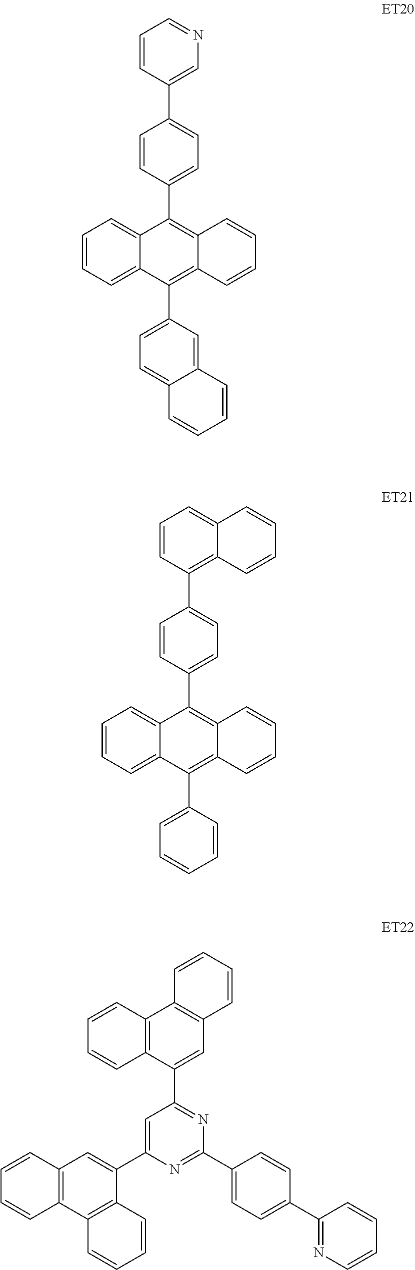

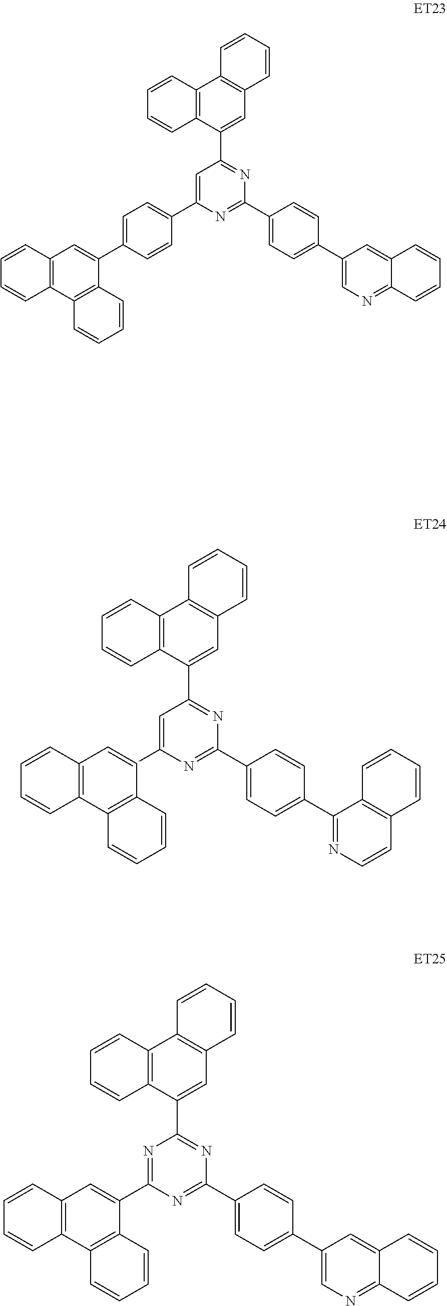

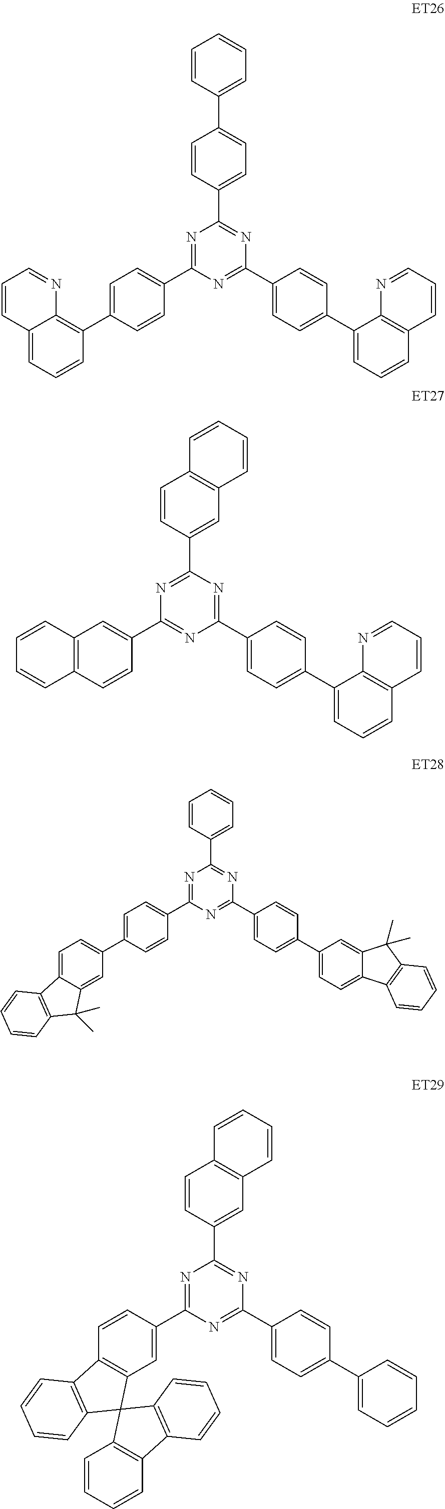 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1]

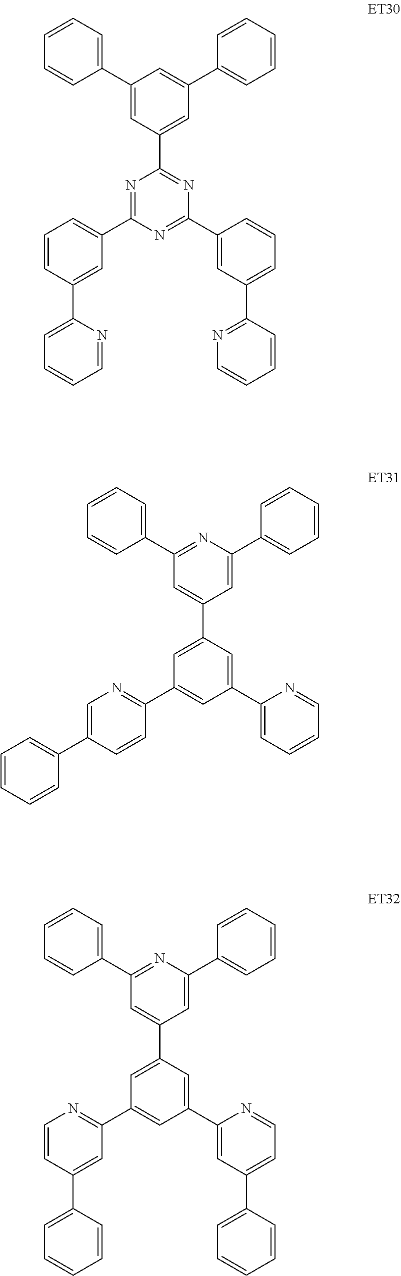 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

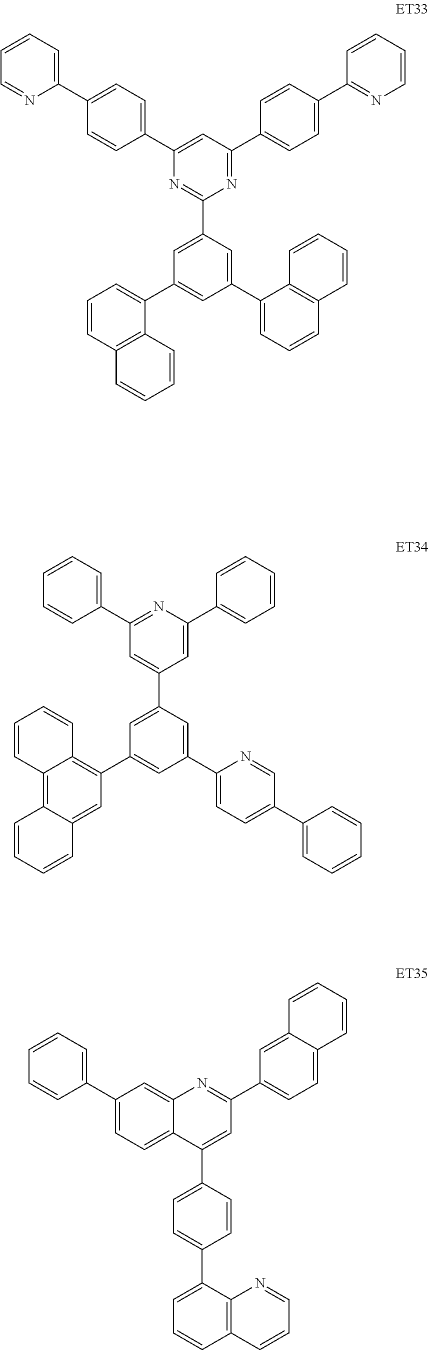 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

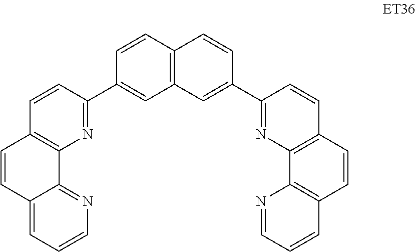 [SMILES: c1cnc2c(c1)ccc1ccc(-c3ccc4ccc(-c5ccc6ccc7cccnc7c6n5)cc4c3)nc12]

In an embodiment, the electron transport region ETR may include: a metal halide such as LiF, NaCl, CsF, RbCl, RbI, CuI and KI; a lanthanide metal such as Yb; or a co-deposited material of a metal halide and a lanthanide metal. For example, the electron transport region ETR may include KI:Yb, RbI:Yb, LiF:Yb, etc., as a co-deposited material. The electron transport region ETR may include a metal oxide such as Li₂O and BaO, or 8-hydroxy-lithium quinolate (Liq). However, embodiments are not limited thereto. The electron transport region ETR may be formed of a mixture material of an electron transport material and an insulating organometallic salt. The insulating organometallic salt may be a material having an energy band gap greater than or equal to about 4 eV. For example, the insulating organometallic salt may include a metal acetate, a metal benzoate, a metal acetoacetate, a metal acetylacetonate, or a metal stearate.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, embodiments are not limited thereto.

The electron transport region ETR may include the above-described compounds of the electron transport region in at least one of an electron injection layer EIL, an electron transport layer ETL, and a hole blocking layer HBL.

If the electron transport region ETR includes an electron transport layer ETL, a thickness of the electron transport layer ETL may be in a range of about 100 Å to about 1,000 Å. For example, the thickness of the electron transport layer ETL may be in a range of about 150 Å to about 500 Å. If the thickness of the electron transport layer ETL satisfies any of the above-described ranges, satisfactory electron transport properties may be obtained without a substantial increase of a driving voltage. If the electron transport region ETR includes an electron injection layer EIL, a thickness of the electron injection layer EIL may be in a range of about 1 Å to about 100 Å. For example, the thickness of the electron injection layer EIL may be in a range of about 3 Å to about 90 Å. If the thickness of the electron injection layer EIL satisfies any of the above described ranges, satisfactory electron injection properties may be obtained without inducing a substantial increase of a driving voltage.

The second electrode EL2 may be provided on the electron transport region ETR. The second electrode EL2 may be a common electrode. The second electrode EL2 may be a cathode or an anode, but embodiments are not limited thereto. For example, if the first electrode EL1 is an anode, the second cathode EL2 may be a cathode, and if the first electrode EL1 is a cathode, the second electrode EL2 may be an anode.

The second electrode EL2 may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, and a mixture thereof.

The second electrode EL2 may be a transmissive electrode, a transflective electrode, or a reflective electrode. If the second electrode EL2 is a transmissive electrode, the second electrode EL2 may include a transparent metal oxide, for example, ITO, IZO, ZnO, ITZO, etc.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, a compound thereof, or a mixture thereof (for example, AgMg, AgYb, or MgAg). In an embodiment, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

Although not shown in the drawings, the second electrode EL2 may be electrically connected to an auxiliary electrode. If the second electrode EL2 is electrically connected to an auxiliary electrode, resistance of the second electrode EL2 may decrease.

In an embodiment, the light emitting element ED may further include a capping layer CPL disposed on the second electrode EL2. The capping layer CPL may be a multilayer or a single layer.

In an embodiment, the capping layer CPL may include an organic layer or an inorganic layer. For example, if the capping layer CPL includes an inorganic material, the inorganic material may include an alkali metal compound such as LiF, an alkaline earth metal compound such as SiON, SiNₓ, SiO_{y}, etc.

For example, if the capping layer CPL includes an organic material, the organic material may include 2,2′-dimethyl-N,N′-di-[(1-naphthyl)-N,N′-diphenyl]-1,1′-biphenyl-4,4′-diamine(α-NPD), NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., or may include an epoxy resin, or an acrylate such as methacrylate. In an embodiment, the capping layer CPL may include at least one of Compounds P1 to P5, but embodiments are not limited thereto.

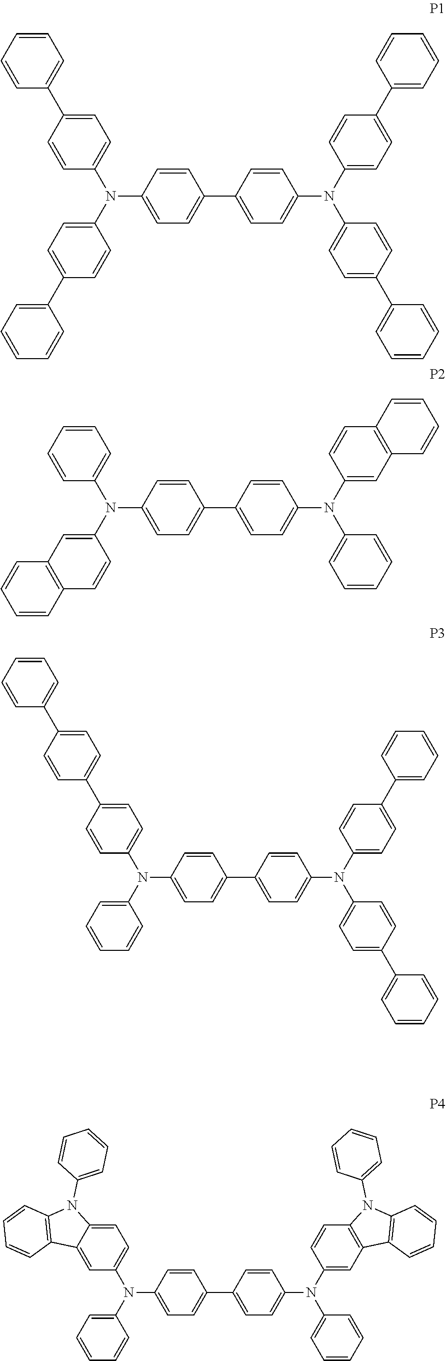 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

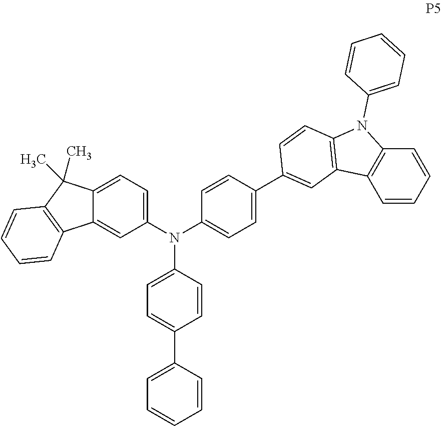 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6, with respect to light in a wavelength range of about 550 nm to about 660 nm.

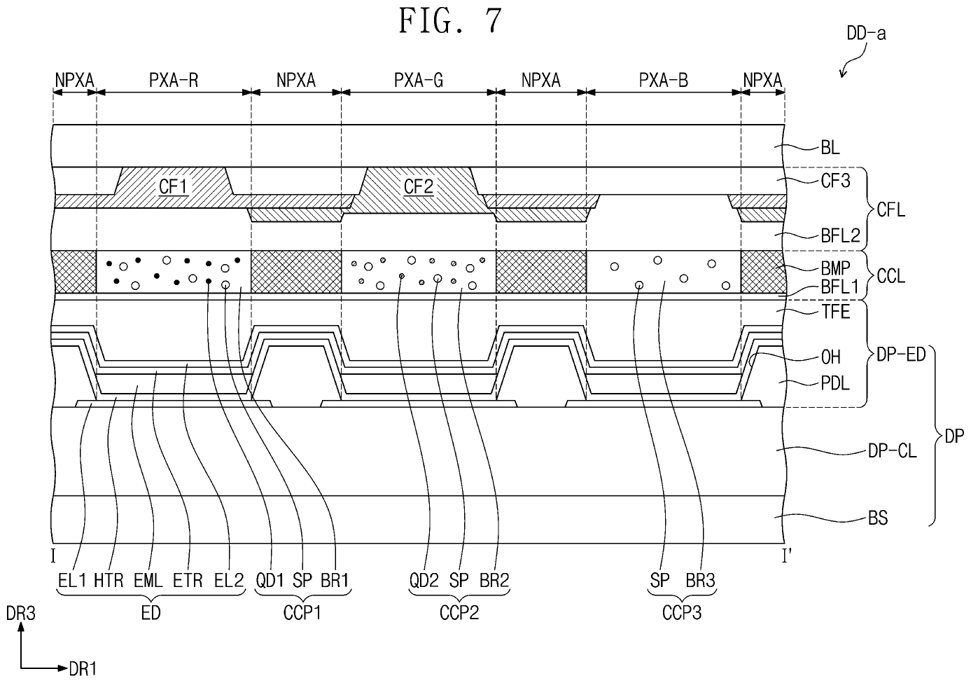

FIG. 7 to FIG. 10 are each a schematic cross-sectional view of a display device according to an embodiment. In the descriptions of the display devices according to embodiments as shown in FIG. 7 to FIG. 10, the features which have been described above with respect to FIG. 1 to FIG. 6 will not be explained again, and the differing features will be explained.

Referring to FIG. 7, the display device DD-a according to an embodiment may include a display panel DP including a display device layer DP-ED, a light controlling layer CCL disposed on the display panel DP, and a color filter layer CFL.

In an embodiment shown in FIG. 7, the display panel DP includes a base layer BS, a circuit layer DP-CL provided on the base layer BS, and a display device layer DP-ED, and the display device layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In embodiments, a structure of the light emitting element ED shown in FIG. 7 may be the same as a structure of a light emitting element ED according to one of FIG. 3 to FIG. 6 as described herein.

The emission layer EML of the light emitting element ED included in the display device DD-a according to an embodiment may include the amine compound according to an embodiment described above.

Referring to FIG. 7, the emission layer EML may be disposed in an opening OH defined in a pixel defining film PDL. For example, the emission layer EML, which is separated by the pixel defining film PDL and correspondingly provided to each of the light emitting regions PXA-R, PXA-G, and PXA-B, may emit light in a same wavelength region. In the display device DD-a, the emission layer EML may emit blue light. Although not shown in the drawings, in an embodiment, the emission layer EML may be provided as a common layer for each of the light emitting regions PXA-R, PXA-G, and PXA-B.

The light controlling layer CCL may be disposed on the display panel DP. The light controlling layer CCL may include a light converter. The light converter may be a quantum dot or a phosphor. The light converter may convert the wavelength of a provided light and emit the resulting light. For example, the light controlling layer CCL may be a layer including a quantum dot or a layer including a phosphor.

The light controlling layer CCL may include light controlling parts CCP1, CCP2, and CCP3. The light controlling parts CCP1, CCP2, and CCP3 may be spaced apart from each other.

Referring to FIG. 7, a partition pattern BMP may be disposed between the light controlling parts CCP1, CCP2, and CCP3, but embodiments are not limited thereto. In FIG. 7, it is shown that the partition pattern BMP does not overlap the light controlling parts CCP1, CCP2, and CCP3, but the edges of the light controlling parts CCP1, CCP2, and CCP3 may overlap at least a portion of the partition pattern BMP.

The light controlling layer CCL may include a first light controlling part CCP1 including a first quantum dot QD1 that converts first color light provided from the light emitting element ED into second color light, a second light controlling part CCP2 including a second quantum dot QD2 that converts first color light into third color light, and a third light controlling part CCP3 that transmits the first color light.

In an embodiment, the first light controlling part CCP1 may provide red light, which is the second color light, and the second light controlling part CCP2 may provide green light, which is the third color light. The third color controlling part CCP3 may transmit and provide blue light, which is the first color light provided from the light emitting element ED. For example, the first quantum dot QD1 may be a red quantum dot, and the second quantum dot QD2 may be a green quantum dot. The quantum dots QD1 and QD2, may each be a quantum dot as described above.

The light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but may include the scatterer SP.

The scatterer SP may be an inorganic particle. For example, the scatterer SP may include at least one of TiO₂, ZnO, Al₂O₃, SiO₂, and hollow silica. The scatterer SP may include at least one of TiO₂, ZnO, Al₂O₃, SiO₂, and hollow silica, or may be a mixture of two or more materials selected from TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3, in which the quantum dots QD1 and QD2 and the scatterer SP are dispersed. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer SP dispersed in the third base resin BR3.

The base resins BR1, BR2, and BR3 are mediums in which the quantum dots QD1 and QD2 and the scatterer SP are dispersed, and may be composed of various resin compositions which may be generally referred to as a binder. For example, the base resins BR1, BR2, and BR3 may be acrylic resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 may be the same as or different from each other.

The light controlling layer CCL may include a barrier layer BFL1. The barrier layer BFL1 may block the penetration of moisture and/or oxygen (hereinafter, will be referred to as “humidity/oxygen”). The barrier layer BFL1 may block the light controlling parts CCP1, CCP2, and CCP3 from exposure to humidity/oxygen. The barrier layer BFL1 may cover the light controlling parts CCP1, CCP2, and CCP3. A color filter layer CFL, which will be explained later, may include a barrier layer BFL2 disposed on the light controlling parts CCP1, CCP2, and CCP3.

The barrier layers BFL1 and BFL2 may each independently include at least one inorganic layer. For example, the barrier layers BFL1 and BFL2 may each independently include an inorganic material. For example, the barrier layers BFL1 and BFL2 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. The barrier layers BFL1 and BFL2 may each independently further include an organic layer. The barrier layers BFL1 and BFL2 may each be formed of a single layer or formed of multiple layers.

In the display device DD-a, the color filter layer CFL may be disposed on the light controlling layer CCL. In an embodiment, the color filter layer CFL may be directly disposed on the light controlling layer CCL. For example, the barrier layer BFL2 may be omitted.

The color filter layer CFL may include filters CF1, CF2, and CF3. The first to third filters CF1, CF2, and CF3 may be disposed to respectively correspond to a red light emitting region PXA-R, a green light emitting region PXA-G, and a blue light emitting region PXA-B.

The color filter layer CFL may include a first filter CF1 that transmits second color light, a second filter CF2 that transmits third color light, and a third filter CF3 that transmits first color light. For example, the first filter CF1 may be a red filter, the second filter CF2 may be a green filter, and the third filter CF3 may be a blue filter. The filters CF1, CF2, and CF3 may each include a polymer photosensitive resin and a pigment or dye. The first filter CF1 may include a red pigment or dye, the second filter CF2 may include a green pigment or dye, and the third filter CF3 may include a blue pigment or dye.

However, embodiments are not limited thereto, and the third filter CF3 may not include a pigment or dye. The third filter CF3 may include a polymer photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may be provided in one body, without distinction.

Although not shown in the drawings, the color filter layer CFL may further include a light blocking part (not shown). The color filter layer CFL may include a light blocking part (not shown) that is disposed so as to overlap the boundaries between adjacent filters CF1, CF2, and CF3. The light blocking part (not shown) may be a black matrix. The light blocking part (not shown) may include an organic light blocking material or an inorganic light blocking material, each including a black pigment or a black dye. The light blocking part (not shown) may separate the boundaries between adjacent filters CF1, CF2, and CF3. In an embodiment, the light blocking part (not shown) may be formed as a blue filter.

A base substrate BL may be disposed on the color filter layer CFL. The base substrate BL may provide a base surface on which the color filter layer CFL, the light controlling layer CCL, etc. are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

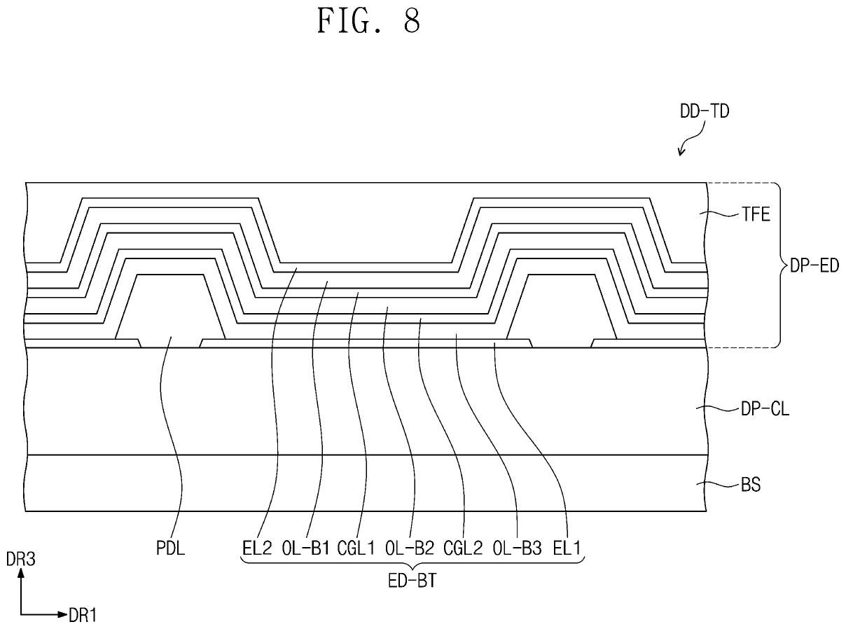

FIG. 8 is a schematic cross-sectional view of a portion of a display device according to an embodiment. In a display device DD-TD according to an embodiment, the light emitting element ED-BT may include light emitting structures OL-B1, OL-B2, and OL-B3.

The light emitting element ED-BT may include a first electrode EL1 and a second electrode EL2 which face each other, and the light emitting structures OL-B1, OL-B2, and OL-B3 stacked in a thickness direction between the first electrode EL1 and the second electrode EL2. The light emitting structures OL-B1, OL-B2, and OL-B3 may each include an emission layer EML (see FIG. 7), and a hole transport region HTR and an electron transport region ETR, disposed with the emission layer (FIG. 7) therebetween.

For example, the light emitting element ED-BT included in the display device DD-TD may be a light emitting element having a tandem structure and including multiple emission layers.

In an embodiment shown in FIG. 8, light emitted from each of the light emitting structures OL-B1, OL-B2, and OL-B3 may be blue light. However, embodiments are not limited thereto, and light emitted from each of the light emitting structures OL-B1, OL-B2, and OL-B3 may have wavelength regions that are different from each other. For example, the light emitting element ED-BT that includes the light emitting structures OL-B1, OL-B2, and OL-B3, which emit light having wavelength regions that are different from each other, may emit white light.

Charge generating layers CGL1 and CGL2 may be disposed between neighboring light emitting structures among the light emitting structures OL-B1, OL-B2, and OL-B3. Charge generating layers CGL1 and CGL2 may each independently include a p-type charge generating layer and/or an n-type charge generating layer.

At least one of the light emitting structures OL-B1, OL-B2, or OL-B3 included in the display device DD-TD may include the above-described amine compound according to an embodiment.

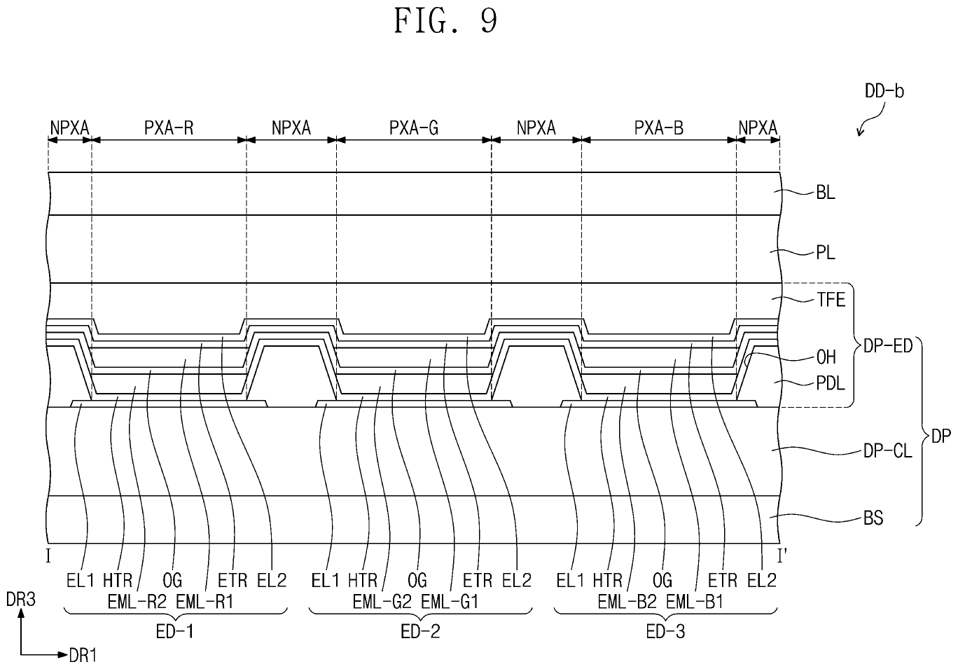

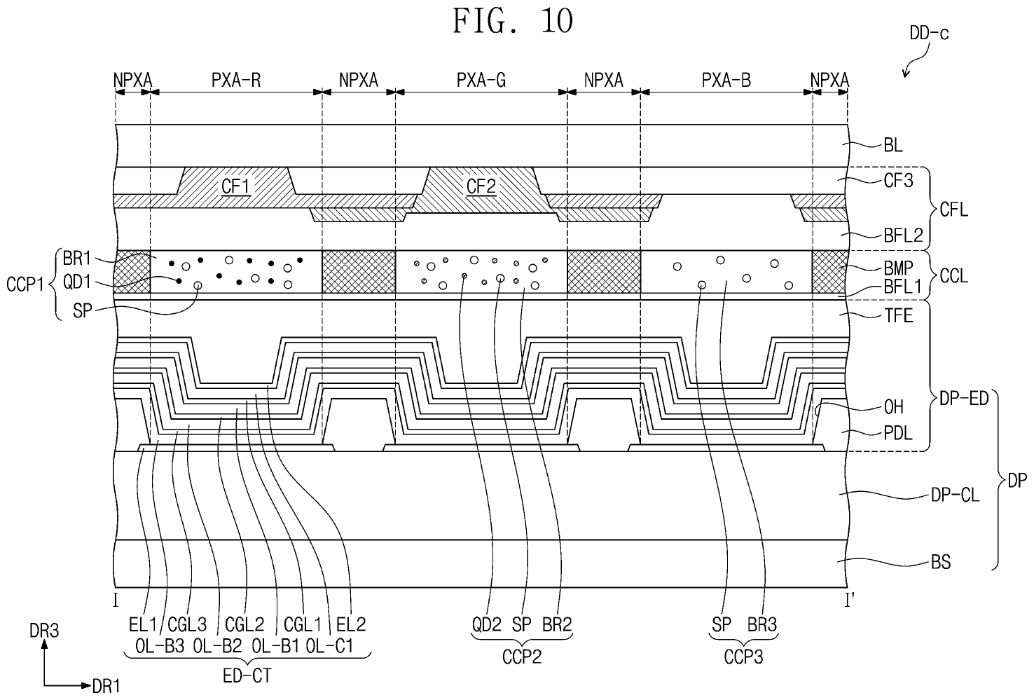

FIG. 9 is a schematic cross-sectional view of a display device DD-b according to an embodiment.

The display device DD-b may include light emitting elements ED-1, ED-2, and ED-3, in which two emission layers are stacked. In comparison to the display device DD shown in FIG. 2, the embodiment shown in FIG. 9 is different at least in that the first to third light emitting elements ED-1, ED-2, and ED-3 each include two emission layers that are stacked in a thickness direction. In each of the first to third light emitting elements ED-1, ED-2, and ED-3, the two emission layers may emit light in a same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. The third light emitting element ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may be a single layer or a multilayer. The emission auxiliary part OG may include a charge generating layer. For example, the emission auxiliary part OG may include an electron transport region, a charge generating layer, and a hole transport region, which may be stacked in that order. The emission auxiliary part OG may be provided as a common layer for each of the first to third light emitting elements ED-1, ED-2, and ED-3. However, embodiments are not limited thereto, and the emission auxiliary part OG may be provided by being patterned in the openings OH defined in the pixel defining film PDL.

The first red emission layer EML-R1, the first green emission layer EML-G1, and the first blue emission layer EML-B1 may each be disposed between the electron transport region ETR and the emission auxiliary part OG. The second red emission layer EML-R2, the second green emission layer EML-G2, and the second blue emission layer EML-B2 may each be disposed between the emission auxiliary part OG and the hole transport region HTR.

For example, the first light emitting device ED-1 may include a first electrode EL1, a hole transport region HTR, a second red emission layer EML-R2, an emission auxiliary part OG, a first red emission layer EML-R1, an electron transport region ETR, and a second electrode EL2, stacked in that order. The second light emitting device ED-2 may include a first electrode EL1, a hole transport region HTR, a second green emission layer EML-G2, an emission auxiliary part OG, a first green emission layer EML-G1, an electron transport region ETR, and a second electrode EL2, stacked in that order. The third light emitting device ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in that order.

An optical auxiliary layer PL may be disposed on the display device layer DP-ED. The optical auxiliary layer PL may include a polarization layer. The optical auxiliary layer PL may be disposed on the display panel DP and may control light that is reflected at the display panel DP from an external light. Although not shown in the drawings, in an embodiment, the optical auxiliary layer PL may be omitted from the display device DD-b.

In contrast to FIG. 8 and FIG. 9, FIG. 10 shows a display device DD-c that is different at least in that it includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are stacked in a thickness direction between the first electrode EL1 and the second electrode EL2. In an embodiment, the third light emitting structure OL-B3, the second light emitting structure OL-B2, the first light emitting structure OL-B1, and the fourth light emitting structure OL-C1 may be stacked in the stated order in a thickness direction.

Charge generating layers CGL1, CGL2, and CGL3 may each be disposed between adjacent light emitting structures among the first to fourth light emitting structures OL-B1, OL-B2, OL-B3, and OL-C1. For example, a first charge generating layer CGL1 may be disposed between the first light emitting structure OL-B1 and the fourth light emitting structure OL-C1. For example, a second charge generating layer CGL2 may be disposed between the first light emitting structure OL-B1 and the second light emitting structure OL-B2. For example, a third charge generating layer CGL3 may be disposed between the second light emitting structure OL-B2 and the third light emitting structure OL-B3.

Among the four light emitting structures, the first to third light emitting structures OL-B1, OL-B2, and OL-B3 may each emit blue light, and the fourth light emitting structure OL-C1 may emit green light. However, embodiments are not limited thereto, and the first to fourth light emitting structures OL-B1, OL-B2, OL-B3, and OL-C1 may emit light having different wavelengths from each other.

In the display device DD-c, at least one of the light emitting structures OL-B1, OL-B2, OL-B3, and OL-C1 may each independently include the amine compound according to an embodiment as described herein.

The light emitting element ED according to an embodiment may include the amine compound according to an embodiment in at least one functional layer disposed between the first electrode EL1 and the second electrode EL2, thereby exhibiting improved luminous efficiency and service life characteristics. The light emitting element ED may include the amine compound according to an embodiment in at least one of the hole transport region HTR, the emission layer EML, and the electron transport region ETR disposed between the first electrode EL1 and the second electrode EL2, or in a capping layer CPL. For example, the amine compound according to an embodiment may be included in the hole transport region HTR of the light emitting element ED, and the light emitting element ED may exhibit high efficiency and long service life characteristics.

The amine compound according to an embodiment includes the core nitrogen atom and the first, second, and third substituents, and thus material stability may be increased and hole transport properties may be improved. Accordingly, the light emitting element including the amine compound according to an embodiment may have improved service life and efficiency. The light emitting element according to an embodiment may include the amine compound in the hole transport layer, thereby exhibiting increased efficiency and service life characteristics.

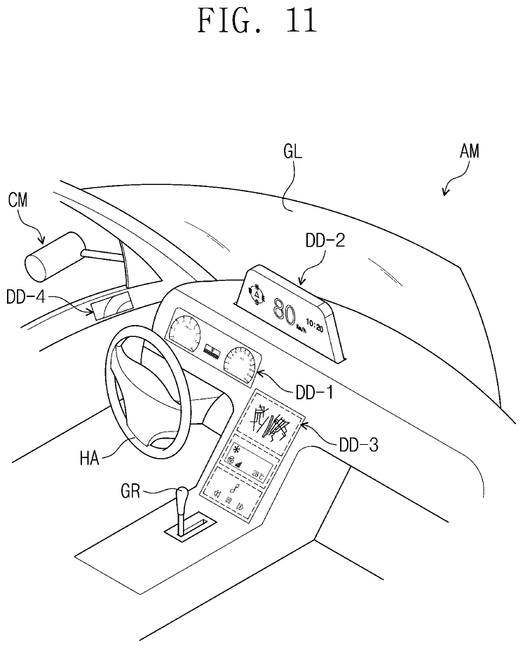

FIG. 11 is a schematic perspective view of a vehicle AM in which first to fourth display devices DD-1, DD-2, DD-3, and DD-4 are disposed. At least one of the first to fourth display devices DD-1, DD-2, DD-3, and DD-4 may have a structure according to one of display devices DD, DD-TD, DD-a, DD-b, and DD-c, as described above with reference to FIGS. 1, 2, and 7 to 10.

In FIG. 11, a vehicle AM is shown as an automobile, but this is only an example, and the first to fourth display devices DD-1, DD-2, DD-3, and DD-4 may be disposed in various transportation means such as bicycles, motorcycles, trains, ships, and airplanes. In an embodiment, at least one of the first to fourth display devices DD-1, DD-2, DD-3, and DD-4 having a structure according to one of display devices DD, DD-TD, DD-a, DD-b, and DD-c may be included in a personal computer, a laptop computer, a personal digital terminal, a game console, a portable electronic device, a television, a monitor, a billboard, or the like. However, these are merely suggested as examples, and the display device may be included in other electronic devices as applicable.

At least one of the first to fourth display devices DD-1, DD-2, DD-3, and DD-4 may each independently include a light emitting element ED according to an embodiment as described with reference to any of FIGS. 3 to 6. The light emitting element ED according to an embodiment may include the amine compound according to an embodiment. At least one of the first to fourth display devices DD-1, DD-2, DD-3, and DD-4 may include a light emitting element ED that includes the amine compound according to an embodiment, thereby exhibiting improved display service life.

Referring to FIG. 11, a vehicle AM may include a steering wheel HA for the operation of the vehicle AM and a gearshift GR. The vehicle AM may include a front window GL disposed to face a driver.

A first display device DD-1 maybe disposed in a first region that overlaps the steering wheel HA. For example, the first display device DD-1 may be a digital cluster that displays first information of the vehicle AM. The first information may include a first scale which indicates a driving speed of the vehicle AM, a second scale which indicates an engine speed (for example, as revolutions per minute (RPM)), a fuel gauge, and the like. The first scale and the second scale may be represented by digital images.

A second display device DD-2 may be disposed in a second region facing a driver's seat and overlapping the front window GL. The driver's seat may be a seat where the steering wheel HA is disposed. For example, the second display device DD-2 may be a head up display (HUD) that displays second information of the vehicle AM. The second display device DD-2 may be optically transparent. The second information may include digital numbers that indicate a driving speed of the vehicle AM and may further include information such as the current time. Although not shown in the drawings, in an embodiment, the second information of the second display device DD-2 may be displayed by being projected on the front window GL.

A third display device DD-3 may be disposed in a third region adjacent to the gearshift GR. For example, the third display device DD-3 may be a center information display (CID) that is disposed between a driver's seat and a passenger seat and which displays third information of the vehicle AM. The passenger seat may be a seat that is spaced apart from the driver's seat with the gearshift GR disposed therebetween. The third information may include information on traffic or road conditions (for example, navigation information), playing music or radio, displaying an image or video, the temperature in the vehicle AM, or the like.

A fourth display device DD-4 may be disposed in a fourth region that is spaced apart from the steering wheel HA and the gearshift GR and adjacent to a side of the vehicle AM. For example, the fourth display device DD-4 may be a digital side-view mirror that displays fourth information. The fourth display device DD-4 may display an image external to the vehicle AM that is taken by a camera module CM disposed outside the vehicle AM. The fourth information may include an image of the exterior of the vehicle AM.

The first to fourth information as described above are only presented as examples, and the first to fourth display devices DD-1, DD-2, DD-3, and DD-4 may further display information about the interior and exterior of the vehicle AM. The first to fourth information may include information that is different from each other. However, embodiments are not limited thereto, and a portion of the first to fourth information may include the same information.

Hereinafter, an amine compound according to an embodiment and a light emitting element according to an embodiment will be described in detail with reference to the Examples and the Comparative Examples. The Examples described below are only provided as illustrations to assist in understanding the disclosure, and the scope thereof is not limited thereto.

### EXAMPLES AND COMPARATIVE EXAMPLES

### 1. Synthesis of Amine Compound

A synthesis method of the amine compound according to embodiments will be explained by describing synthesis methods for Compounds 27, 29, 52, 55, 71, 72, 90, 103, and 497. The synthesis methods of the amine compounds are provided only as examples, and the synthesis methods of the amine compound according to embodiments are not limited to the Examples below. In the synthesis of the amine compounds, the molecular weights of the synthesized compounds were obtained by measuring FAB-MS using JMS-700V made by JEOL, Ltd.

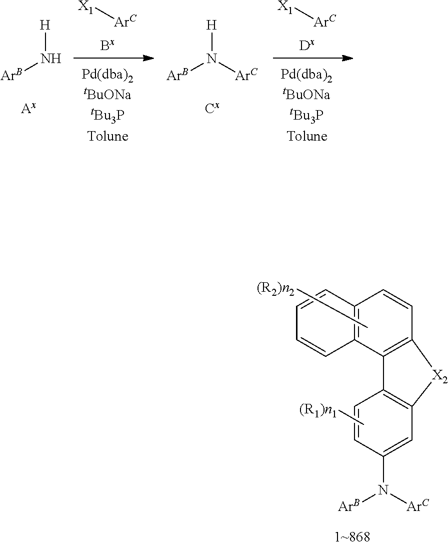

Compounds 1 to 868 may be synthesized by a method according to a general reaction scheme as shown above.

Arylamine A^{x} was heated in a solvent together with arylhalide B^{x}, a phosphine ligand, and a base in the presence of a palladium catalyst to obtain diarylamine C^{x}. The obtained diarylamine C^{x} was heated in a solvent together with arylhalide D^{x}, a phosphine ligand, and a base in the presence of palladium catalyst. Ar^{B} and Ar^{C} are the same substituents as the above-mentioned substituents, X₁ is a substituent, —Cl, —Br, —I, and -OTf, and X₂ is an O or S atom. The superscript number^{x} is an any integer from 1 to 868.

For example, Compounds 90, 71, 29, 55, 103, 500, 72, 52, and 27 were synthesized as follows. Diarylamine C^{x} is a commercially available product unless otherwise described, or synthesized by a method of the related art. The structures of A⁷¹, A¹⁰³, B⁷¹, B¹⁰³, D⁷¹, D⁴⁹⁷, C⁹⁰, C⁷¹, C²⁹, C⁵⁵, C¹⁰³, C⁴⁹⁷, C⁷², C⁵², and C²⁷ used in the Synthesis Examples are described below. The structures of C⁷¹ and C⁴⁹⁷ are the same.

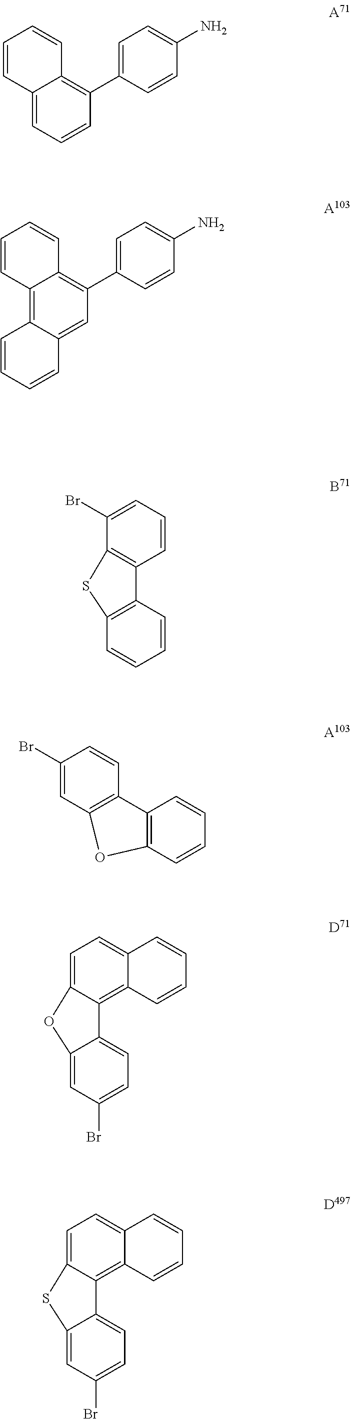 [SMILES: Nc1ccc(-c2cccc3ccccc23)cc1 ; Nc1ccc(-c2cc3ccccc3c3ccccc23)cc1 ; Brc1cccc2c1sc1ccccc12 ; Brc1ccc2c(c1)oc1ccccc12 ; Brc1ccc2c(c1)oc1ccc3ccccc3c12 ; Brc1ccc2c(c1)sc1ccc3ccccc3c12]

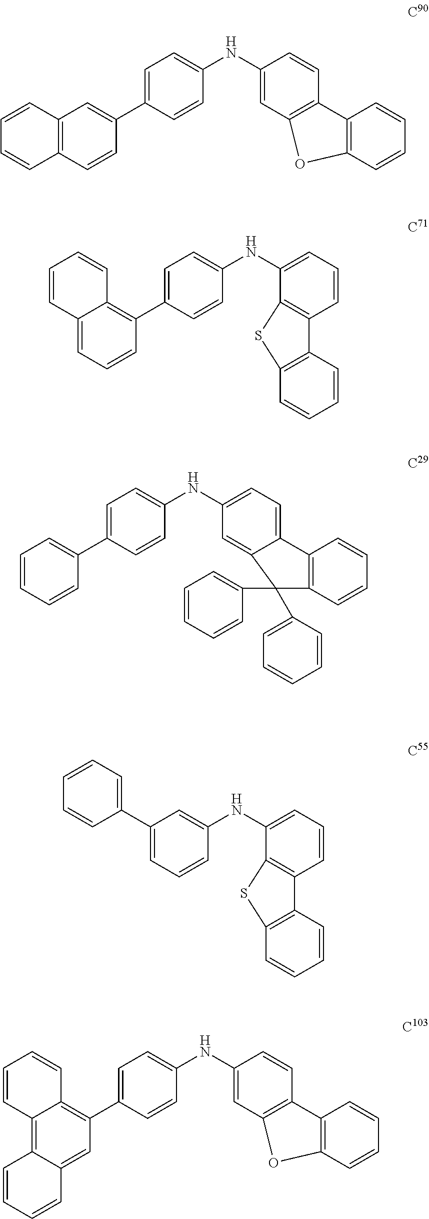 [SMILES: c1ccc2cc(-c3ccc(Nc4ccc5c(c4)oc4ccccc45)cc3)ccc2c1 ; c1ccc2c(-c3ccc(Nc4cccc5c4sc4ccccc45)cc3)cccc2c1 ; c1ccc(-c2ccc(Nc3ccc4c(c3)C(c3ccccc3)(c3ccccc3)c3ccccc3-4)cc2)cc1 ; c1ccc(-c2cccc(Nc3cccc4c3sc3ccccc34)c2)cc1 ; c1ccc2c(c1)cc(-c1ccc(Nc3ccc4c(c3)oc3ccccc34)cc1)c1ccccc12]

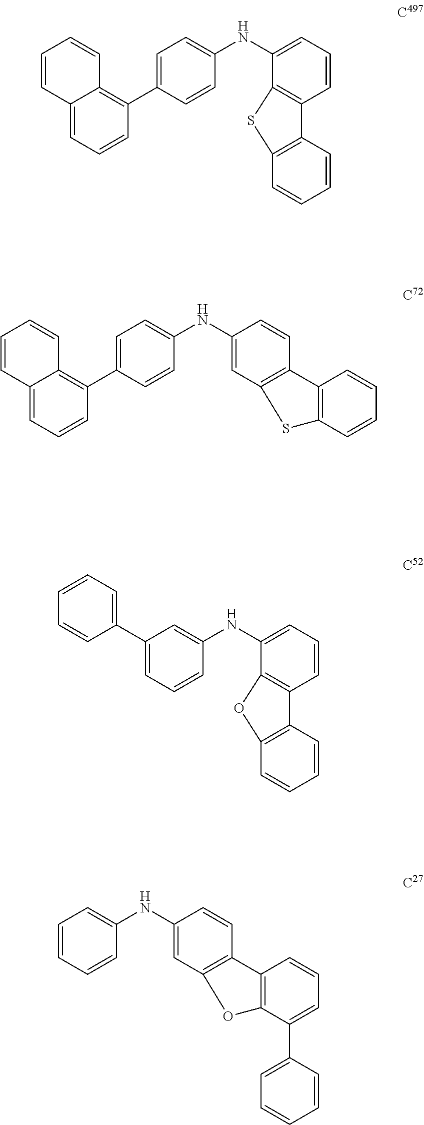 [SMILES: c1ccc2c(-c3ccc(Nc4cccc5c4sc4ccccc45)cc3)cccc2c1 ; c1ccc2c(-c3ccc(Nc4ccc5c(c4)sc4ccccc45)cc3)cccc2c1 ; c1ccc(-c2cccc(Nc3cccc4c3oc3ccccc34)c2)cc1 ; c1ccc(Nc2ccc3c(c2)oc2c(-c4ccccc4)cccc23)cc1]

### (1) Synthesis of Compound 71

Compound 71 was obtained by reacting C⁷¹, obtained from A⁷¹ and B⁷¹, with D⁷¹.

### (Synthesis of C⁷¹)

Toluene (800 mL) was added to a mixture of A⁷¹ (30.0 g, 137 mmol), B⁷¹ (36.0 g, 137 mmol), bis(dibezilideneacetone)palladium (0) (787 mg, 1.37 mmol), and sodium tert-butoxide (19.7 g, 3205 mmol), and tri tert-butylphosphine (2M in toluene, 2.74 mL, 5.47 mmol) was added dropwise thereto, followed by stirring at about 110° C. for about 5 hours. The reaction mixture was cooled, subjected to celite filtering, concentrated, and crystalized by toluene-ethanol to obtain C⁷¹ (35.6 g, yield: 65%). By FAB-MS, m/z=401.1 was confirmed, thereby identifying the production of C⁷¹.

### (Synthesis of Compound 71)

Toluene (300 mL) was added to a mixture of C⁷¹ (10.0 g, 24.9 mmol), D⁷¹ (7.40 g, 24.9 mmol), bis(dibezilideneacetone)palladium (0) (143 mg, 0.25 mmol), and sodium tert-butoxide (3.59 g, 37.4 mmol), and tri tert-butylphosphine (2M in toluene, 0.500 mL, 1.00 mmol) was added dropwise thereto, followed by stirring at about 110° C. for about 7 hours. The reaction mixture was cooled, subjected to celite filtering, and concentrated, and the residue was purified by column chromatography to obtain Compound 71 (13.7 g, yield: 89%). By FAB-MS, m/z=617.2 was confirmed, thereby identifying the production of Compound 71.

### (2) Synthesis of Compound 90

Compound 90 was synthesized in the same manner as in the Synthesis of Compound 71 except for using C⁹⁰ instead of C⁷¹. By FAB-MS, m/z=617.2 was confirmed, thereby identifying the production of Compound 90.

### (3) Synthesis of Compound 29

Compound 29 was synthesized in the same manner as in the Synthesis of Compound 71 except for using C²⁹ instead of C⁷¹. By FAB-MS, m/z=701.3 was confirmed, thereby identifying the production of Compound 29.

### (4) Synthesis of Compound 55

Compound 55 was synthesized in the same manner as in the Synthesis of Compound 71 except for using C⁵⁵ instead of C⁷¹. By FAB-MS, m/z=567.2 was confirmed, thereby identifying the production of Compound 55.

### (5) Synthesis of Compound 103

### (Synthesis of C¹⁰³)

C¹⁰³ was synthesized in the same manner as in the Synthesis of C⁷¹ except for using A¹⁰³ instead of A⁷¹ and using B¹⁰³ instead of B⁷¹. By FAB-MS, m/z=435.2 was confirmed, thereby identifying the production of C¹⁰³.

### (Synthesis of Compound 103)

Compound 103 was synthesized in the same manner as in the Synthesis of Compound 71 except for using C¹⁰³ instead of C⁷¹. By FAB-MS, m/z=651.2 was confirmed, thereby identifying the production of Compound 103.

### (6) Synthesis of Compound 497

Compound 497 was synthesized in the same manner as in the Synthesis of Compound 71 except for using C⁴⁹⁷ instead of C⁷¹ and using D497 instead of D⁷¹. By FAB-MS, m/z=633.2 was confirmed, thereby identifying the production of Compound 497.

### (7) Synthesis of Compound 72

Compound 72 was synthesized in the same manner as in the Synthesis of Compound 71 except for using C⁷² instead of C⁷¹. By FAB-MS, m/z=617.2 was confirmed, thereby identifying the production of Compound 72.

### (8) Synthesis of Compound 52

Compound 52 was synthesized in the same manner as in the Synthesis of Compound 71 except for using C⁵² instead of C⁷¹. By FAB-MS, m/z=551.2 was confirmed, thereby identifying the production of Compound 52.

### (9) Synthesis of Compound 27

Compound 27 was synthesized in the same manner as in the Synthesis of Compound 71 except for using C²⁷ instead of C⁷¹. By FAB-MS, m/z=551.2 was confirmed, thereby identifying the production of Compound 27.

### 2. Manufacture and Evaluation of Light Emitting Element

A light emitting element according to an embodiment including an amine compound according to an embodiment in a hole transport layer was manufactured as follows. Compounds 90, 71, 29, 55, 103, 497, 72, 52, and 27, which are Example Compounds as described above, were used as materials for the hole transport layers to manufacture the light emitting elements of Examples 1 to 9, respectively. Comparative Examples 1 to 16 correspond to the light emitting elements manufactured by using Comparative Example Compounds c1 to c16 as a hole transport layer material.

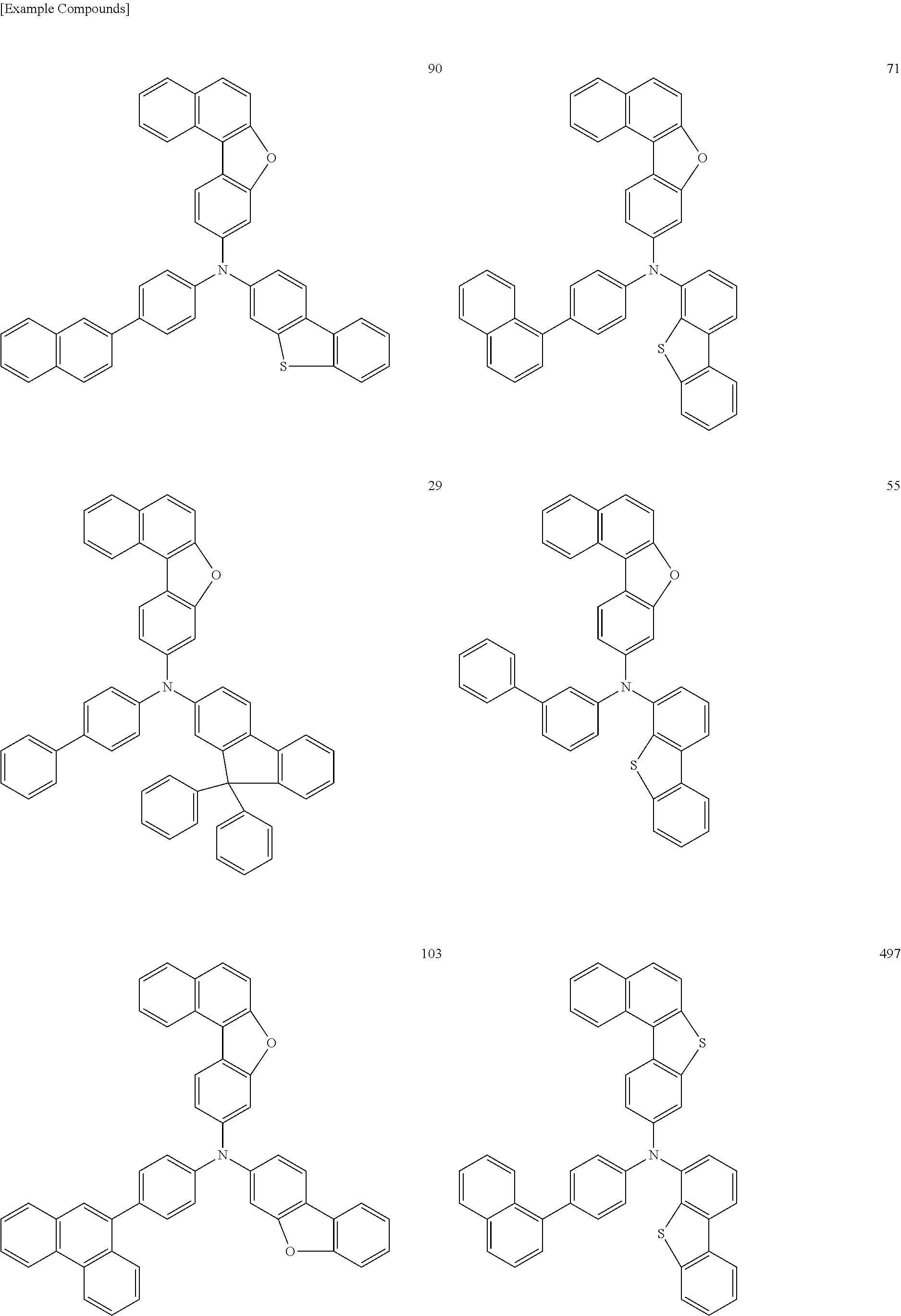 [SMILES: c1ccc2cc(-c3ccc(N(c4ccc5c(c4)oc4ccc6ccccc6c45)c4ccc5c(c4)sc4ccccc45)cc3)ccc2c1 ; c1ccc2c(-c3ccc(N(c4ccc5c(c4)oc4ccc6ccccc6c45)c4cccc5c4sc4ccccc45)cc3)cccc2c1 ; c1ccc(-c2ccc(N(c3ccc4c(c3)C(c3ccccc3)(c3ccccc3)c3ccccc3-4)c3ccc4c(c3)oc3ccc5ccccc5c34)cc2)cc1 ; c1ccc(-c2cccc(N(c3ccc4c(c3)oc3ccc5ccccc5c34)c3cccc4c3sc3ccccc34)c2)cc1 ; c1ccc2c(c1)cc(-c1ccc(N(c3ccc4c(c3)oc3ccccc34)c3ccc4c(c3)oc3ccc5ccccc5c34)cc1)c1ccccc12 ; c1ccc2c(-c3ccc(N(c4ccc5c(c4)sc4ccc6ccccc6c45)c4cccc5c4sc4ccccc45)cc3)cccc2c1]

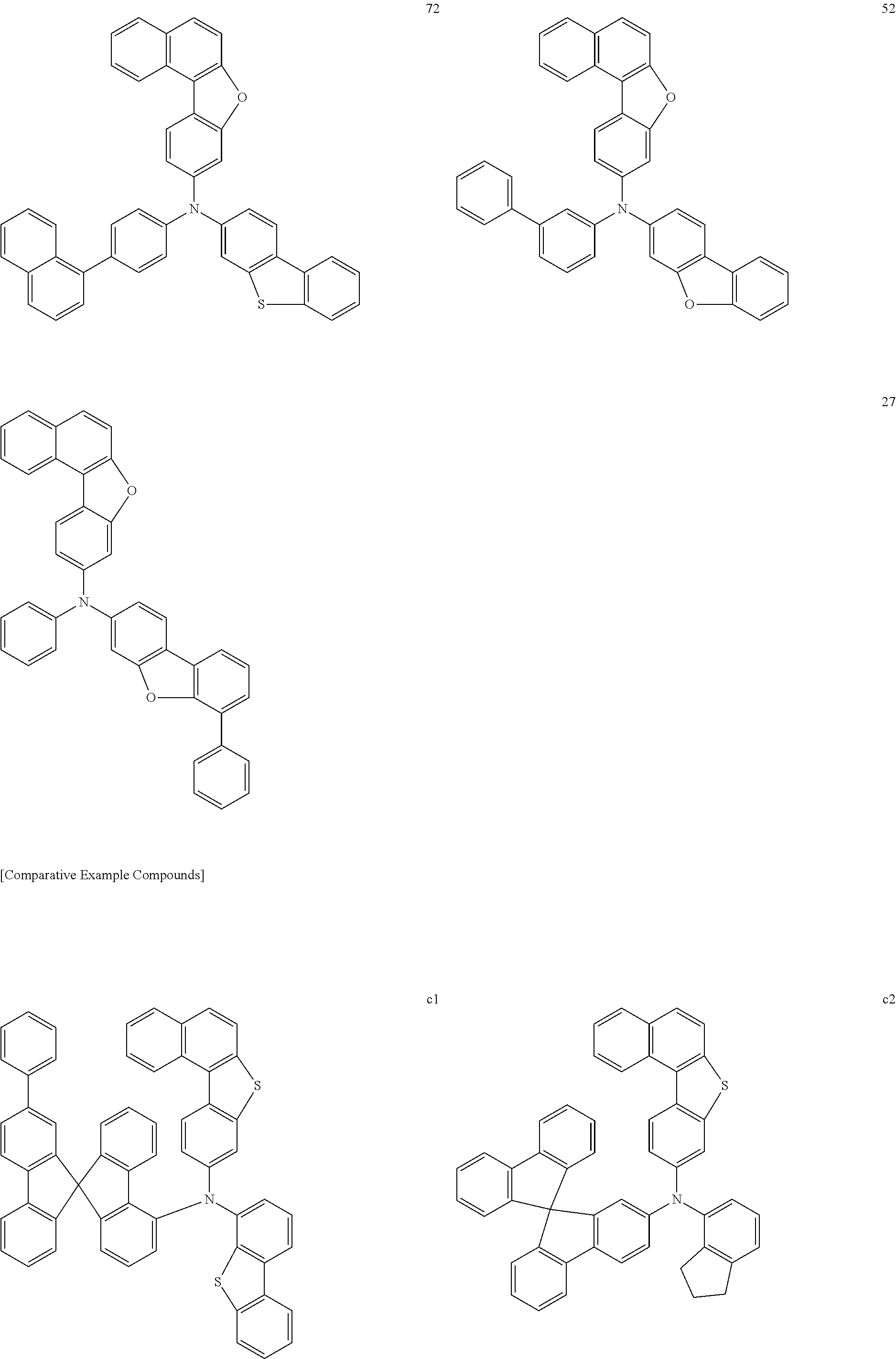

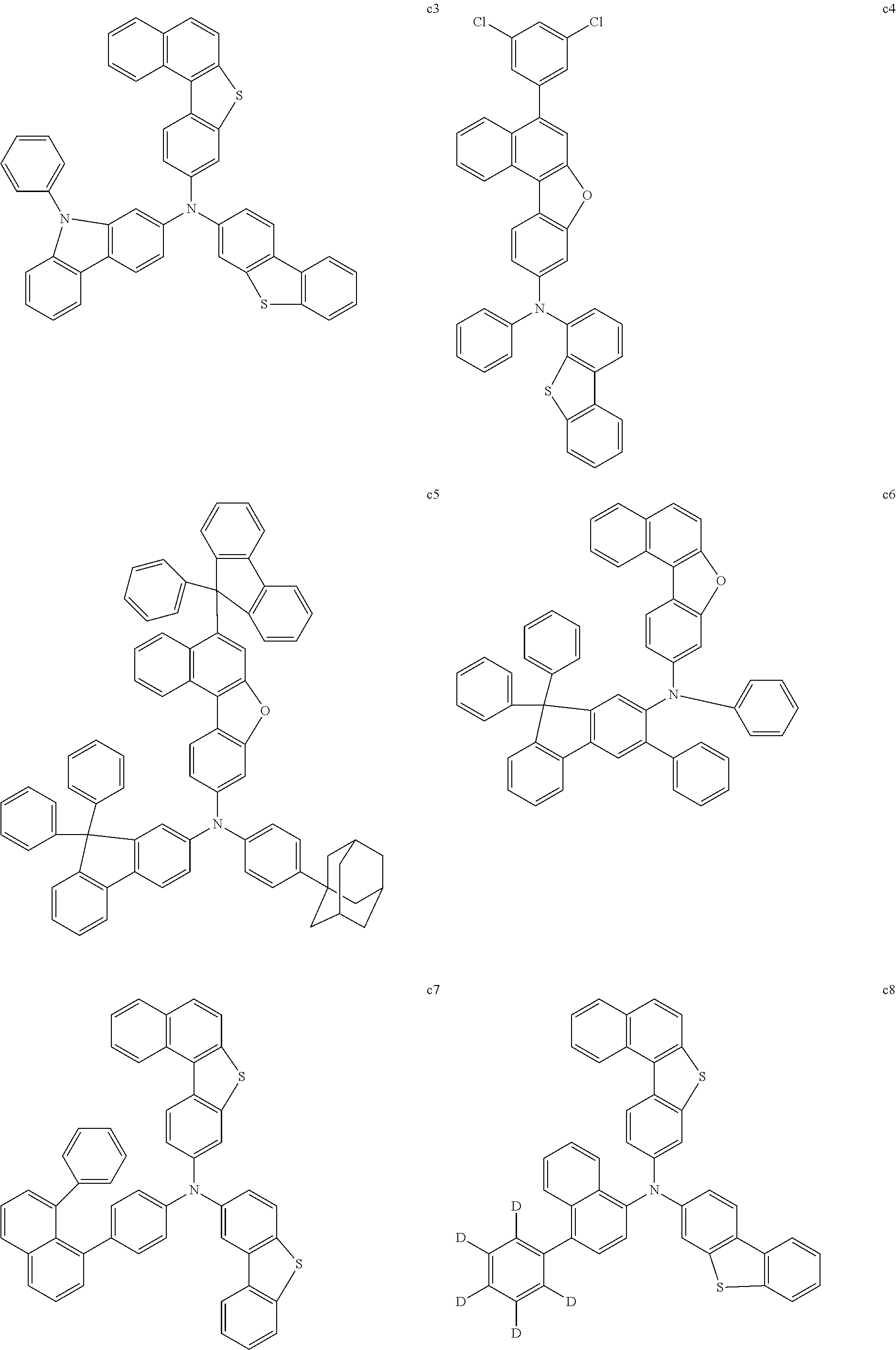

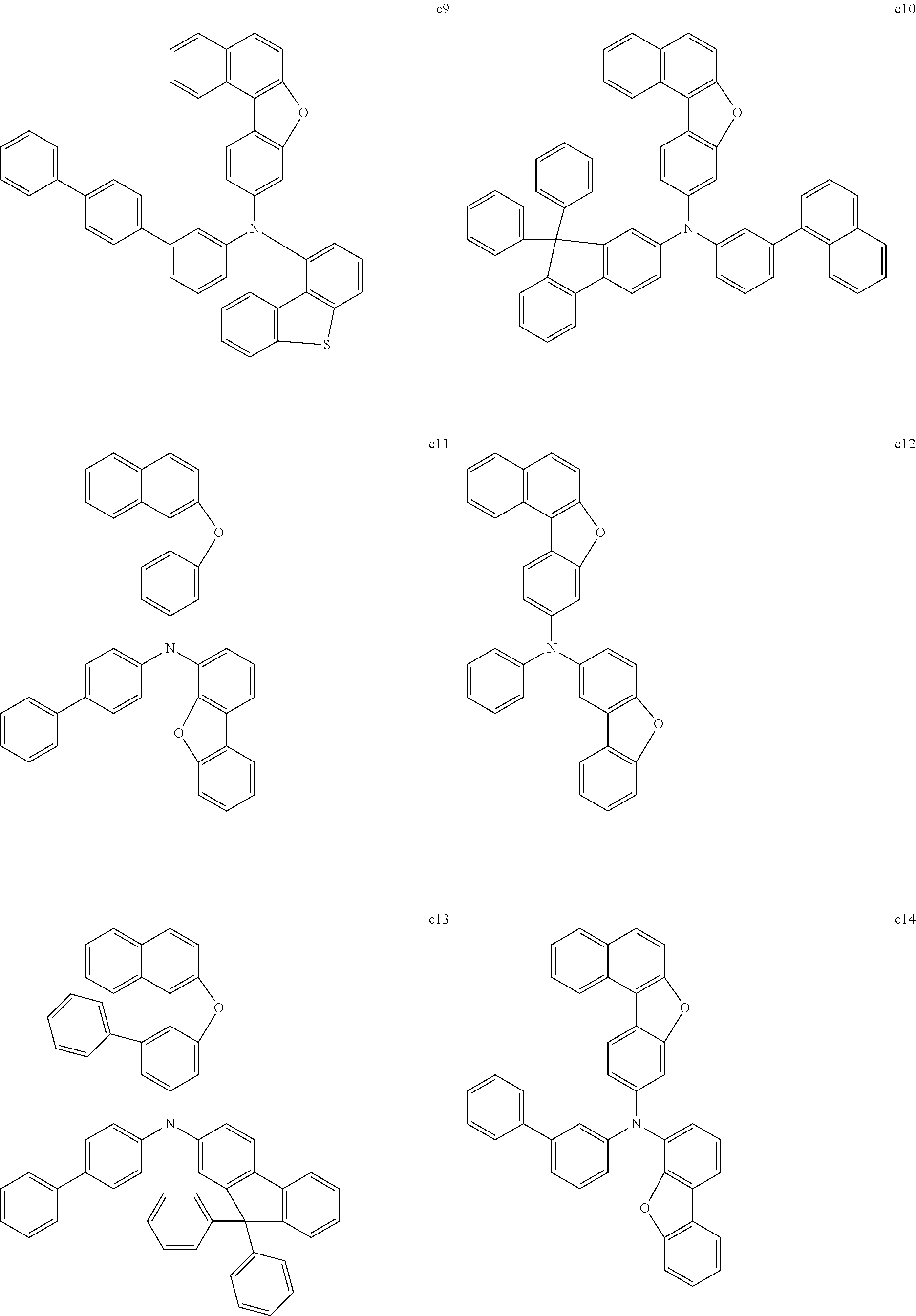 [SMILES: c1ccc(-c2ccc(-c3cccc(N(c4ccc5c(c4)oc4ccc6ccccc6c45)c4cccc5sc6ccccc6c45)c3)cc2)cc1 ; c1ccc(C2(c3ccccc3)c3ccccc3-c3ccc(N(c4cccc(-c5cccc6ccccc56)c4)c4ccc5c(c4)oc4ccc6ccccc6c45)cc32)cc1 ; c1ccc(-c2ccc(N(c3ccc4c(c3)oc3ccc5ccccc5c34)c3cccc4c3oc3ccccc34)cc2)cc1 ; c1ccc(N(c2ccc3c(c2)oc2ccc4ccccc4c23)c2ccc3oc4ccccc4c3c2)cc1 ; c1ccc(-c2ccc(N(c3ccc4c(c3)C(c3ccccc3)(c3ccccc3)c3ccccc3-4)c3cc(-c4ccccc4)c4c(c3)oc3ccc5ccccc5c34)cc2)cc1 ; c1ccc(-c2cccc(N(c3ccc4c(c3)oc3ccc5ccccc5c34)c3cccc4c3oc3ccccc34)c2)cc1]

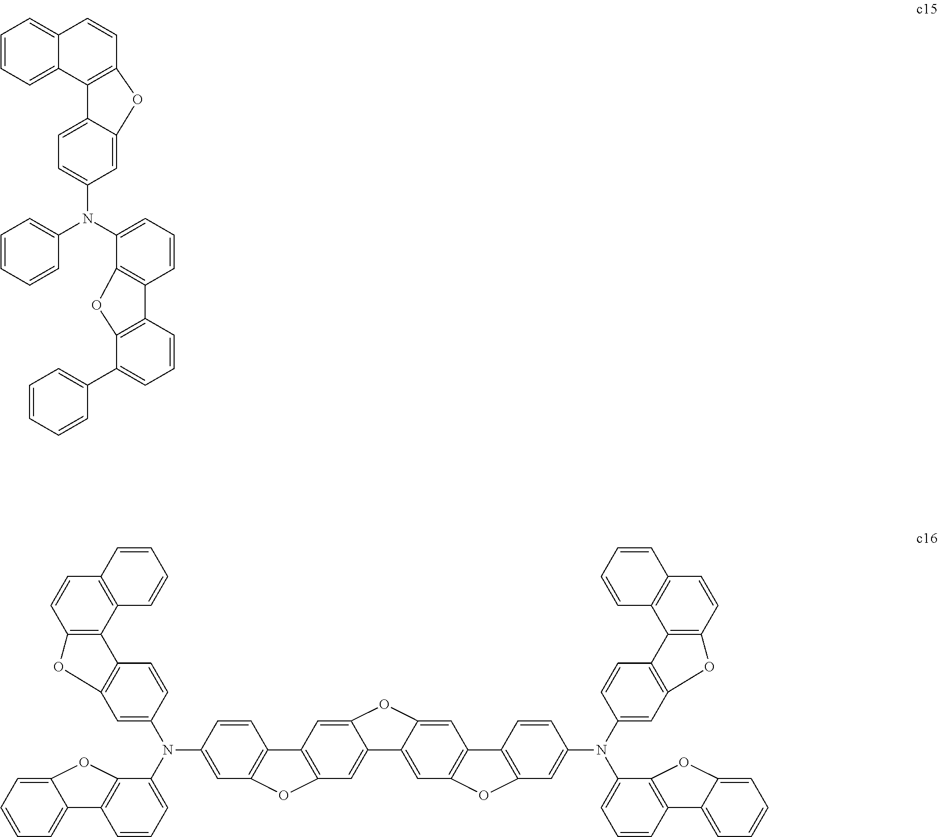 [SMILES: c1ccc(-c2cccc3c2oc2c(N(c4ccccc4)c4ccc5c(c4)oc4ccc6ccccc6c45)cccc23)cc1 ; c1ccc2c(c1)ccc1oc3cc(N(c4ccc5c(c4)oc4cc6c(cc45)oc4cc5c(cc46)oc4cc(N(c6ccc7c(c6)oc6ccc8ccccc8c67)c6cccc7c6oc6ccccc67)ccc45)c4cccc5c4oc4ccccc45)ccc3c12]

### (Manufacture of Light Emitting Elements)

An ITO glass substrate of about 15 Ω/cm² (about 1,500 Å) from Corning Co. was cut to a size of 50 mm×50 mm×0.7 mm, washed with isopropyl alcohol and ultrapure water, and cleansed by ultrasonic waves for about 5 minutes, and irradiated with ultraviolet rays for about 30 minutes and treated with ozone. (4,4′,4″-tris{N-(2-naphthyl)-N-phenylamino}-triphenylamine (2-TNATA) was deposited in vacuum to form a 600 Å-thick hole injection layer, and the Example Compound or Comparative Example Compound was deposited in vacuum to form a 300 Å-thick hole transport layer.

On the hole transport layer, 9,10-di(naphthalen-2-yl)anthracene (ADN) as a blue fluorescent host and 2,5,8,11-Tetra-t-butylperylene (TBP) as a fluorescent dopant were co-deposited in a ratio of 97:3 to form a 250 Å-thick emission layer.

On the emission layer, a 250 Å-thick electron transport layer was formed with tris(8-hydroxyquinolino)aluminum (Alq₃), and LiF was deposited to form a 10 Å-thick electron injection layer. On the electron injection layer, a 1,000 Å-thick second electrode was formed with aluminum (Al).

The compounds of each functional layer used to manufacture light emitting elements are as follows:

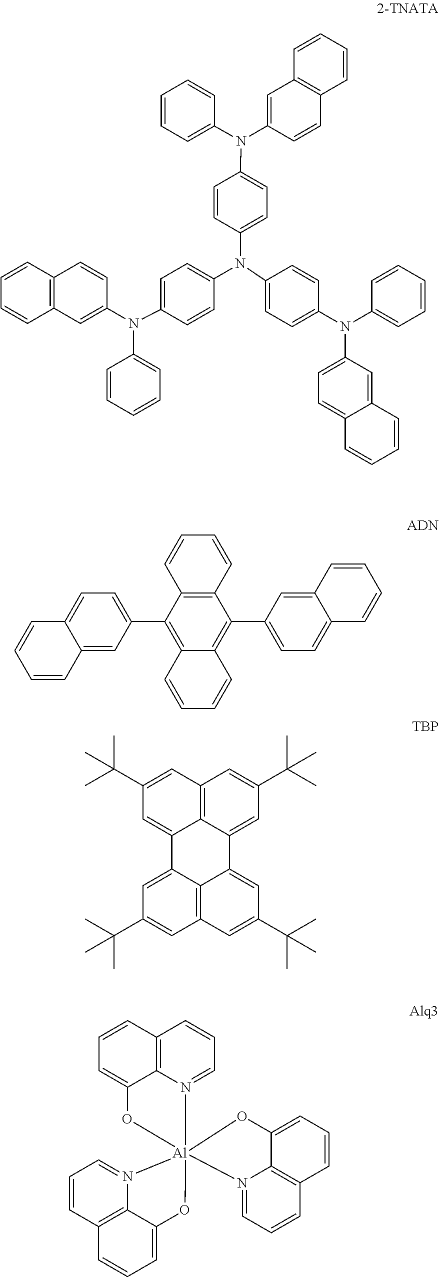

### (Evaluation of the Light Emitting Element)

Evaluation results of the light emitting elements in Examples 1 to 9 and Comparative Examples 1 to 16 are listed in Table 1. Luminous efficiencies and relative service lives of the manufactured light emitting elements are listed in Table 1.

In the characteristic evaluation results of the Examples and Comparative Examples listed in Table 1, the luminous efficiency represents an efficiency value measured at a current density of 10 mA/cm². The half service life represents a half service life value obtained by measuring, at a current density of 10 mA/cm², a point at which an initial brightness, 1000 cd/m², is reduced by half. The relative efficiency represents a relative efficiency when the luminous efficiency of Comparative Example 1 is set as 1, the relative service life represents a relative service life when an element service life of Comparative Example 1 is set as 1.

The evaluation of the current density and luminous efficiency of the light emitting element was performed in a dark room using 2400 Series Source Meter from Keithley Instruments Inc., Color and Luminance Meter CS-200 from Konica Minolta, Inc., PC Program LAbVIEW 8.2 for the measurement from Japan National Instrument, Inc.

TABLE 1

| | | | Relative |
|---|---|---|---|
| | Hole transport | Relative | service |
| | layer | efficiency | life |
| Example 1 | Compound 90 | 110% | 130% |
| Example 2 | Compound 71 | 120% | 130% |
| Example 3 | Compound 29 | 110% | 130% |
| Example 4 | Compound 55 | 105% | 125% |
| Example 5 | Compound 103 | 110% | 130% |
| Example 6 | Compound 497 | 120% | 125% |
| Example 7 | Compound 72 | 115% | 130% |
| Example 8 | Compound 52 | 115% | 125% |
| Example 9 | Compound 27 | 120% | 120% |
| Comparative Example 1 | Compound c1 | 100% | 100% |
| Comparative Example 2 | Compound c2 | 95% | 20% |
| Comparative Example 3 | Compound c3 | 70% | 50% |
| Comparative Example 4 | Compound c4 | 70% | 10% |
| Comparative Example 5 | Compound c5 | 80% | 30% |
| Comparative Example 6 | Compound c6 | 100% | 70% |
| Comparative Example 7 | Compound c7 | 95% | 70% |
| Comparative Example 8 | Compound c8 | 95% | 70% |
| Comparative Example 9 | Compound c9 | 100% | 80% |
| Comparative Example 10 | Compound c10 | 100% | 90% |
| Comparative Example 11 | Compound c11 | 95% | 80% |
| Comparative Example 12 | Compound c12 | 95% | 75% |
| Comparative Example 13 | Compound c13 | 100% | 65% |
| Comparative Example 14 | Compound c14 | 95% | 75% |
| Comparative Example 15 | Compound c15 | 100% | 70% |
| Comparative Example 16 | Compound c16 | 70% | 70% |

Referring to the results of Table 1, it can be seen that the Examples of the light emitting elements in which the amine compounds according to embodiments are used as a hole transport layer material exhibit high luminous efficiencies, and long element service lives compared to the Comparative Examples. The Example Compounds are tertiary amine compounds containing the first substituent, the second substituent, and the third substituent that are linked to the core nitrogen atom, the core nitrogen is bonded at carbon 9 of benzo[b]naphtho[2,1-d]furan or benzo[b]naphtho[2,1-d]thiophene which is the first substituent, and thus the charge density in the compound is increased, thereby improving hole transport ability. The amine compound according to an embodiment further contains the second substituent, and thus the hole transport ability may further be improved, and the stability in a radical cation state may be improved. Therefore, it may be expected that the light emitting elements of the Examples that include the Example Compounds as a hole transport layer material exhibit high luminous efficiency and long element service life.

Comparative Examples c1, c5, and c6 used in Comparative Examples 1, 5, and 6 include a fluorene moiety having a spiro structure, and tend to be vulnerable to heat. Accordingly, Comparative Examples 1, 5, and 6 exhibited characteristics in which the luminous efficiencies and element service lives are deteriorated as compared to Example 3. The 9,9-diphenylfluorene moiety exhibits improved thermal stability compared to a 9,9-spirobifluorene moiety. Thus, the light emitting element of Example 3 exhibited excellent luminous efficiency and element service life compared to the light emitting elements of Comparative Examples 1, 5, and 6.

Comparative Example Compound c5 used in Comparative Example 5 has a structure in which the fluorene group is substituted via carbon 9 at the benzonaphthofuran moiety, and the structure deteriorates the stability of the compound due to the additional quaternary carbon and the twist of the molecule. Accordingly, it may be confirmed that Comparative Example 5 exhibited characteristics in which the luminous efficiency and element service life are deteriorated as compared to the Examples.

Comparative Example Compound c2 used in Comparative Example 2 contains an aryl group in which a cycloalkyl ring is fused in the molecule, and contains benzylic hydrogen having high activity, and thus exhibits deteriorated stability. Accordingly, Comparative Example 2 exhibited characteristics in which both the luminous efficiency and the element service life are deteriorated as compared to the Examples.

Comparative Example Compound c3 used in Comparative Example 3 is different from the Example Compounds in that a carbazole group, which is a nitrogen-containing heterocycle, is included in the molecule. The carbazole group has a great effect on the charge transport property of the molecule, and as a result, charge balance of the emission layer is disturbed, and when the compound is applied to the light emitting element, Comparative Example 3 exhibited characteristics in which luminous efficiency and service life are deteriorated as compared to the Examples.

Comparative Example Compound c4 used in Comparative Example 4 includes a halogen atom in the molecule, and the halogenated molecule has high reaction activity, so that chemical stability is deteriorated. Thus, it is thought that Comparative Example 4 exhibits deterioration in luminous efficiency and element service life as compared to the Examples.

Comparative Example Compound c7 used in Comparative Example 7 has a structure in which a substituent having a largely twisted phenyl-naphthyl-phenyl structure is linked to the nitrogen atom of the amine group, and it is thought that the stability of the compound is deteriorated by the twist due to the structure, and thus luminous efficiency and service life are deteriorated when the compound was applied to the light emitting element.

Comparative Example Compound c8 used in Comparative Example 8 has a structure in which a naphthalene skeleton is directly linked to the core nitrogen, and in this structure, a twist between the naphthalene skeleton and the nitrogen of the amine group occurs, and thus the stability of the compound is deteriorated. Accordingly, it is believed that Comparative Example 8 exhibited deteriorated element service life characteristics as compared to the Examples.

Comparative Example Compound c9 used in Comparative Example 9 includes a m-phenylene part in which the dibenzothiophene group is bonded to the amine group at carbon 1 and directly linked to the nitrogen atom of the arylamine. When the dibenzothiophene group is linked to the core nitrogen via carbon 1, a relatively large twist occurs in the arylamine part due to the large atomic radius of sulfur, and when the m-phenylene group is additionally bonded to the core nitrogen, the stability of the molecule deteriorates. Accordingly, it is thought that Comparative Example 9 exhibited characteristics in which the luminous efficiency and element service life are deteriorated as compared to the Examples.

Comparative Example Compound c10 used in Comparative Example 10 contained a relatively large 9,9-spirobifluorene moiety and a naphthalene part in the molecule. Due to this structure, it is thought that the stability of the compound is not sufficient, and thus luminous efficiency and service life are deteriorated when the compound was applied to the element.

Comparative Example Compounds c11, c12, c14, and c15 used in Comparative Examples 11, 12, 14, and 15 correspond to compounds in which the oxygen atom of the dibenzofuran moiety is disposed at an ortho- or a para-position to the core nitrogen of the amine group. For example, Comparative Example Compounds c11, c12, c14, and c15 have a structure in which the dibenzofuran moiety is linked to the core nitrogen atom at carbon 2 or carbon 4, and a phenyl group and a biphenyl group are additionally substituted at the nitrogen atom of the arylamine. When nitrogen and oxygen having electron donating properties are disposed at an ortho- or a para-position, the substituted phenyl group at the core nitrogen is unstable due to the interaction between the nitrogen of the amine group and the unpaired electron of oxygen of the dibenzofuran, and when the compounds are applied to the element, luminous efficiency and service life may deteriorate.

Comparative Example Compound c13 used in Comparative Example 13 includes a benzonaphthofuran moiety and a 9,9-diphenylfluorene moiety, and has a structure in which a phenyl group is additionally linked to a benzene ring linked to a nitrogen atom in the benzonaphthofuran moiety. Due to this structure, stability is not sufficient due to the effect of the twist of the molecule, and thus it is thought that luminous efficiency and service life are deteriorated when the compound was applied to the element.

Comparative Example Compound c16 used in Comparative Example 16 includes a heterocycle which contains multiple oxygen atoms and has a large planar structure. It is thought that the stacking between molecules is very large, resulting in deterioration of charge balance and stability of the element, thereby deteriorating the luminous efficiency and service life.

The light emitting element may include the amine compound according to an embodiment, thereby exhibiting high efficiency and long service life characteristics.

The amine compound according to an embodiment may exhibit high efficiency and long service life characteristics when applied to a light emitting element.

Embodiments have been disclosed herein, and although terms are employed, they are used and are to be interpreted in a generic and descriptive sense only and not for purposes of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure.

What is claimed is:

1. A light emitting element comprising:
a first electrode;
a second electrode disposed on the first electrode; and
at least one functional layer disposed between the first electrode and the second electrode and comprising an amine compound represented by Formula 1: 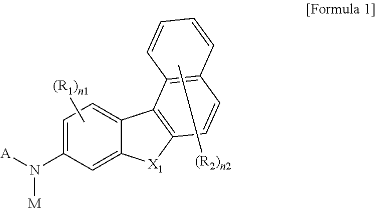
wherein in Formula 1,
A is a group represented by one of Formula 2-1 to Formula 2-3,
M is a group represented by Formula 3,
X₁ is O or S,
R₁ and R₂ are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 is an integer from 0 to 3, and
n2 is an integer from 0 to 6, 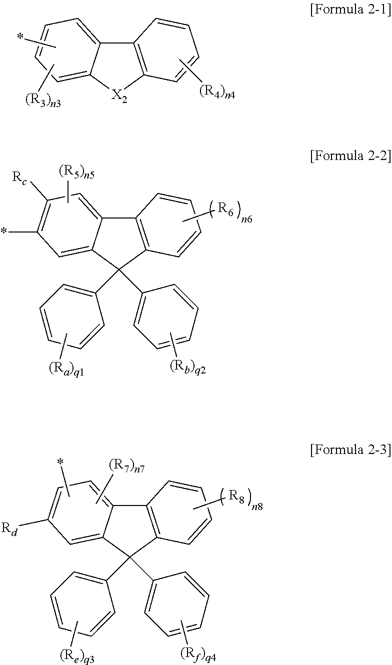
wherein in Formula 2-1 to Formula 2-3,
X₂ is O or S,
R_{c} is a hydrogen atom, a deuterium atom, or a substituted or unsubstituted alkyl group having 1 to 20 carbon atoms,
Rₐ, R_{b}, R_{d} to R_{f}, and R₃ to R₈ are each independently a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,
n3 is an integer from 0 to 3,
n4, n6, and n8 are each independently an integer from 0 to 4,
n5 and n7 are each independently an integer from 0 to 2,
q1 to q4 are each independently an integer from 0 to 5, and
—* represents a bond to Formula 1; 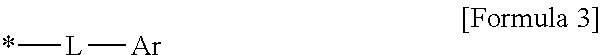
wherein in Formula 3,
L is a direct linkage, a substituted or unsubstituted arylene group having 6 to 30 ring-forming carbon atoms, or a substituted or unsubstituted heteroarylene group having 2 to 30 ring-forming carbon atoms,
Ar is a substituted or unsubstituted aryl group having 6 to 30 ring-forming carbon atoms, or a substituted or unsubstituted heteroaryl group having 2 to 30 ring-forming carbon atoms and 1 or 2 ring-forming heteroatoms, a group represented by Formula 2-2, or a group represented by Formula 2-3, and
—* represents a bond to Formula 1,
wherein in the amine compound represented by Formula 1,
a case where the amine compound comprises an aromatic fused ring containing sp³ carbon in addition to sp³ carbon contained in a fluorene skeleton represented by Formula 2-2 or Formula 2-3 is excluded,
a case where the amine compound comprises a nitrogen-containing heterocycle and a halogen atom is excluded, and
a case where M is a group represented by Formula a-1 or Formula a-2 is excluded: 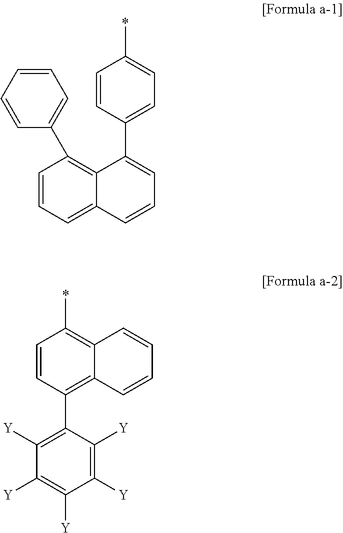
wherein in Formula a-1 and Formula a-2,
Y is a hydrogen atom or a deuterium atom, and
—* represents a bond to Formula 1, and
wherein in Formula 1,
when A is a group represented by Formula 2-2 or Formula 2-3, M does not comprise a naphthylene moiety,
when A is a group represented by Formula 2-1 and the group represented by Formula 2-1 is linked to N in Formula 1 at carbon 4, n1 is 0,
when A is a group represented by Formula 2-2 or Formula 2-3, n1 is 0,
when A is a group represented by Formula 2-1, X₂ in Formula 2-1 is S, and the group represented by Formula 2-1 is linked to N in Formula 1 at carbon 1, a case where L is a direct linkage or a group represented by Formula a-3 is excluded: 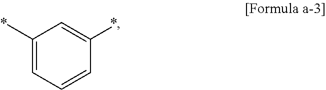
and
when A is a group represented by Formula 2-1, X₂ in Formula 2-1 is O, and the group represented by Formula 2-1 is linked to N in Formula 1 at carbon 2 or carbon 4, a case where M in Formula 1 is an unsubstituted phenyl group or a phenyl group having a substituent having 0 to 9 carbon atoms is excluded.

2. The light emitting element of claim 1, wherein
the at least one functional layer comprises:
an emission layer;
a hole transport region disposed between the first electrode and the emission layer; and
an electron transport region disposed between the emission layer and the second electrode, and
the hole transport region comprises the amine compound.

3. The light emitting element of claim 2, wherein
the hole transport region comprises:
a hole injection layer disposed on the first electrode; and
a hole transport layer disposed on the hole injection layer; and
the hole transport layer comprises the amine compound.

4. The light emitting element of claim 2, wherein a functional layer that is included in the hole transport region is adjacent to the emission layer and comprises the amine compound.

5. The light emitting element of claim 1, wherein the amine compound is a monoamine compound.

6. The light emitting element of claim 1, wherein the amine compound is represented by one of Formula 1-1-1 to Formula 1-1-3: 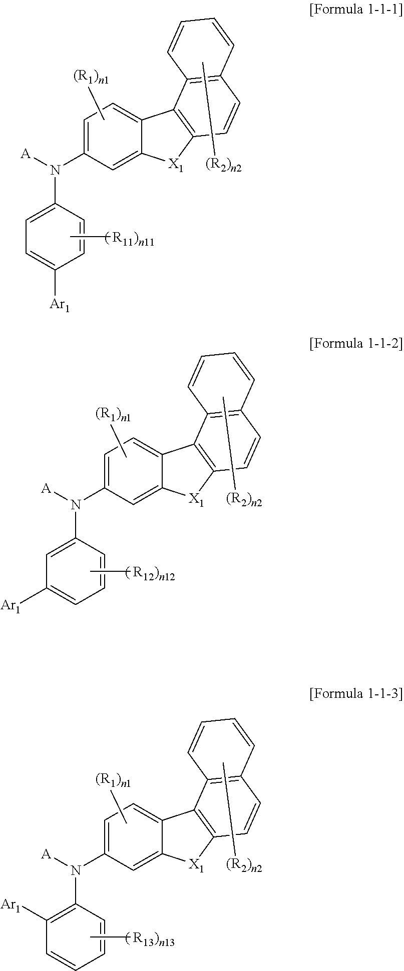
wherein in Formula 1-1-1 to Formula 1-1-3,
R₁₁ to R₁₃ are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n11 to n13 are each independently an integer from 0 to 4, and
Ar₁ is a group represented by one of Formula A-1 to Formula A-4: 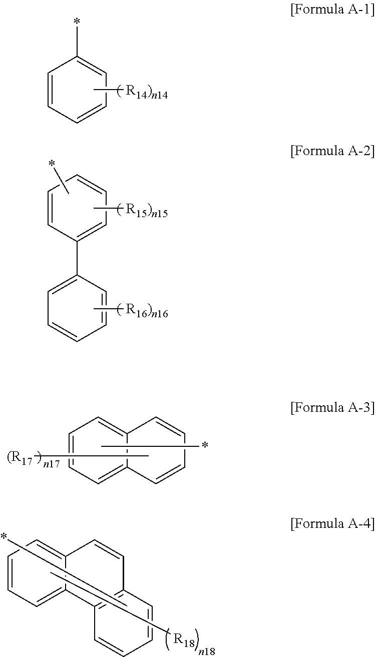
wherein in Formula A-1 to Formula A-4,
R₁₄ to R₁₈ are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n14 and n16 are each independently an integer from 0 to 5,
n15 is an integer from 0 to 4,
n17 is an integer from 0 to 7, and
n18 is an integer from 0 to 9, and
wherein in Formula 1-1-1 to Formula 1-1-3,
when A is a group represented by Formula 2-2 or Formula 2-3, Ar₁ is a group represented by Formula A-1, Formula A-2, or Formula A-4, and
A, X₁, R₁, R₂, n1, and n2 are the same as defined in Formula 1.

7. The light emitting element of claim 1, wherein the amine compound is represented by one of Formula 1-2-1 to Formula 1-2-7: 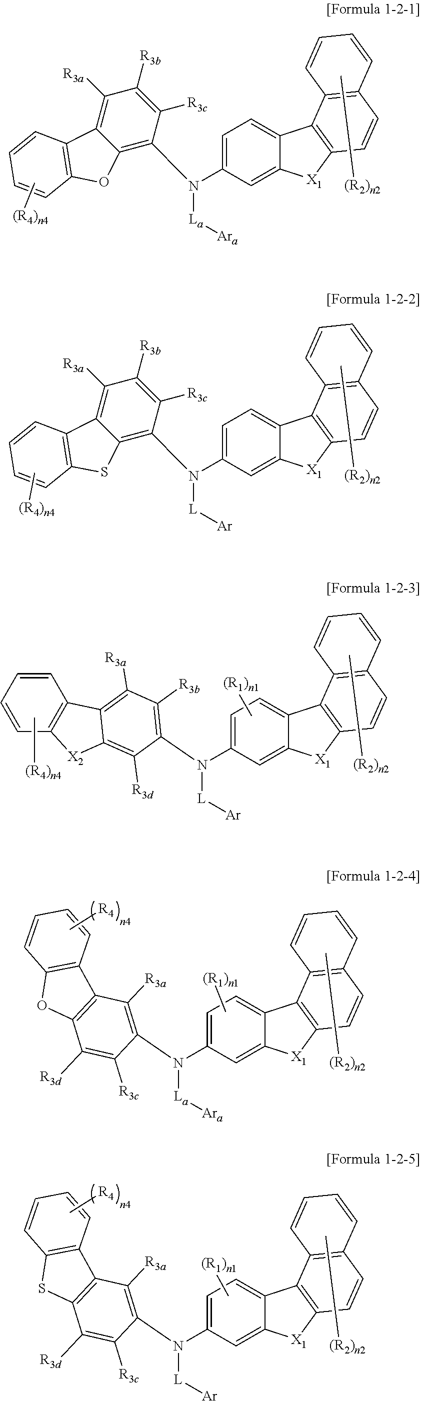 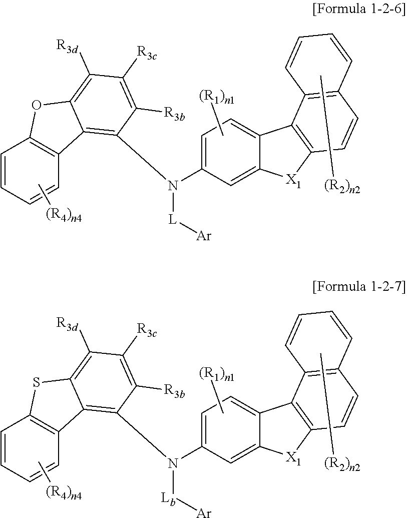
wherein in Formula 1-2-1 to Formula 1-2-7,
R₃ₐ to R_{3d} are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Lₐ is a substituted or unsubstituted arylene group having 6 to 30 ring-forming carbon atoms or a substituted or unsubstituted heteroarylene group having 2 to 30 ring-forming carbon atoms, and
Arₐ is a substituted or unsubstituted aryl group having 6 to 30 ring-forming carbon atoms or a substituted or unsubstituted heteroaryl group having 2 to 30 ring-forming carbon atoms,
except that Arₐ is not a substituted or unsubstituted phenyl group,
wherein in Formula 1-2-7,
L_{b} is a substituted or unsubstituted arylene group having 6 to 30 ring-forming carbon atoms or a substituted or unsubstituted heteroarylene group having 2 to 30 ring-forming carbon atoms, and
a case where L_{b} is a group represented by Formula a-3 is excluded, and
wherein in Formula 1-2-1 to Formula 1-2-7,
L, Ar, X₁, R₁, R₂, n1, n2, X₂, R₄, and n4 are the same as defined in Formula 1 and Formula 2.

8. The light emitting element of claim 1, wherein the amine compound is represented by one of Formula 1-3-1 to Formula 1-3-4: 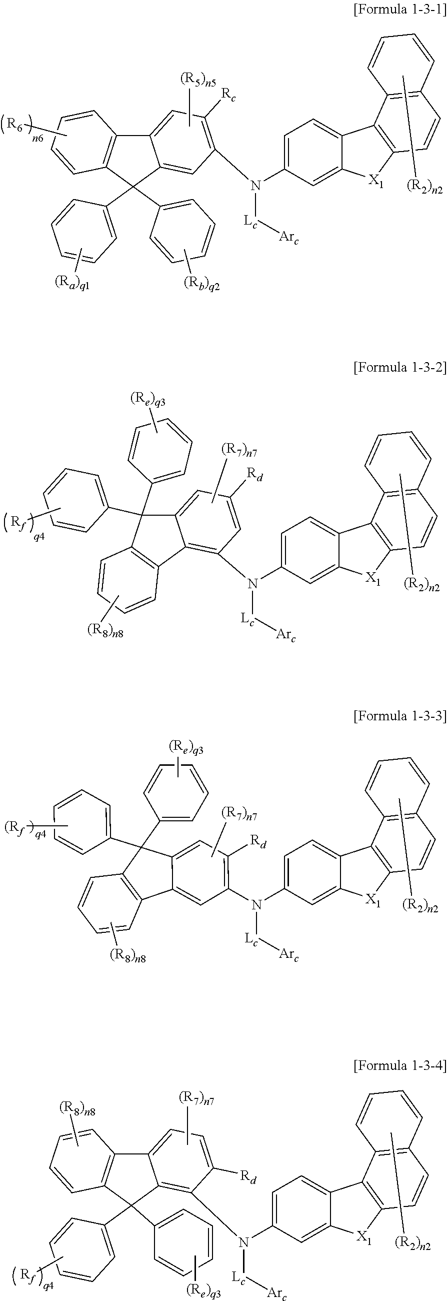
wherein in Formula 1-3-1 to Formula 1-3-4,
L_{c} is a direct linkage, a substituted or unsubstituted arylene group having 6 to 30 ring-forming carbon atoms, or a substituted or unsubstituted heteroarylene group having 2 to 30 ring-forming carbon atoms,
except that L4 is not a substituted or unsubstituted naphthalene group,
Ar_{c} is a substituted or unsubstituted aryl group having 6 to 30 ring-forming carbon atoms or a substituted or unsubstituted heteroaryl group having 2 to 30 ring-forming carbon atoms,
except that Ar_{c} is not a substituted or unsubstituted naphthyl group, and
X₁, R₂, n2, R₅ to R₈, n5 to n8, Rₑ to R_{f}, and q1 to q4 are the same as defined in Formula 1, Formula 2-2, and Formula 2-3.

9. The light emitting element of claim 1, wherein the amine compound is represented by Formula 1-4-1 or Formula 1-4-2: 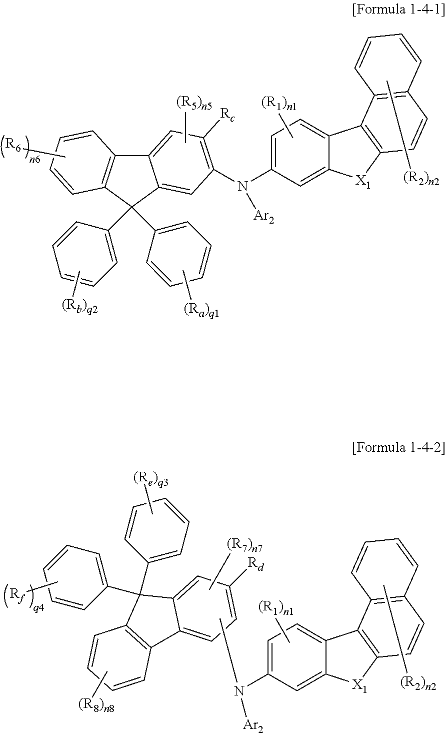
wherein in Formula 1-4-1 and Formula 1-4-2,
Ar₂ is a group represented by one of Formula B-1 to Formula B-7: 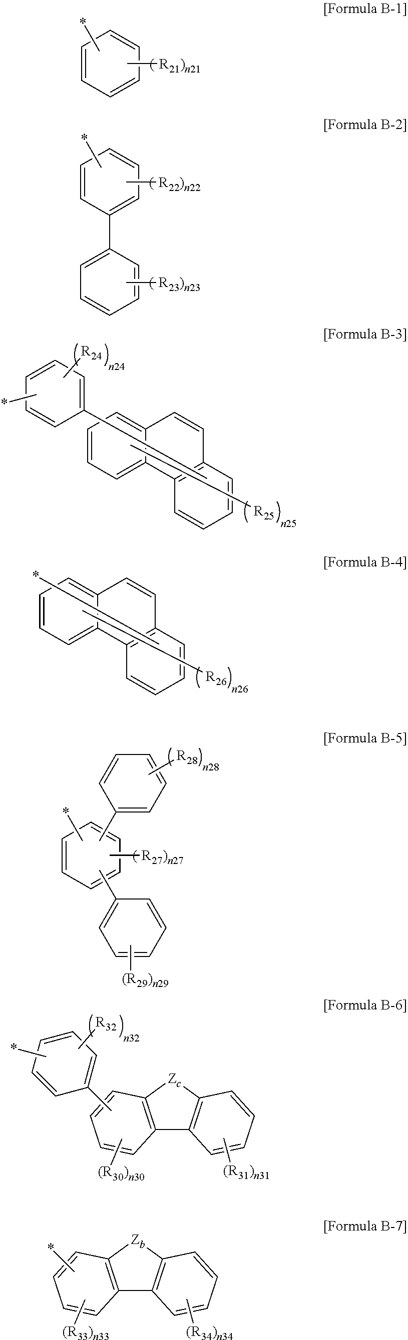
wherein in Formula B-1 to Formula B-7,
Zₐ and Z_{b} are each independently O or S,
R₂₁ to R₃₄ are each independently a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,
n21, n23, n28, and n29 are each independently an integer from 0 to 5,
n22, n24, n31, n32, and n34 are each independently an integer from 0 to 4,
n25 and n26 are each independently an integer from 0 to 9, and
n27, n30, and n33 are each independently an integer from 0 to 3, and
wherein in Formula 1-4-1 and Formula 1-4-2,
X₁, R₁, R₂, n1, n2, R₅ to R₈, n5 to n8, Rₐ to R_{f}, and q1 to q4 are the same as defined in Formula 1, Formula 2-2, and Formula 2-3.

10. The light emitting element of claim 1, wherein the amine compound is represented by Formula 1-5-1 or Formula 1-5-2: 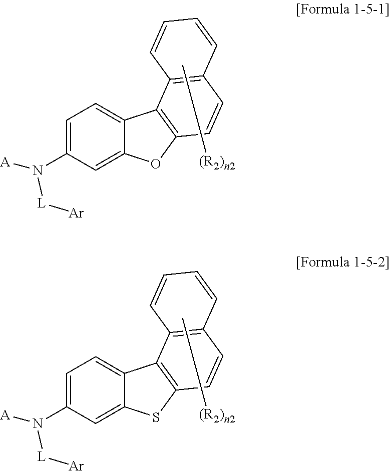
wherein in Formula 1-5-1 and Formula 1-5-2,
A, L, Ar, R₂, and n2 are the same as defined in Formula 1.

11. The light emitting element of claim 1, wherein the amine compound is represented by one of Formula 1-6-1 to Formula 1-6-4: 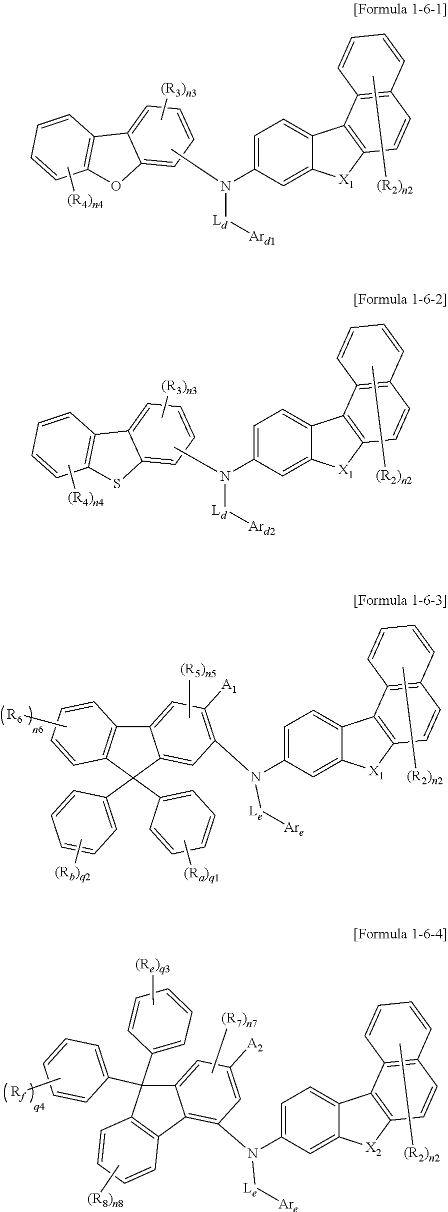
wherein in Formula 1-6-1 to Formula 1-6-4,
A₁ and A₂ are each independently a hydrogen atom or a deuterium atom,
L_{d} is a group represented by one of Formula L-1 to Formula L-5,
Ar_{d1} is a group represented by Formula 2-2, a group represented by Formula 2-3, a substituted or unsubstituted naphthyl group, a substituted or unsubstituted anthracene group, a substituted or unsubstituted phenanthrene group, a substituted or unsubstituted dibenzofuran group, or a substituted or unsubstituted dibenzothiophene group,
Ar_{d2} is a group represented by Formula 2-2, a group represented by Formula 2-3, a substituted or unsubstituted phenyl group, a substituted or unsubstituted biphenyl group, a substituted or unsubstituted naphthyl group, a substituted or unsubstituted phenanthrene group, a substituted or unsubstituted anthracene group, a substituted or unsubstituted dibenzofuran group, or a substituted or unsubstituted dibenzothiophene group,
Lₑ is a direct linkage, or a group represented by one of Formula L-1 to Formula L-5, and
Arₑ is a group represented by Formula 2-2, a group represented by Formula 2-3, or a group represented by one of Formula C-1 to Formula C-5: 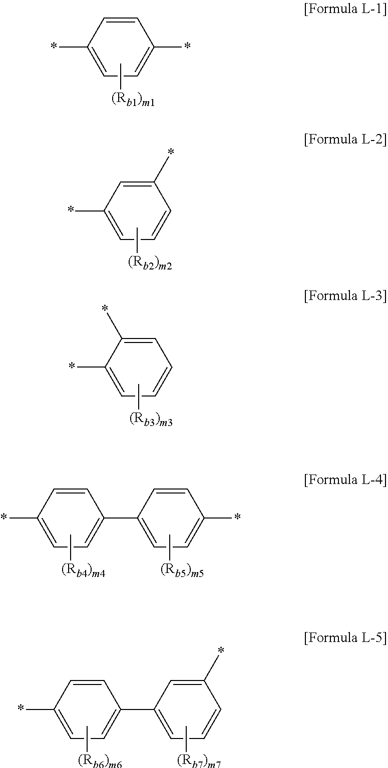
wherein in Formula L-1 to Formula L-5,
R_{b1} to R_{b7} are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
m1 to m7 are each independently an integer from 0 to 4; 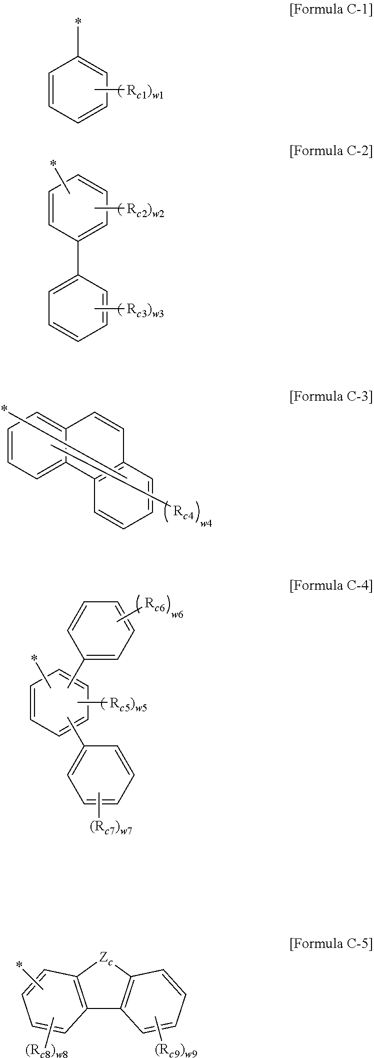
wherein in Formula C-1 to Formula C-5,
Z_{c} is O or S,
R_{c1} to R_{c9} are each independently a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,
w1, w3, w6, and w7 are each independently an integer from 0 to 5,
w2 and w9 are each independently an integer from 0 to 4,
w4 is an integer from 0 to 9, and
w5 and w8 are each independently an integer from 0 to 3, and
wherein in Formula 1-6-1 to Formula 1-6-4,
X₁, R₂ to R₈, n2 to n8, Rₐ, R_{b}, Rₑ, R_{f}, and q1 to q4 are the same as defined in Formula 1, Formula 2-2, and Formula 2-3.

12. The light emitting element of claim 1, wherein in Formula 1,
M is a group selected from Substituent Group B, and
Ar is a group selected from Substituent Group C:

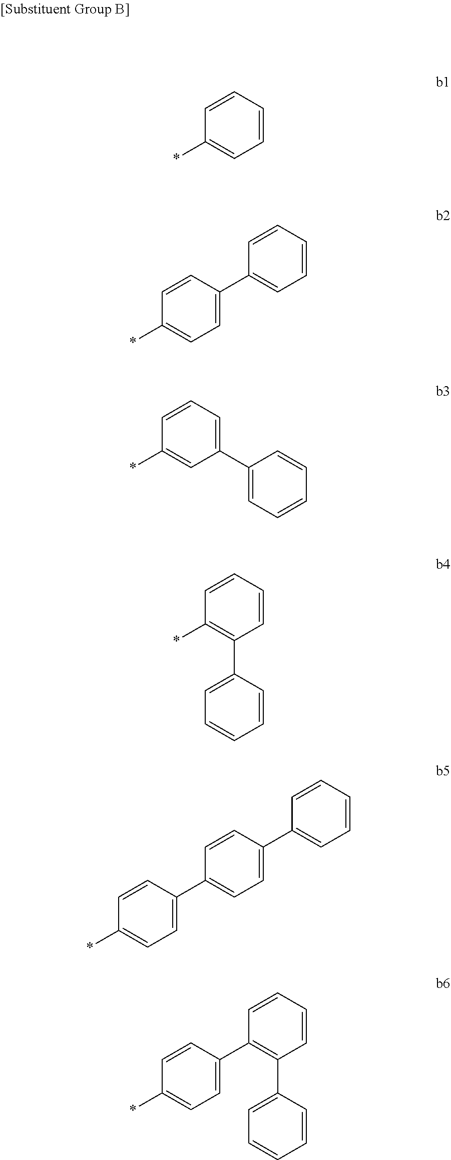 [SMILES: *c1ccccc1 ; *c1ccc(-c2ccccc2)cc1 ; *c1cccc(-c2ccccc2)c1 ; *c1ccccc1-c1ccccc1 ; *c1ccc(-c2ccc(-c3ccccc3)cc2)cc1 ; *c1ccc(-c2ccccc2-c2ccccc2)cc1]

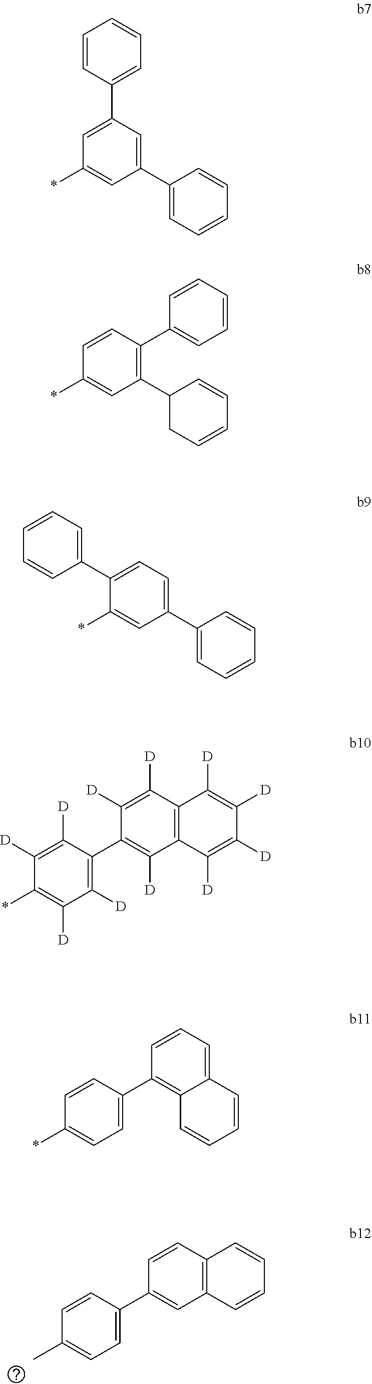

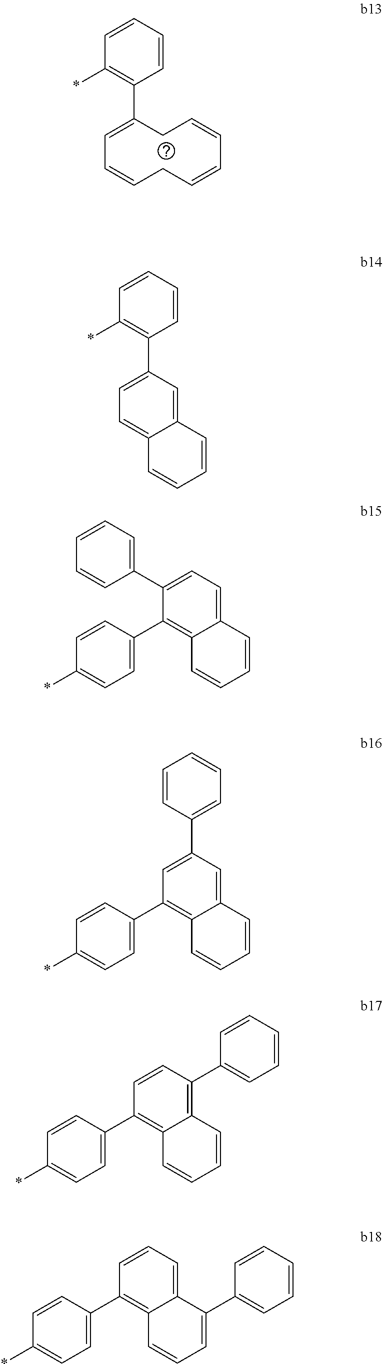 [SMILES: *c1ccccc1/C1=C/C=C\C/C=C\C=C/C1 ; *c1ccccc1-c1ccc2ccccc2c1 ; *c1ccc(-c2c(-c3ccccc3)ccc3ccccc23)cc1 ; *c1ccc(-c2cc(-c3ccccc3)cc3ccccc23)cc1 ; *c1ccc(-c2ccc(-c3ccccc3)c3ccccc23)cc1 ; *c1ccc(-c2cccc3c(-c4ccccc4)cccc23)cc1]

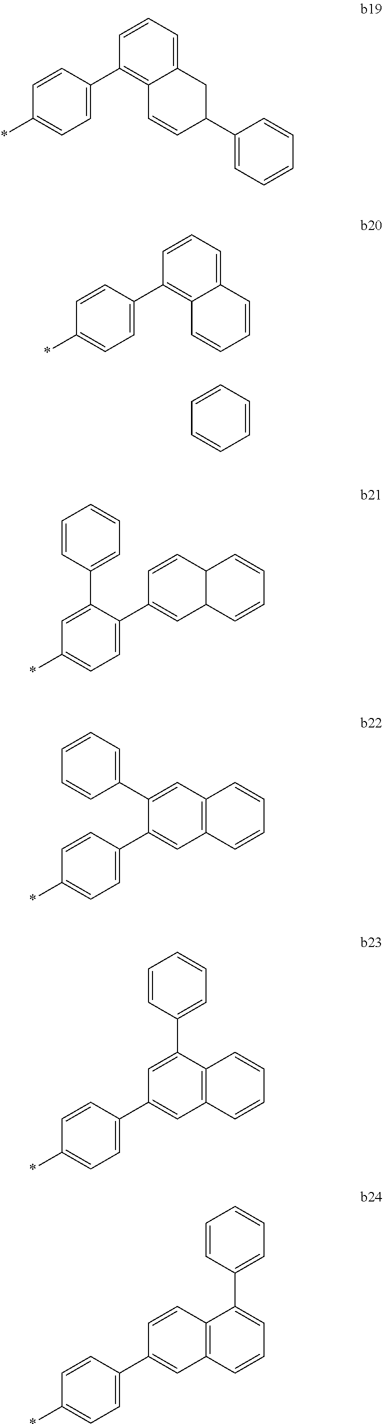

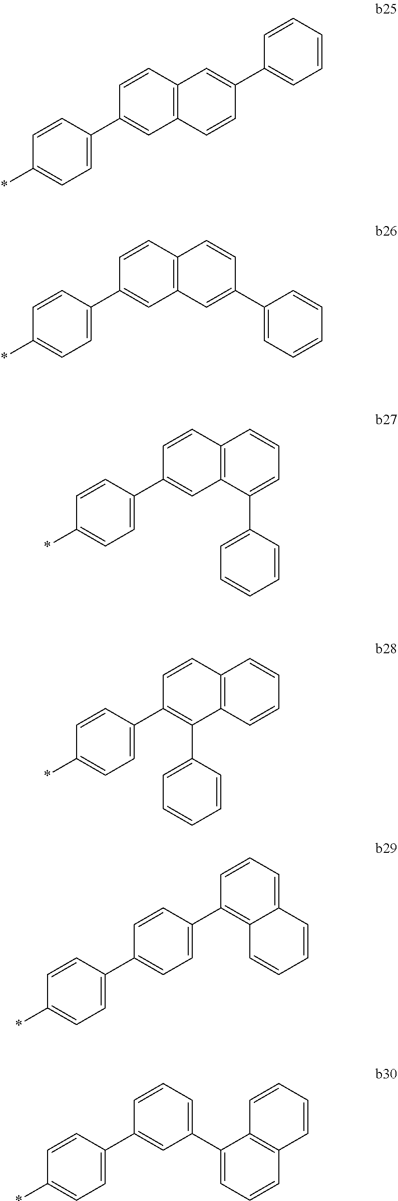 [SMILES: *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1 ; *c1ccc(-c2ccc3ccccc3c2-c2ccccc2)cc1 ; *c1ccc(-c2ccc(-c3cccc4ccccc34)cc2)cc1 ; *c1ccc(-c2cccc(-c3cccc4ccccc34)c2)cc1]

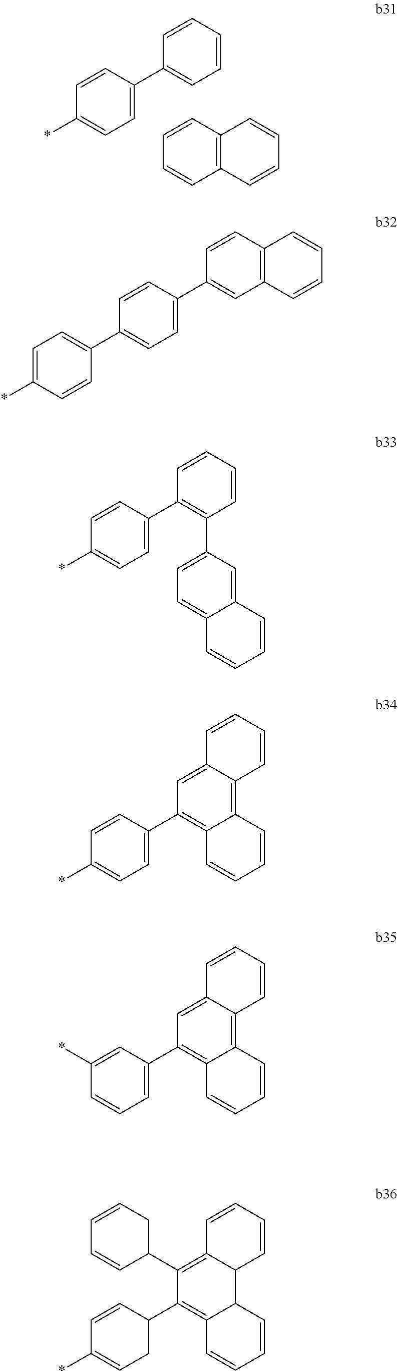

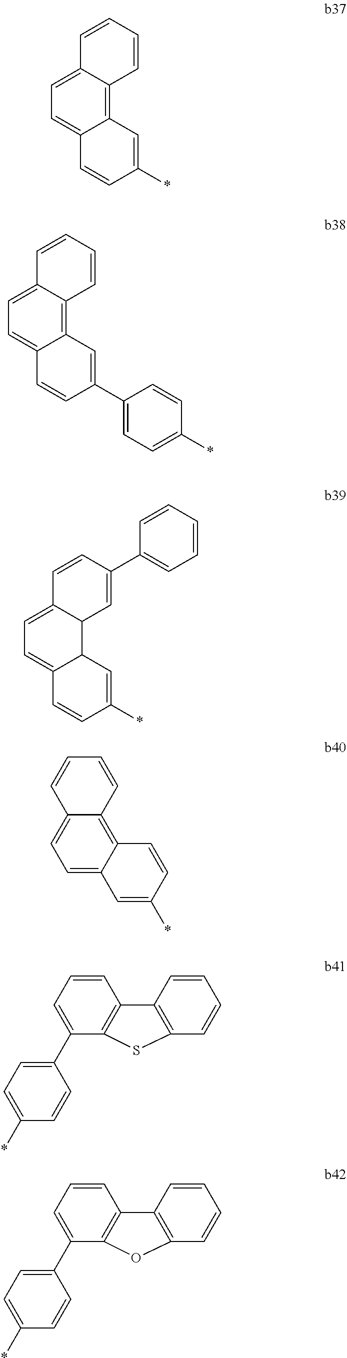

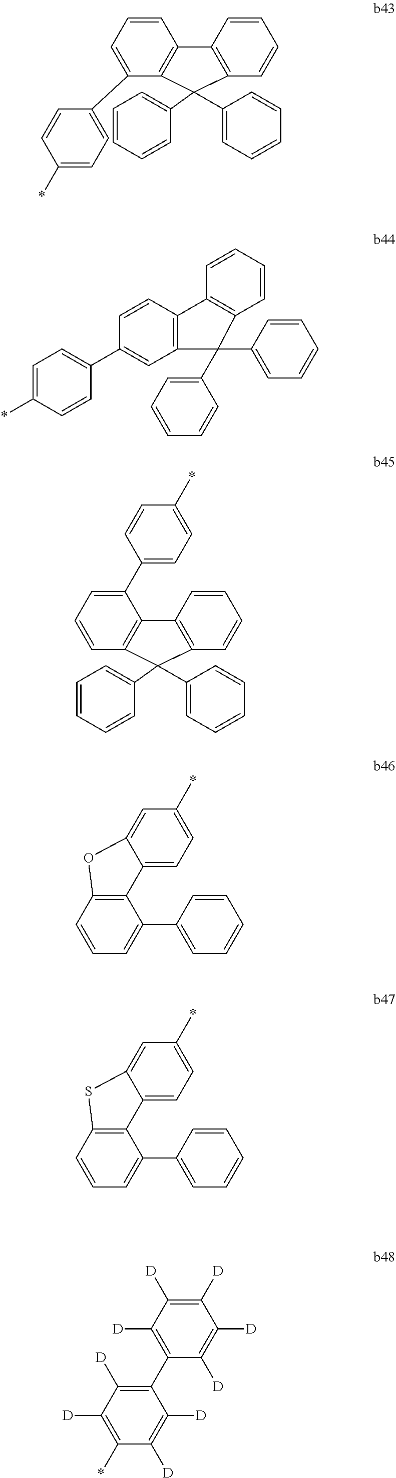

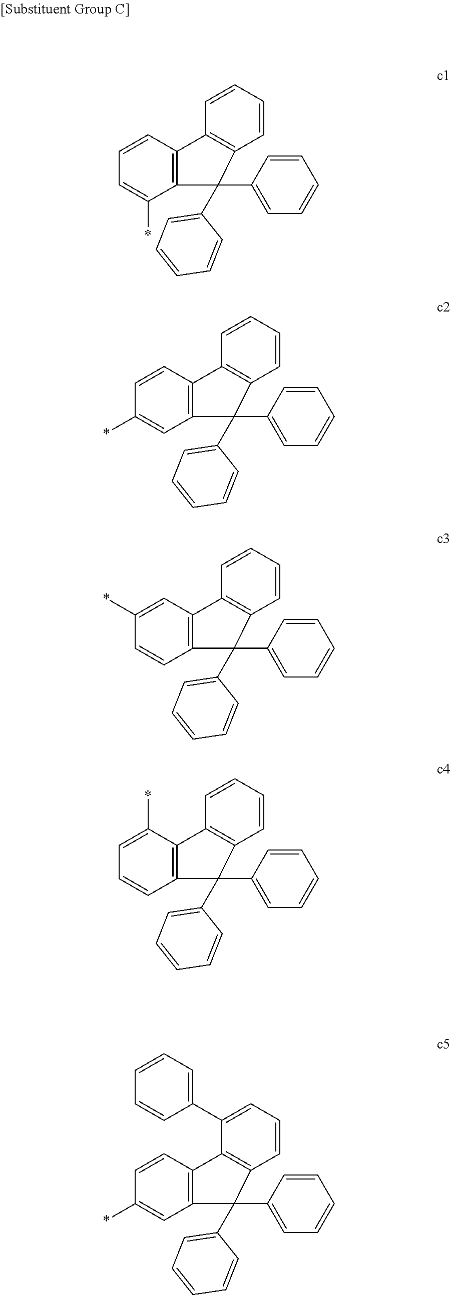

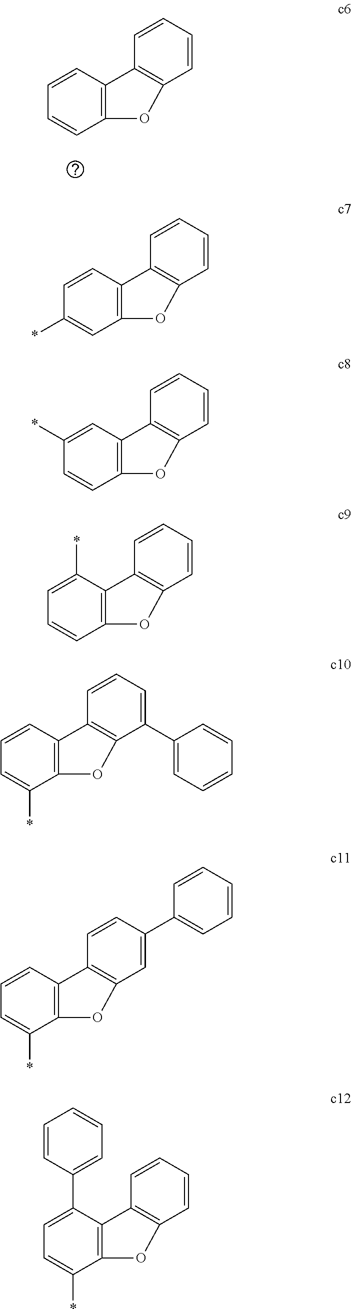

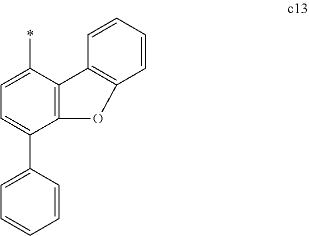 [SMILES: *c1ccc(-c2ccccc2)c2oc3ccccc3c12]

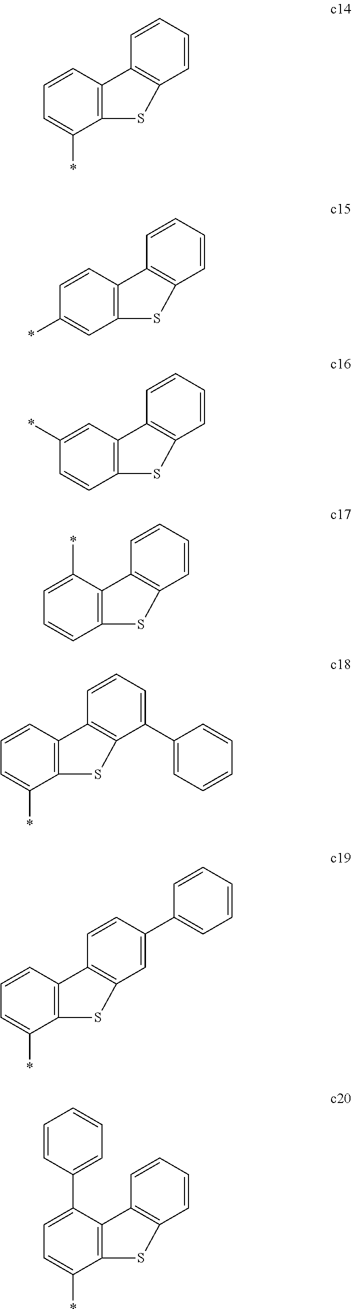

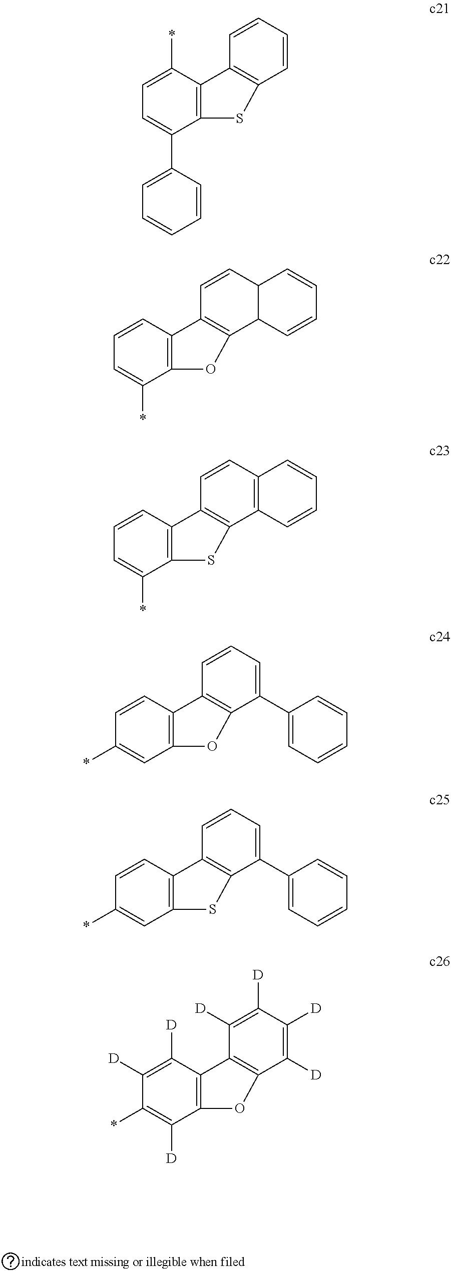 [SMILES: *c1ccc(-c2ccccc2)c2sc3ccccc3c12 ; *c1cccc2c3c(oc12)C1C=CC=CC1C=C3 ; *c1cccc2c1sc1c3ccccc3ccc21 ; *c1ccc2c(c1)oc1c(-c3ccccc3)cccc12 ; *c1ccc2c(c1)sc1c(-c3ccccc3)cccc12 ; *c1c([2H])c([2H])c2c(oc3c([2H])c([2H])c([2H])c([2H])c32)c1[2H]]

13. The light emitting element of claim 1, wherein
the amine compound is represented by Formula 1-7, and
the amine compound meets one of the combinations in Compound Combination Table 1: 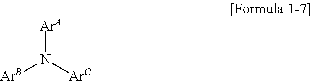
wherein in Formula 1-7,
Ar^{A} is a group selected from Substituent Group A,
Ar^{B} is a group selected from Substituent Group B, and
Ar^{C} is a group selected from Substituent Group C: 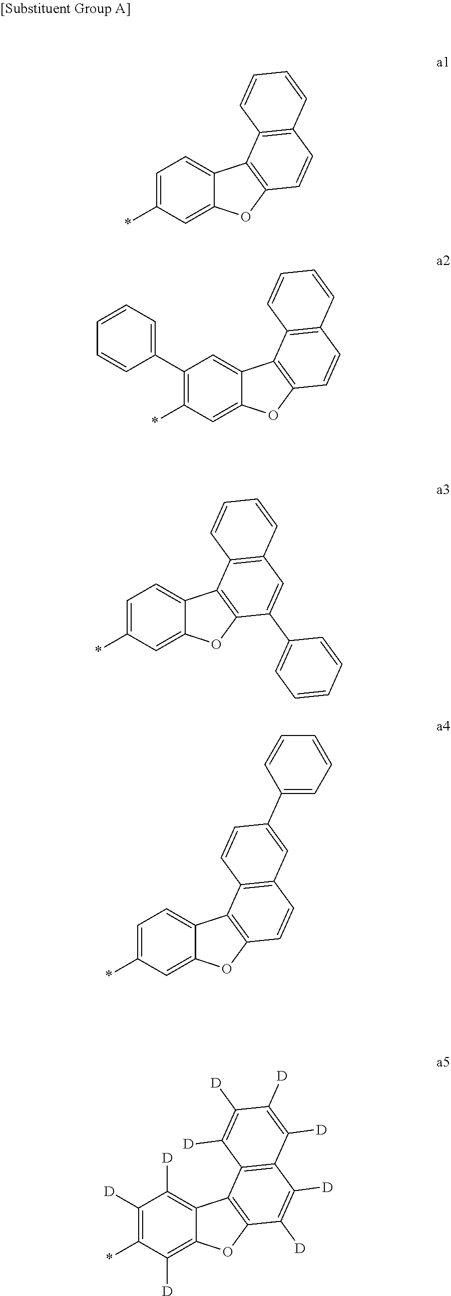 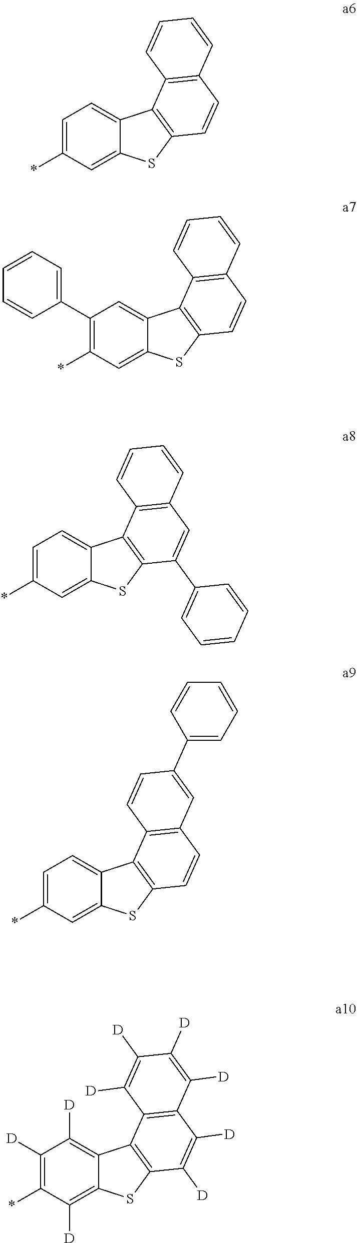 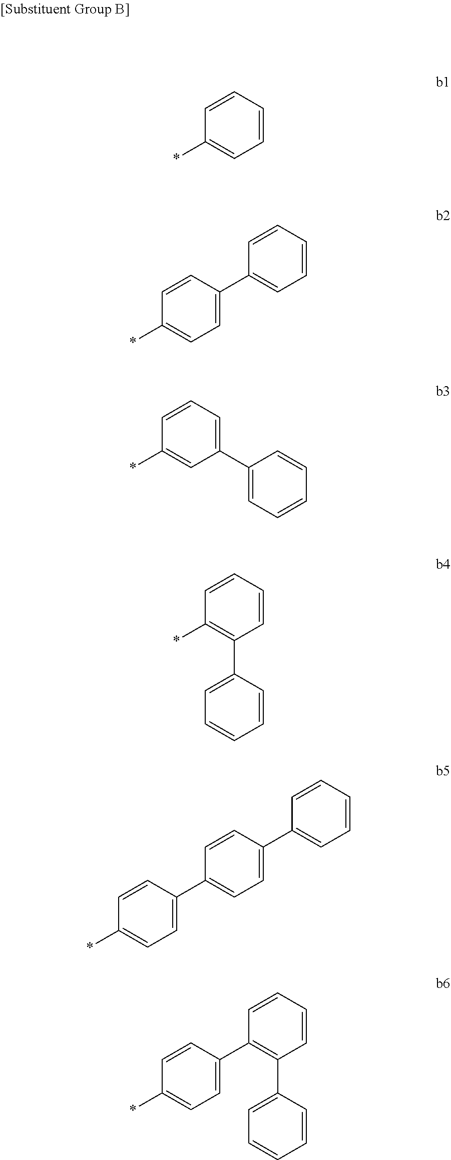 [SMILES: *c1ccccc1 ; *c1ccc(-c2ccccc2)cc1 ; *c1cccc(-c2ccccc2)c1 ; *c1ccccc1-c1ccccc1 ; *c1ccc(-c2ccc(-c3ccccc3)cc2)cc1 ; *c1ccc(-c2ccccc2-c2ccccc2)cc1] 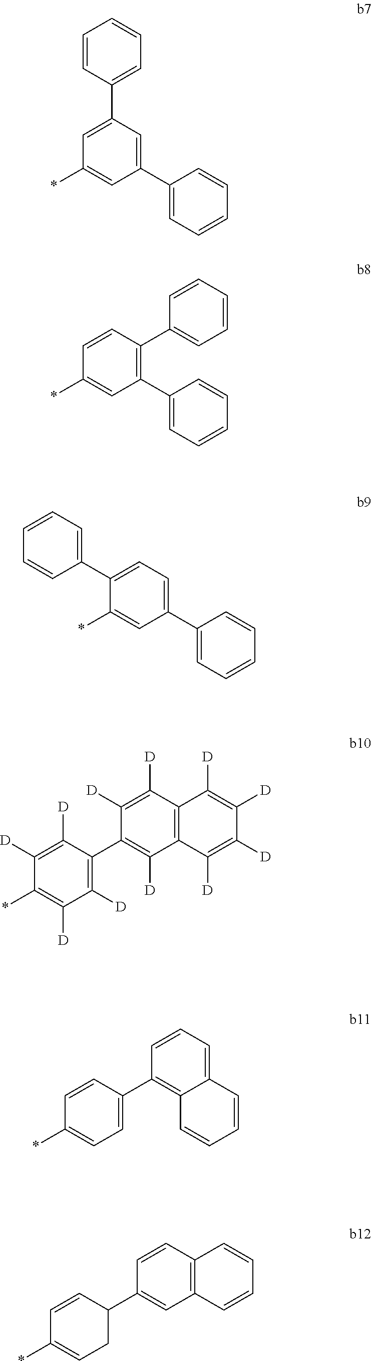 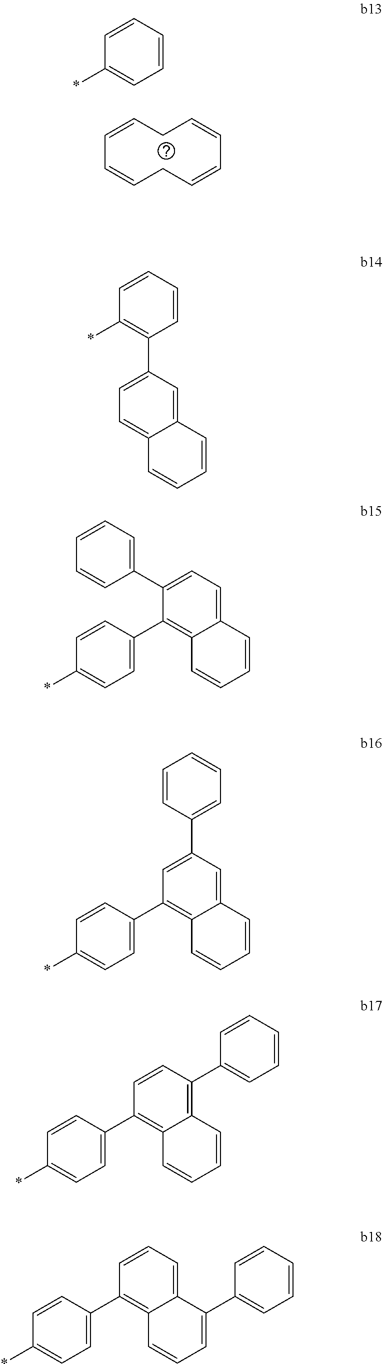 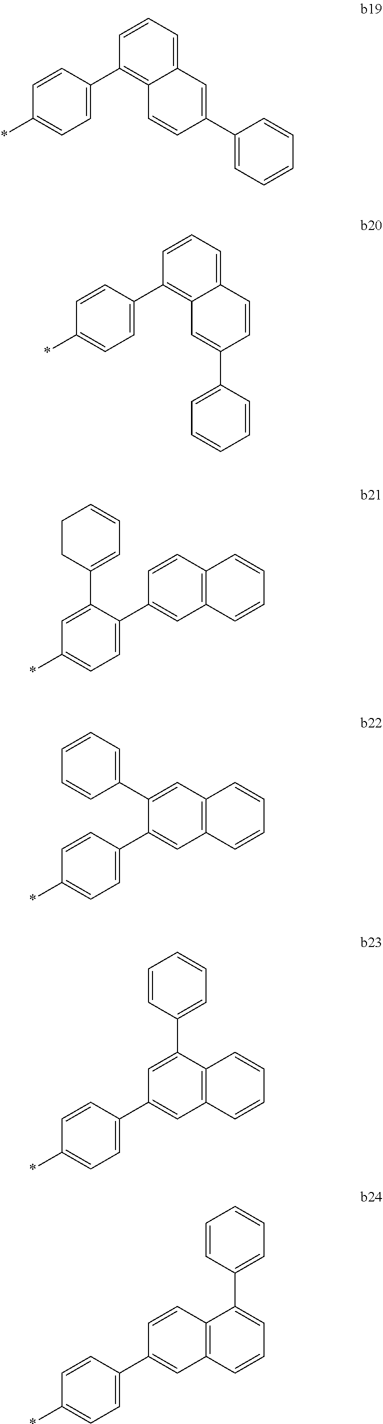 [SMILES: *c1ccc(-c2cccc3cc(-c4ccccc4)ccc23)cc1 ; *c1ccc(-c2cccc3ccc(-c4ccccc4)cc23)cc1 ; *c1ccc(-c2ccc3ccccc3c2)c(C2=CC=CCC2)c1 ; *c1ccc(-c2cc3ccccc3cc2-c2ccccc2)cc1 ; *c1ccc(-c2cc(-c3ccccc3)c3ccccc3c2)cc1 ; *c1ccc(-c2ccc3c(-c4ccccc4)cccc3c2)cc1] 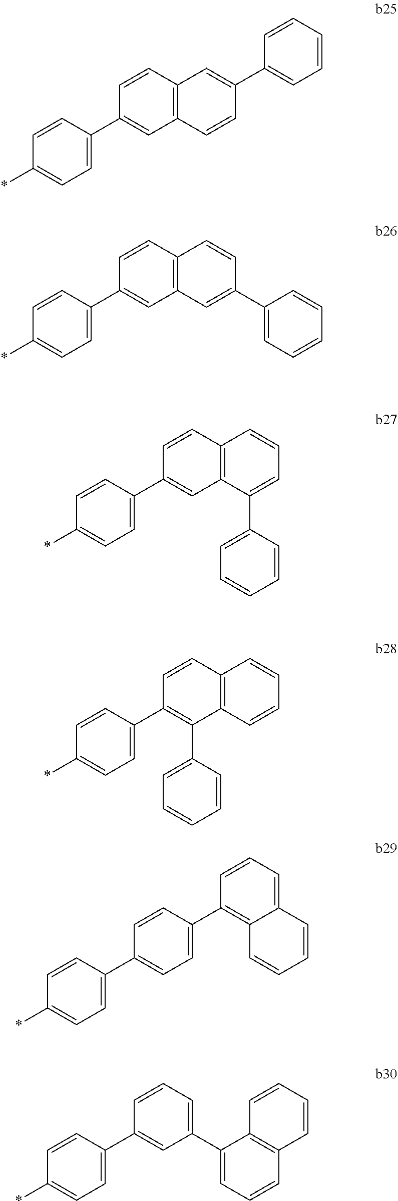 [SMILES: *c1ccc(-c2ccc3cc(-c4ccccc4)ccc3c2)cc1 ; *c1ccc(-c2ccc3ccc(-c4ccccc4)cc3c2)cc1 ; *c1ccc(-c2ccc3cccc(-c4ccccc4)c3c2)cc1 ; *c1ccc(-c2ccc3ccccc3c2-c2ccccc2)cc1 ; *c1ccc(-c2ccc(-c3cccc4ccccc34)cc2)cc1 ; *c1ccc(-c2cccc(-c3cccc4ccccc34)c2)cc1] 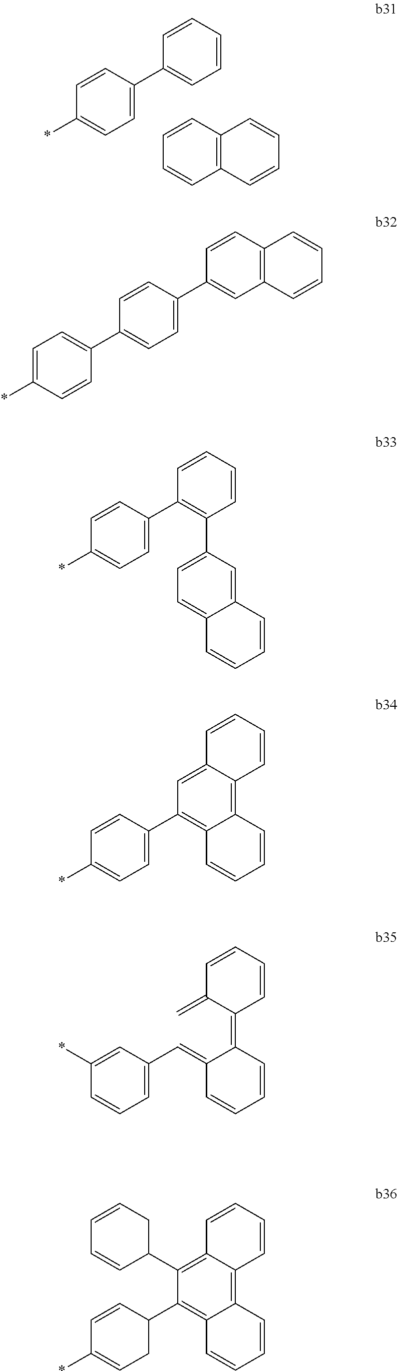 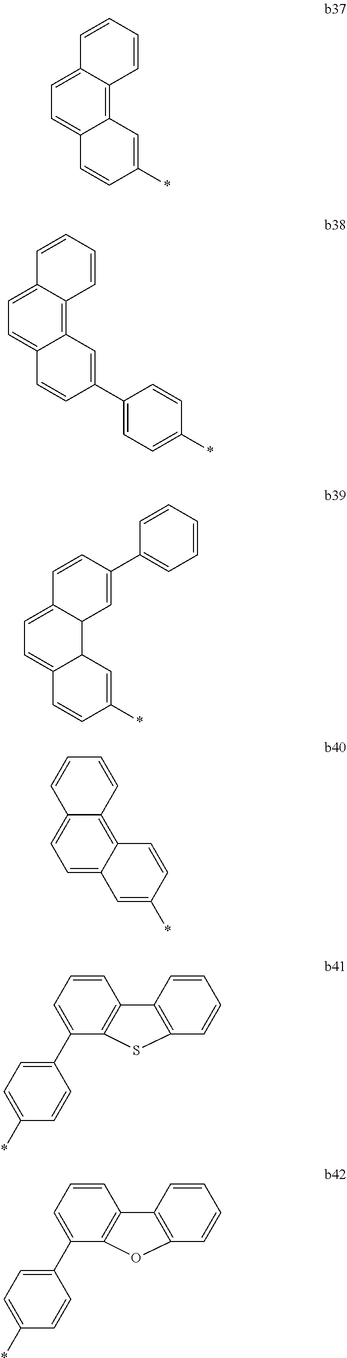 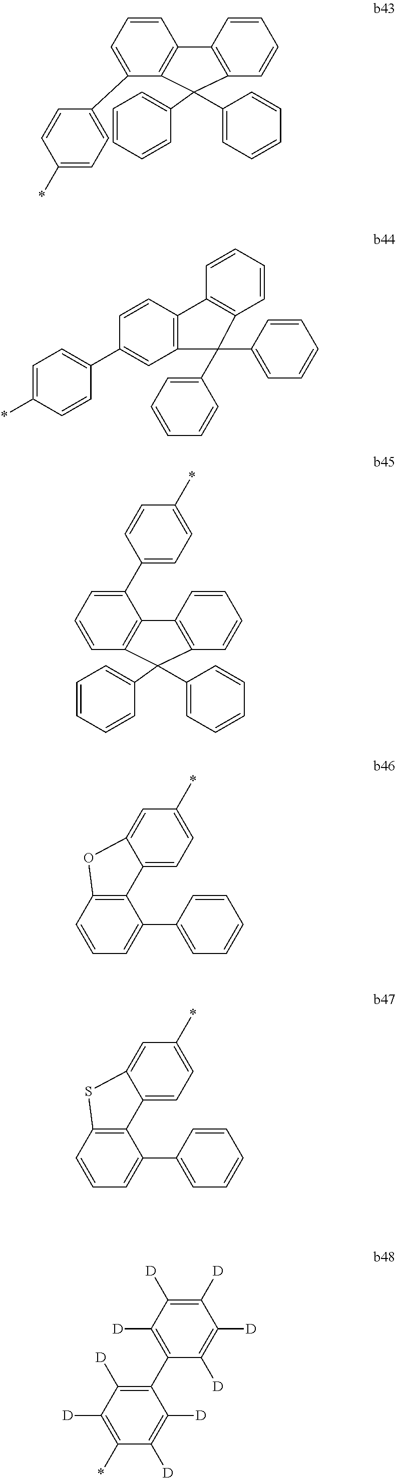 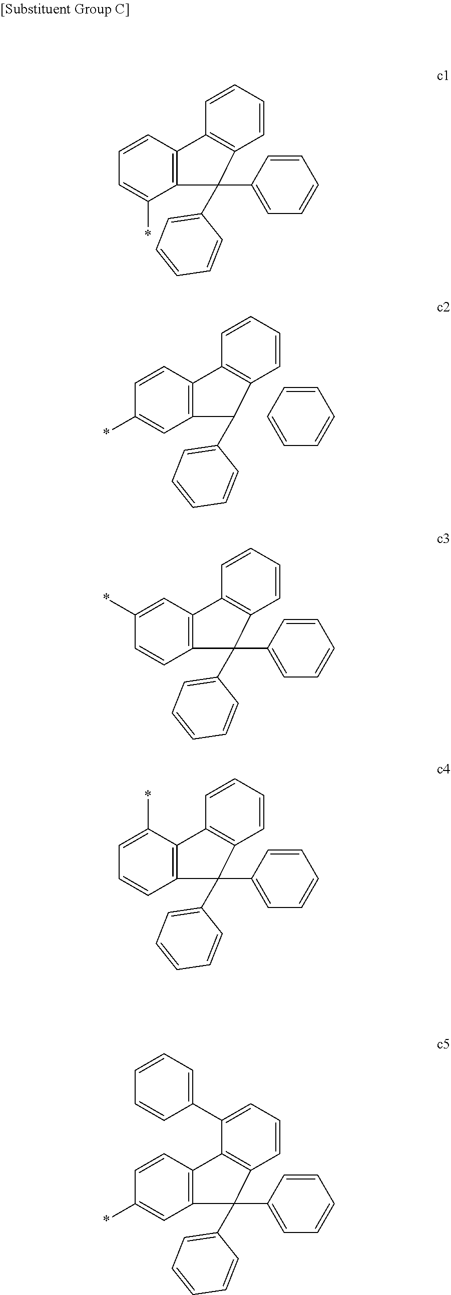 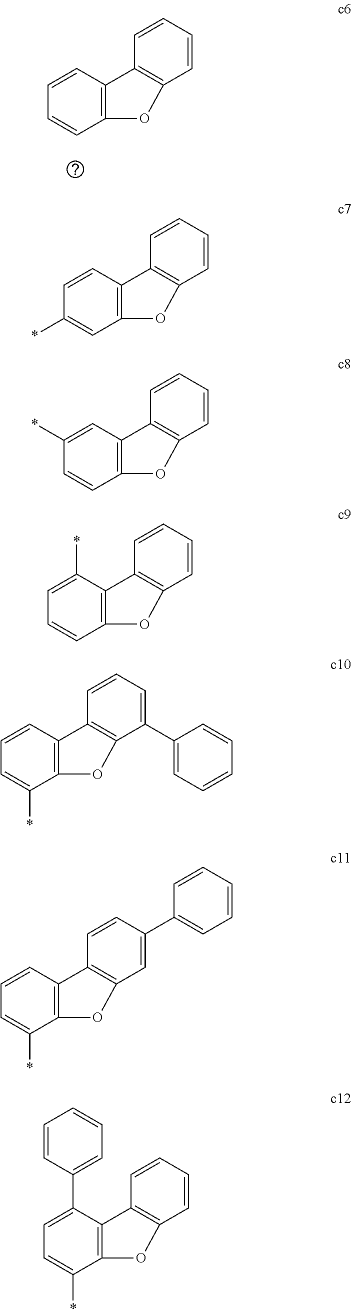 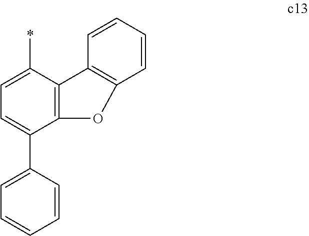 [SMILES: *c1ccc(-c2ccccc2)c2oc3ccccc3c12] 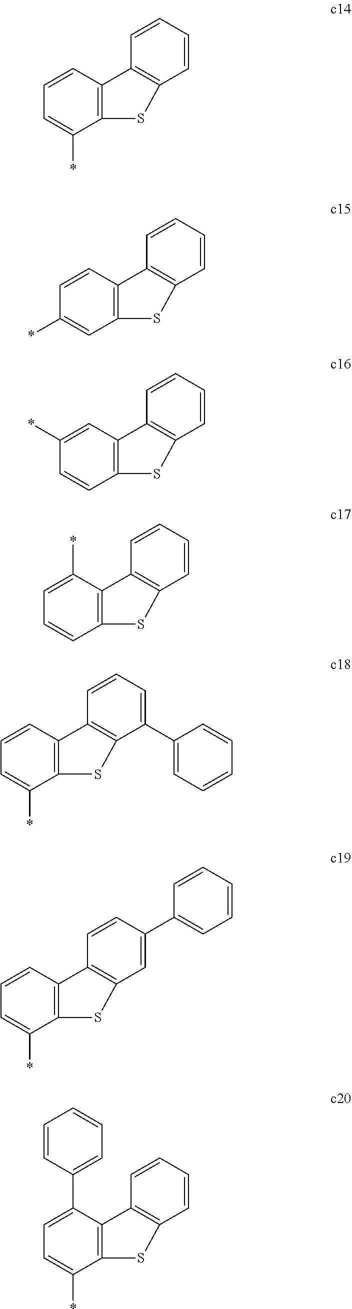 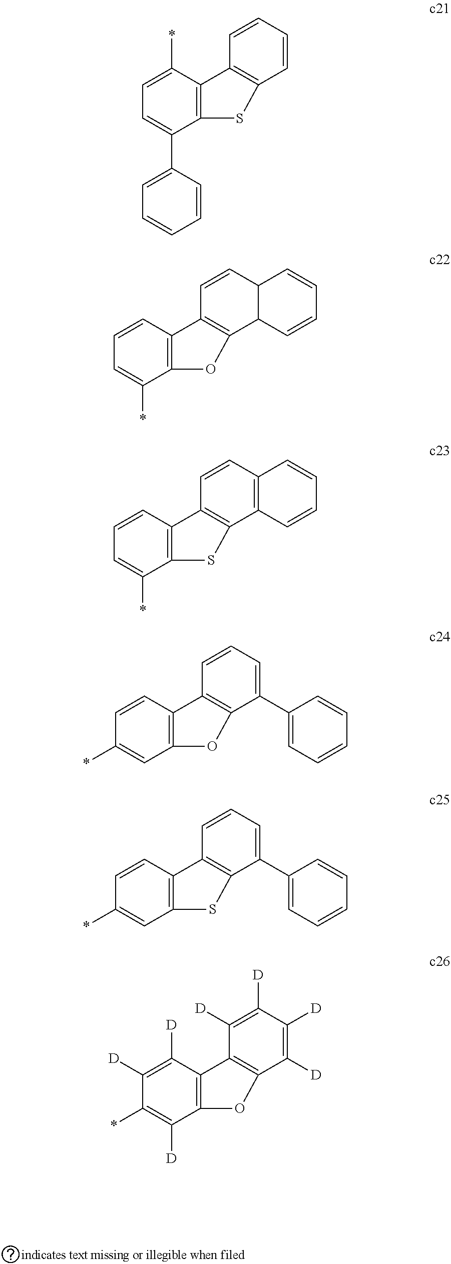 [SMILES: *c1ccc(-c2ccccc2)c2sc3ccccc3c12 ; *c1cccc2c3c(oc12)C1C=CC=CC1C=C3 ; *c1cccc2c1sc1c3ccccc3ccc21 ; *c1ccc2c(c1)oc1c(-c3ccccc3)cccc12 ; *c1ccc2c(c1)sc1c(-c3ccccc3)cccc12 ; *c1c([2H])c([2H])c2c(oc3c([2H])c([2H])c([2H])c([2H])c32)c1[2H]]
[Compound Combination Table 1]

| No. | Ar^{A} | Ar^{B} | Ar^{C} |
|---|---|---|---|
| 1 | a1 | b11 | c7 |
| 2 | a2 | b11 | c7 |
| 3 | a3 | b11 | c7 |
| 4 | a4 | b11 | c7 |
| 5 | a5 | b11 | c7 |
| 6 | a6 | b11 | c7 |
| 7 | a7 | b11 | c7 |
| 8 | a8 | b11 | c7 |
| 9 | a9 | b11 | c7 |
| 10 | a10 | b11 | c7 |
| 11 | a1 | b1 | c1 |
| 12 | a1 | b1 | c2 |
| 13 | a1 | b1 | c3 |
| 14 | a1 | b1 | c4 |
| 15 | a1 | b1 | c5 |
| 16 | a1 | b1 | c7 |
| 17 | a1 | b1 | c9 |
| 18 | a1 | b1 | c13 |
| 19 | a1 | b1 | c14 |
| 20 | a1 | b1 | c15 |
| 21 | a1 | b1 | c16 |
| 22 | a1 | b1 | c17 |
| 23 | a1 | b1 | c18 |
| 24 | a1 | b1 | c19 |
| 25 | a1 | b1 | c20 |
| 26 | a1 | b1 | c21 |
| 27 | a1 | b1 | c24 |
| 28 | a1 | b2 | c1 |
| 29 | a1 | b2 | c2 |
| 30 | a1 | b2 | c3 |
| 31 | a1 | b2 | c4 |
| 32 | a1 | b2 | c5 |
| 33 | a1 | b2 | c7 |
| 34 | a1 | b2 | c9 |
| 35 | a1 | b2 | c13 |
| 36 | a1 | b2 | c14 |
| 37 | a1 | b2 | c15 |
| 38 | a1 | b2 | c16 |
| 39 | a1 | b2 | c17 |
| 40 | a1 | b2 | c18 |
| 41 | a1 | b2 | c19 |
| 42 | a1 | b2 | c20 |
| 43 | a1 | b2 | c21 |
| 44 | a1 | b2 | c24 |
| 45 | a1 | b2 | c25 |
| 46 | a1 | b2 | c26 |
| 47 | a1 | b3 | c1 |
| 48 | a1 | b3 | c2 |
| 49 | a1 | b3 | c3 |
| 50 | a1 | b3 | c4 |
| 51 | a1 | b3 | c5 |
| 52 | a1 | b3 | c7 |
| 53 | a1 | b3 | c9 |
| 54 | a1 | b3 | c13 |
| 55 | a1 | b3 | c14 |
| 56 | a1 | b3 | c15 |
| 57 | a1 | b3 | c16 |
| 58 | a1 | b3 | c18 |
| 59 | a1 | b3 | c19 |
| 60 | a1 | b3 | c20 |
| 61 | a1 | b3 | c21 |
| 62 | a1 | b3 | c24 |
| 63 | a1 | b11 | c25 |
| 64 | a1 | b11 | c26 |
| 65 | a1 | b11 | c6 |
| 66 | a1 | b11 | c9 |
| 67 | a1 | b11 | c10 |
| 68 | a1 | b11 | c11 |
| 69 | a1 | b11 | c12 |
| 70 | a1 | b11 | c13 |
| 71 | a1 | b11 | c14 |
| 72 | a1 | b11 | c15 |
| 73 | a1 | b11 | c16 |
| 74 | a1 | b11 | c17 |
| 75 | a1 | b11 | c18 |
| 76 | a1 | b11 | c19 |
| 77 | a1 | b11 | c20 |
| 78 | a1 | b11 | c21 |
| 79 | a1 | b11 | c22 |
| 80 | a1 | b11 | c23 |
| 81 | a1 | b11 | c24 |
| 82 | a1 | b12 | c6 |
| 83 | a1 | b12 | c7 |
| 84 | a1 | b12 | c9 |
| 85 | a1 | b12 | c10 |
| 86 | a1 | b12 | c11 |
| 87 | a1 | b12 | c12 |
| 88 | a1 | b12 | c13 |
| 89 | a1 | b12 | c14 |
| 90 | a1 | b12 | c15 |
| 91 | a1 | b12 | c16 |
| 92 | a1 | b12 | c17 |
| 93 | a1 | b12 | c18 |
| 94 | a1 | b12 | c19 |
| 95 | a1 | b12 | c20 |
| 96 | a1 | b12 | c21 |
| 97 | a1 | b12 | c22 |
| 98 | a1 | b12 | c23 |
| 99 | a1 | b12 | c24 |
| 100 | a1 | b12 | c25 |
| 101 | a1 | b12 | c26 |
| 102 | a1 | b34 | c6 |
| 103 | a1 | b34 | c7 |
| 104 | a1 | b34 | c8 |
| 105 | a1 | b34 | c9 |
| 106 | a1 | b34 | c10 |
| 107 | a1 | b34 | c11 |
| 108 | a1 | b34 | c12 |
| 109 | a1 | b34 | c13 |
| 110 | a1 | b34 | c14 |
| 111 | a1 | b34 | c15 |
| 112 | a1 | b34 | c16 |
| 113 | a1 | b34 | c17 |
| 114 | a1 | b34 | c18 |
| 115 | a1 | b34 | c19 |
| 116 | a1 | b34 | c20 |
| 117 | a1 | b34 | c21 |
| 118 | a1 | b34 | c22 |
| 119 | a1 | b34 | c23 |
| 120 | a1 | b34 | c24 |
| 121 | a1 | b34 | c25 |
| 122 | a1 | b34 | c26 |
| 123 | a1 | b42 | c1 |
| 124 | a1 | b42 | c2 |
| 125 | a1 | b42 | c3 |
| 126 | a1 | b42 | c4 |
| 127 | a1 | b42 | c5 |
| 128 | a1 | b42 | c6 |
| 129 | a1 | b42 | c7 |
| 130 | a1 | b42 | c8 |
| 131 | a1 | b42 | c9 |
| 132 | a1 | b42 | c10 |
| 133 | a1 | b42 | c11 |
| 134 | a1 | b42 | c12 |
| 135 | a1 | b42 | c13 |
| 136 | a1 | b42 | c14 |
| 137 | a1 | b42 | c15 |
| 138 | a1 | b42 | c16 |
| 139 | a1 | b42 | c18 |
| 140 | a1 | b42 | c19 |
| 141 | a1 | b42 | c20 |
| 142 | a1 | b42 | c21 |
| 143 | a1 | b42 | c22 |
| 144 | a1 | b42 | c23 |
| 145 | a1 | b42 | c24 |
| 146 | a1 | b42 | c25 |
| 147 | a1 | b42 | c26 |
| 148 | a1 | c2 | b4 |
| 149 | a1 | c2 | b5 |
| 150 | a1 | c2 | b6 |
| 151 | a1 | c2 | b7 |
| 152 | a1 | c2 | b8 |
| 153 | a1 | c2 | b9 |
| 154 | a1 | c2 | b41 |
| 155 | a1 | c2 | b43 |
| 156 | a1 | c2 | b44 |
| 157 | a1 | c2 | b45 |
| 158 | a1 | c2 | b46 |
| 159 | a1 | c2 | b47 |
| 160 | a1 | c2 | c1 |
| 161 | a1 | c2 | c2 |
| 162 | a1 | c2 | c3 |
| 163 | a1 | c2 | c4 |
| 164 | a1 | c2 | c5 |
| 165 | a1 | c2 | c6 |
| 166 | a1 | c2 | c7 |
| 167 | a1 | c2 | c8 |
| 168 | a1 | c2 | c9 |
| 169 | a1 | c2 | c10 |
| 170 | a1 | c2 | c11 |
| 171 | a1 | c2 | c12 |
| 172 | a1 | c2 | c13 |
| 173 | a1 | c2 | c14 |
| 174 | a1 | c2 | c15 |
| 175 | a1 | c2 | c16 |
| 176 | a1 | c2 | c17 |
| 177 | a1 | c2 | c18 |
| 178 | a1 | c2 | c19 |
| 179 | a1 | c2 | c20 |
| 180 | a1 | c2 | c21 |
| 181 | a1 | c2 | c24 |
| 182 | a1 | c2 | c25 |
| 183 | a1 | c2 | c26 |
| 184 | a1 | c7 | b4 |
| 185 | a1 | c7 | b5 |
| 186 | a1 | c7 | b6 |
| 187 | a1 | c7 | b7 |
| 188 | a1 | c7 | b8 |
| 189 | a1 | c7 | b9 |
| 190 | a1 | c7 | b10 |
| 191 | a1 | c7 | b13 |
| 192 | a1 | c7 | b14 |
| 193 | a1 | c7 | b15 |
| 194 | a1 | c7 | b16 |
| 195 | a1 | c7 | b17 |
| 196 | a1 | c7 | b18 |
| 197 | a1 | c7 | b19 |
| 198 | a1 | c7 | b20 |
| 199 | a1 | c7 | b21 |
| 200 | a1 | c7 | b22 |
| 201 | a1 | c7 | b23 |
| 202 | a1 | c7 | b24 |
| 203 | a1 | c7 | b25 |
| 204 | a1 | c7 | b26 |
| 205 | a1 | c7 | b27 |
| 206 | a1 | c7 | b28 |
| 207 | a1 | c7 | b29 |
| 208 | a1 | c7 | b30 |
| 209 | a1 | c7 | b31 |
| 210 | a1 | c7 | b32 |
| 211 | a1 | c7 | b33 |
| 212 | a1 | c7 | b35 |
| 213 | a1 | c7 | b36 |
| 214 | a1 | c7 | b37 |
| 215 | a1 | c7 | b38 |
| 216 | a1 | c7 | b39 |
| 217 | a1 | c7 | b40 |
| 218 | a1 | c7 | b41 |
| 219 | a1 | c7 | b43 |
| 220 | a1 | c7 | b44 |
| 221 | a1 | c7 | b45 |
| 222 | a1 | c7 | b46 |
| 223 | a1 | c7 | b47 |
| 224 | a1 | c7 | c1 |
| 225 | a1 | c7 | c3 |
| 226 | a1 | c7 | c4 |
| 227 | a1 | c7 | c5 |
| 228 | a1 | c7 | c6 |
| 229 | a1 | c7 | c7 |
| 230 | a1 | c7 | c8 |
| 231 | a1 | c7 | c9 |
| 232 | a1 | c7 | c10 |
| 233 | a1 | c7 | c11 |
| 234 | a1 | c7 | c12 |
| 235 | a1 | c7 | c13 |
| 236 | a1 | c7 | c14 |
| 237 | a1 | c7 | c15 |
| 238 | a1 | c7 | c16 |
| 239 | a1 | c7 | c17 |
| 240 | a1 | c7 | c18 |
| 241 | a1 | c7 | c19 |
| 242 | a1 | c7 | c20 |
| 243 | a1 | c7 | c21 |
| 244 | a1 | c7 | c22 |
| 245 | a1 | c7 | c23 |
| 246 | a1 | c7 | c24 |
| 247 | a1 | c7 | c25 |
| 248 | a1 | c7 | c26 |
| 249 | a1 | c14 | b4 |
| 250 | a1 | c14 | b5 |
| 251 | a1 | c14 | b6 |
| 252 | a1 | c14 | b7 |
| 253 | a1 | c14 | b8 |
| 254 | a1 | c14 | b9 |
| 255 | a1 | c14 | b10 |
| 256 | a1 | c14 | b13 |
| 257 | a1 | c14 | b114 |
| 258 | a1 | c14 | b15 |
| 259 | a1 | c14 | b16 |
| 260 | a1 | c14 | b17 |
| 261 | a1 | c14 | b18 |
| 262 | a1 | c14 | b19 |
| 263 | a1 | c14 | b20 |
| 264 | a1 | c14 | b21 |
| 265 | a1 | c14 | b22 |
| 266 | a1 | c14 | b23 |
| 267 | a1 | c14 | b24 |
| 268 | a1 | c14 | b25 |
| 269 | a1 | c14 | b26 |
| 270 | a1 | c14 | b27 |
| 271 | a1 | c14 | b28 |
| 272 | a1 | c14 | b29 |
| 273 | a1 | c14 | b30 |
| 274 | a1 | c14 | b31 |
| 275 | a1 | c14 | b32 |
| 276 | a1 | c14 | b33 |
| 277 | a1 | c14 | b35 |
| 278 | a1 | c14 | b36 |
| 279 | a1 | c14 | b37 |
| 280 | a1 | c14 | b38 |
| 281 | a1 | c14 | b39 |
| 282 | a1 | c14 | b40 |
| 283 | a1 | c14 | b41 |
| 284 | a1 | c14 | b43 |
| 285 | a1 | c14 | b44 |
| 286 | a1 | c14 | b45 |
| 287 | a1 | c14 | b46 |
| 288 | a1 | c14 | b47 |
| 289 | a1 | c14 | c1 |
| 290 | a1 | c14 | c3 |
| 291 | a1 | c14 | c4 |
| 292 | a1 | c14 | c5 |
| 293 | a1 | c14 | c6 |
| 294 | a1 | c14 | c8 |
| 295 | a1 | c14 | c9 |
| 296 | a1 | c14 | c10 |
| 297 | a1 | c14 | c11 |
| 298 | a1 | c14 | c12 |
| 299 | a1 | c14 | c13 |
| 300 | a1 | c14 | c14 |
| 301 | a1 | c14 | c15 |
| 302 | a1 | c14 | c16 |
| 303 | a1 | c14 | c18 |
| 304 | a1 | c14 | c19 |
| 305 | a1 | c14 | c20 |
| 306 | a1 | c14 | c21 |
| 307 | a1 | c14 | c22 |
| 308 | a1 | c14 | c23 |
| 309 | a1 | c14 | c24 |
| 310 | a1 | c14 | c25 |
| 311 | a1 | c14 | c26 |
| 312 | a1 | c15 | b4 |
| 313 | a1 | c15 | b5 |
| 314 | a1 | c15 | b6 |
| 315 | a1 | c15 | b7 |
| 316 | a1 | c15 | b8 |
| 317 | a1 | c15 | b9 |
| 318 | a1 | c15 | b10 |
| 319 | a1 | c15 | b13 |
| 320 | a1 | c15 | b14 |
| 321 | a1 | c15 | b15 |
| 322 | a1 | c15 | b16 |
| 323 | a1 | c15 | b17 |
| 324 | a1 | c15 | b18 |
| 325 | a1 | c15 | b19 |
| 326 | a1 | c15 | b20 |
| 327 | a1 | c15 | b21 |
| 328 | a1 | c15 | b22 |
| 329 | a1 | c15 | b23 |
| 330 | a1 | c15 | b24 |
| 331 | a1 | c15 | b25 |
| 332 | a1 | c15 | b26 |
| 333 | a1 | c15 | b27 |
| 334 | a1 | c15 | b28 |
| 335 | a1 | c15 | b29 |
| 336 | a1 | c15 | b30 |
| 337 | a1 | c15 | b31 |
| 338 | a1 | c15 | b32 |
| 339 | a1 | c15 | b33 |
| 340 | a1 | c15 | b35 |
| 341 | a1 | c15 | b36 |
| 342 | a1 | c15 | b37 |
| 343 | a1 | c15 | b38 |
| 344 | a1 | c15 | b39 |
| 345 | a1 | c15 | b40 |
| 346 | a1 | c15 | b41 |
| 347 | a1 | c15 | b43 |
| 348 | a1 | c15 | b44 |
| 349 | a1 | c15 | b45 |
| 350 | a1 | c15 | b46 |
| 351 | a1 | c15 | b47 |
| 352 | a1 | c15 | c1 |
| 353 | a1 | c15 | c3 |
| 354 | a1 | c15 | c4 |
| 355 | a1 | c15 | c5 |
| 356 | a1 | c15 | c6 |
| 357 | a1 | c15 | c8 |
| 358 | a1 | c15 | c9 |
| 359 | a1 | c15 | c10 |
| 360 | a1 | c15 | c11 |
| 361 | a1 | c15 | c12 |
| 362 | a1 | c15 | c13 |
| 363 | a1 | c15 | c15 |
| 364 | a1 | c15 | c16 |
| 365 | a1 | c15 | c17 |
| 366 | a1 | c15 | c18 |
| 367 | a1 | c15 | c19 |
| 368 | a1 | c15 | c20 |
| 369 | a1 | c15 | c21 |
| 370 | a1 | c15 | c22 |
| 371 | a1 | c15 | c23 |
| 372 | a1 | c15 | c24 |
| 373 | a1 | c15 | c25 |
| 374 | a1 | c15 | c26 |
| 375 | a1 | c24 | b4 |
| 376 | a1 | c24 | b5 |
| 377 | a1 | c24 | b6 |
| 378 | a1 | c24 | b7 |
| 379 | a1 | c24 | b8 |
| 380 | a1 | c24 | b9 |
| 381 | a1 | c24 | b10 |
| 382 | a1 | c24 | b13 |
| 383 | a1 | c24 | b14 |
| 384 | a1 | c24 | b15 |
| 385 | a1 | c24 | b16 |
| 386 | a1 | c24 | b17 |
| 387 | a1 | c24 | b18 |
| 388 | a1 | c24 | b19 |
| 389 | a1 | c24 | b20 |
| 390 | a1 | c24 | b21 |
| 391 | a1 | c24 | b22 |
| 392 | a1 | c24 | b23 |
| 393 | a1 | c24 | b24 |
| 394 | a1 | c24 | b25 |
| 395 | a1 | c24 | b26 |
| 396 | a1 | c24 | b27 |
| 397 | a1 | c24 | b28 |
| 398 | a1 | c24 | b29 |
| 399 | a1 | c24 | b30 |
| 400 | a1 | c24 | b31 |
| 401 | a1 | c24 | b32 |
| 402 | a1 | c24 | b33 |
| 403 | a1 | c24 | b35 |
| 404 | a1 | c24 | b36 |
| 405 | a1 | c24 | b37 |
| 406 | a1 | c24 | b38 |
| 407 | a1 | c24 | b39 |
| 408 | a1 | c24 | b40 |
| 409 | a1 | c24 | b41 |
| 410 | a1 | c24 | b43 |
| 411 | a1 | c24 | b44 |
| 412 | a1 | c24 | b45 |
| 413 | a1 | c24 | b46 |
| 414 | a1 | c24 | b47 |
| 415 | a1 | c24 | c1 |
| 416 | a1 | c24 | c3 |
| 417 | a1 | c24 | c4 |
| 418 | a1 | c24 | c5 |
| 419 | a1 | c24 | c6 |
| 420 | a1 | c24 | c8 |
| 421 | a1 | c24 | c9 |
| 422 | a1 | c24 | c10 |
| 423 | a1 | c24 | c11 |
| 424 | a1 | c24 | c1 |
| 425 | a1 | c24 | c13 |
| 426 | a1 | c24 | c16 |
| 427 | a1 | c24 | c17 |
| 428 | a1 | c24 | c18 |
| 429 | a1 | c24 | c19 |
| 430 | a1 | c24 | c20 |
| 431 | a1 | c24 | c21 |
| 432 | a1 | c24 | c22 |
| 433 | a1 | c24 | c23 |
| 434 | a1 | c24 | c24 |
| 435 | a1 | c24 | c25 |
| 436 | a1 | c24 | c26 |
| 437 | a6 | b1 | c1 |
| 438 | a6 | b1 | c2 |
| 439 | a6 | b1 | c3 |
| 440 | a6 | b1 | c4 |
| 441 | a6 | b1 | c5 |
| 442 | a6 | b1 | c7 |
| 443 | a6 | b1 | c9 |
| 444 | a6 | b1 | c13 |
| 445 | a6 | b1 | c14 |
| 446 | a6 | b1 | c15 |
| 447 | a6 | b1 | c16 |
| 448 | a6 | b1 | c17 |
| 449 | a6 | b1 | c18 |
| 450 | a6 | b1 | c19 |
| 451 | a6 | b1 | c20 |
| 452 | a6 | b1 | c21 |
| 453 | a6 | b1 | c24 |
| 454 | a6 | b2 | c1 |
| 455 | a6 | b2 | c2 |
| 456 | a6 | b2 | c3 |
| 457 | a6 | b2 | c4 |
| 458 | a6 | b2 | c5 |
| 459 | a6 | b2 | c7 |
| 460 | a6 | b2 | c9 |
| 461 | a6 | b2 | c13 |
| 462 | a6 | b2 | c14 |
| 463 | a6 | b2 | c15 |
| 464 | a6 | b2 | c16 |
| 465 | a6 | b2 | c17 |
| 466 | a6 | b2 | c18 |
| 467 | a6 | b2 | c19 |
| 468 | a6 | b2 | c20 |
| 469 | a6 | b2 | c21 |
| 470 | a6 | b2 | c24 |
| 471 | a6 | b2 | c25 |
| 472 | a6 | b2 | c26 |
| 473 | a6 | b3 | c1 |
| 474 | a6 | b3 | c2 |
| 475 | a6 | b3 | c3 |
| 476 | a6 | b3 | c4 |
| 477 | a6 | b3 | c5 |
| 478 | a6 | b3 | c7 |
| 479 | a6 | b3 | c9 |
| 480 | a6 | b3 | c13 |
| 481 | a6 | b3 | c16 |
| 482 | a6 | b3 | c15 |
| 483 | a6 | b3 | c16 |
| 484 | a6 | b3 | c18 |
| 485 | a6 | b3 | c19 |
| 486 | a6 | b3 | c20 |
| 487 | a6 | b3 | c21 |
| 488 | a6 | b3 | c24 |
| 489 | a6 | b11 | c25 |
| 490 | a6 | b11 | c26 |
| 491 | a6 | b11 | c6 |
| 492 | a6 | b11 | c9 |
| 493 | a6 | b11 | c10 |
| 494 | a6 | b11 | c11 |
| 495 | a6 | b11 | c12 |
| 496 | a6 | b11 | c13 |
| 497 | a6 | b11 | c14 |
| 498 | a6 | b11 | c15 |
| 499 | a6 | b11 | c16 |
| 500 | a6 | b11 | c17 |
| 501 | a6 | b11 | c18 |
| 502 | a6 | b11 | c19 |
| 503 | a6 | b11 | c20 |
| 504 | a6 | b11 | c21 |
| 505 | a6 | b11 | c22 |
| 506 | a6 | b11 | c23 |
| 507 | a6 | b11 | c24 |
| 508 | a6 | b12 | c25 |
| 509 | a6 | b12 | c60 |
| 510 | a6 | b12 | c7 |
| 511 | a6 | b12 | c9 |
| 512 | a6 | b12 | c10 |
| 513 | a6 | b12 | c11 |
| 514 | a6 | b12 | c12 |
| 515 | a6 | b12 | c13 |
| 516 | a6 | b12 | c14 |
| 517 | a6 | b12 | c15 |
| 518 | a6 | b12 | c16 |
| 519 | a6 | b12 | c17 |
| 520 | a6 | b12 | c18 |
| 521 | a6 | b12 | c19 |
| 522 | a6 | b12 | c20 |
| 523 | a6 | b12 | c21 |
| 524 | a6 | b12 | c22 |
| 525 | a6 | b12 | c23 |
| 526 | a6 | b12 | c24 |
| 527 | a6 | b12 | c25 |
| 528 | a6 | b34 | c26 |
| 021 | a6 | b34 | c6 |
| 530 | a6 | b34 | c7 |
| 531 | a6 | b34 | c8 |
| 532 | a6 | b34 | c9 |
| 533 | a6 | b34 | c10 |
| 534 | a6 | b34 | c11 |
| 535 | a6 | b34 | c12 |
| 536 | a6 | b34 | c14 |
| 537 | a6 | b34 | c15 |
| 538 | a6 | b34 | c16 |
| 539 | a6 | b34 | c17 |
| 540 | a6 | b34 | c18 |
| 541 | a6 | b34 | c19 |
| 542 | a6 | b34 | c20 |
| 543 | a6 | b34 | c21 |
| 544 | a6 | b34 | c22 |
| 545 | a6 | b34 | c23 |
| 546 | a6 | b34 | c24 |
| 547 | a6 | b34 | c25 |
| 548 | a6 | b34 | c26 |
| 549 | a6 | b42 | c1 |
| 550 | a6 | b42 | c2 |
| 551 | a6 | b42 | c3 |
| 552 | a6 | b42 | c4 |
| 553 | a6 | b42 | c5 |
| 554 | a6 | b42 | c6 |
| 555 | a6 | b42 | c7 |
| 556 | a6 | b42 | c8 |
| 557 | a6 | b42 | c9 |
| 558 | a6 | b42 | c10 |
| 559 | a6 | b42 | c11 |
| 560 | a6 | b42 | c12 |
| 561 | a6 | b42 | c13 |
| 562 | a6 | b42 | c14 |
| 563 | a6 | b42 | c15 |
| 564 | a6 | b42 | c16 |
| 565 | a6 | b42 | c18 |
| 566 | a6 | b42 | c19 |
| 567 | a6 | b42 | c20 |
| 568 | a6 | b42 | c21 |
| 569 | a6 | b42 | c22 |
| 570 | a6 | b42 | c23 |
| 571 | a6 | b42 | c24 |
| 572 | a6 | b42 | c25 |
| 573 | a6 | b42 | c26 |
| 574 | a6 | c2 | c4 |
| 575 | a6 | c2 | c5 |
| 576 | a6 | c2 | c6 |
| 577 | a6 | c2 | c7 |
| 578 | a6 | c2 | c8 |
| 579 | a6 | c2 | c9 |
| 580 | a6 | c2 | c41 |
| 581 | a6 | c2 | c43 |
| 582 | a6 | c2 | c44 |
| 583 | a6 | c2 | c45 |
| 584 | a6 | c2 | c46 |
| 585 | a6 | c2 | c47 |
| 586 | a6 | c2 | c1 |
| 587 | a6 | c2 | c2 |
| 588 | a6 | c2 | c3 |
| 589 | a6 | c2 | c4 |
| 590 | a6 | c2 | c5 |
| 591 | a6 | c2 | c6 |
| 592 | a6 | c2 | c7 |
| 593 | a6 | c2 | c8 |
| 594 | a6 | c2 | c9 |
| 595 | a6 | c2 | c10 |
| 596 | a6 | c2 | c11 |
| 597 | a6 | c2 | c12 |
| 598 | a6 | c2 | c13 |
| 599 | a6 | c2 | c14 |
| 600 | a6 | c2 | c15 |
| 601 | a6 | c2 | c16 |
| 602 | a6 | c2 | c17 |
| 603 | a6 | c2 | c18 |
| 604 | a6 | c2 | c19 |
| 605 | a6 | c2 | c20 |
| 606 | a6 | c2 | c21 |
| 607 | a6 | c2 | c24 |
| 608 | a6 | c2 | c25 |
| 609 | a6 | c2 | c26 |
| 610 | a6 | c2 | b4 |
| 611 | a6 | c7 | b5 |
| 612 | a6 | c7 | b6 |
| 613 | a6 | c7 | b7 |
| 614 | a6 | c7 | b8 |
| 615 | a6 | c7 | b9 |
| 616 | a6 | c7 | b10 |
| 617 | a6 | c7 | b13 |
| 618 | a6 | c7 | b14 |
| 619 | a6 | c7 | b15 |
| 620 | a6 | c7 | b16 |
| 621 | a6 | c7 | b17 |
| 622 | a6 | c7 | b18 |
| 623 | a6 | c7 | b19 |
| 624 | a6 | c7 | b20 |
| 625 | a6 | c7 | b21 |
| 626 | a6 | c7 | b22 |
| 627 | a6 | c7 | b23 |
| 628 | a6 | c7 | b24 |
| 629 | a6 | c7 | b25 |
| 630 | a6 | c7 | b26 |
| 631 | a6 | c7 | b27 |
| 632 | a6 | c7 | b28 |
| 633 | a6 | c7 | b29 |
| 634 | a6 | c7 | b30 |
| 635 | a6 | c7 | b31 |
| 636 | a6 | c7 | b32 |
| 637 | a6 | c7 | b33 |
| 638 | a6 | c7 | b35 |
| 639 | a6 | c7 | b36 |
| 640 | a6 | c7 | b37 |
| 641 | a6 | c7 | b38 |
| 642 | a6 | c7 | b39 |
| 643 | a6 | c7 | b40 |
| 644 | a6 | c7 | b41 |
| 645 | a6 | c7 | b43 |
| 646 | a6 | c7 | b44 |
| 647 | a6 | c7 | b45 |
| 648 | a6 | c7 | b46 |
| 649 | a6 | c7 | b47 |
| 650 | a6 | c7 | c1 |
| 651 | a6 | c7 | c3 |
| 652 | a6 | c7 | c4 |
| 653 | a6 | c7 | c5 |
| 654 | a6 | c7 | c6 |
| 655 | a6 | c7 | c7 |
| 656 | a6 | c7 | c8 |
| 657 | a6 | c7 | c9 |
| 658 | a6 | c7 | c10 |
| 659 | a6 | c7 | c11 |
| 660 | a6 | c7 | c12 |
| 661 | a6 | c7 | c13 |
| 662 | a6 | c7 | c14 |
| 663 | a6 | c7 | c15 |
| 664 | a6 | c7 | c16 |
| 665 | a6 | c7 | c17 |
| 666 | a6 | c7 | c18 |
| 667 | a6 | c7 | c19 |
| 668 | a6 | c7 | c20 |
| 669 | a6 | c7 | c21 |
| 670 | a6 | c7 | c22 |
| 671 | a6 | c7 | c23 |
| 672 | a6 | c7 | c24 |
| 673 | a6 | c7 | c25 |
| 674 | a6 | c7 | c26 |
| 675 | a6 | c14 | c4 |
| 676 | a6 | c14 | c5 |
| 677 | a6 | c14 | b6 |
| 678 | a6 | c14 | b7 |
| 679 | a6 | c14 | b8 |
| 680 | a6 | c14 | b9 |
| 681 | a6 | c14 | b10 |
| 682 | a6 | c14 | b13 |
| 683 | a6 | c14 | b14 |
| 684 | a6 | c14 | b15 |
| 685 | a6 | c14 | b16 |
| 686 | a6 | c14 | b17 |
| 687 | a6 | c14 | b18 |
| 688 | a6 | c14 | b19 |
| 689 | a6 | c14 | b20 |
| 690 | a6 | c14 | b21 |
| 691 | a6 | c14 | b22 |
| 692 | a6 | c14 | b23 |
| 693 | a6 | c14 | b24 |
| 694 | a6 | c14 | b25 |
| 695 | a6 | c14 | b26 |
| 696 | a6 | c14 | b27 |
| 697 | a6 | c14 | b28 |
| 698 | a6 | c14 | b29 |
| 699 | a6 | c14 | b30 |
| 700 | a6 | c14 | b31 |
| 701 | a6 | c14 | b32 |
| 702 | a6 | c14 | b33 |
| 703 | a6 | c14 | b35 |
| 704 | a6 | c14 | b36 |
| 705 | a6 | c14 | b37 |
| 706 | a6 | c14 | b38 |
| 707 | a6 | c14 | b39 |
| 708 | a6 | c14 | b40 |
| 709 | a6 | c14 | b41 |
| 710 | a6 | c14 | b43 |
| 711 | a6 | c14 | b44 |
| 712 | a6 | c14 | b45 |
| 713 | a6 | c14 | b46 |
| 714 | a6 | c14 | b47 |
| 715 | a6 | c14 | b1 |
| 716 | a6 | c14 | b3 |
| 717 | a6 | c14 | b4 |
| 718 | a6 | c14 | c5 |
| 719 | a6 | c14 | c6 |
| 720 | a6 | c14 | c8 |
| 721 | a6 | c14 | c9 |
| 722 | a6 | c14 | c10 |
| 723 | a6 | c14 | c11 |
| 724 | a6 | c14 | c12 |
| 725 | a6 | c14 | c13 |
| 726 | a6 | c14 | c14 |
| 727 | a6 | c14 | c15 |
| 728 | a6 | c14 | c16 |
| 729 | a6 | c14 | c18 |
| 730 | a6 | c14 | c19 |
| 731 | a6 | c14 | c20 |
| 732 | a6 | c14 | c21 |
| 733 | a6 | c14 | c22 |
| 734 | a6 | c14 | c23 |
| 735 | a6 | c14 | c24 |
| 736 | a6 | c14 | c25 |
| 737 | a6 | c14 | c26 |
| 738 | a6 | c15 | b4 |
| 739 | a6 | c15 | b5 |
| 740 | a6 | c15 | b6 |
| 741 | a6 | c15 | b7 |
| 742 | a6 | c15 | b8 |
| 743 | a6 | c15 | b9 |
| 744 | a6 | c15 | b10 |
| 745 | a6 | c15 | b13 |
| 746 | a6 | c15 | b14 |
| 747 | a6 | c15 | b15 |
| 748 | a6 | c15 | b16 |
| 749 | a6 | c15 | b17 |
| 750 | a6 | c15 | b18 |
| 751 | a6 | c15 | b19 |
| 752 | a6 | c15 | b20 |
| 753 | a6 | c15 | b21 |
| 754 | a6 | c15 | b22 |
| 755 | a6 | c15 | b23 |
| 756 | a6 | c15 | b24 |
| 757 | a6 | c15 | b25 |
| 758 | a6 | c15 | b26 |
| 759 | a6 | c15 | b27 |
| 760 | a6 | c15 | b28 |
| 761 | a6 | c15 | b29 |
| 762 | a6 | c15 | b30 |
| 763 | a6 | c15 | b31 |
| 764 | a6 | c15 | b32 |
| 765 | a6 | c15 | b33 |
| 766 | a6 | c15 | b35 |
| 767 | a6 | c15 | b36 |
| 768 | a6 | c15 | b37 |
| 769 | a6 | c15 | b38 |
| 770 | a6 | c15 | b39 |
| 771 | a6 | c15 | b40 |
| 772 | a6 | c15 | b41 |
| 773 | a6 | c15 | b43 |
| 774 | a6 | c15 | b44 |
| 775 | a6 | c15 | b45 |
| 776 | a6 | c15 | b46 |
| 777 | a6 | c15 | b47 |
| 778 | a6 | c15 | c1 |
| 779 | a6 | c15 | c3 |
| 780 | a6 | c15 | c4 |
| 781 | a6 | c15 | c5 |
| 782 | a6 | c15 | c6 |
| 783 | a6 | c15 | c8 |
| 784 | a6 | c15 | c9 |
| 785 | a6 | c15 | c10 |
| 786 | a6 | c15 | c11 |
| 787 | a6 | c15 | c12 |
| 788 | a6 | c15 | c13 |
| 789 | a6 | c15 | c15 |
| 790 | a6 | c15 | c16 |
| 791 | a6 | c15 | c17 |
| 792 | a6 | c15 | c18 |
| 793 | a6 | c15 | c19 |
| 794 | a6 | c15 | c20 |
| 795 | a6 | c15 | c21 |
| 796 | a6 | c15 | c22 |
| 797 | a6 | c15 | c23 |
| 798 | a6 | c15 | c24 |
| 799 | a6 | c15 | c25 |
| 800 | a6 | c15 | c26 |
| 801 | a6 | c24 | b4 |
| 802 | a6 | c24 | b5 |
| 803 | a6 | c24 | b6 |
| 804 | a6 | c24 | b7 |
| 805 | a6 | c24 | b8 |
| 806 | a6 | c24 | b9 |
| 807 | a6 | c24 | b10 |
| 808 | a6 | c24 | b13 |
| 809 | a6 | c24 | b14 |
| 810 | a6 | c24 | b15 |
| 811 | a6 | c24 | b16 |
| 812 | a6 | c24 | b17 |
| 813 | a6 | c24 | b18 |
| 814 | a6 | c24 | b19 |
| 815 | a6 | c24 | b20 |
| 816 | a6 | c24 | b21 |
| 817 | a6 | c24 | b22 |
| 818 | a6 | c24 | b23 |
| 819 | a6 | c24 | b24 |
| 820 | a6 | c24 | b25 |
| 821 | a6 | c24 | b26 |
| 822 | a6 | c24 | b27 |
| 823 | a6 | c24 | b28 |
| 824 | a6 | c24 | b29 |
| 825 | a6 | c24 | b30 |
| 826 | a6 | c24 | b31 |
| 827 | a6 | c24 | b32 |
| 828 | a6 | c24 | b33 |
| 349 | a6 | c24 | b35 |
| 830 | a6 | c24 | b36 |
| 831 | a6 | c24 | b37 |
| 832 | a6 | c24 | b38 |
| 833 | a6 | c24 | b39 |
| 834 | a6 | c24 | b40 |
| 835 | a6 | c24 | b41 |
| 836 | a6 | c24 | b43 |
| 837 | a6 | c24 | b44 |
| 838 | a6 | c24 | b45 |
| 839 | a6 | c24 | b46 |
| 840 | a6 | c24 | b47 |
| 841 | a6 | c24 | c1 |
| 842 | a6 | c24 | c3 |
| 843 | a6 | c24 | c4 |
| 844 | a6 | c24 | c5 |
| 845 | a6 | c24 | c6 |
| 846 | a6 | c24 | c8 |
| 847 | a6 | c24 | c9 |
| 848 | a6 | c24 | c10 |
| 849 | a6 | c24 | c11 |
| 850 | a6 | c24 | c12 |
| 851 | a6 | c24 | c13 |
| 852 | a6 | c24 | c16 |
| 853 | a6 | c24 | c17 |
| 854 | a6 | c24 | c18 |
| 855 | a6 | c24 | c19 |
| 856 | a6 | c24 | c20 |
| 857 | a6 | c24 | c21 |
| 858 | a6 | c24 | c22 |
| 859 | a6 | c24 | c23 |
| 860 | a6 | c24 | c24 |
| 861 | a6 | c24 | c25 |
| 862 | a6 | c24 | c26 |
| 863 | a5 | b10 | c26 |
| 864 | a1 | b10 | c7 |
| 865 | a1 | b48 | c7 |
| 866 | a1 | b48 | c14 |
| 867 | a1 | b10 | c14 |
| 868 | a1 | b48 | c14 |

14. An amine compound represented by Formula 1: 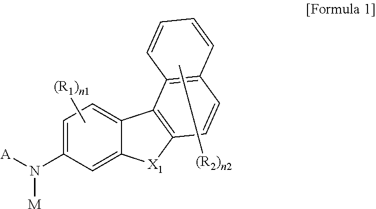
wherein in Formula 1,
A is a group represented by one of Formula 2-1 to Formula 2-3,
M is a group represented by Formula 3,
X₁ is O or S,
R₁ and R₂ are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 is an integer from 0 to 3, and
n2 is an integer from 0 to 6, 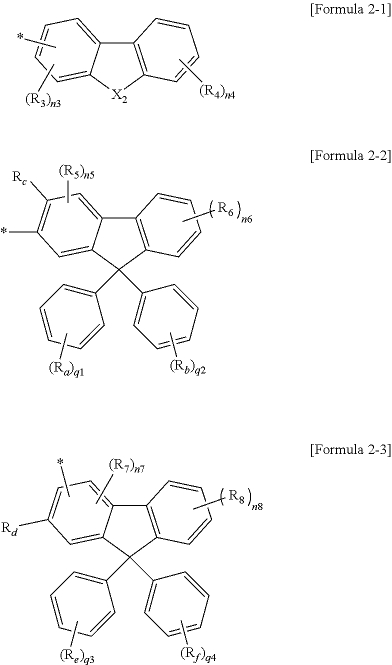
wherein in Formula 2-1 to Formula 2-3,
X₂ is O or S,
R_{c} is a hydrogen atom, a deuterium atom, or a substituted or unsubstituted alkyl group having 1 to 20 carbon atoms,
Rₐ, R_{b}, R_{d} to R_{f}, and R₃ to R₈ are each independently a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,
n3 is an integer from 0 to 3,
n4, n6, and n8 are each independently an integer from 0 to 4,
n5 and n7 are each independently an integer from 0 to 2,
q1 to q4 are each independently an integer from 0 to 5, and
—* represents a bond to Formula 1; 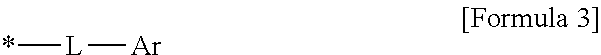
wherein in Formula 3,
L is a direct linkage, a substituted or unsubstituted arylene group having 6 to 30 ring-forming carbon atoms, or a substituted or unsubstituted heteroarylene group having 2 to 30 ring-forming carbon atoms,
Ar is a substituted or unsubstituted aryl group having 6 to 30 ring-forming carbon atoms, or a substituted or unsubstituted heteroaryl group having 2 to 30 ring-forming carbon atoms and 1 or 2 ring-forming heteroatoms, a group represented by Formula 2-2, or a group represented by Formula 2-3, and
—* represents a bond to Formula 1,
wherein in the amine compound represented by Formula 1,
a case where the amine compound comprises an aromatic fused ring containing sp³ carbon in addition to sp³ carbon contained in a fluorene skeleton represented by Formula 2-2 or Formula 2-3 is excluded,
a case where the amine compound comprises a nitrogen-containing heterocycle and a halogen atom is excluded, and
a case where M is a group represented by Formula a-1 or Formula a-2 is excluded: 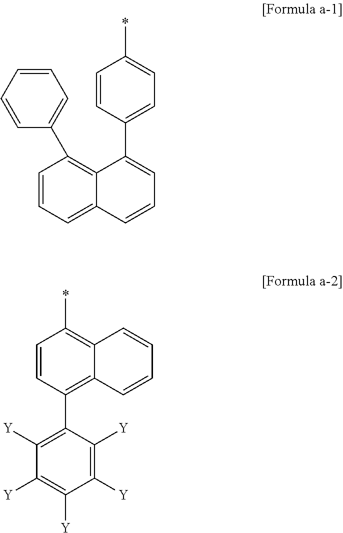
wherein in Formula a-1 and Formula a-2,
Y is a hydrogen atom or a deuterium atom, and
—* represents a bond to Formula 1, and
wherein in Formula 1,
when A is a group represented by Formula 2-2 or Formula 2-3, M does not comprise a naphthylene moiety,
when A is a group represented by Formula 2-1 and the group represented by Formula 2-1 is linked to N in Formula 1 at carbon 4, n1 is 0,
when A is a group represented by Formula 2-2 or Formula 2-3, n1 is 0,
when A is a group represented by Formula 2-1, X₂ in Formula 2-1 is S, and the group represented by Formula 2-1 is linked to N in Formula 1 at carbon 1, a case where L is a direct linkage or a group represented by Formula a-3 is excluded: 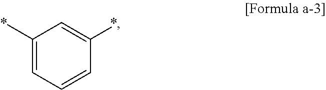
and
when A is a group represented by Formula 2-1, X₂ in Formula 2-1 is O, and the group represented by Formula 2-1 is linked to N in Formula 1 at carbon 2 or carbon 4, a case where M in Formula 1 is an unsubstituted phenyl group or a phenyl group having a substituent having 0 to 9 carbon atoms is excluded.

15. The amine compound of claim 14, wherein the amine compound represented by Formula 1 is represented by one of Formula 1-1-1 to Formula 1-1-3: 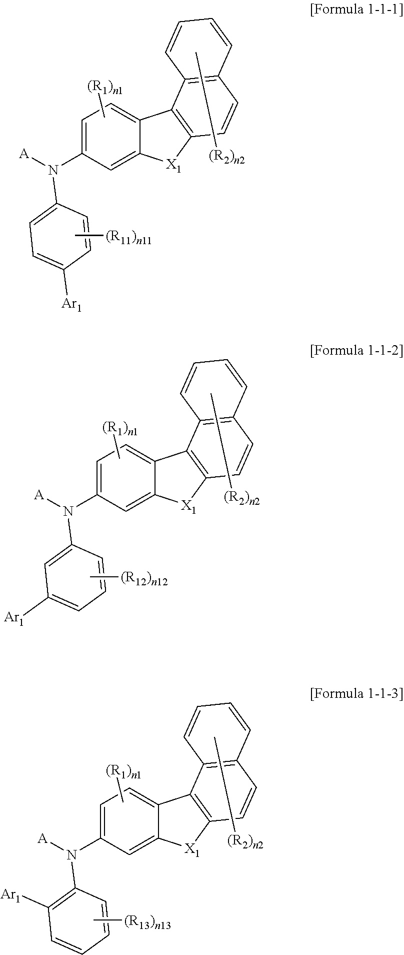
wherein in Formula 1-1-1 to Formula 1-1-3,
R₁₁ to R₁₃ are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n11 to n13 are each independently an integer from 0 to 4, and
Ar₁ is a group represented by one of Formula A-1 to Formula A-4: 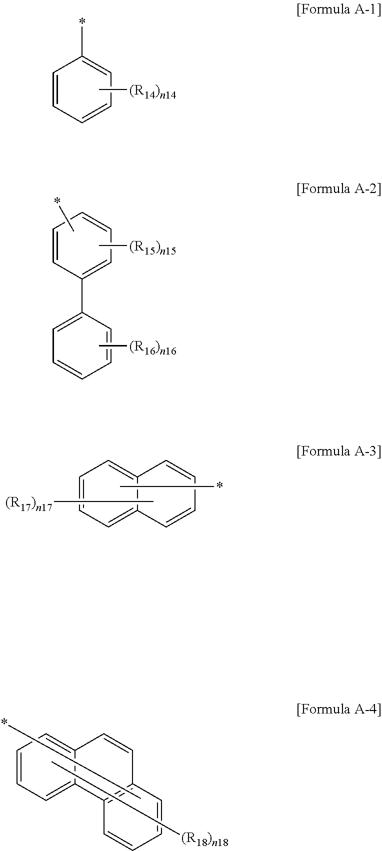
wherein in Formula A-1 to Formula A-4,
R₁₄ to R₁₈ are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n14 and n16 are each independently an integer from 0 to 5,
n15 is an integer from 0 to 4,
n17 is an integer from 0 to 7, and
n18 is an integer from 0 to 9, and
wherein in Formula 1-1-1 to Formula 1-1-3,
when A is a group represented by Formula 2-2 or Formula 2-3, Ar₁ is a group represented by Formula A-1, Formula A-2, or Formula A-4, and
A, X₁, R₁, R₂, n1, and n2 are the same as defined in Formula 1.

16. The amine compound of claim 14, wherein the amine compound represented by Formula 1 is represented by one of Formula 1-2-1 to Formula 1-2-7: 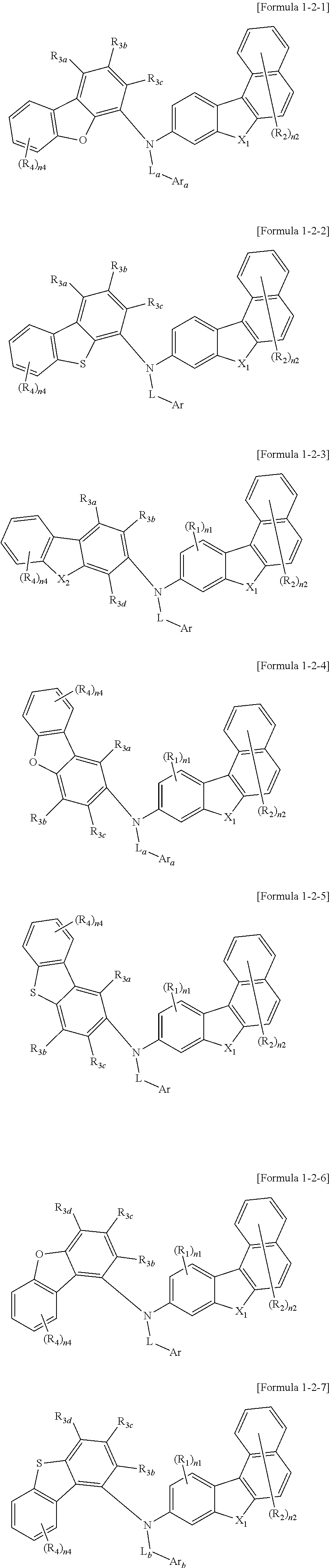
wherein in Formula 1-2-1 to Formula 1-2-7,
R₃ₐ to R_{3d} are each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Lₐ is a substituted or unsubstituted arylene group having 6 to 30 ring-forming carbon atoms or a substituted or unsubstituted heteroarylene group having 2 to 30 ring-forming carbon atoms, and
Arₐ is a substituted or unsubstituted aryl group having 6 to 30 ring-forming carbon atoms or a substituted or unsubstituted heteroaryl group having 2 to 30 ring-forming carbon atoms,
except that Arₐ is not a substituted or unsubstituted phenyl group,
wherein in Formula 1-2-7,
L_{b} is a substituted or unsubstituted arylene group having 6 to 30 ring-forming carbon atoms or a substituted or unsubstituted heteroarylene group having 2 to 30 ring-forming carbon atoms, and
a case where L_{b} is a group represented by Formula a-3 is excluded, and
wherein in Formula 1-2-1 to Formula 1-2-7,
L, Ar, X₁, R₁, R₂, n1, n2, X₂, R₄, and n4 are the same as defined in Formula 1 and Formula 2.

17. The amine compound of claim 14, wherein the amine compound represented by Formula 1 is represented by one of Formula 1-3-1 to Formula 1-3-4: 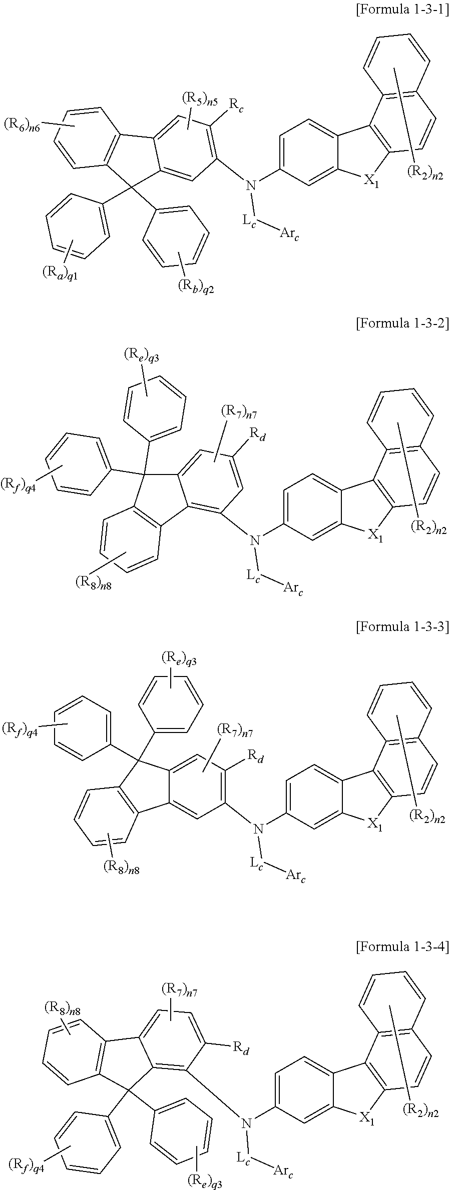
wherein in Formula 1-3-1 to Formula 1-3-4,
L_{c} is a direct linkage, a substituted or unsubstituted arylene group having 6 to 30 ring-forming carbon atoms, or a substituted or unsubstituted heteroarylene group having 2 to 30 ring-forming carbon atoms,
except that L4 is not a substituted or unsubstituted naphthalene group,
Ar_{c} is a substituted or unsubstituted aryl group having 6 to 30 ring-forming carbon atoms or a substituted or unsubstituted heteroaryl group having 2 to 30 ring-forming carbon atoms,
except that Ar_{c} is not a substituted or unsubstituted naphthyl group, and
X₁, R₂, n2, R₅ to R₈, n5 to n8, Rₐ to R_{f}, and q1 to q4 are the same as defined in Formula 1, Formula 2-2, and Formula 2-3.

18. The amine compound of claim 14, wherein the amine compound represented by Formula 1 is represented by Formula 1-4-1 or Formula 1-4-2: 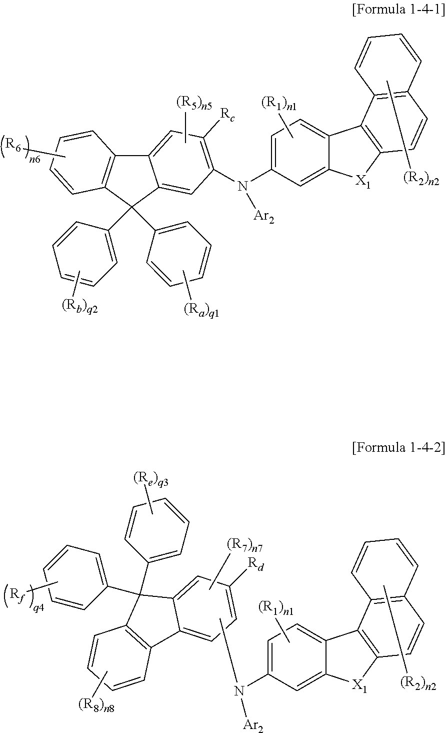
wherein in Formula 1-4-1 and Formula 1-4-2,
Ar₂ is a group represented by one of Formula B-1 to Formula B-7: 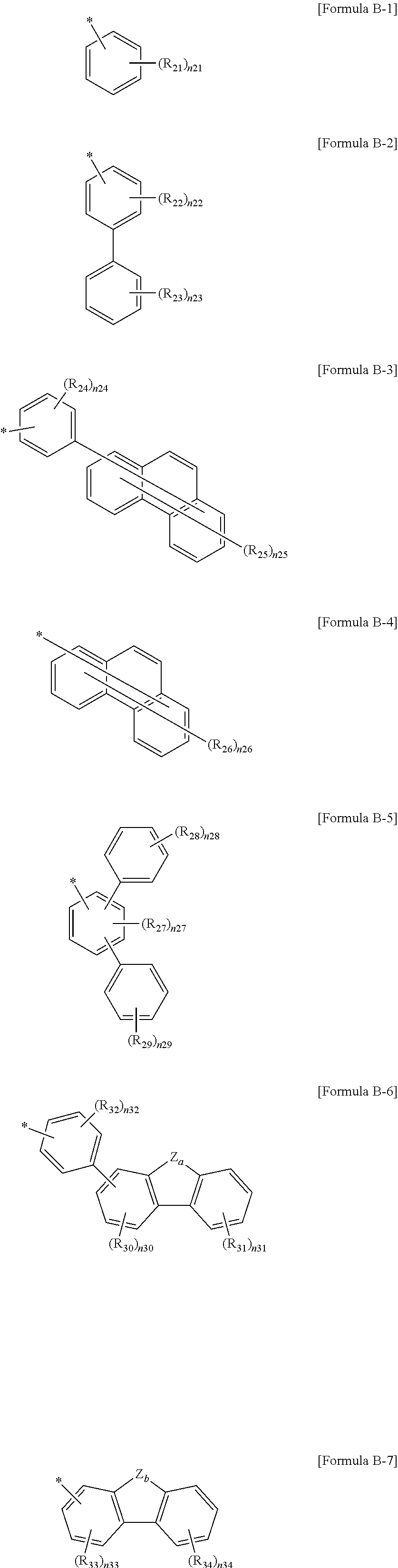
wherein in Formula B-1 to Formula B-7,
Zₐ and Z_{b} are each independently O or S,
R₂₁ to R₃₄ are each independently a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,
n21, n23, n28, and n29 are each independently an integer from 0 to 5,
n22, n24, n31, n32, and n34 are each independently an integer from 0 to 4,
n25 and n26 are each independently an integer from 0 to 9, and
n27, n30, and n33 are each independently an integer from 0 to 3, and
wherein in Formula 1-4-1 and Formula 1-4-2,
X₁, R₁, R₂, n1, n2, R₅ to R₈, n5 to n8, Rₐ to R_{f}, and q1 to q4 are the same as defined in Formula 1, Formula 2-2, and Formula 2-3.

19. The amine compound of claim 14, wherein the amine compound represented by Formula 1 is represented by one of Formula 1-6-1 to Formula 1-6-4: 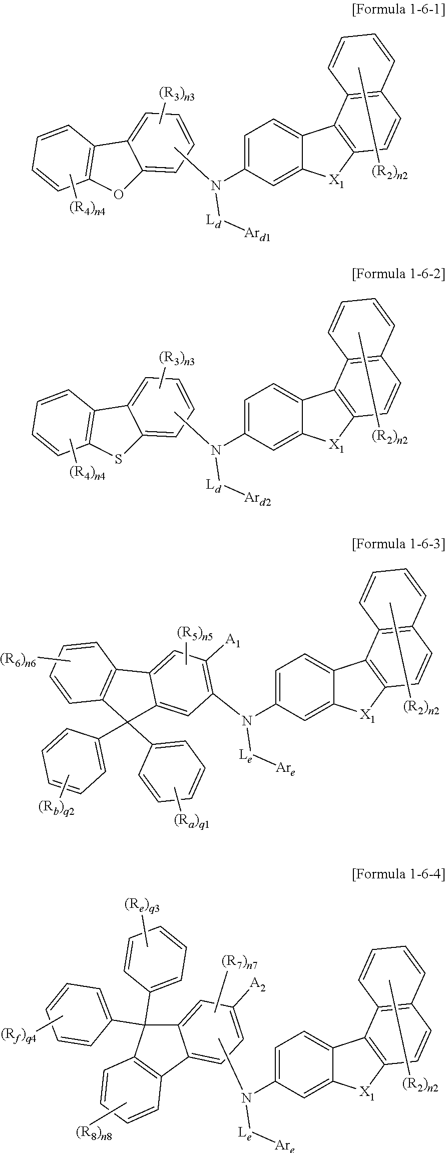
wherein in Formula 1-6-1 to Formula 1-6-4,
A₁ and A₂ are each independently a hydrogen atom or a deuterium atom,
L_{d} is a group represented by one of Formula L-1 to Formula L-5,
Ar_{d1} is a group represented by Formula 2-2, a group represented by Formula 2-3, a substituted or unsubstituted naphthyl group, a substituted or unsubstituted anthracene group, a substituted or unsubstituted phenanthrene group, a substituted or unsubstituted dibenzofuran group, or a substituted or unsubstituted dibenzothiophene group,
Ar_{d2} is a group represented by Formula 2-2, a group represented by Formula 2-3, a substituted or unsubstituted phenyl group, a substituted or unsubstituted biphenyl group, a substituted or unsubstituted naphthyl group, a substituted or unsubstituted phenanthrene group, a substituted or unsubstituted anthracene group, a substituted or unsubstituted dibenzofuran group, or a substituted or unsubstituted dibenzothiophene group,
Lₑ is a direct linkage, or a group represented by one of Formula L-1 to Formula L-5, and
Arₑ is a group represented by Formula 2-2, a group represented by Formula 2-3, or a group represented by one of Formula C-1 to Formula C-5: 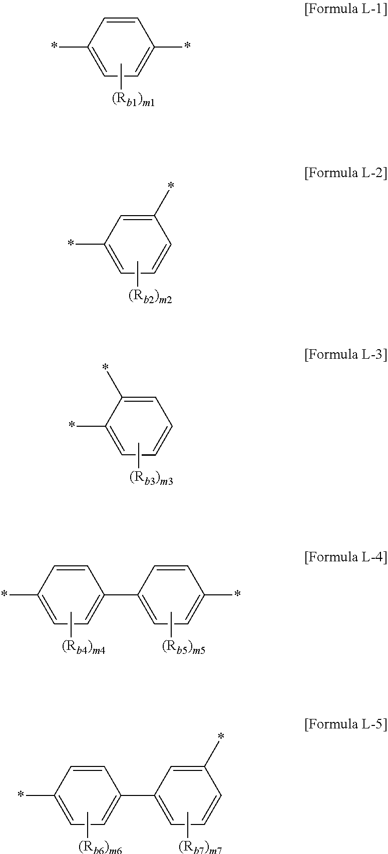
wherein in Formula L-1 to Formula L-5,
R_{b1} to R_{b7} are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and
m1 to m7 are each independently an integer from 0 to 4: 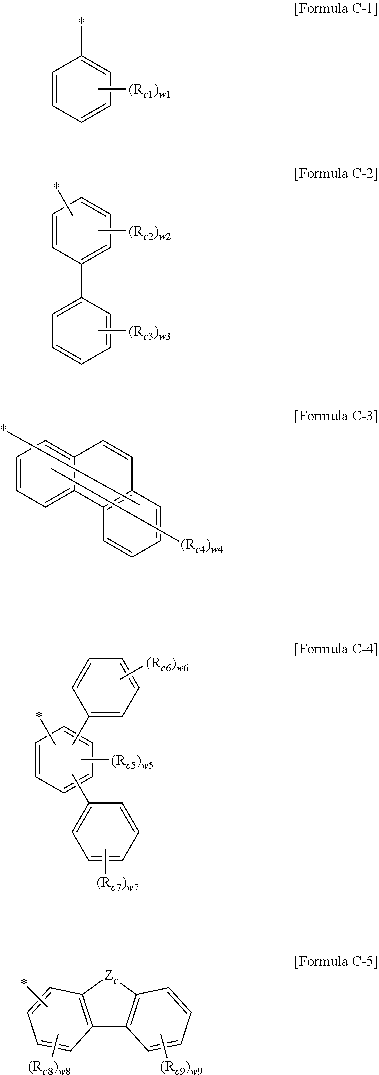
wherein in Formula C-1 to Formula C-5,
Z_{c} is O or S,
R_{c1} to R_{c9} are each independently a hydrogen atom, a deuterium atom, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring,
w1, w3, w6, and w7 are each independently an integer from 0 to 5,
w2 and w9 are each independently an integer from 0 to 4,
w4 is an integer from 0 to 9, and
w5 and w8 are each independently an integer from 0 to 3, and
wherein in Formula 1-6-1 to Formula 1-6-4,
X₁, R₂ to R₈, n2 to n8, Rₐ, R_{b}, Rₑ, R_{f}, and q1 to q4 are the same as defined in Formula 1, Formula 2-2, and Formula 2-3.

20. The amine compound of claim 14, wherein
the amine compound represented by Formula 1 is represented by Formula 1-7, and
the amine compound meets one of the combinations in Compound Combination Table 1: 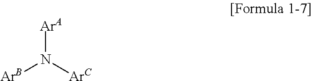
wherein in Formula 1-7,
Ar^{A} is a group selected from Substituent Group A,
Ar^{B} is a group selected from Substituent Group B, and
Ar^{C} is a group selected from Substituent Group C: 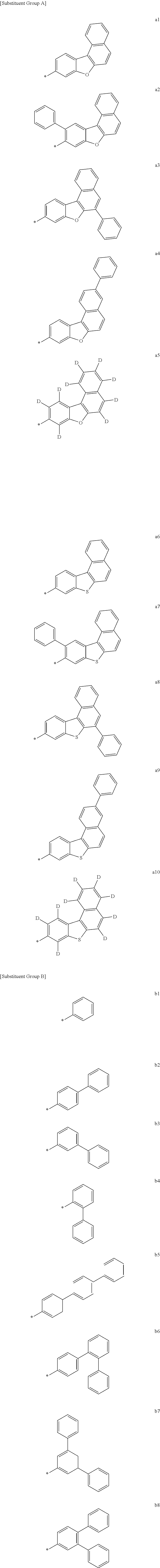 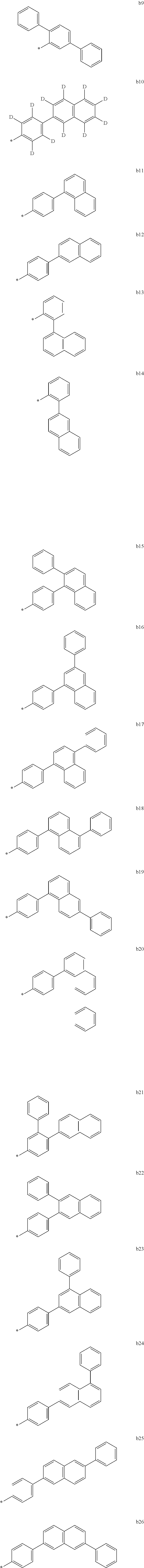 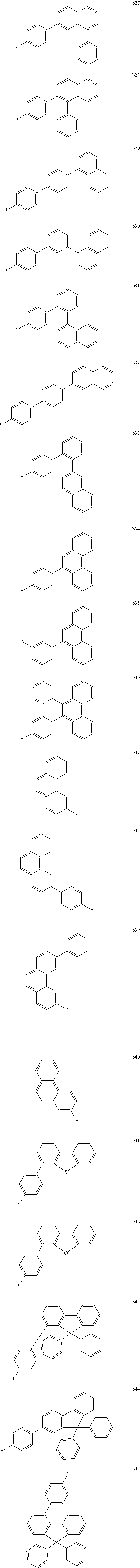 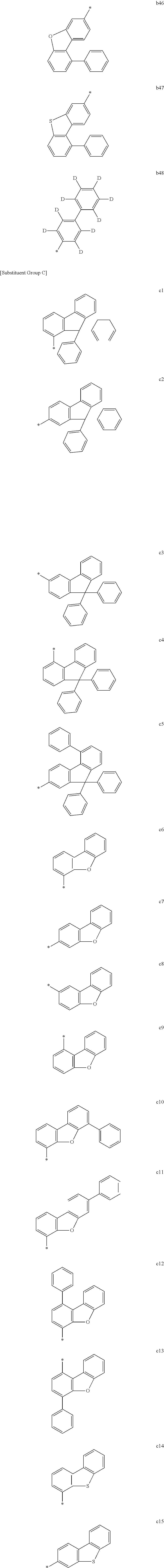 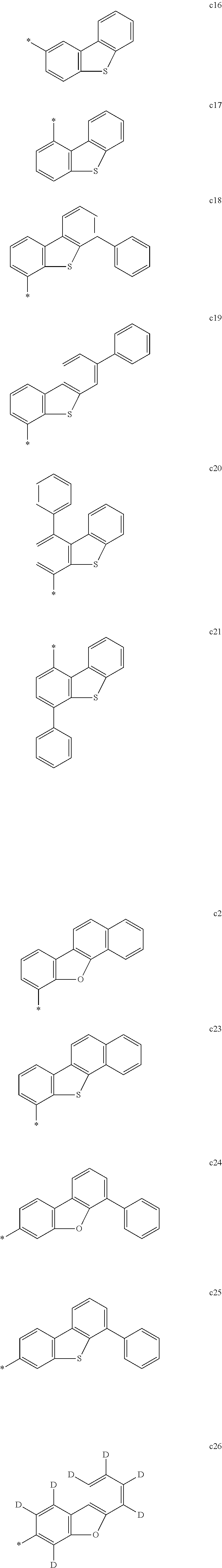
[Compound Combination Table 1]

| No. | Ar^{A} | Ar^{B} | Ar^{C} |
|---|---|---|---|
| 1 | a1 | b11 | c7 |
| 2 | a2 | b11 | c7 |
| 3 | a3 | b11 | c7 |
| 4 | a4 | b11 | c7 |
| 5 | a5 | b11 | c7 |
| 6 | a6 | b11 | c7 |
| 7 | a7 | b11 | c7 |
| 8 | a8 | b11 | c7 |
| 9 | a9 | b11 | c7 |
| 10 | a10 | b11 | c7 |
| 11 | a1 | b1 | c1 |
| 12 | a1 | b1 | c2 |
| 13 | a1 | b1 | c3 |
| 14 | a1 | b1 | c4 |
| 15 | a1 | b1 | c5 |
| 16 | a1 | b1 | c7 |
| 17 | a1 | b1 | c9 |
| 18 | a1 | b1 | c13 |
| 19 | a1 | b1 | c14 |
| 20 | a1 | b1 | c15 |
| 21 | a1 | b1 | c16 |
| 22 | a1 | b1 | c17 |
| 23 | a1 | b1 | c18 |
| 24 | a1 | b1 | c19 |
| 25 | a1 | b1 | c20 |
| 26 | a1 | b1 | c21 |
| 27 | a1 | b1 | c24 |
| 28 | a1 | b2 | c1 |
| 29 | a1 | b2 | c2 |
| 30 | a1 | b2 | c3 |
| 31 | a1 | b2 | c4 |
| 32 | a1 | b2 | c5 |
| 33 | a1 | b2 | c7 |
| 34 | a1 | b2 | c9 |
| 35 | a1 | b2 | c13 |
| 36 | a1 | b2 | c14 |
| 37 | a1 | b2 | c15 |
| 38 | a1 | b2 | c16 |
| 39 | a1 | b2 | c17 |
| 40 | a1 | b2 | c18 |
| 41 | a1 | b2 | c19 |
| 42 | a1 | b2 | c20 |
| 43 | a1 | b2 | c21 |
| 44 | a1 | b2 | c24 |
| 45 | a1 | b2 | c25 |
| 46 | a1 | b2 | c26 |
| 47 | a1 | b3 | c1 |
| 48 | a1 | b3 | c2 |
| 49 | a1 | b3 | c3 |
| 50 | a1 | b3 | c4 |
| 51 | a1 | b3 | c5 |
| 52 | a1 | b3 | c7 |
| 53 | a1 | b3 | c9 |
| 54 | a1 | b3 | c13 |
| 55 | a1 | b3 | c14 |
| 56 | a1 | b3 | c15 |
| 57 | a1 | b3 | c16 |
| 58 | a1 | b3 | c18 |
| 59 | a1 | b3 | c19 |
| 60 | a1 | b3 | c20 |
| 61 | a1 | b3 | c21 |
| 62 | a1 | b3 | c24 |
| 63 | a1 | b11 | c25 |
| 64 | a1 | b11 | c26 |
| 65 | a1 | b11 | c6 |
| 66 | a1 | b11 | c9 |
| 67 | a1 | b11 | c10 |
| 68 | a1 | b11 | c11 |
| 69 | a1 | b11 | c12 |
| 70 | a1 | b11 | c13 |
| 71 | a1 | b11 | c14 |
| 72 | a1 | b11 | c15 |
| 73 | a1 | b11 | c16 |
| 74 | a1 | b11 | c17 |
| 75 | a1 | b11 | c18 |
| 76 | a1 | b11 | c19 |
| 77 | a1 | b11 | c20 |
| 78 | a1 | b11 | c21 |
| 79 | a1 | b11 | c22 |
| 80 | a1 | b11 | c23 |
| 81 | a1 | b11 | c24 |
| 82 | a1 | b12 | c6 |
| 83 | a1 | b12 | c7 |
| 84 | a1 | b12 | c9 |
| 85 | a1 | b12 | c10 |
| 86 | a1 | b12 | c11 |
| 87 | a1 | b12 | c12 |
| 88 | a1 | b12 | c13 |
| 89 | a1 | b12 | c14 |
| 90 | a1 | b12 | c15 |
| 91 | a1 | b12 | c16 |
| 92 | a1 | b12 | c17 |
| 93 | a1 | b12 | c18 |
| 94 | a1 | b12 | c19 |
| 95 | a1 | b12 | c20 |
| 96 | a1 | b12 | c21 |
| 97 | a1 | b12 | c22 |
| 98 | a1 | b12 | c23 |
| 99 | a1 | b12 | c24 |
| 100 | a1 | b12 | c25 |
| 101 | a1 | b12 | c26 |
| 102 | a1 | b34 | c6 |
| 103 | a1 | b34 | c7 |
| 104 | a1 | b34 | c8 |
| 105 | a1 | b34 | c9 |
| 106 | a1 | b34 | c10 |
| 107 | a1 | b34 | c11 |
| 108 | a1 | b34 | c12 |
| 109 | a1 | b34 | c13 |
| 110 | a1 | b34 | c14 |
| 111 | a1 | b34 | c15 |
| 112 | a1 | b34 | c16 |
| 113 | a1 | b34 | c17 |
| 114 | a1 | b34 | c18 |
| 115 | a1 | b34 | c19 |
| 116 | a1 | b34 | c20 |
| 117 | a1 | b34 | c21 |
| 118 | a1 | b34 | c22 |
| 119 | a1 | b34 | c23 |
| 120 | a1 | b34 | c24 |
| 121 | a1 | b34 | c25 |
| 122 | a1 | b34 | c26 |
| 123 | a1 | b42 | c1 |
| 124 | a1 | b42 | c2 |
| 125 | a1 | b42 | c3 |
| 126 | a1 | b42 | c4 |
| 127 | a1 | b42 | c5 |
| 128 | a1 | b42 | c6 |
| 129 | a1 | b42 | c7 |
| 130 | a1 | b42 | c8 |
| 131 | a1 | b42 | c9 |
| 132 | a1 | b42 | c10 |
| 133 | a1 | b42 | c11 |
| 134 | a1 | b42 | c12 |
| 135 | a1 | b42 | c13 |
| 136 | a1 | b42 | c14 |
| 137 | a1 | b42 | c15 |
| 138 | a1 | b42 | c16 |
| 139 | a1 | b42 | c18 |
| 140 | a1 | b42 | c19 |
| 141 | a1 | b42 | c20 |
| 142 | a1 | b42 | c21 |
| 143 | a1 | b42 | c22 |
| 144 | a1 | b42 | c23 |
| 145 | a1 | b42 | c24 |
| 146 | a1 | b42 | c25 |
| 147 | a1 | b42 | c26 |
| 148 | a1 | c2 | b4 |
| 149 | a1 | c2 | b5 |
| 150 | a1 | c2 | b6 |
| 151 | a1 | c2 | b7 |
| 152 | a1 | c2 | b8 |
| 153 | a1 | c2 | b9 |
| 154 | a1 | c2 | b41 |
| 155 | a1 | c2 | b43 |
| 156 | a1 | c2 | b44 |
| 157 | a1 | c2 | b45 |
| 158 | a1 | c2 | b46 |
| 159 | a1 | c2 | b47 |
| 160 | a1 | c2 | c1 |
| 161 | a1 | c2 | c2 |
| 162 | a1 | c2 | c3 |
| 163 | a1 | c2 | c4 |
| 164 | a1 | c2 | c5 |
| 165 | a1 | c2 | c6 |
| 166 | a1 | c2 | c7 |
| 167 | a1 | c2 | c8 |
| 168 | a1 | c2 | c9 |
| 169 | a1 | c2 | c10 |
| 170 | a1 | c2 | c11 |
| 171 | a1 | c2 | c12 |
| 172 | a1 | c2 | c13 |
| 173 | a1 | c2 | c14 |
| 174 | a1 | c2 | c15 |
| 175 | a1 | c2 | c16 |
| 176 | a1 | c2 | c17 |
| 177 | a1 | c2 | c18 |
| 178 | a1 | c2 | c19 |
| 179 | a1 | c2 | c20 |
| 180 | a1 | c2 | c21 |
| 181 | a1 | c2 | c24 |
| 182 | a1 | c2 | c25 |
| 183 | a1 | c2 | c26 |
| 184 | a1 | c7 | b4 |
| 185 | a1 | c7 | b5 |
| 186 | a1 | c7 | b6 |
| 187 | a1 | c7 | b7 |
| 188 | a1 | c7 | b8 |
| 189 | a1 | c7 | b9 |
| 190 | a1 | c7 | b10 |
| 191 | a1 | c7 | b13 |
| 192 | a1 | c7 | b14 |
| 193 | a1 | c7 | b15 |
| 194 | a1 | c7 | b16 |
| 195 | a1 | c7 | b17 |
| 196 | a1 | c7 | b18 |
| 197 | a1 | c7 | b19 |
| 198 | a1 | c7 | b20 |
| 199 | a1 | c7 | b21 |
| 200 | a1 | c7 | b22 |
| 201 | a1 | c7 | b23 |
| 202 | a1 | c7 | b24 |
| 203 | a1 | c7 | b25 |
| 204 | a1 | c7 | b26 |
| 205 | a1 | c7 | b27 |
| 206 | a1 | c7 | b28 |
| 207 | a1 | c7 | b29 |
| 208 | a1 | c7 | b30 |
| 209 | a1 | c7 | b31 |
| 210 | a1 | c7 | b32 |
| 211 | a1 | c7 | b33 |
| 212 | a1 | c7 | b35 |
| 213 | a1 | c7 | b36 |
| 214 | a1 | c7 | b37 |
| 215 | a1 | c7 | b38 |
| 216 | a1 | c7 | b39 |
| 217 | a1 | c7 | b40 |
| 218 | a1 | c7 | b41 |
| 219 | a1 | c7 | b43 |
| 220 | a1 | c7 | b44 |
| 221 | a1 | c7 | b45 |
| 222 | a1 | c7 | b46 |
| 223 | a1 | c7 | b47 |
| 224 | a1 | c7 | c1 |
| 225 | a1 | c7 | c3 |
| 226 | a1 | c7 | c4 |
| 227 | a1 | c7 | c5 |
| 228 | a1 | c7 | c6 |
| 229 | a1 | c7 | c7 |
| 230 | a1 | c7 | c8 |
| 231 | a1 | c7 | c9 |
| 232 | a1 | c7 | c10 |
| 233 | a1 | c7 | c11 |
| 234 | a1 | c7 | c12 |
| 235 | a1 | c7 | c13 |
| 236 | a1 | c7 | c14 |
| 237 | a1 | c7 | c15 |
| 238 | a1 | c7 | c16 |
| 239 | a1 | c7 | c17 |
| 240 | a1 | c7 | c18 |
| 241 | a1 | c7 | c19 |
| 242 | a1 | c7 | c20 |
| 243 | a1 | c7 | c21 |
| 244 | a1 | c7 | c22 |
| 245 | a1 | c7 | c23 |
| 246 | a1 | c7 | c24 |
| 247 | a1 | c7 | c25 |
| 248 | a1 | c7 | c26 |
| 249 | a1 | c14 | b4 |
| 250 | a1 | c14 | b5 |
| 251 | a1 | c14 | b6 |
| 252 | a1 | c14 | b7 |
| 253 | a1 | c14 | b8 |
| 254 | a1 | c14 | b9 |
| 255 | a1 | c14 | b10 |
| 256 | a1 | c14 | b13 |
| 257 | a1 | c14 | b114 |
| 258 | a1 | c14 | b15 |
| 259 | a1 | c14 | b16 |
| 260 | a1 | c14 | b17 |
| 261 | a1 | c14 | b18 |
| 262 | a1 | c14 | b19 |
| 263 | a1 | c14 | b20 |
| 264 | a1 | c14 | b21 |
| 265 | a1 | c14 | b22 |
| 266 | a1 | c14 | b23 |
| 267 | a1 | c14 | b24 |
| 268 | a1 | c14 | b25 |
| 269 | a1 | c14 | b26 |
| 270 | a1 | c14 | b27 |
| 271 | a1 | c14 | b28 |
| 272 | a1 | c14 | b29 |
| 273 | a1 | c14 | b30 |
| 274 | a1 | c14 | b31 |
| 275 | a1 | c14 | b32 |
| 276 | a1 | c14 | b33 |
| 277 | a1 | c14 | b35 |
| 278 | a1 | c14 | b36 |
| 279 | a1 | c14 | b37 |
| 280 | a1 | c14 | b38 |
| 281 | a1 | c14 | b39 |
| 282 | a1 | c14 | b40 |
| 283 | a1 | c14 | b41 |
| 284 | a1 | c14 | b43 |
| 285 | a1 | c14 | b44 |
| 286 | a1 | c14 | b45 |
| 287 | a1 | c14 | b46 |
| 288 | a1 | c14 | b47 |
| 289 | a1 | c14 | c1 |
| 290 | a1 | c14 | c3 |
| 291 | a1 | c14 | c4 |
| 292 | a1 | c14 | c5 |
| 293 | a1 | c14 | c6 |
| 294 | a1 | c14 | c8 |
| 295 | a1 | c14 | c9 |
| 296 | a1 | c14 | c10 |
| 297 | a1 | c14 | c11 |
| 298 | a1 | c14 | c12 |
| 299 | a1 | c14 | c13 |
| 300 | a1 | c14 | c14 |
| 301 | a1 | c14 | c15 |
| 302 | a1 | c14 | c16 |
| 303 | a1 | c14 | c18 |
| 304 | a1 | c14 | c19 |
| 305 | a1 | c14 | c20 |
| 306 | a1 | c14 | c21 |
| 307 | a1 | c14 | c22 |
| 308 | a1 | c14 | c23 |
| 309 | a1 | c14 | c24 |
| 310 | a1 | c14 | c25 |
| 311 | a1 | c14 | c26 |
| 312 | a1 | c15 | b4 |
| 313 | a1 | c15 | b5 |
| 314 | a1 | c15 | b6 |
| 315 | a1 | c15 | b7 |
| 316 | a1 | c15 | b8 |
| 317 | a1 | c15 | b9 |
| 318 | a1 | c15 | b10 |
| 319 | a1 | c15 | b13 |
| 320 | a1 | c15 | b14 |
| 321 | a1 | c15 | b15 |
| 322 | a1 | c15 | b16 |
| 323 | a1 | c15 | b17 |
| 324 | a1 | c15 | b18 |
| 325 | a1 | c15 | b19 |
| 326 | a1 | c15 | b20 |
| 327 | a1 | c15 | b21 |
| 328 | a1 | c15 | b22 |
| 329 | a1 | c15 | b23 |
| 330 | a1 | c15 | b24 |
| 331 | a1 | c15 | b25 |
| 332 | a1 | c15 | b26 |
| 333 | a1 | c15 | b27 |
| 334 | a1 | c15 | b28 |
| 335 | a1 | c15 | b29 |
| 336 | a1 | c15 | b30 |
| 337 | a1 | c15 | b31 |
| 338 | a1 | c15 | b32 |
| 339 | a1 | c15 | b33 |
| 340 | a1 | c15 | b35 |
| 341 | a1 | c15 | b36 |
| 342 | a1 | c15 | b37 |
| 343 | a1 | c15 | b38 |
| 344 | a1 | c15 | b39 |
| 345 | a1 | c15 | b40 |
| 346 | a1 | c15 | b41 |
| 347 | a1 | c15 | b43 |
| 348 | a1 | c15 | b44 |
| 349 | a1 | c15 | b45 |
| 350 | a1 | c15 | b46 |
| 351 | a1 | c15 | b47 |
| 352 | a1 | c15 | c1 |
| 353 | a1 | c15 | c3 |
| 354 | a1 | c15 | c4 |
| 355 | a1 | c15 | c5 |
| 356 | a1 | c15 | c6 |
| 357 | a1 | c15 | c8 |
| 358 | a1 | c15 | c9 |
| 359 | a1 | c15 | c10 |
| 360 | a1 | c15 | c11 |
| 361 | a1 | c15 | c12 |
| 362 | a1 | c15 | c13 |
| 363 | a1 | c15 | c15 |
| 364 | a1 | c15 | c16 |
| 365 | a1 | c15 | c17 |
| 366 | a1 | c15 | c18 |
| 367 | a1 | c15 | c19 |
| 368 | a1 | c15 | c20 |
| 369 | a1 | c15 | c21 |
| 370 | a1 | c15 | c22 |
| 371 | a1 | c15 | c23 |
| 372 | a1 | c15 | c24 |
| 373 | a1 | c15 | c25 |
| 374 | a1 | c15 | c26 |
| 375 | a1 | c24 | b4 |
| 376 | a1 | c24 | b5 |
| 377 | a1 | c24 | b6 |
| 378 | a1 | c24 | b7 |
| 379 | a1 | c24 | b8 |
| 380 | a1 | c24 | b9 |
| 381 | a1 | c24 | b10 |
| 382 | a1 | c24 | b13 |
| 383 | a1 | c24 | b14 |
| 384 | a1 | c24 | b15 |
| 385 | a1 | c24 | b16 |
| 386 | a1 | c24 | b17 |
| 387 | a1 | c24 | b18 |
| 388 | a1 | c24 | b19 |
| 389 | a1 | c24 | b20 |
| 390 | a1 | c24 | b21 |
| 391 | a1 | c24 | b22 |
| 392 | a1 | c24 | b23 |
| 393 | a1 | c24 | b24 |
| 394 | a1 | c24 | b25 |
| 395 | a1 | c24 | b26 |
| 396 | a1 | c24 | b27 |
| 397 | a1 | c24 | b28 |
| 398 | a1 | c24 | b29 |
| 399 | a1 | c24 | b30 |
| 400 | a1 | c24 | b31 |
| 401 | a1 | c24 | b32 |
| 402 | a1 | c24 | b33 |
| 403 | a1 | c24 | b35 |
| 404 | a1 | c24 | b36 |
| 405 | a1 | c24 | b37 |
| 406 | a1 | c24 | b38 |
| 407 | a1 | c24 | b39 |
| 408 | a1 | c24 | b40 |
| 409 | a1 | c24 | b41 |
| 410 | a1 | c24 | b43 |
| 411 | a1 | c24 | b44 |
| 412 | a1 | c24 | b45 |
| 413 | a1 | c24 | b46 |
| 414 | a1 | c24 | b47 |
| 415 | a1 | c24 | c1 |
| 416 | a1 | c24 | c3 |
| 417 | a1 | c24 | c4 |
| 418 | a1 | c24 | c5 |
| 419 | a1 | c24 | c6 |
| 420 | a1 | c24 | c8 |
| 421 | a1 | c24 | c9 |
| 422 | a1 | c24 | c10 |
| 423 | a1 | c24 | c11 |
| 424 | a1 | c24 | c1 |
| 425 | a1 | c24 | c13 |
| 426 | a1 | c24 | c16 |
| 427 | a1 | c24 | c17 |
| 428 | a1 | c24 | c18 |
| 429 | a1 | c24 | c19 |
| 430 | a1 | c24 | c20 |
| 431 | a1 | c24 | c21 |
| 432 | a1 | c24 | c22 |
| 433 | a1 | c24 | c23 |
| 434 | a1 | c24 | c24 |
| 435 | a1 | c24 | c25 |
| 436 | a1 | c24 | c26 |
| 437 | a6 | b1 | c1 |
| 438 | a6 | b1 | c2 |
| 439 | a6 | b1 | c3 |
| 440 | a6 | b1 | c4 |
| 441 | a6 | b1 | c5 |
| 442 | a6 | b1 | c7 |
| 443 | a6 | b1 | c9 |
| 444 | a6 | b1 | c13 |
| 445 | a6 | b1 | c14 |
| 446 | a6 | b1 | c15 |
| 447 | a6 | b1 | c16 |
| 448 | a6 | b1 | c17 |
| 449 | a6 | b1 | c18 |
| 450 | a6 | b1 | c19 |
| 451 | a6 | b1 | c20 |
| 452 | a6 | b1 | c21 |
| 453 | a6 | b1 | c24 |
| 454 | a6 | b2 | c1 |
| 455 | a6 | b2 | c2 |
| 456 | a6 | b2 | c3 |
| 457 | a6 | b2 | c4 |
| 458 | a6 | b2 | c5 |
| 459 | a6 | b2 | c7 |
| 460 | a6 | b2 | c9 |
| 461 | a6 | b2 | c13 |
| 462 | a6 | b2 | c14 |
| 463 | a6 | b2 | c15 |
| 464 | a6 | b2 | c16 |
| 465 | a6 | b2 | c17 |
| 466 | a6 | b2 | c18 |
| 467 | a6 | b2 | c19 |
| 468 | a6 | b2 | c20 |
| 469 | a6 | b2 | c21 |
| 470 | a6 | b2 | c24 |
| 471 | a6 | b2 | c25 |
| 472 | a6 | b2 | c26 |
| 473 | a6 | b3 | c1 |
| 474 | a6 | b3 | c2 |
| 475 | a6 | b3 | c3 |
| 476 | a6 | b3 | c4 |
| 477 | a6 | b3 | c5 |
| 478 | a6 | b3 | c7 |
| 479 | a6 | b3 | c9 |
| 480 | a6 | b3 | c13 |
| 481 | a6 | b3 | c16 |
| 482 | a6 | b3 | c15 |
| 483 | a6 | b3 | c16 |
| 484 | a6 | b3 | c18 |
| 485 | a6 | b3 | c19 |
| 486 | a6 | b3 | c20 |
| 487 | a6 | b3 | c21 |
| 488 | a6 | b3 | c24 |
| 489 | a6 | b11 | c25 |
| 490 | a6 | b11 | c26 |
| 491 | a6 | b11 | c6 |
| 492 | a6 | b11 | c9 |
| 493 | a6 | b11 | c10 |
| 494 | a6 | b11 | c11 |
| 495 | a6 | b11 | c12 |
| 496 | a6 | b11 | c13 |
| 497 | a6 | b11 | c14 |
| 498 | a6 | b11 | c15 |
| 499 | a6 | b11 | c16 |
| 500 | a6 | b11 | c17 |
| 501 | a6 | b11 | c18 |
| 502 | a6 | b11 | c19 |
| 503 | a6 | b11 | c20 |
| 504 | a6 | b11 | c21 |
| 505 | a6 | b11 | c22 |
| 506 | a6 | b11 | c23 |
| 507 | a6 | b11 | c24 |
| 508 | a6 | b12 | c25 |
| 509 | a6 | b12 | c60 |
| 510 | a6 | b12 | c7 |
| 511 | a6 | b12 | c9 |
| 512 | a6 | b12 | c10 |
| 513 | a6 | b12 | c11 |
| 514 | a6 | b12 | c12 |
| 515 | a6 | b12 | c13 |
| 516 | a6 | b12 | c14 |
| 517 | a6 | b12 | c15 |
| 518 | a6 | b12 | c16 |
| 519 | a6 | b12 | c17 |
| 520 | a6 | b12 | c18 |
| 521 | a6 | b12 | c19 |
| 522 | a6 | b12 | c20 |
| 523 | a6 | b12 | c21 |
| 524 | a6 | b12 | c22 |
| 525 | a6 | b12 | c23 |
| 526 | a6 | b12 | c24 |
| 527 | a6 | b12 | c25 |
| 528 | a6 | b34 | c26 |
| 021 | a6 | b34 | c6 |
| 530 | a6 | b34 | c7 |
| 531 | a6 | b34 | c8 |
| 532 | a6 | b34 | c9 |
| 533 | a6 | b34 | c10 |
| 534 | a6 | b34 | c11 |
| 535 | a6 | b34 | c12 |
| 536 | a6 | b34 | c14 |
| 537 | a6 | b34 | c15 |
| 538 | a6 | b34 | c16 |
| 539 | a6 | b34 | c17 |
| 540 | a6 | b34 | c18 |
| 541 | a6 | b34 | c19 |
| 542 | a6 | b34 | c20 |
| 543 | a6 | b34 | c21 |
| 544 | a6 | b34 | c22 |
| 545 | a6 | b34 | c23 |
| 546 | a6 | b34 | c24 |
| 547 | a6 | b34 | c25 |
| 548 | a6 | b34 | c26 |
| 549 | a6 | b42 | c1 |
| 550 | a6 | b42 | c2 |
| 551 | a6 | b42 | c3 |
| 552 | a6 | b42 | c4 |
| 553 | a6 | b42 | c5 |
| 554 | a6 | b42 | c6 |
| 555 | a6 | b42 | c7 |
| 556 | a6 | b42 | c8 |
| 557 | a6 | b42 | c9 |
| 558 | a6 | b42 | c10 |
| 559 | a6 | b42 | c11 |
| 560 | a6 | b42 | c12 |
| 561 | a6 | b42 | c13 |
| 562 | a6 | b42 | c14 |
| 563 | a6 | b42 | c15 |
| 564 | a6 | b42 | c16 |
| 565 | a6 | b42 | c18 |
| 566 | a6 | b42 | c19 |
| 567 | a6 | b42 | c20 |
| 568 | a6 | b42 | c21 |
| 569 | a6 | b42 | c22 |
| 570 | a6 | b42 | c23 |
| 571 | a6 | b42 | c24 |
| 572 | a6 | b42 | c25 |
| 573 | a6 | b42 | c26 |
| 574 | a6 | c2 | c4 |
| 575 | a6 | c2 | c5 |
| 576 | a6 | c2 | c6 |
| 577 | a6 | c2 | c7 |
| 578 | a6 | c2 | c8 |
| 579 | a6 | c2 | c9 |
| 580 | a6 | c2 | c41 |
| 581 | a6 | c2 | c43 |
| 582 | a6 | c2 | c44 |
| 583 | a6 | c2 | c45 |
| 584 | a6 | c2 | c46 |
| 585 | a6 | c2 | c47 |
| 586 | a6 | c2 | c1 |
| 587 | a6 | c2 | c2 |
| 588 | a6 | c2 | c3 |
| 589 | a6 | c2 | c4 |
| 590 | a6 | c2 | c5 |
| 591 | a6 | c2 | c6 |
| 592 | a6 | c2 | c7 |
| 593 | a6 | c2 | c8 |
| 594 | a6 | c2 | c9 |
| 595 | a6 | c2 | c10 |
| 596 | a6 | c2 | c11 |
| 597 | a6 | c2 | c12 |
| 598 | a6 | c2 | c13 |
| 599 | a6 | c2 | c14 |
| 600 | a6 | c2 | c15 |
| 601 | a6 | c2 | c16 |
| 602 | a6 | c2 | c17 |
| 603 | a6 | c2 | c18 |
| 604 | a6 | c2 | c19 |
| 605 | a6 | c2 | c20 |
| 606 | a6 | c2 | c21 |
| 607 | a6 | c2 | c24 |
| 608 | a6 | c2 | c25 |
| 609 | a6 | c2 | c26 |
| 610 | a6 | c2 | b4 |
| 611 | a6 | c7 | b5 |
| 612 | a6 | c7 | b6 |
| 613 | a6 | c7 | b7 |
| 614 | a6 | c7 | b8 |
| 615 | a6 | c7 | b9 |
| 616 | a6 | c7 | b10 |
| 617 | a6 | c7 | b13 |
| 618 | a6 | c7 | b14 |
| 619 | a6 | c7 | b15 |
| 620 | a6 | c7 | b16 |
| 621 | a6 | c7 | b17 |
| 622 | a6 | c7 | b18 |
| 623 | a6 | c7 | b19 |
| 624 | a6 | c7 | b20 |
| 625 | a6 | c7 | b21 |
| 626 | a6 | c7 | b22 |
| 627 | a6 | c7 | b23 |
| 628 | a6 | c7 | b24 |
| 629 | a6 | c7 | b25 |
| 630 | a6 | c7 | b26 |
| 631 | a6 | c7 | b27 |
| 632 | a6 | c7 | b28 |
| 633 | a6 | c7 | b29 |
| 634 | a6 | c7 | b30 |
| 635 | a6 | c7 | b31 |
| 636 | a6 | c7 | b32 |
| 637 | a6 | c7 | b33 |
| 638 | a6 | c7 | b35 |
| 639 | a6 | c7 | b36 |
| 640 | a6 | c7 | b37 |
| 641 | a6 | c7 | b38 |
| 642 | a6 | c7 | b39 |
| 643 | a6 | c7 | b40 |
| 644 | a6 | c7 | b41 |
| 645 | a6 | c7 | b43 |
| 646 | a6 | c7 | b44 |
| 647 | a6 | c7 | b45 |
| 648 | a6 | c7 | b46 |
| 649 | a6 | c7 | b47 |
| 650 | a6 | c7 | c1 |
| 651 | a6 | c7 | c3 |
| 652 | a6 | c7 | c4 |
| 653 | a6 | c7 | c5 |
| 654 | a6 | c7 | c6 |
| 655 | a6 | c7 | c7 |
| 656 | a6 | c7 | c8 |
| 657 | a6 | c7 | c9 |
| 658 | a6 | c7 | c10 |
| 659 | a6 | c7 | c11 |
| 660 | a6 | c7 | c12 |
| 661 | a6 | c7 | c13 |
| 662 | a6 | c7 | c14 |
| 663 | a6 | c7 | c15 |
| 664 | a6 | c7 | c16 |
| 665 | a6 | c7 | c17 |
| 666 | a6 | c7 | c18 |
| 667 | a6 | c7 | c19 |
| 668 | a6 | c7 | c20 |
| 669 | a6 | c7 | c21 |
| 670 | a6 | c7 | c22 |
| 671 | a6 | c7 | c23 |
| 672 | a6 | c7 | c24 |
| 673 | a6 | c7 | c25 |
| 674 | a6 | c7 | c26 |
| 675 | a6 | c14 | c4 |
| 676 | a6 | c14 | c5 |
| 677 | a6 | c14 | b6 |
| 678 | a6 | c14 | b7 |
| 679 | a6 | c14 | b8 |
| 680 | a6 | c14 | b9 |
| 681 | a6 | c14 | b10 |
| 682 | a6 | c14 | b13 |
| 683 | a6 | c14 | b14 |
| 684 | a6 | c14 | b15 |
| 685 | a6 | c14 | b16 |
| 686 | a6 | c14 | b17 |
| 687 | a6 | c14 | b18 |
| 688 | a6 | c14 | b19 |
| 689 | a6 | c14 | b20 |
| 690 | a6 | c14 | b21 |
| 691 | a6 | c14 | b22 |
| 692 | a6 | c14 | b23 |
| 693 | a6 | c14 | b24 |
| 694 | a6 | c14 | b25 |
| 695 | a6 | c14 | b26 |
| 696 | a6 | c14 | b27 |
| 697 | a6 | c14 | b28 |
| 698 | a6 | c14 | b29 |
| 699 | a6 | c14 | b30 |
| 700 | a6 | c14 | b31 |
| 701 | a6 | c14 | b32 |
| 702 | a6 | c14 | b33 |
| 703 | a6 | c14 | b35 |
| 704 | a6 | c14 | b36 |
| 705 | a6 | c14 | b37 |
| 706 | a6 | c14 | b38 |
| 707 | a6 | c14 | b39 |
| 708 | a6 | c14 | b40 |
| 709 | a6 | c14 | b41 |
| 710 | a6 | c14 | b43 |
| 711 | a6 | c14 | b44 |
| 712 | a6 | c14 | b45 |
| 713 | a6 | c14 | b46 |
| 714 | a6 | c14 | b47 |
| 715 | a6 | c14 | b1 |
| 716 | a6 | c14 | b3 |
| 717 | a6 | c14 | b4 |
| 718 | a6 | c14 | c5 |
| 719 | a6 | c14 | c6 |
| 720 | a6 | c14 | c8 |
| 721 | a6 | c14 | c9 |
| 722 | a6 | c14 | c10 |
| 723 | a6 | c14 | c11 |
| 724 | a6 | c14 | c12 |
| 725 | a6 | c14 | c13 |
| 726 | a6 | c14 | c14 |
| 727 | a6 | c14 | c15 |
| 728 | a6 | c14 | c16 |
| 729 | a6 | c14 | c18 |
| 730 | a6 | c14 | c19 |
| 731 | a6 | c14 | c20 |
| 732 | a6 | c14 | c21 |
| 733 | a6 | c14 | c22 |
| 734 | a6 | c14 | c23 |
| 735 | a6 | c14 | c24 |
| 736 | a6 | c14 | c25 |
| 737 | a6 | c14 | c26 |
| 738 | a6 | c15 | b4 |
| 739 | a6 | c15 | b5 |
| 740 | a6 | c15 | b6 |
| 741 | a6 | c15 | b7 |
| 742 | a6 | c15 | b8 |
| 743 | a6 | c15 | b9 |
| 744 | a6 | c15 | b10 |
| 745 | a6 | c15 | b13 |
| 746 | a6 | c15 | b14 |
| 747 | a6 | c15 | b15 |
| 748 | a6 | c15 | b16 |
| 749 | a6 | c15 | b17 |
| 750 | a6 | c15 | b18 |
| 751 | a6 | c15 | b19 |
| 752 | a6 | c15 | b20 |
| 753 | a6 | c15 | b21 |
| 754 | a6 | c15 | b22 |
| 755 | a6 | c15 | b23 |
| 756 | a6 | c15 | b24 |
| 757 | a6 | c15 | b25 |
| 758 | a6 | c15 | b26 |
| 759 | a6 | c15 | b27 |
| 760 | a6 | c15 | b28 |
| 761 | a6 | c15 | b29 |
| 762 | a6 | c15 | b30 |
| 763 | a6 | c15 | b31 |
| 764 | a6 | c15 | b32 |
| 765 | a6 | c15 | b33 |
| 766 | a6 | c15 | b35 |
| 767 | a6 | c15 | b36 |
| 768 | a6 | c15 | b37 |
| 769 | a6 | c15 | b38 |
| 770 | a6 | c15 | b39 |
| 771 | a6 | c15 | b40 |
| 772 | a6 | c15 | b41 |
| 773 | a6 | c15 | b43 |
| 774 | a6 | c15 | b44 |
| 775 | a6 | c15 | b45 |
| 776 | a6 | c15 | b46 |
| 777 | a6 | c15 | b47 |
| 778 | a6 | c15 | c1 |
| 779 | a6 | c15 | c3 |
| 780 | a6 | c15 | c4 |
| 781 | a6 | c15 | c5 |
| 782 | a6 | c15 | c6 |
| 783 | a6 | c15 | c8 |
| 784 | a6 | c15 | c9 |
| 785 | a6 | c15 | c10 |
| 786 | a6 | c15 | c11 |
| 787 | a6 | c15 | c12 |
| 788 | a6 | c15 | c13 |
| 789 | a6 | c15 | c15 |
| 790 | a6 | c15 | c16 |
| 791 | a6 | c15 | c17 |
| 792 | a6 | c15 | c18 |
| 793 | a6 | c15 | c19 |
| 794 | a6 | c15 | c20 |
| 795 | a6 | c15 | c21 |
| 796 | a6 | c15 | c22 |
| 797 | a6 | c15 | c23 |
| 798 | a6 | c15 | c24 |
| 799 | a6 | c15 | c25 |
| 800 | a6 | c15 | c26 |
| 801 | a6 | c24 | b4 |
| 802 | a6 | c24 | b5 |
| 803 | a6 | c24 | b6 |
| 804 | a6 | c24 | b7 |
| 805 | a6 | c24 | b8 |
| 806 | a6 | c24 | b9 |
| 807 | a6 | c24 | b10 |
| 808 | a6 | c24 | b13 |
| 809 | a6 | c24 | b14 |
| 810 | a6 | c24 | b15 |
| 811 | a6 | c24 | b16 |
| 812 | a6 | c24 | b17 |
| 813 | a6 | c24 | b18 |
| 814 | a6 | c24 | b19 |
| 815 | a6 | c24 | b20 |
| 816 | a6 | c24 | b21 |
| 817 | a6 | c24 | b22 |
| 818 | a6 | c24 | b23 |
| 819 | a6 | c24 | b24 |
| 820 | a6 | c24 | b25 |
| 821 | a6 | c24 | b26 |
| 822 | a6 | c24 | b27 |
| 823 | a6 | c24 | b28 |
| 824 | a6 | c24 | b29 |
| 825 | a6 | c24 | b30 |
| 826 | a6 | c24 | b31 |
| 827 | a6 | c24 | b32 |
| 828 | a6 | c24 | b33 |
| 349 | a6 | c24 | b35 |
| 830 | a6 | c24 | b36 |
| 831 | a6 | c24 | b37 |
| 832 | a6 | c24 | b38 |
| 833 | a6 | c24 | b39 |
| 834 | a6 | c24 | b40 |
| 835 | a6 | c24 | b41 |
| 836 | a6 | c24 | b43 |
| 837 | a6 | c24 | b44 |
| 838 | a6 | c24 | b45 |
| 839 | a6 | c24 | b46 |
| 840 | a6 | c24 | b47 |
| 841 | a6 | c24 | c1 |
| 842 | a6 | c24 | c3 |
| 843 | a6 | c24 | c4 |
| 844 | a6 | c24 | c5 |
| 845 | a6 | c24 | c6 |
| 846 | a6 | c24 | c8 |
| 847 | a6 | c24 | c9 |
| 848 | a6 | c24 | c10 |
| 849 | a6 | c24 | c11 |
| 850 | a6 | c24 | c12 |
| 851 | a6 | c24 | c13 |
| 852 | a6 | c24 | c16 |
| 853 | a6 | c24 | c17 |
| 854 | a6 | c24 | c18 |
| 855 | a6 | c24 | c19 |
| 856 | a6 | c24 | c20 |
| 857 | a6 | c24 | c21 |
| 858 | a6 | c24 | c22 |
| 859 | a6 | c24 | c23 |
| 860 | a6 | c24 | c24 |
| 861 | a6 | c24 | c25 |
| 862 | a6 | c24 | c26 |
| 863 | a5 | b10 | c26 |
| 864 | a1 | b10 | c7 |
| 865 | a1 | b48 | c7 |
| 866 | a1 | b48 | c14 |
| 867 | a1 | b10 | c14 |
| 868 | a1 | b48 | c14 |
